(19) United States
(12) Patent Application Publication
(10) Pub. No.: US 2021/0370019 A1
(43) Pub. Date: Dec. 2, 2021

(54) Method of Treatment Using Negative Pressure Renal Therapy and Medicament(s)

(71) Applicant: Strataca Systems Limited, Floriana (MT)

(72) Inventors: John R. Erbey, II, Milton, GA (US); Jacob L. Upperco, Atlanta, GA (US); Bryan J. Tucker, Chapel Hill, NC (US)

(21) Appl. No.: 17/238,454

(22) Filed: Apr. 23, 2021

Related U.S. Application Data
(60) Provisional application No. 63/163,315, filed on Mar. 19, 2021.
(60) Provisional application No. 63/031,341, filed on May 28, 2020.

(51) Int. Cl. A61M 25/00; A61M 1/00; A61K 31/341

(52) U.S. Cl. CPC A61M 25/0017; A61M 25/005; A61M 25/007; A61M 1/90; A61M 2210/1089; A61K 31/341; A61M 2210/1082; A61M 2210/1085; A61M 1/84

(57) ABSTRACT

Disclosed herein is a method for increasing urine output and/or sodium output from a patient having venous congestion. The method includes (a) administering at least one medicament to a patient, wherein the medicament increases urine output and/or sodium output from the patient; and (b) applying negative pressure to a drainage lumen of a urinary catheter such that flow of urine from a ureter and/or kidney of the patient is transported within the drainage lumen to extract urine from the patient; wherein administering the at least one medicament occurs before, during and/or after applying negative pressure.

### CROSS REFERENCE TO RELATED APPLICATIONS

The present application claims priority to U.S. Provisional Patent Application No. 63/163,315, filed Mar. 19, 2021, and U.S. Provisional Patent Application No. 63/031,341, filed May 28, 2020, the disclosures of each of which are hereby incorporated by reference herein in their entireties.

### BACKGROUND

### Technical Field

The present disclosure relates to methods of treating patients with renal impairment and/or other medical conditions using one or more medicaments in coordination with inducing negative pressure in a patient's urinary tract through a catheter or stent.

### Background

The renal or urinary system includes a pair of kidneys, each kidney being connected by a ureter to the bladder, and a urethra for draining fluid or urine produced by the kidneys from the bladder. The kidneys perform several vital functions for the human body including, for example, filtering the blood to eliminate waste in the form of urine. The kidneys also regulate electrolytes (e.g., sodium, potassium and calcium) and metabolites, blood volume, blood pressure, blood pH, fluid volume, production of red blood cells, and bone metabolism. Adequate understanding of the anatomy and physiology of the kidneys is useful for understanding the impact that altered hemodynamics other fluid overload conditions have on their function.

In normal anatomy, the two kidneys are located retroperitoneally in the abdominal cavity. The kidneys are bean-shaped encapsulated organs. Urine is formed by nephrons, the functional unit of the kidney, and then flows through a system of converging tubules called collecting ducts. The collecting ducts join together to form minor calyces, then major calyces, which ultimately join near the concave portion of the kidney (renal pelvis). A major function of the renal pelvis is to direct urine flow to the ureter. Urine flows from the renal pelvis into the ureter, a tube-like structure that carries the urine from the kidneys into the bladder. The outer layer of the kidney is called the cortex, and is a rigid fibrous encapsulation. The interior of the kidney is called the medulla. The medulla structures are arranged in pyramids.

Each kidney is made up of approximately one million nephrons. Each nephron includes the glomerulus, Bowman's capsule, and tubules. The tubules include the proximal convoluted tubule, the loop of Henle, the distal convoluted tubule, and the collecting duct. The nephrons contained in the cortex layer of the kidney are distinct from the anatomy of those contained in the medulla. The principal difference is the length of the loop of Henle. Medullary nephrons contain a longer loop of Henle, which, under normal circumstances, allows greater regulation of water and sodium reabsorption than in the cortex nephrons.

The glomerulus is the beginning of the nephron, and is responsible for the initial filtration of blood. Afferent arterioles pass blood into the glomerular capillaries, where hydrostatic pressure pushes water and solutes into Bowman's capsule. Net filtration pressure is expressed as the hydrostatic pressure in the afferent arteriole minus the hydrostatic pressure in Bowman's space minus the osmotic pressure in the efferent arteriole.

Net Filtration Pressure=Hydrostatic Pressure (Afferent Arteriole)−Hydrostatic Pressure (Bowman's Space)−Osmotic Pressure (Efferent Arteriole) (Equation 1)

The magnitude of this net filtration pressure defined by Equation 1 determines how much ultra-filtrate is formed in Bowman's space and delivered to the tubules. The remaining blood exits the glomerulus via the efferent arteriole. Normal glomerular filtration, or delivery of ultra-filtrate into the tubules, is about 90 ml/min/1.73 m².

The glomerulus has a three-layer filtration structure, which includes the vascular endothelium, a glomerular basement membrane, and podocytes. Normally, large proteins such as albumin and red blood cells, are not filtered into Bowman's space. However, elevated glomerular pressures and mesangial expansion create surface area changes on the basement membrane and larger fenestrations between the podocytes allowing larger proteins to pass into Bowman's space.

Ultra-filtrate collected in Bowman's space is delivered first to the proximal convoluted tubule. Re-absorption and secretion of water and solutes in the tubules is performed by a mix of active transport channels and passive pressure gradients. The proximal convoluted tubules normally reabsorb a majority of the sodium chloride and water, and nearly all glucose and amino acids that were filtered by the glomerulus. The loop of Henle has two components that are designed to concentrate wastes in the urine. The descending limb is highly water permeable and reabsorbs most of the remaining water. The ascending limb reabsorbs 25% of the remaining sodium chloride, creating a concentrated urine, for example, in terms of urea and creatinine. The distal convoluted tubule normally reabsorbs a small proportion of sodium chloride, and the osmotic gradient creates conditions for the water to follow.

Under normal conditions, there is a net filtration of approximately 14 mm Hg. The impact of venous congestion can be a significant decrease in net filtration, down to approximately 4 mm Hg. See Jessup M., The cardiorenal syndrome: Do we need a change of strategy or a change of tactics?, JACC 53(7):597-600, 2009 (hereinafter “Jessup”). Venous congestion is a common complication of renal insufficiency, heart failure, traumatic injuries and surgery. Prolonged elevated venous pressure can result in distention, edema, stasis, ischemia and/or cellular death. Venous congestion can be determined by observation of symptoms, such as edema, or direct or indirect measurement, as is well known to those skilled in the art. For example, the central venous pressure, which is a measure of pressure in the vena cava, can be measured using a central venous catheter advanced via the internal jugular vein and placed in the superior vena cava near the right atrium. A normal central venous pressure reading is between 0 to 6 mmHg. This value is altered by volume status and/or venous compliance. Alternatively, venous congestion can be measured by jugular venous distension (JVD). While the patient is lying down on an exam table, with the head of the table at a 45-degree angle and head turned to the side, the doctor measures the highest point at which pulsations can be detected in the internal jugular vein. Alternatively, the Venus Excess Ultrasound (VExUS) score (0-3) can be determined using ultrasound, or the distensibility of the inferior vena cava can be measured via ultrasound. A NT-proB-type Natriuretic Peptide (BNP) blood test can provide an assessment of congestion caused by elevated venous pressures.

The second filtration stage occurs at the proximal tubules. Most of the secretion and absorption from urine occurs in tubules in the medullary nephrons. Active transport of sodium from the tubule into the interstitial space initiates this process. However, the hydrostatic forces dominate the net exchange of solutes and water. Under normal circumstances, it is believed that 75% of the sodium is reabsorbed back into lymphatic or venous circulation. However, because the kidney is encapsulated, it is sensitive to changes in hydrostatic pressures from both venous and lymphatic congestion. During venous congestion the retention of sodium and water can exceed 85%, further perpetuating the renal congestion. See Verbrugge, et al., The kidney in congestive heart failure: Are natriuresis, sodium, and diuretics really the good, the bad and the ugly? European Journal of Heart Failure 2014:16, 133-42 (hereinafter “Verbrugge”).

Venous congestion can lead to a prerenal form of acute kidney injury (AKI). Prerenal AKI is due to a loss of perfusion (or loss of blood flow) through the kidney. Many clinicians focus on the lack of flow into the kidney due to shock. However, there is also evidence that a lack of blood flow out of the organ due to venous congestion can be a clinically important sustaining injury. See Damman K, Importance of venous congestion for worsening renal function in advanced decompensated heart failure, JACC 17:589-96, 2009 (hereinafter “Damman”).

Prerenal AKI occurs across a wide variety of diagnoses requiring critical care admissions. The most prominent admissions are for sepsis and Acute Decompensated Heart Failure (ADHF). Additional admissions include cardiovascular surgery, general surgery, cirrhosis, trauma, burns, and pancreatitis. While there is wide clinical variability in the presentation of these disease states, a common denominator is an elevated central venous pressure. In the case of ADHF, the elevated central venous pressure caused by heart failure leads to pulmonary edema, and, subsequently, dyspnea in turn precipitating the admission. In the case of sepsis, the elevated central venous pressure is largely a result of aggressive fluid resuscitation. Whether the primary insult was low perfusion due to hypovolemia or sodium and fluid retention, the sustaining injury is the venous congestion resulting in inadequate perfusion.

Hypertension is another widely recognized state that creates perturbations within the active and passive transport systems of the kidney(s). Hypertension directly impacts afferent arteriole pressure and results in a proportional increase in net filtration pressure within the glomerulus. The increased filtration fraction also elevates the peritubular capillary pressure, which stimulates sodium and water re-absorption. See Verbrugge.

Because the kidney is an encapsulated organ, it is sensitive to pressure changes in the medullary pyramids. The elevated renal venous pressure creates congestion that leads to a rise in the interstitial pressures. The elevated interstitial pressures exert forces upon both the glomerulus and tubules. See Verbrugge. In the glomerulus, the elevated interstitial pressures directly oppose filtration. The increased pressures increase the interstitial fluid, thereby increasing the hydrostatic pressures in the interstitial fluid and peritubular capillaries in the medulla of the kidney. In both instances, hypoxia can ensue leading to cellular injury and further loss of perfusion. The net result is a further exacerbation of the sodium and water re-absorption creating a negative feedback. See Verbrugge, 133-42. Fluid overload, particularly in the abdominal cavity is associated with many diseases and conditions, including elevated intra-abdominal pressure, abdominal compartment syndrome, and acute renal failure. Fluid overload can be addressed through renal replacement therapy. See Peters, C. D., Short and Long-Term Effects of the Angiotensin II Receptor Blocker Irbesartanon Intradialytic Central Hemodynamics: A Randomized Double-Blind Placebo-Controlled One-Year Intervention Trial (the SAFIR Study), PLoS ONE (2015) 10(6): e0126882. doi:10.1371/journal.pone.0126882 (hereinafter “Peters”). However, such a clinical strategy provides no improvement in renal function for patients with the cardiorenal syndrome. See Bart B, Ultrafiltration in decompensated heart failure with cardiorenal syndrome, NEJM 2012; 367:2296-2304 (hereinafter “Bart”).

Even among the best medical centers, nearly half of all patients admitted for congestion due to ADHF are discharged without achieving clinical decongestion, even with administration of high dose intravenous diuretics. Circ Heart Fail., 8(4), 741-748 (2015). The success in achieving decongestion or avoiding major clinical events for patients with any form of diuretic resistance are significantly worse.

Impaired renal sodium excretion secondary to neurohumoral upregulation is the primary abnormality. The body is composed of semipermeable membranes that allow water, but not ions, to move freely. Sodium accumulation, therefore, is required to precipitate volume overload. Presentation with clinical congestion, therefore, underscores the inability of the kidneys to appropriately regulate sodium and water in the body.

Heart failure is a medical condition where the heart is unable to maintain a sufficient blood flow to support the body. The signs and symptoms of heart failure include, but are not limited to, shortness of breath, fatigue, weakness, swelling in the legs, ankles and feet, rapid and/or irregular heartbeat, persistent cough or wheezing, blood tinged phlegm, increased urine output (especially at night), abdominal swelling, fluid retention, loss of appetite and nausea, loss of concentration and alertness, sudden and/or severe shortness of breath and/or chest pain.

One common symptom in heart failure is edema (i.e., fluid buildup in the patient). This occurs when excess fluid is trapped in the tissues of the body. When blood is not pumped properly during heart failure, blood and fluid can back up in the legs, ankles and feet of a patient. It can also result in swelling in the abdomen along with sudden weight gain due to fluid buildup. Pulmonary edema occurs when fluid builds up in the lungs of a patient which contributes to shortness of breath and respiratory symptoms.

There is a need for improved methods for treating patients with renal impairment and/or other medical conditions, including but not limited to venous congestion, fluid overload, fluid retention, and edema. Also, there is also a need to increase renal blood flow and diuretic effectiveness in a patient.

### SUMMARY

The present disclosure improves upon previous methods of treating patients with renal impairment and/or other medical conditions by providing at least one medicament for use in coordination with application of negative pressure to a urinary tract of a patient to facilitate urine and/or sodium output.

In some examples, provided herein is a method for increasing urine output and/or sodium output from a patient having venous congestion, the method comprising: (a) administering at least one medicament to a patient, wherein the medicament increases urine output and/or sodium output from the patient; and (b) applying negative pressure to a drainage lumen of a urinary catheter such that flow of urine from a ureter and/or kidney of the patient is transported within the drainage lumen to extract urine from the patient; wherein administering the at least one medicament occurs before, during and/or after applying negative pressure.

In some examples, provided herein is a method for increasing urine output and/or sodium output from a patient having venous congestion, the method comprising: (a) administering at least one medicament to a patient, wherein the medicament increases urine output and/or sodium output from the patient; (b) deploying at least one urinary catheter into the patient such that flow of urine from the ureter and/or kidney of the patient is transported within a drainage lumen of the catheter, and (c) applying negative pressure to the drainage lumen of the catheter to extract urine from the patient; wherein administering the at least one medicament occurs before, during and/or after applying negative pressure.

Non-limiting examples, aspects or embodiments of the present invention will now be described in the following numbered clauses:

Clause 1: A method for increasing urine output and/or sodium output from a patient having venous congestion, the method comprising: (a) administering at least one medicament to a patient, wherein the medicament increases urine output and/or sodium output from the patient; and (b) applying negative pressure to a drainage lumen of a urinary catheter such that flow of urine from a ureter and/or kidney of the patient is transported within the drainage lumen to extract urine from the patient; wherein administering the at least one medicament occurs before, during and/or after applying negative pressure.

Clause 2: The method according to clause 1, wherein the at least one medicament is selected from the group consisting of diuretic(s), SGLT-2 inhibitor(s), and combinations thereof.

Clause 3: The method according to clause 2, wherein the at least one medicament comprises at least one diuretic.

Clause 4: The method according to clauses 2 or 3, wherein the at least one diuretic is selected from the group consisting of loop diuretic(s), carbonic anhydrase inhibitor(s), potassium-sparing diuretic(s), calcium-sparing diuretic(s), osmotic diuretic(s), thiazide diuretic(s), and combinations thereof.

Clause 5: The method according to any of clauses 2 to 4, wherein the at least one diuretic comprises at least one loop diuretic.

Clause 6: The method according to any of clauses 4 or 5, wherein the at least one loop diuretic is selected from the group consisting of bumetanide, ethacrynic acid, torsemide, furosemide, and combinations thereof.

Clause 7. The method according to any of clauses 4 to 6, wherein the at least one loop diuretic is furosemide.

Clause 8. The method according to clause 2, wherein the at least one medicament comprises at least one SGLT-2 inhibitor.

Clause 9. The method according to clause 8, wherein the at least one SGLT-2 inhibitor is selected from the group consisting of ertugliflozin, canagliflozin, empagliflozin, dapagliflozin, and combinations thereof.

Clause 10. The method according to any of clauses 2 to 9, wherein the patient is administered at least two medicaments, wherein each of the at least two medicaments is independently selected from the group consisting diuretic(s), SGLT-2 inhibitor(s), and combinations thereof.

Clause 11. The method according to clause 10, wherein the patient is administered at least one diuretic and at least one SGLT-2 inhibitor.

Clause 12. The method according to any of clauses 10 or 11, wherein the at least one diuretic is furosemide.

Clause 13. The method according to any of the preceding clauses, wherein the patient has fluid overload.

Clause 14. The method according to any of the preceding clauses, wherein the patient has kidney impairment and/or reduced kidney function.

Clause 15. The method according to any of the preceding clauses, wherein the patient has chronic kidney disease or acute kidney injury.

Clause 16. The method according to any of the preceding clauses, wherein the patient has a GFR of 90 or lower, or a GFR of 60 or lower, or GFR of 44 or lower, or GFR of 30 or lower, prior to treatment.

Clause 17. The method according to any of the preceding clauses, wherein the patient has heart failure or acute decompensated heart failure.

Clause 18. The method according to any of the preceding clauses, wherein the patient has sepsis.

Clause 19. The method according to any of the preceding clauses, wherein the urinary catheter further comprises: a proximal portion, a distal portion, and a drainage lumen, the distal portion, the distal portion comprising a retention portion that comprises at least one protected drainage hole(s), port(s) or perforation(s) and is configured to establish an outer periphery or protective surface area that inhibits mucosal tissue from occluding the at least one protected drainage hole(s), port(s) or perforation(s) upon application of negative pressure through the catheter.

Clause 20. The method according to clause 19, wherein the proximal portion of the urinary catheter is configured to pass through a percutaneous opening.

Clause 21. The method according to clauses 19 or 20, wherein the at least one protected drainage hole(s), port(s) or perforation(s) are disposed on a protected surface area or inner surface area of the retention portion, and wherein the outer periphery or protective surface area of the retention portion of the catheter is configured to support the mucosal tissue and thereby prevent occlusion of the one or more of the protected drainage holes, ports or perforations upon application of negative pressure through the ureteral catheter.

Clause 22. The method according to any of clauses 19 to 21, wherein the retention portion comprises one or more helical coils, each coil having an outwardly facing side and an inwardly facing side, and wherein the outer periphery or protective surface area comprises the outwardly facing side(s) of the one or more helical coil(s), and the at least one protected drainage hole(s), port(s) or perforation(s) are disposed on the inwardly facing side(s) of the one or more helical coil(s).

Clause 23. The method according to any of clauses 19 to 22, wherein the retention portion is configured to be extended into a deployed position in which a diameter of the retention portion is greater than a diameter of the drainage lumen portion.

Clause 24. The method according to any of clauses 19 to 23, wherein a number of the drainage holes, ports or perforations towards a distal end of the retention portion is greater that a number of the drainage holes, ports or perforations towards a proximal end of the retention portion.

Clause 25. The method according to any of clauses 19 to 24, wherein a size of one or more of the drainage holes, ports or perforations towards a distal end of the retention portion is greater that a size of one or more of the drainage holes, ports or perforations towards a proximal end of the retention portion.

Clause 26. The method according to any of clauses 19 to 25, wherein a total area of the drainage holes, ports or perforations towards a distal end of the retention portion is greater than a total area of the drainage holes, ports or perforations towards a proximal end of the retention portion.

Clause 27. The method according to any of clauses 19 to 26, wherein a sidewall of the drainage lumen is essentially free or free of openings.

Clause 28. The method according to any of clauses 1-27, wherein the at least one urinary catheter comprises at least one ureteral catheter and/or a bladder catheter.

Clause 29. The method according to any of clauses 19 to 28, further comprising a negative pressure source operatively connected to the catheter, the negative pressure source being configured to induce negative pressure in a portion of the urinary tract of the patient which causes fluid from the urinary tract to be drawn into the catheter at least partially through the one or more protected drainage holes, ports or perforations.

Clause 30. The method according to any of clauses 1-29, wherein the negative pressure source is positioned within or outside of the patient's body.

Clause 31. The method according to any of clauses 1-30, wherein the negative pressure source is a pump.

Clause 32. The method according to clause 29, further comprising a controller that is operatively connected to the negative pressure source and configured to actuate the negative pressure source to control the application of the negative pressure to a proximal end of the at least one urinary catheter.

Clause 33. The method according to clause 32, wherein the controller is positioned within or outside of the patient's body.

Clause 34. The method according to any of clauses 29-33, further comprising one or more physiological sensors associated with the patient, the physiological sensors being configured to provide information representative of at least one physical parameter to the controller, and wherein the controller is configured to actuate or cease operation of the pump based on the information representative of the at least one physical parameter.

Clause 35. The method according to any of clauses 1-34, wherein negative pressure is provided with a range of about 0.5 mm Hg to about 150 mm Hg.

Clause 36. The method according to any of clauses 1 to 35, wherein the catheter is a ureteral catheter.

Clause 37. The method according to any of clauses 1 to 35, wherein the catheter is a bladder catheter.

Clause 38. A method for increasing urine output and/or sodium output from a patient having venous congestion, the method comprising: (a) administering a therapeutically effective amount of a medicament comprising furosemide to the patient; and (b) applying negative pressure to a drainage lumen of a ureteral catheter to extract urine from the patient, the ureteral catheter comprising a proximal portion; a distal portion, and a drainage lumen, wherein the distal portion of the catheter comprises a retention portion comprising at least one protected drainage hole(s), port(s) or perforation(s) and is configured to establish an outer periphery or protective surface area that inhibits mucosal tissue from occluding the at least one protected drainage hole(s), port(s) or perforation(s) upon application of negative pressure through the catheter; wherein administering the loop diuretic occurs before inducing negative pressure in the urinary tract of the patient.

### BRIEF DESCRIPTION OF THE DRAWINGS

These and other features and characteristics of the present disclosure, as well as the methods of operation and functions of the related elements of structures and the combination of parts and economies of manufacture, will become more apparent upon consideration of the following description and the appended clauses with reference to the accompanying drawings, all of which form a part of this specification, wherein like reference numerals designate corresponding parts in the various figures. It is to be expressly understood, however, that the drawings are for the purpose of illustration and description only and are not intended as a definition of the limit of the invention.

Further features and other examples and advantages will become apparent from the following detailed description made with reference to the drawings in which:

FIG. 1A is a schematic drawing of an indwelling portion of a system comprising a ureteral stent and a bladder catheter deployed in a urinary tract of a patient, according to an example of the present invention;

FIG. 1B is a schematic drawing of an indwelling portion of a system comprising a ureteral catheter and a bladder catheter deployed in a urinary tract of a patient, according to an example of the present invention;

FIG. 1C is a schematic drawing of an indwelling portion of a system comprising a ureteral catheter and a bladder catheter deployed in a urinary tract of a patient, according to an example of the present invention;

FIG. 1D is a perspective view of a retention portion of a bladder catheter, according to an example of the present invention;

FIG. 1E is a cross sectional view of the retention portion of FIG. 1D, taken along line 1E-1E of FIG. 1D, according to an example of the present invention;

FIG. 1F is a schematic drawing of an indwelling portion of a system comprising a ureteral catheter and a bladder catheter deployed in a urinary tract of a patient, according to an example of the present invention;

FIG. 1G is a perspective view of a retention portion of a bladder catheter, according to an example of the present invention;

FIG. 1H is a side elevational view of the retention portion of FIG. 1G, according to an example of the present invention;

FIG. 1I is a top plan view of the retention portion of FIG. 1G, according to an example of the present invention;

FIG. 1J is a perspective view of a retention portion of a bladder catheter, according to an example of the present invention;

FIG. 1K is a perspective view of a retention portion of a bladder catheter, according to an example of the present invention;

FIG. 1L is a side elevational view of a retention portion of a bladder catheter prior to deployment, according to an example of the present invention;

FIG. 1M is a side elevational view of the retention portion of FIG. 1L after deployment, according to an example of the present invention;

FIG. 1N is a perspective view of a retention portion of a bladder catheter, according to an example of the present invention;

FIG. 1O is a cross-sectional view of a portion of the retention portion of FIG. 1N, according to an example of the present invention;

FIG. 1P is a schematic drawing of an indwelling portion of a system comprising a ureteral catheter and a bladder catheter deployed in a urinary tract of a patient, according to an example of the present invention;

FIG. 1Q is a perspective view of a retention portion of a bladder catheter, according to an example of the present invention;

FIG. 1R is a cross-sectional view of a portion of the retention portion of FIG. 1Q, according to an example of the present invention;

FIG. 1S is a perspective view of a retention portion of a bladder catheter, according to an example of the present invention;

FIG. 1T is a cross-sectional view of a portion of the retention portion of FIG. 1S, according to an example of the present invention;

FIG. 1U is a schematic drawing of an indwelling portion of a system comprising a ureteral catheter and a bladder catheter deployed in a urinary tract of a patient, according to an example of the present invention;

FIG. 1V is a perspective view of a retention portion of a bladder catheter, according to an example of the present invention;

FIG. 1W is a cross sectional view of the retention portion of FIG. 1V, taken along line 1W-1W of FIG. 1V, according to an example of the present invention;

FIG. 2A is a schematic drawing of an indwelling portion of a system comprising a ureteral catheter deployed in a urinary tract of a patient, according to an example of the present invention;

FIG. 2B is a schematic drawing of an indwelling portion of a system comprising a ureteral catheter deployed in a urinary tract of a patient, according to an example of the present invention;

FIG. 3 is a dimetric view of an example of a prior art transformable ureteral stent according to FIG. 1 of PCT Patent Application Publication WO 2017/019974, wherein the image on the left represents the uncompressed state of the stent and the image on the right represents the compressed state of the stent;

FIG. 4 is a perspective view of an example of a prior art ureteral stent according to FIG. 4 of US Patent Application Publication No. 2002/0183853 A1;

FIG. 5 is a perspective view of an example of a prior art ureteral stent according to FIG. 5 of US Patent Application Publication No. 2002/0183853 A1;

FIG. 6 is a perspective view of an example of a prior art ureteral stent according to FIG. 7 of US Patent Application Publication No. 2002/0183853 A1;

FIG. 7A is a schematic drawing of another example of an indwelling portion of a system comprising a ureteral catheter and a bladder catheter deployed in a urinary tract of a patient, according to an example of the present invention;

FIG. 7B is a schematic drawing of a system for inducing negative pressure to the urinary tract of a patient according to an example of the present invention;

FIG. 7C is a an enlarged schematic drawing of a portion of a ureteral catheter according to the present invention positioned in the renal pelvis region of the kidney showing in phantom general changes believed to occur in the renal pelvis tissue in response to application of negative pressure through the ureteral catheter;

FIG. 8A is a perspective view of an exemplary catheter according to an example of the present invention;

FIG. 8B is a front view of the catheter of FIG. 8A;

FIG. 9A is a schematic drawing of an example of a retention portion for a catheter according to an example of the present invention;

FIG. 9B is a schematic drawing of another example of a retention portion for a catheter according to an example of the present invention;

FIG. 9C is a schematic drawing of another example of a retention portion for a catheter according to an example of the present invention;

FIG. 9D is a schematic drawing of another example of a retention portion for a catheter according to an example of the present invention;

FIG. 9E is a schematic drawing of another example of a retention portion for a catheter according to an example of the present invention;

FIG. 10 is a front view of another example of a catheter according to an example of the present invention;

FIG. 10A is a perspective view of the retention portion of the catheter of FIG. 10 enclosed by circle 10A according to an example of the present invention;

FIG. 10B is a front view of the retention portion of FIG. 10A according to an example of the present invention;

FIG. 10C is a rear view of the retention portion of FIG. 10A according to an example of the present invention;

FIG. 10D is a top view of the retention portion of FIG. 10A according to an example of the present invention;

FIG. 10E is a cross sectional view of the retention portion of FIG. 10A taken along line 10E-10E according to an example of the present invention;

FIG. 10F is s a cross sectional view of the retention portion of FIG. 10A taken along line 10E-10E according to an example of the present invention positioned in the renal pelvis region of the kidney showing in general changes believed to occur in the renal pelvis tissue in response to application of negative pressure through the ureteral catheter;

FIG. 10G is s a cross sectional view of the retention portion of FIG. 10A taken along line 10E-10E according to an example of the present invention positioned in the bladder showing in general changes believed to occur in the bladder tissue in response to application of negative pressure through the bladder catheter;

FIG. 11 is a schematic drawing of a retention portion of a catheter in a constrained or linear position according to an example of the present invention;

FIG. 12 is a schematic drawing of another example of a retention portion of a catheter in a constrained or linear position according to an example of the present invention;

FIG. 13 is a schematic drawing of another example of a retention portion of a ureteral catheter in a constrained or linear position according to an example of the present invention;

FIG. 14 is a schematic drawing of another example of a retention portion of a catheter in a constrained or linear position according to an example of the present invention;

FIG. 15A is a graph showing a percentage of fluid flow through openings of an exemplary catheter as a function of position according to an example of the present invention;

FIG. 15B is a graph showing a percentage of fluid flow through openings of another exemplary catheter as a function of position according to an example of the present invention;

FIG. 15C is a graph showing a percentage of fluid flow through openings of another exemplary catheter as a function of position according to an example of the present invention;

FIG. 16 is a schematic drawing of a retention portion of a catheter showing stations for calculating fluid flow coefficients for a mass transfer balance evaluation according to an example of the present invention;

FIG. 17 is a schematic drawing of an indwelling portion of a system comprising a ureteral catheter and a bladder catheter deployed in a urinary tract of a patient, according to another example of the present invention;

FIG. 18A is side elevational view of a retention portion of a catheter according to an example of the present invention;

FIG. 18B is cross-sectional view of the retention portion of the catheter of FIG. 18A taken along lines B-B of FIG. 18A;

FIG. 18C is a top plan view of the retention portion of the catheter of FIG. 18A taken along lines C-C of FIG. 18A;

FIG. 18D is cross sectional view of a retention portion of a ureteral catheter according to an example of the present invention positioned in the renal pelvis region of the kidney showing in general changes believed to occur in the renal pelvis tissue in response to application of negative pressure through the ureteral catheter;

FIG. 18E is cross sectional view of a retention portion of a bladder catheter according to an example of the present invention positioned in the bladder showing in general changes believed to occur in the bladder tissue in response to application of negative pressure through the bladder catheter;

FIG. 19 is a side elevational view of a retention portion of another catheter according to an example of the present invention;

FIG. 20 is a side elevational view of a retention portion of another catheter according to an example of the present invention;

FIG. 21 is a side elevational view of a retention portion of another catheter according to an example of the present invention;

FIG. 22A is a perspective view of a retention portion of another ureteral catheter according to an example of the present invention;

FIG. 22B is a top plan view of the retention portion of the catheter of FIG. 22A taken along lines 22B-22B of FIG. 22A;

FIG. 23A is a perspective view of a retention portion of another catheter according to an example of the present invention;

FIG. 23B is a top plan view of the retention portion of the catheter of FIG. 23A taken along lines 23B-23B of FIG. 23A;

FIG. 24A is a perspective view of a retention portion of another catheter according to an example of the present invention;

FIG. 24B is a cross sectional view of a retention portion of a ureteral catheter according to an example of the present invention positioned in the renal pelvis region of the kidney showing in general changes believed to occur in the renal pelvis tissue in response to application of negative pressure through the ureteral catheter;

FIG. 24C is a cross sectional view of a retention portion of a bladder catheter according to an example of the present invention positioned in the bladder showing in general changes believed to occur in the bladder tissue in response to application of negative pressure through the bladder catheter;

FIG. 25 is a side elevational view of a retention portion of another catheter according to an example of the present invention;

FIG. 26 is a side elevational view of a retention portion of another catheter according to an example of the present invention;

FIG. 27 is a cross-sectional side view of a retention portion of another catheter according to an example of the present invention;

FIG. 28A is a perspective view of a retention portion of another catheter according to an example of the present invention;

FIG. 28B is a top plan view of the retention portion of the catheter of FIG. 28A;

FIG. 29A is a perspective view of a retention portion of another catheter according to an example of the present invention;

FIG. 29B is a top plan view of the retention portion of the catheter of FIG. 29A;

FIG. 29C is a cross sectional view of a retention portion of a ureteral catheter according to an example of the present invention positioned in the renal pelvis region of the kidney showing in general changes believed to occur in the renal pelvis tissue in response to application of negative pressure through the ureteral catheter;

FIG. 30 is a perspective view of a retention portion of another catheter according to an example of the present invention;

FIG. 31 is a top plan view of the retention portion of the catheter of FIG. 30;

FIG. 32A is a perspective view of a retention portion of another catheter according to an example of the present invention;

FIG. 32B is a top plan view of the retention portion of the catheter of FIG. 32A;

FIG. 33 is a cross-sectional side elevational view of a retention portion of another catheter according to an example of the present invention;

FIG. 34 is a cross-sectional side elevational view of a retention portion of another catheter according to an example of the present invention;

FIG. 35A is a perspective view of a retention portion of another catheter according to an example of the present invention;

FIG. 35B is a cross-sectional side elevational view of the retention portion of the catheter of FIG. 35A taken along lines B-B of FIG. 35A;

FIG. 36 is a side elevational view showing a cut away cross-sectional view of the sheath surrounding a catheter according to an example of the present invention in a contracted configuration for insertion into a patient's ureter;

FIG. 37A is a schematic drawing of another example of a retention portion for a catheter according to an example of the present invention;

FIG. 37B is a schematic drawing of a cross-sectional view of a portion of the retention portion of FIG. 37A, taken along lines B-B of FIG. 37A;

FIG. 38A is a schematic drawing of another example of a retention portion for a catheter according to an example of the present invention;

FIG. 38B is a schematic drawing of a portion of a cross-sectional view of the retention portion of FIG. 5A, taken along lines B-B of FIG. 38A;

FIG. 39A is a schematic drawing of another example of a retention portion for a catheter according to an example of the present invention;

FIG. 39B is a schematic drawing of a cross section of another example of a retention portion for a ureteral catheter according to an example of the present invention positioned in the renal pelvis region of the kidney showing in general changes believed to occur in the renal pelvis tissue in response to application of negative pressure through the ureteral catheter;

FIG. 39C is a schematic drawing of a cross section of another example of a retention portion for a bladder catheter according to an example of the present invention positioned in the bladder showing in general changes believed to occur in the bladder tissue in response to application of negative pressure through the bladder catheter;

FIG. 40A is a schematic drawing of a cross section of another example of a retention portion for a catheter according to an example of the present invention;

FIG. 40B is a schematic drawing of a cross section of another example of a retention portion for a ureteral catheter according to an example of the present invention positioned in the renal pelvis region of the kidney showing in general changes believed to occur in the renal pelvis tissue in response to application of negative pressure through the ureteral catheter;

FIG. 40C is a schematic drawing of a cross section of another example of a retention portion for a bladder catheter according to an example of the present invention positioned in the bladder showing in general changes believed to occur in the bladder tissue in response to application of negative pressure through the bladder catheter;

FIG. 41A is a schematic drawing of another example of a retention portion for a catheter according to an example of the present invention;

FIG. 41B is a schematic drawing of a cross section of another example of a retention portion for a ureteral catheter according to an example of the present invention positioned in the renal pelvis region of the kidney showing in general changes believed to occur in the renal pelvis tissue in response to application of negative pressure through the ureteral catheter;

FIG. 41C is a schematic drawing of a cross section of another example of a retention portion for a bladder catheter according to an example of the present invention positioned in the bladder showing in general changes believed to occur in the bladder tissue in response to application of negative pressure through the bladder catheter;

FIG. 42A is a flow chart illustrating a process for insertion and deployment of a system according to an example of the present invention;

FIG. 42B is a flow chart illustrating a process for applying negative pressure using a system according to an example of the present invention;

FIG. 43 is a schematic drawing of a nephron and surrounding vasculature showing a position of the capillary bed and convoluted tubules;

FIG. 44 is a schematic drawing of a system for inducing negative pressure to the urinary tract of a patient according to an example of the present invention;

FIG. 45A is a plan view of a pump for use with the system of FIG. 44 according to an example of the present invention;

FIG. 45B is a side elevation view of the pump of FIG. 45A;

FIG. 46 is a schematic drawing of an experimental set-up for evaluating negative pressure therapy in a swine model according to the present invention;

FIG. 47 is a graph of creatinine clearance rates for tests conducted using the experimental set-up shown in FIG. 21;

FIG. 48A is a low magnification photomicrograph of kidney tissue from a congested kidney treated with negative pressure therapy;

FIG. 48B is a high magnification photomicrograph of the kidney tissue shown in FIG. 48A;

FIG. 48C is a low magnification photomicrograph of kidney tissue from a congested and untreated (e.g., control) kidney;

FIG. 48D is a high magnification photomicrograph of the kidney tissue shown in FIG. 23C

FIG. 49 is a flow chart illustrating a process for reducing creatinine and/or protein levels of a patient according to an example of the present invention;

FIG. 50 is a flow chart illustrating a process for treating a patient undergoing fluid resuscitation according to an example of the present invention;

FIG. 51 is a graph of serum albumin relative to baseline for tests conduct on swine using the experimental method described herein;

FIG. 52A is a perspective view of a ureteral catheter including a multi-functional coating, according to an aspect of the invention;

FIG. 52B is a side view of the catheter of FIG. 52A;

FIG. 53 is a cross-sectional view of a portion of the ureteral catheter of FIG. 52A in a linear, uncoiled state;

FIG. 54 is a cross-sectional view of a portion of the ureteral catheter of FIG. 52A in a deployed or coiled state;

FIG. 55 is a flow chart of a method of manufacture for a coated ureteral catheter, according to an aspect of the disclosure;

FIG. 56 is a cross-sectional view of a portion of a ureteral catheter in a linear, uncoiled state, including another exemplary multi-functional coating, according to an aspect of the disclosure;

FIG. 57 is a cross sectional view of a portion of a ureteral catheter in a linear, uncoiled state, including another exemplary multi-functional coating, according to an aspect of the disclosure;

FIG. 58 is a flow chart of the experimental procedure of Example 4, according to an aspect of the disclosure;

FIG. 59A is a graph of the urine output (ml) comparing pigs with administration of a diuretic with and without negative pressure renal treatment, according to an aspect of the disclosure;

FIG. 59B is a graph of the sodium levels (mmol) comparing pigs with administration of a diuretic with and without negative pressure renal treatment, according to an aspect of the disclosure;

FIG. 59C is a graph of the creatinine clearance comparing pigs with administration of a diuretic with and without negative pressure renal treatment, according to an aspect of the disclosure; and

FIG. 59D is a graph of the pressure profile of CO (L/min) in pigs with and without the administration of a diuretic and negative pressure renal therapy, according to an aspect of the disclosure.

FIG. 60 is a schematic drawing showing a ureteral catheter inserted through a percutaneous access site and deployed in a renal pelvis of a patient;

FIG. 61 is a schematic drawing of a urinary tract of a patient showing a system for collecting fluid including the ureteral catheter of FIG. 54;

FIG. 62 is a flow chart of a method for deploying a ureteral catheter in the renal pelvis through a percutaneous access site;

FIGS. 63A-63E are schematic drawings showing steps for inserting a ureteral catheter to a renal pelvis of a patient;

FIG. 64A is a perspective view of another example of a catheter configured to be inserted to the renal pelvis through a percutaneous access site, according to an aspect of the disclosure;

FIG. 64B is a cross-sectional view of the catheter of FIG. 58A;

FIGS. 65A-65F are graphs showing measured hemodynamic variables for a 15-minute baseline period without renal negative pressure therapy and a 15-minute period of renal negative pressure therapy in a no heart failure (No-HF) state, as described in Example 5; and

FIGS. 66A-66D are graphs showing measured hemodynamic variables measured in Example 5 for a pre-fluid state, a No-HF state, and an HF state.

### DETAILED DESCRIPTION OF THE INVENTION

As used herein, the singular form of “a”, “an”, and “the” include plural referents unless the context clearly states otherwise.

As used herein, the terms “right”, “left”, “top”, and derivatives thereof shall relate to the invention as it is oriented in the drawing figures. The term “proximal” refers to the portion of the catheter device that is manipulated or contacted by a user and/or to a portion of an indwelling catheter nearest to the urinary tract access site. The term “distal” refers to the opposite end of the catheter device that is configured to be inserted into a patient and/or to the portion of the device that is inserted farthest into the patient's urinary tract. However, it is to be understood that the invention can assume various alternative orientations and, accordingly, such terms are not to be considered as limiting. Also, it is to be understood that the invention can assume various alternative variations and stage sequences, except where expressly specified to the contrary. It is also to be understood that the specific devices and processes illustrated in the attached drawings, and described in the following specification, are examples. Hence, specific dimensions and other physical characteristics related to the embodiments disclosed herein are not to be considered as limiting.

For the purposes of this specification, unless otherwise indicated, all numbers expressing quantities of ingredients, reaction conditions, dimensions, physical characteristics, and so forth used in the specification and claims are to be understood as being modified in all instances by the term “about.” Unless indicated to the contrary, the numerical parameters set forth in the following specification and attached claims are approximations that can vary depending upon the desired properties sought to be obtained by the present invention.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

Also, it should be understood that any numerical range recited herein is intended to include all sub-ranges subsumed therein. For example, a range of “1 to 10” is intended to include any and all sub-ranges between and including the recited minimum value of 1 and the recited maximum value of 10, that is, all subranges beginning with a minimum value equal to or greater than 1 and ending with a maximum value equal to or less than 10, and all subranges in between, e.g., 1 to 6.3, or 5.5 to 10, or 2.7 to 6.1.

Fluid retention and venous congestion are central problems in the progression to advanced renal disease. Excess sodium ingestion coupled with relative decreases in excretion leads to isotonic volume expansion and secondary compartment involvement. In some examples, the present invention is generally directed to devices and methods for facilitating drainage of urine or waste from the bladder, ureter, and/or kidney(s) of a patient. In some examples, the present invention is generally directed to systems and methods for inducing a negative pressure in at least a portion of the bladder, ureter, and/or kidney(s), e.g., urinary system, of a patient. While not intending to be bound by any theory, it is believed that applying a negative pressure to at least a portion of the bladder, ureter, and/or kidney(s), e.g., urinary system, can offset the medullary nephron tubule re-absorption of sodium and water in some situations. Offsetting re-absorption of sodium and water can increase urine production, decrease total body sodium, and improve erythrocyte production. Since the intra-medullary pressures are driven by sodium and, therefore, volume overload, the targeted removal of excess sodium enables maintenance of volume loss. Removal of volume restores medullary hemostasis. Normal urine production is 1.48-1.96 L/day (or 1-1.4 ml/min).

Fluid retention and venous congestion are also central problems in the progression of prerenal Acute Kidney Injury (AKI). Specifically, AKI can be related to loss of perfusion or blood flow through the kidney(s). Accordingly, in some examples, the present invention facilitates improved renal hemodynamics and increases urine output for the purpose of relieving or reducing venous congestion. Further, it is anticipated that treatment and/or inhibition of AKI positively impacts and/or reduces the occurrence of other conditions, for example, reduction or inhibition of worsening renal function in patients with NYHA Class III and/or Class IV heart failure. Classification of different levels of heart failure are described in The Criteria Committee of the New York Heart Association, (1994), Nomenclature and Criteria for Diagnosis of Diseases of the Heart and Great Vessels, (9th ed.), Boston: Little, Brown & Co. pp. 253-256, the disclosure of which is incorporated by reference herein in its entirety. Reduction or inhibition of episodes of AKI and/or chronically decreased perfusion may also be a treatment for Stage 4 and/or Stage 5 chronic kidney disease. Chronic kidney disease progression is described in National Kidney Foundation, K/DOQI Clinical Practice Guidelines for Chronic Kidney Disease: Evaluation, Classification and Stratification. Am. J. Kidney Dis. 39:S1-S266, 2002 (Suppl. 1), the disclosure of which is incorporated by reference herein in its entirety.

Also, the ureteral catheters, ureteral stents and/or bladder catheters disclosed herein can be useful for preventing, delaying the onset of, and/or treating end-stage renal disease (“ESRD”). The average dialysis patient consumes about $90,000 per year in healthcare utilization for a total cost to the US government of $33.9 Billion. Today, ESRD patients comprise only 2.9% of Medicare's total beneficiaries, yet they account over 13% of total spending. While the incidence and costs per patient have stabilized in recent years, the volume of active patients continues to rise.

The five stages of advanced chronic kidney disease (“CKD”) are based upon glomerular filtration rate (GFR). Stage 1 (GFR>90) patients have normal filtration, while stage 5 (GFR<15) have kidney failure. Like many chronic diseases, the diagnosis capture improves with increasing symptom and disease severity. As used herein, “GFR” is defined as the glomerular filtration rate of a patient. The GFR is an important test to determine how well the filtration is working within the kidney, the level of which correlates to the severity and status of chronic kidney disease (CKD) or other medical condition. As shown in Table 1, the lower the GFR, the more serious the CKD or other medical ailment. The GFR can be estimated based on a blood test measuring the blood creatinine level in combination with other factors. The GFR can be determined in a patient using various methods including, but not limited to, plasma or urinary clearance of an exogenous filtration marker, such as inulin, or serum creatinine and/or cystatin C level. As renal function declines, the loss of blood flow also limits diuretic efficiency thereby worsening an already serious medical condition.

| Stage | Description | GFR |
|---|---|---|
| At increased | Increase of risk factors (e.g., diabetes, high blood | >90 |
| risk | pressure, family history, age, ethnicity) | |
| 1 | Kidney damage with normal kidney function | >90 |
| 2 | Kidney damage with mild loss of kidney function | 60-89 |
| 3a | Mild to moderate loss of kidney function | 44-59 |
| 3b | Moderate to severe loss of kidney function | 30-44 |
| 4 | Severe loss of kidney function | 15-29 |
| 5 | Kidney failure; dialysis required | <15 |

In some examples, the patient being treated by the methods disclosed herein has a GFR prior to treatment of 90 or lower, or 60 or lower, or 44 or lower, or 30 or lower, or 15 or lower.

The CKD 3b/4 subgroup is a smaller subgroup that reflects important changes in disease progression, healthcare system engagement and transition to ESRD. Presentation to the emergency department rises with severity of CKD. Among the US Veteran's Administration population, nearly 86% of the incident dialysis patients had a hospital admission within the five years preceding the admission. Of those, 63% were hospitalized at initiation of dialysis. This suggests a tremendous opportunity to intervene prior to dialysis.

Despite being further down the arterial tree than other organs, the kidneys receive a disproportionate amount of cardiac output at rest. The glomerular membrane represents a path of least resistance of filtrate into the tubules. In healthy states, the nephron has multiple, intricate, redundant means of auto-regulating within normal ranges of arterial pressure.

Venous congestion has been implicated in reduced renal function and is associated with the systemic hypervolemia found in later stages of CKD. Since the kidney is covered with a semi-rigid capsule, small changes in venous pressure translate into direct changes in the intratubule pressures. This shift in intratubule pressure has been shown to upregulate reabsorption of sodium and water, perpetuating the vicious cycle.

Regardless of the initial insult and early progression, more advanced CKD is associated with decreased filtration (by definition) and greater azotemia. Regardless of whether the remaining nephrons are hyperabsorbing water or they are just unable to filtrate sufficiently, this nephron loss is associated with fluid retention and a progressive decline in renal function.

The kidney is sensitive to subtle shifts in volume. As pressure in either the tubule or capillary bed rises, the pressure in the other follows. As the capillary bed pressure rises, the production of filtrate and elimination of urine can decline dramatically. While not intending to be bound by any theory, it is believe that mild and regulated negative pressure delivered to the renal pelvis decreases the pressure among each of the functioning nephrons. In healthy anatomy, the renal pelvis is connected via a network of calyces and collecting ducts to approximately one million individual nephrons. Each of these nephrons are essentially fluid columns connecting Bowman's space to the renal pelvis. Pressure transmitted to the renal pelvis translates throughout. It is believed that, as negative pressure is applied to the renal pelvis, the glomerular capillary pressure forces more filtrate across the glomerular membrane, leading to increased urine output.

It is important to note that the tissues of the urinary tract are lined with urothelium, a type of transitional epithelium. The tissues lining the inside of the urinary tract are also referred to as uroendothelial or urothelial tissues, such as mucosal tissue 1003 of the ureter and/or kidney and bladder tissue 1004. Urothelium has a very high elasticity, enabling a remarkable range of collapsibility and distensibility. The urothelium lining the ureter lumen is surrounded first by the lamina propria, a thin layer of loose connective tissue, which together comprise the urothelial mucosa. This mucosa is then surrounded by a layer of longitudinal muscle fibers. These longitudinal muscle fibers surrounding the urothelial mucosa and the elasticity of the urothelial mucosa itself allow the ureter to relax into a collapsed stellate cross-section and then expand to full distention during diuresis. Histology of any normal ureteral cross-section reveals this star-shaped lumen in humans and other mammals generally used in translational medical research. Wolf, et al., “Comparative Ureteral Microanatomy”, JEU 10: 527-31 (1996).

The process of transporting urine from the kidney to the bladder is driven by contractions through the renal pelvis and peristalsis distally through the rest of the ureter. The renal pelvis is the widening of the proximal ureter into a funnel-shape where the ureter enters the kidney. The renal pelvis has actually been shown to be a continuation of the ureter, comprised of the same tissue but with one additional muscle layer that allows it to contract. Dixon and Gosling, The Musculature of the Human Renal Calyces, Pelvis and Upper Ureter, J. Anat. 135: 129-37 (1982). These contractions push urine through the renal pelvis funnel to allow peristaltic waves to propagate the fluid through the ureter to the bladder.

Imaging studies have shown that the ureter of the dog can readily increase to up to 17× its resting cross-sectional area to accommodate large volumes of urine during diuresis. Woodburne and Lapides, The Ureteral Lumen During Peristalsis, AJA 133: 255-8 (1972). Among swine, considered to be the closest animal model for the human upper urinary tract, the renal pelvis and most proximal ureter are actually shown to be the most compliant of all ureteral sections. Gregersen, et al., Regional Differences Exist in Elastic Wall Properties in the Ureter, SJUN 30: 343-8 (1996). Wolf's comparative analysis of various research animals' ureteral microanatomy to that of humans revealed comparable thickness of lamina propria layer relative to whole ureter diameter in dogs (29.5% in humans and 34% in dogs) and comparable percentage of smooth muscle relative to total muscular cross sectional area in pigs (54% in humans and 45% in pigs). While there are certainly limitations to the comparisons between species, dogs and pigs have historically been strong foci in studying and understanding human ureter anatomy and physiology, and these reference values support this high level of translatability.

There is much more data available on structure and mechanics of pig and dog ureters and renal pelvises than on human ureters. This is due partly to the invasiveness required for such detailed analyses as well as the inherent limitations of various imaging modalities (MRI, CT, ultrasound, etc.) to attempt to accurately identify size and composition of such small, flexible, and dynamic structures clinically. Nevertheless, this ability for the renal pelvis to distend or completely collapse in humans is a hurdle for nephrologists and urologists seeking to improve urine flow.

In some examples, a method is provided for increasing urine output and/or sodium output from a patient having venous congestion. The method comprises: (a) administering at least one medicament to a patient, wherein the medicament increases urine output and/or sodium output from the patient; and (b) applying negative pressure to a drainage lumen of a urinary catheter such that flow of urine from a ureter and/or kidney of the patient is transported within the drainage lumen to extract urine from the patient; wherein administering the at least one medicament occurs before, during and/or after applying negative pressure.

In some examples, a method is provided for treating venous congestion and/or renal dysfunction in a patient in need thereof. The method comprises: (a) administering at least one medicament to a patient, wherein the medicament modulates at least one of electrolyte reabsorption, electrolyte excretion or renal blood flow in the patient; and (b) applying negative pressure to a drainage lumen of a urinary catheter such that flow of urine from a ureter and/or kidney of the patient is transported within the drainage lumen to extract urine from the patient; wherein administering the at least one medicament occurs before, during and/or after applying negative pressure.

In some examples, a method is provided for reducing fluid overload in a patient in need thereof. The method comprises: (a) administering at least one medicament to a patient, wherein the medicament modulates at least one of electrolyte reabsorption, electrolyte excretion or renal blood flow in the patient; and (b) applying negative pressure to a drainage lumen of a urinary catheter such that flow of urine from a ureter and/or kidney of the patient is transported within the drainage lumen to extract urine from the patient, wherein administering the at least one medicament occurs before, during and/or after applying negative pressure.

In some examples, a method is provided for increasing renal blood flow in a patient in need thereof. The method comprises: (a) administering at least one medicament to a patient, wherein the medicament modulates renal blood flow in the patient; and (b) applying negative pressure to a drainage lumen of a urinary catheter such that flow of urine from a ureter and/or kidney of the patient is transported within the drainage lumen to extract urine from the patient; wherein administering the at least one medicament occurs before, during and/or after applying negative pressure. Renal blood flow refers to the volume of blood reaching the kidneys of a patient per unit time. Blood passing through the kidneys is then filtered in glomerulus which in turn gives rise to the glomerular filtrate rate (GFR) which measures the efficiency in which a patient's kidneys are functioning. Thus, an increased blood volume passing through the glomerulus increases the opportunity for the blood to be filtered and/or excess fluids to be removed from the blood stream. In some examples, the medicament is a vasodilator as discussed elsewhere herein which increases the amount of blood that flows through the kidneys of a patient. In some examples, the medicament is one which increases renal blood flow.

In some examples, a method is provided for modulating electrolyte reabsorption and/or electrolyte excretion in a patient in need thereof. The method comprises: (a) administering at least one medicament to a patient, wherein the medicament modulates electrolyte reabsorption and/or electrolyte excretion in the patient; and (b) applying negative pressure to a drainage lumen of a urinary catheter such that flow of urine from a ureter and/or kidney of the patient is transported within the drainage lumen to extract urine from the patient; wherein administering the at least one medicament occurs before, during and/or after applying negative pressure.

Electrolyte reabsorption and/or electrolyte excretion refer to a two-step process where (1) water and dissolved substances are passively or actively moved inside the tubule of the kidney through the tubule wall and into the space outside the tubule, and (2) water and/or dissolved substances move through the capillary walls back into the bloodstream of the patient. The movement can be via active or passive transport in either direction. Sodium is the most important essential substance that is reabsorbed because other nutrients (e.g., glucose, phosphate, amino acids, lactate, citrate, etc.) piggy-back on the sodium co-transport proteins. When the proper sodium gradient is maintained, this process continues properly. When it is disrupted, reabsorption of vital and essential nutrients is likewise disrupted. In some examples, medicaments that help maintain this balance are used with the methods disclosed herein. In some examples, diuretic medicaments as discussed elsewhere herein are used to modulate electrolyte reabsorption and/or electrolyte excretion. In some examples, vasodilators as discussed elsewhere herein are used to modulate electrolyte reabsorption and/or electrolyte excretion.

In some examples, vasodilators and/or diuretic medicaments are provided for use in a method of inducing negative pressure in at least one location within the urinary tract of a patient having venous congestion and/or fluid overload.

In some examples, furosemide, or a pharmaceutically salt or formulation thereof, is provided for use in a method of inducing negative pressure in at least one location within the urinary tract of a patient to increase urine output from the patient.

In some examples, the use of a medicament is provided in a method for inducing negative pressure in at least one location within the urinary tract of a patient having venous congestion and/or fluid overload.

In some examples, the use of a medicament is provided in a method for inducing negative pressure in at least one location within the urinary tract of a patient having edema. In some examples, the medicament comprises one or more diuretic(s) and/or one or more vasodilator(s).

As used herein, the term “treating” or “treatment” of a medical condition or ailment is defined as: (1) preventing or delaying the appearance or development of one or more clinical symptoms of the state, disease, disorder or condition associated with or caused by said medical condition or ailment in the patient that may be afflicted with or predisposed to the state, disease, disorder or condition but does not yet experience or display clinical or subclinical symptoms of the state, disease, disorder or condition, (2) inhibiting the state, disease, disorder or condition associated with or caused by the medical condition or ailment, e.g., arresting or reducing the development of the state, disease, disorder or condition associated with or caused by the medical condition or ailment or at least one clinical or subclinical symptom thereof, and/or (3) relieving or ameliorating the state, disease, disorder or condition associated with or caused by the medical condition or ailment, e.g., causing regression or amelioration of the state, the state, disease, disorder or condition associated with or caused by the medical condition or ailment or at least one of its clinical or subclinical symptoms. The benefit to a subject to be treated is either statistically significant or at least perceptible to the patient or to the physician (e.g., decreased edema). “Treating” or “treatment” does not imply that the medical condition is cured or eliminated although that is one of several possible patient outcomes. Additional patient outcomes from being treated include the alleviation and/or reduction in severity of one or more symptoms of the medical condition or ailment. Thus, the methods contemplated herein are suitable to treat any form of venous congestion, edema and/or heart failure, or any other disease state or medical condition discussed herein. The methods contemplated herein are also be suitable to treat any medical condition or ailment where diuresis is desirable and/or would provide a medical benefit to the patient.

As used herein, “improving” or “improvement” with respect to a medical condition or ailment means reducing the severity of at least one symptom associated with a specific medical condition or ailment. Such an improvement may completely alleviate at least one symptom or it may provide partial relief from at least one symptom. In some examples, the medical condition is one in which increased urine output and/or sodium output is desirable or would provide a medical benefit to the patient. In some examples, the medical condition is venous congestion, and/or heart failure. In some examples, the medical condition exhibits edema as one of the symptoms. In some examples, “improving” means reducing edema in a patient in need thereof.

Edema can be categorized as trace/mild (0 points), moderate (1 point), or severe (2 points). Orthopnea can be assessed by determining if the patient needs at least 2 pillows to breathe comfortably (2 points) or absent (0 points). An Orthodema Score can be generated by the sum of the individual orthopnea and edema scores (below). A total score of 1 represents the presence of moderate edema without orthopnea. A score of 2 indicates the presence of orthopnea or severe peripheral edema, but not both. Scores of 1 to 2 represent low-grade congestion. High-grade congestion includes orthopnea and edema, with a score of 3 for orthopnea plus moderate edema, and a score of 4 if orthopnea is accompanied by severe edema.

Orthodema Scores

| | | |
|---|---|---|
| Mild edema, no orthopnea | 0 | No congestion |
| Moderate edema, no orthopnea | 1 | Low-grade orthodema/congestion |
| Severe edema OR orthopnea | 2 | |
| Moderate edema and orthopnea | 3 | High-grade orthodema/congestion |
| Severe edema and orthopnea | 4 | |

As used herein, the term “therapeutically effective amount” or “therapeutically effective dose” means the amount of a medicament or drug, that, when administered to a patient in need thereof for treating a medical condition or ailment, is sufficient to treat such medical condition. The “therapeutically effective amount” will vary depending on the specific medicament and the particular state, disease, disorder or condition being treated and its severity. It will also depend on the age, weight, physical condition and responsiveness of the patient to be treated. Thus, one or more of these parameters can be used to select and adjust the therapeutically effective amount of the medicament. Also, the amount can be determined using pharmacologic methods known in the art such as dose response curves. In some examples, the therapeutically effective dose is selected by the medical professional overseeing or administering the treatment of the patient and is based on the professional medical judgement of the medical professional. In some examples, the therapeutically effective dose of the medicament administered in the methods used in conjunction with at least one medical device as described elsewhere herein will be lower than the therapeutically effective dose when said medicament is administered alone (i.e., not in combination with a medical device as described herein). In some examples, the therapeutically effective dose is based on the Prescribing Information for the medicament administered to the patient. In some examples, the dose is the minimum dose listed as being effective for the medicament as described in the Prescribing Information for that medicament. In some examples, the dose is within the suggested dosage range for the medicament as included in the Prescribing Information for that medicament.

Venous congestion, heart impairment or heart failure are complex medical ailments where treatment may require administering one or more different medicaments to a patient. Patients are often administered multiple medicaments based on the nature and/or severity of their symptoms and medical condition.

In some examples of the present methods, a patient is administered at least one (one, two, or more) medicament(s). In some examples, when a patient is administered two or more medicaments, the medicaments may be in the same class or from different classes, and may be administered at the same time, or at different times as determined by the medical practitioner.

The administration of the at least one medicament can occur before, during and/or after applying negative pressure, at any time as determined by the medical practitioner. For example, the medicament(s) can be administered in a range of about two months before application of negative pressure to about 2 months after application of negative pressure, or at any time therebetween. In some examples, the medicament(s) can be administered in a range of about one week, or about 3 days, or about 1 day, or about 12 hours, or about 8 hours, or about 6 hours, or about 4 hours, or about 2 hours, or about 1 hour before application of negative pressure, or 0 to 60 minutes before application of negative pressure, or at any time during application of negative pressure, or 0 to 60 minutes after application of negative pressure, or about 1 hour, or about 2 hours, or about 4 hours, or about 6 hours, or about 8 hours, or about 12 hours, or about 1 day, or about 3 days, or about one week after application of negative pressure, or at any time therebetween. In some examples, the medicament is administered from about 1 to about 300 minutes before the application of negative pressure. In some examples, the medicament is administered about 15 minutes, or about 30 minutes, or about 45 minutes, or about 60 minutes, or about 90 minutes, or about 120 minutes, or about 150 minutes, or about 180 minutes, or about 2 hours, or about 2.5 hours, or about 3.5 hours, or about 4 hours, or about 5 hours, or about 6 hours, or about 9 hours, or about 12 hours before the application of negative pressure.

Different medicaments each have a different time period to reach its peak effectiveness. This time period is known to persons skilled in the art and the medical professional overseeing the treatment of the patient. It is often included in the Prescribing Information for the medicament. In some examples, the medicament is administered at such a time that the peak effectiveness of said medicament occurs while negative pressure is being induced in the urinary tract of the patient.

The medicament(s) can be administered orally, subcutaneously, intravenously, transdermally, by inhalation, etc. In some examples, the medicament is in a unit dosage form. In such form, the preparation is subdivided into suitably sized unit doses containing appropriate quantities of the active component, e.g., an effective amount to achieve the desired purpose.

The quantity of active compound in a unit dose of preparation may be varied or adjusted from about 1 mg to about 500 mg, or from about 1 mg to about 120 mg, or about 40 mg to about 120 mg, or from about 1 mg to about 25 mg, according to the particular application.

The actual dosage employed may be varied depending upon the requirements of the patient and the severity of the condition being treated. Determination of the proper dosage regimen for a particular situation is within the skill of the art. For convenience, the total daily dosage may be divided and administered in portions during the day as required.

The amount and frequency of administration of the compounds of the invention and/or the pharmaceutically acceptable salts thereof will be regulated according to the judgment of the attending clinician considering such factors as age, condition and size of the patient as well as severity of the symptoms being treated. A typical recommended daily dosage regimen for oral administration can range from about 1 mg/day to about 1000 mg/day, preferably 1 mg/day to 200 mg/day, given in a single dose or 2-4 divided doses. The exact dose, however, is determined by the attending clinician and is dependent on the potency of the compound administered, the age, weight, condition and response of the patient.

For administration of pharmaceutically acceptable salts of the above compounds, the weights indicated above refer to the weight of the acid equivalent or the base equivalent of the therapeutic compound derived from the salt.

A useful dosage can be about 0.001 to 500 mg/kg of body weight/day of the medicament(s), or about 0.01 to 25 mg/kg of body weight/day. In some examples, when the patient is administered a medicament(s), the dose is administered as a single dosage unit or it is divided into multiple doses. In some examples, the total daily dosage is administered in two, three, four or more divided doses. The exact timing and amount of each dose is determined by the attending medical professional based on the needs of the patient. For example, a first dose can be administered before the induction of negative pressure in the urinary tract of the patient and a second dose can be administered while negative pressure is being induced in the urinary tract of the patient. In some examples, the timing of the dose or doses is determined based on the Prescribing Information for the specific medicament administered to the patient.

Non-limiting examples of suitable medicaments for use in the present methods include, but are not limited to, one or more of angiotensin-converting enzyme inhibitor(s) (ACE inhibitor(s)), angiotensin II receptor blocker(s) (ARB(s)), beta blocker(s), diuretic(s), aldosterone antagonist(s), inotrope(s), angiotensin-receptor-neprilysin-inhibitor(s) (ARNI(s)), sodium glucose co-transporter(s) (SGLT-2), vasodilator(s), or combinations thereof. In some examples, the at least one medicament is selected from the group consisting of diuretic(s), SGLT-2 inhibitor(s), and combinations thereof. In some examples, the at least one medicament comprises at least one diuretic.

Diuretics, colloquially called water pills, are medicaments that increase the amount of water and salt expelled from the body as urine (i.e., by diuresis). Non-limiting examples of suitable diuretics for use in the present methods include, but are not limited to, one or more of loop diuretic(s), carbonic anhydrase inhibitor(s), potassium-sparing diuretic(s), calcium-sparing diuretic(s), osmotic diuretic(s), thiazide diuretic(s), miscellaneous diuretics or combinations thereof.

Loop diuretics are medicaments that act on the ascending limb of Henle in the kidney of a patient. They inhibit the reabsorption of sodium potassium chloride (NKCC2) co-transporter in the thick limb of the loop of Henle. By inhibiting reabsorption of sodium, the hypertonic filtrate inhibits the reabsorption of water via diffusion leading to volume removal. Non-limiting examples of suitable loop diuretics for use in the present methods include, but are not limited to, one or more of bumetanide, ethacrynic acid, torsemide, or furosemide. In some examples, the patient is administered bumetanide. In some examples, the patient is administered ethacrynic acid. In some examples, the patient is administered torsemide. In some examples, the patient is administered furosemide.

In some examples where the medicament is furosemide, a patient is administered from about 20 to about 600 mg/day, or about 20 to about 500 mg/day, or about 20 to about 400 mg/day, or about 20 to about 300 mg/day, or about 20 to about 200 mg/day, or about 20 to about 100 mg/day, or about 20 to 80 mg/day, or about 20 mg/day, or about 40 mg/day, or about 60 mg/day, or about 80 mg/day, or about 100 mg/day, or about 120 mg/day, or about 140 mg/day, or about 160 mg/day, or about 180 mg/day, or about 200 mg/day, or about 300 mg/day, or about 400 mg/day, or about 500 mg/day, or about 600 mg/day, in a single dose or divided into multiple doses.

In some examples where the medicament is bumetanide, a patient is administered from about 0.5 to 10 mg/day, or about 0.5 mg/day, or about 1 mg/day, or about 1.5 mg/day, or about 2 mg/day, or about 3 mg/day, or about 4 mg/day, or about 5 mg/day, or about 6 mg/day, or about 7 mg/day, or about 8 mg/day, or about 9 mg/day, or about 10 mg/day, in a single dose or divided into multiple doses.

In some examples where the medicament is torsemide, a patient is administered from about 1.25 to about 200 mg/day, or about 10 mg/day, or about 20 mg/day, or about 30 mg/day, or about 40 mg/day, or about 50 mg/day, or about 60 mg/day, or about 70 mg/day, or about 80 mg/day, or about 90 mg/day, or about 100 mg/day, or about 120 mg/day, or about 140 mg/day, or about 160 mg/day, or about 180 mg/day, or about 200 mg/day, in a single dose or divided into multiple doses.

In some examples where the medicament is ethacrynic acid, a patient is administered from about 25 to about 400 mg/day, or about 50 to about 200 mg/day, or about 50 mg/day, or about 75 mg/day, or about 100 mg/day, or about 125 mg/day, or about 150 mg/day, or about 175 mg/day, or about 200 mg/day, in a single dose or divided into multiple doses.

Thiazide diuretics act directly on the kidney and promote diuresis by inhibiting the sodium/chloride cotransporter in the distal tubule of the nephrons in the kidney of a patient. They decrease sodium reabsorption which decreases extracellular fluid and plasma volume. Non-limiting examples of suitable thiazide diuretics include, but are not limited to, one or more of indapamide, hydrochlorothiazide, chlorthalidone, metolazone, methyclothiazide, chlorothiazide, bendroflumethiazide, polythiazide, hydroflumethiazide, or combinations thereof. In some examples, the patient is administered indapamide. In some examples, the patient is administered hydrochlorothiazide. In some examples, the patient is administered chlorthalidone. In some examples, the patient is administered metolazone. In some examples, the patient is administered methyclothiazide. In some examples, the patient is administered chlorothiazide. In some examples, the patient is administered bendroflumethiazide. In some examples, the patient is administered polythiazide. In some examples, the patient is administered hydroflumethiazide. In some examples, the patient is administered from about 0.5 to about 1000 mg/day, or about 1 to 500 mg/day, or about 2 to 400 mg/, or about 3 to 300 mg/day, in a single dose or divided into multiple doses.

Carbonic anhydrase inhibitors reduce the activity of carbonic anhydrase, the enzyme that catalyzes the reaction between carbon dioxide and water to form carbonic acid and eventually bicarbonate. This reduces the reabsorption of bicarbonate in the proximal tubules of the kidneys of a patient which increases bicarbonate extraction. This causes an increase in both sodium and potassium extraction also. Non-limiting examples of suitable carbonic anhydrase inhibitors include, but are not limited to, one or more of acetazolamide, dichlorphenamide, methazolamide and combinations thereof. In some examples, the patient is administered acetazolamide. In some examples, the patient is administered dichlorphenamide. In some examples, the patient is administered methazolamide. In some examples, the patient is administered from about 0.5 to about 1000 mg/day, or about 1 to about 500 mg/day, or about 2 to about 400 mg/day, or about 3 to about 300 mg/day, in a single dose or divided into multiple doses

Potassium-sparing diuretics increase diuresis without also causing an increase in potassium excretion. They function by inhibiting the sodium-potassium exchange in the distal convoluted tubules in the kidneys of a patient. Non-limiting examples of suitable potassium-sparing diuretics include, but are not limited to, one or more of eplerenone, triamterene, spironolactone, amiloride, or combinations thereof. In some examples, the patient is administered eplerenone. In some examples, the patient is administered triamterene. In some examples, the patient is administered spironolactone. In some examples, the patient is administered amiloride. In some examples, the patient is administered from about 0.5 to about 1000 mg/day, or about 1 to 500 mg/day, or about 2 to 400 mg/, or about 3 to 300 mg/day, in a single dose or divided into multiple doses.

Calcium-sparing diuretics reduce the rate of excretion of calcium by a patient. Certain thiazide and potassium-sparing diuretics are also calcium-sparing. The thiazide diuretics and potassium-sparing diuretics are also considered as calcium-sparing diuretics.

Osmotic diuretics inhibit the reabsorption of water and sodium. They are generally inert but function by increasing the osmolarity of the blood and renal filtrate in a patient. Non-limiting examples of suitable osmotic diuretics include, but are not limited to, one or more of mannitol and/or isosorbide. In some examples, the patient is administered mannitol. In some examples, the patient is administered isosorbide. In some examples, the patient is administered from about 0.5 to about 1000 mg/day, or about 1 to about 500 mg/day, or about 2 to about 400 mg/day, or about 3 to about 300 mg/day, in a single dose or divided into multiple doses.

Sodium-glucose cotransporter-2 (SGLT-2) inhibitors, also called gliflozins, inhibit the SGLT-2 proteins in the renal tubules in the kidneys that are responsible for reabsorbing glucose back into the bloodstream. As a result, more glucose is excreted in the urine. This helps lower the level of hemoglobin A1c which improves weight loss and lowers blood pressure. Non-limiting examples of suitable SGLT-2 inhibitors include, but are not limited to, one or more of ertugliflozin, canagliflozin, empagliflozin, dapagliflozin or combinations thereof. In some examples, the patient is administered ertugliflozin. In some examples, the patient is administered canagliflozin. In some examples, the patient is administered empagliflozin. In some examples, the patient is administered dapagliflozin. In some examples, the patient is administered from about 0.5 to about 1000 mg/day, or about 1 to about 500 mg/day, or about 2 to about 400 mg/day, or about 3 to about 300 mg/day, in a single dose or divided into multiple doses.

Non-limiting examples of suitable miscellaneous diuretics include, but are not limited to, one or more of pamabrom, glucose, mannitol, or combinations thereof. In some examples, the patient is administered pamabrom. In some examples, the patient is administered mannitol. In some examples, the patient is administered glucose. In most instances, the miscellaneous diuretics are over-the-counter medicaments where a doctor's prescription is not necessary. As such, a patient should carefully follow any instructions and warnings with respect thereto before taking any such medicament. In some examples, the dose taken by or administered to a patient should closely follow the recommended dosing regimen as provided with the medicament.

As used herein, the term “vasodilator” is defined as a drug that dilates (widens) blood vessels, allowing blood to flow more easily therethrough. Some vasodilators act directly on the smooth muscle cells lining the blood vessels. Other have a central effect, and regulate blood pressure most likely through the vasomotor center located within the medulla oblongata of the brain. Non-limiting examples of suitable vasodilators include, but are not limited to, one or more of nitrovasodilator(s) (such as nitroglycerin, isosorbide mononitrate, isosorbide dinitrate or sodium nitroprusside), ACE inhibitor(s), angiotensin receptor antagonist(s), phosphodiesterase inhibitor(s), direct vasodilator(s), adrenergic receptor antagonist(s), calcium channel blocking drug(s), alpha blocker(s), beta blocker(s), lymphthomimetic(s), vitamin(s), organic nitrate(s), serotonin receptor-blocking agent(s), angina blocking agent(s), other hypertensive agent(s), cardiac stimulating agent(s), agent(s) which improve renal, vascular function, sympathomimetic amine, and salts, derivatives, precursors, pharmaceutically active sequences or regions, natriuretic peptides (such as ularitide, cenderitide or serelaxin), peptidomimetic(s), mimetic(s), and mixtures thereof. In some examples, the patient is administered from about 0.5 to about 1000 mg/day, or about 1 to about 500 mg/day, or about 2 to about 400 mg/day, or about 3 to about 300 mg/day, in a single dose or divided into multiple doses.

As used herein, the term “RAAS inhibitor” refers to drugs that inhibit the renin-antiotensin-aldosterone system in a patient. In many instances RAAS inhibitors are also vasodilators as disclosed elsewhere herein. Non-limiting examples of suitable RAAS inhibitors diuretics include, but are not limited to, one or more of ACE inhibitor(s), angiotensin receptor antagonist(s), beta blocker(s), calcium channel blocker(s), and angiotensin receptor neprilysin inhibitors (ARNIs). In some examples, the patient is administered from about 0.5 to about 1000 mg/day, or about 1 to about 500 mg/day, or about 2 to about 400 mg/day, or about 3 to about 300 mg/day, in a single dose or divided into multiple doses.

Any of the medicaments disclosed can be used alone or in combination, and can be administered at the same time or at different times, for example as discussed herein.

In all aspects for the one or more medicaments administered in the methods disclosed herein, each medicament can be present in the form of a pharmaceutically acceptable formulation, and can include pharmaceutically acceptable excipients. In some examples, the medicament is in the form of one or more salt(s), ester(s), polymorph(s), or prodrug(s), as they exist. The pharmaceutically acceptable formulation may have received regulatory approval for commercial marketing or it may still be under development (e.g., clinical trials). In all aspects, the pharmaceutically acceptable formulation is deemed appropriate and suitable for administration to human patients.”

While not intending to be bound by any theory, the present inventors theorized that the application of negative pressure might help to facilitate fluid flow from the kidney, and that a very particular tool, designed to deploy a protective surface area in order to open or maintain the opening of the interior of the renal pelvis while inhibiting the surrounding tissues from contracting or collapsing into the fluid column under negative pressure, is needed to facilitate the application of negative pressure within the renal pelvis. While not intending to be bound by any theory, the present inventors theorized that application of negative pressure before, during and/or after the use of medicaments as disclosed herein can unexpectedly and/or synergistically enhance the flow of fluid from the kidney. For example, Loop diuretics are medicaments that inhibit the reabsorption of sodium in the thick limb of the loop of Henle. By inhibiting the reabsorption of sodium, the hypertonic filtrate inhibits the reabsorption of water via solvent drag leading to increased urine volume. However, during congestion, renal blood flow is reduced and delivery of the medicament to the lumen of the tubule is reduced. As a consequence, the effectiveness of the loop diuretics is diminished. The application of negative pressure into the collection system of the kidney results in an increase in renal blood flow and filtrate delivered to the tubules, even during congestion. Combining these approaches leads to an augmentation of the urine produced via either method alone. Negative pressure will increase the production of filtrate, hence sodium delivery to the tubule. Negative pressure will also increase renal blood flow, hence delivery of more loop diuretic to the tubule. Therefore, more sodium can be blocked from reabsorption and more urine is produced.

As used herein, “maintain patency of fluid flow between a kidney and a bladder of the patient” means establishing, increasing or maintaining flow of fluid, such as urine, from the kidneys through the ureter(s), ureteral stent(s) and/or ureteral catheter(s) to the bladder and outside of the body. In some examples, the fluid flow is facilitated or maintained by providing a protective surface area 1001 in the upper urinary tract and/or bladder to prevent the uroendothelium from contracting or collapsing into the fluid column or stream. As used herein, “fluid” means urine and any other fluid from the urinary tract.

As used herein, “negative pressure” means that the pressure applied to the proximal end of the bladder catheter or the proximal end of the ureteral catheter, respectively, is below the existing pressure at the proximal end of the bladder catheter or the proximal end of the ureteral catheter, respectively, prior to application of the negative pressure, e.g., there is a pressure differential between the proximal end of the bladder catheter or the proximal end of the ureteral catheter, respectively, and the existing pressure at the proximal end of the bladder catheter or the proximal end of the ureteral catheter, respectively, prior to application of the negative pressure. This pressure differential causes fluid from the kidney to be drawn into the ureteral catheter or bladder catheter, respectively, or through both the ureteral catheter and the bladder catheter, and then outside of the patient's body. For example, negative pressure applied to the proximal end of the bladder catheter or the proximal end of the ureteral catheter can be less than atmospheric pressure (less than about 760 mm Hg or about 1 atm), or less than the pressure measured at the proximal end of the bladder catheter or the proximal end of the ureteral catheter prior to the application of negative pressure, such that fluid is drawn from the kidney and/or bladder. In some examples, the negative pressure applied to the proximal end of the bladder catheter or the proximal end of the ureteral catheter can range from about 0.1 mm Hg to about 150 mm Hg, or about 0.1 mm Hg to about 50 mm Hg, or about 0.1 mm Hg to about 10 mm Hg, or about 5 mm Hg to about 20 mm Hg, or about 45 mm Hg (gauge pressure at the pump 710 or at a gauge at the negative pressure source). In some examples, the negative pressure source comprises a pump external to the patient's body for application of negative pressure through both the bladder catheter and the ureteral catheter, which in turn causes fluid from the kidney to be drawn into the ureteral catheter, through both the ureteral catheter and the bladder catheter, and then outside of the patient's body. In some examples, the negative pressure source comprises a vacuum source external to the patient's body for application and regulation of negative pressure through both the bladder catheter and the ureteral catheter, which in turn causes fluid from the kidney to be drawn into the ureteral catheter, through both the ureteral catheter and the bladder catheter, and then outside of the patient's body. In some examples, the vacuum source is selected from the group consisting of a wall suction source, vacuum bottle, and manual vacuum source, or the vacuum source is provided by a pressure differential. In some examples, the negative pressure received from the negative pressure source can be controlled manually, automatically, or combinations thereof. In some examples, a controller is used to regulate negative pressure from the negative pressure source. Non-limiting examples of negative and positive pressure sources are discussed in detail below.

As used herein, “positive pressure” means that the pressure applied to the proximal end of the bladder catheter or the proximal end of the ureteral catheter, respectively, is above the existing pressure at the proximal end of the bladder catheter or the proximal end of the ureteral catheter, respectively, prior to application of the negative pressure, and causes fluid present in the ureteral catheter or bladder catheter, respectively, or through both the ureteral catheter and the bladder catheter, to flow back towards the bladder or kidney. In some examples, the positive pressure applied to the proximal end of the bladder catheter or the proximal end of the ureteral catheter can range from about 0.1 mm Hg to about 150 mm Hg, or about 0.1 mm Hg to about 50 mm Hg, or about 0.1 mm Hg to about 10 mm Hg, or about 5 mm Hg to about 20 mm Hg, or about 45 mm Hg (gauge pressure at the pump 710 or at a gauge at the positive pressure source). The positive pressure source can be provided by a pump or wall pressure source, or pressurized bottle, for example, and can be controlled manually, automatically, or combinations thereof. In some examples, a controller is used to regulate positive pressure from the positive pressure source.

The catheter designs of the present invention disclosed herein provide a protective surface area to inhibit surrounding urothelial tissues from contracting or collapsing into the fluid column under negative pressure. It is believed that the catheter designs of the present invention disclosed herein can successfully maintain the stellate longitudinal folding of the ureteral wall away from the central axis and protected holes of the catheter drainage lumen, and can inhibit natural sliding of the catheter down the stellate cross-sectional area of the ureteral lumen and/or downward migration by peristaltic waves.

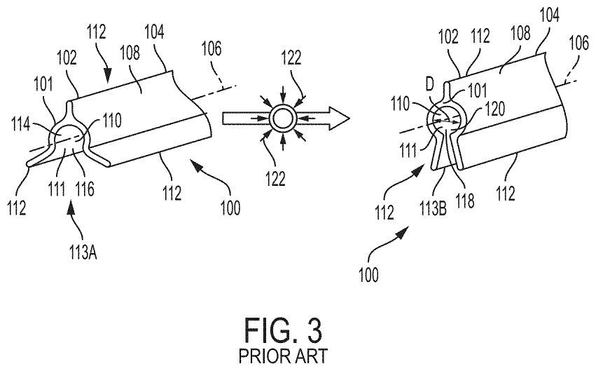

Also, catheter designs of the present invention disclosed herein can avoid an unprotected open hole at the distal end of the drainage lumen which fails to protect surrounding tissues during suction. While it is convenient to think of the ureter as a straight tube, the true ureter and renal pelvis can enter the kidney at a variety of angles. Lippincott Williams & Wilkins, Annals of Surgery, 58, FIGS. 3 & 9 (1913). Therefore, it would be difficult to control the orientation of an unprotected open hole at the distal end of the drainage lumen when deploying such a catheter in the renal pelvis. This single hole may present a localized suction point that has no means of either reliable or consistent distancing from tissue walls, thereby permitting tissue to occlude the unprotected open hole and risking damage to the tissue. Also, catheter designs of the present invention disclosed herein can avoid placement of a balloon having an unprotected open hole at the distal end of the drainage lumen close to the kidney which may result in suction against and/or occlusion of the calyces. Placement of a balloon having an unprotected open hole at the distal end of the drainage lumen at the very base of the uretero—renal pelvis junction may result in suction against and occlusion by renal pelvis tissue. Also, a rounded balloon may present a risk of ureteral avulsion or other damage from incidental pulling forces on the balloon.

Delivering negative pressure into the kidney area of a patient has a number of anatomical challenges for at least three reasons. First, the urinary system is composed of highly pliable tissues that are easily deformed. Medical textbooks often depict the bladder as a thick muscular structure that can remain in a fixed shape regardless of the volume of urine contained within the bladder. However, in reality, the bladder is a soft deformable structure. The bladder shrinks to conform to the volume of urine contained in the bladder. An empty bladder more closely resembles a deflated latex balloon than a ball. In addition, the mucosal lining on the interior of the bladder is soft and susceptible to irritation and damage. It is desirable to avoid drawing the urinary system tissue into the orifices of the catheter to maintain adequate fluid flow therethrough and avoid injury to the surrounding tissue.

Second, the ureters are small tube-like structures that can expand and contract to transport urine from the renal pelvis to the bladder. This transport occurs in two ways: peristaltic activity and by a pressure gradient in an open system. In the peristaltic activity, a urine portion is pushed ahead of a contractile wave, which almost completely obliterates the lumen. The wave pattern initiates in the renal pelvis area, propagates along the ureter, and terminates in the bladder. Such a complete occlusion interrupts the fluid flow and can prevent negative pressure delivered in the bladder from reaching the renal pelvis without assistance. The second type of transport, by pressure gradient through a wide-open ureter, may be present during large urine flow. During such periods of high urine production, the pressure head in the renal pelvis would not need to be caused by contraction of the smooth muscles of the upper urinary tract, but rather is generated by the forward flow of urine, and therefore reflects arterial blood pressure. Kiil F., Urinary Flow and Ureteral Peristalsis in: Lutzeyer W., Melchior H. (Eds.) Urodynamics. Springer, Berlin, Heidelberg (pp. 57-70) (1973).

Third, the renal pelvis is at least as pliable as the bladder. The thin wall of the renal pelvis can expand to accommodate multiple times the normal volume, for example as occurs in patients having hydronephrosis.

More recently, the use of negative pressure in the renal pelvis to remove blood clots from the renal pelvis by the use of suction has been cautioned against because of the inevitable collapse of the renal pelvis, and as such discourages the use of negative pressure in the renal pelvis region. Webb, Percutaneous Renal Surgery: A Practical Clinical Handbook. p 92. Springer (2016).

While not intending to be bound by any theory, the tissues of the renal pelvis and bladder are flexible enough to be drawn inwardly during delivery of negative pressure to conform to the shape and volume of the tool being used to deliver negative pressure. Analogous to the vacuum sealing of a husked ear of corn, the urothelial tissue will collapse around and conform to the source of negative pressure. To prevent the tissue from occluding the lumen and impeding the flow of urine, the present inventors theorized that a protective surface area sufficient to maintain the fluid column when mild negative pressure is applied would prevent or inhibit occlusion.

The present inventors determined that there are specific features that enable a catheter tool to be deployed successfully in and deliver negative pressure through the urological region that have not been previously described. These require a deep understanding of the anatomy and physiology of the treatment zone and adjacent tissues. The catheter must comprise a protective surface area within the renal pelvis by supporting the urothelium and inhibiting the urothelial tissue from occluding openings in the catheter during application of negative pressure through the catheter lumen. For example, establishing a three dimensional shape or void volume, that is free or essentially free from urothelial tissue, ensures the patency of the fluid column or flow from each of the million nephrons into the drainage lumen of the catheter.

Since the renal pelvis is comprised of longitudinally oriented smooth muscle cells, the protective surface area would ideally incorporate a multi-planar approach to establishing the protected surface area. Anatomy is often described in three planes, sagittal (vertical front to back that divides the body into right and left parts), coronal (vertical side to side dividing the body into dorsal and ventral parts) and transverse (horizontal or axial that divides the body into superior and inferior parts, and is perpendicular to the sagittal and coronal planes). The smooth muscle cells in the renal pelvis are oriented vertically. It is desirable for the catheter to also maintain a radial surface area across the many transverse planes between the kidney and the ureter. This enables a catheter to account for both longitudinal and horizontal portions of the renal pelvis in the establishment of a protective surface area 1001. In addition, given the flexibility of the tissues, the protection of these tissues from the openings or orifices that lead to the lumen of the catheter tool is desirable. The catheters discussed herein can be useful for delivering negative pressure, positive pressure, or can be used at ambient pressure, or any combination thereof.

In some examples, a deployable/retractable expansion mechanism is utilized that, when deployed, creates and/or maintains a patent fluid column or flow between the kidney and the catheter drainage lumen. This deployable/retractable mechanism, when deployed, creates the protective surface area 1001 within the renal pelvis by supporting the urothelium and inhibiting the urothelial tissue from occluding openings in the catheter during application of negative pressure through the catheter lumen. In some examples, the retention portion is configured to be extended into a deployed position in which a diameter of the retention portion is greater than a diameter of the drainage lumen portion.

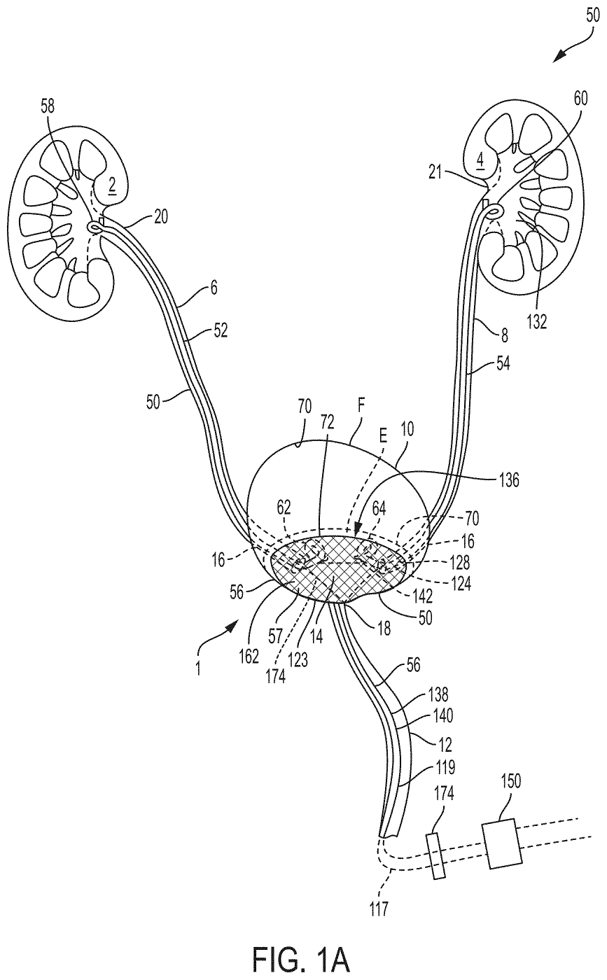

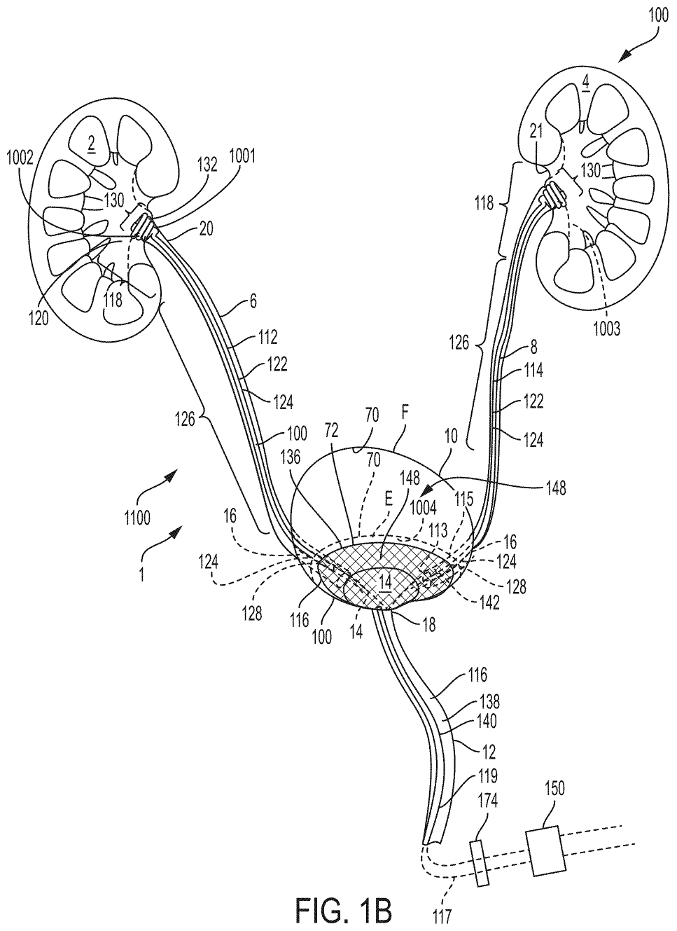

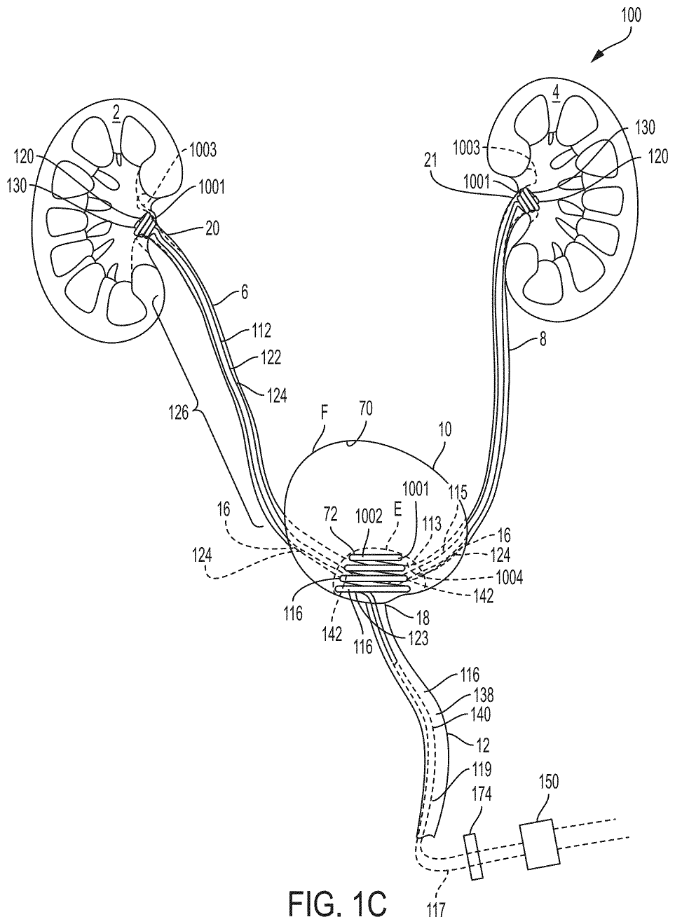

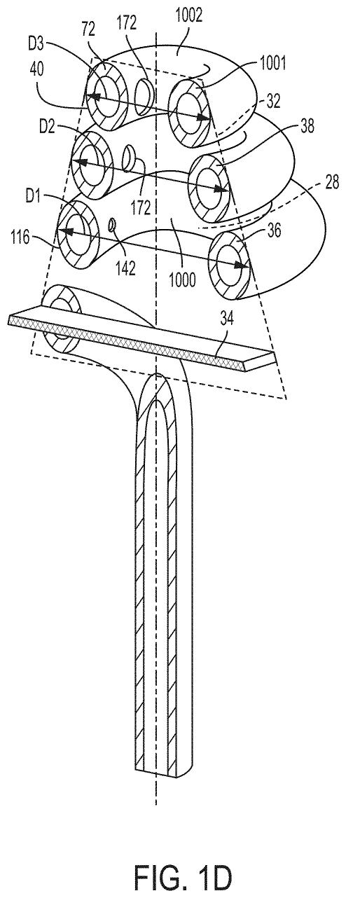

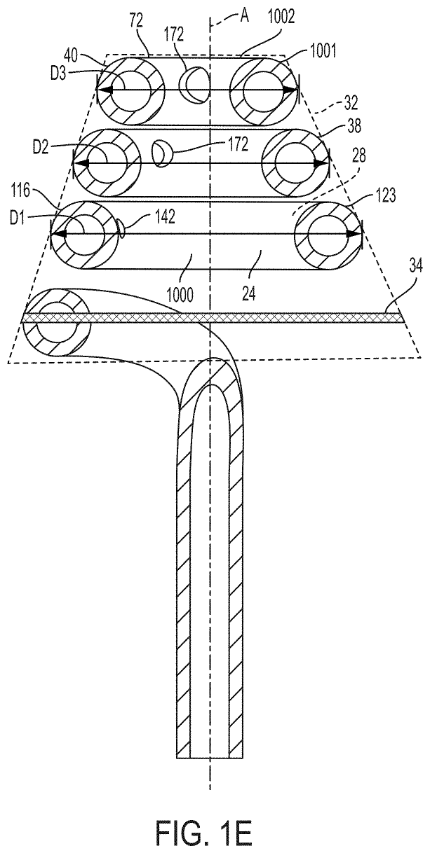

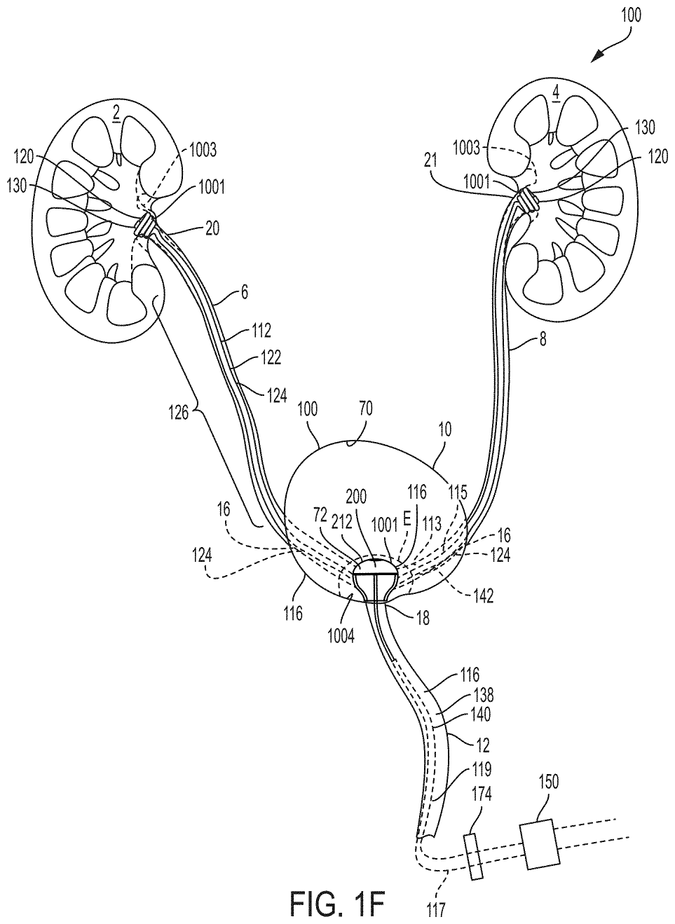

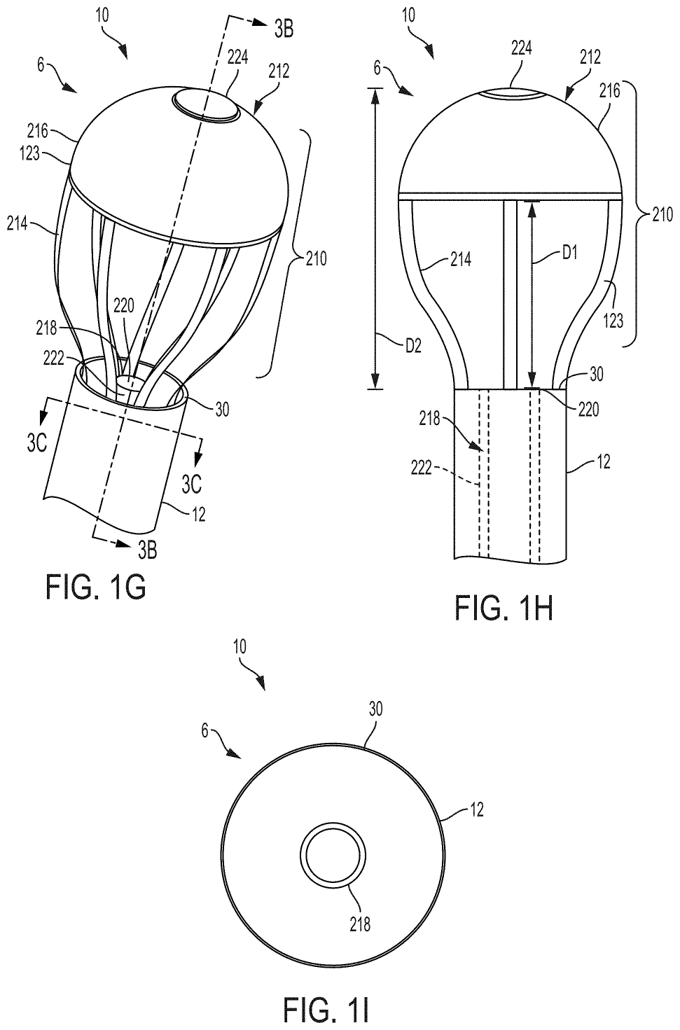

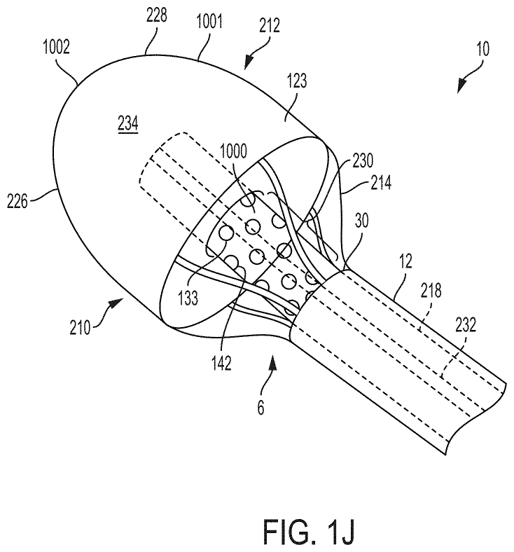

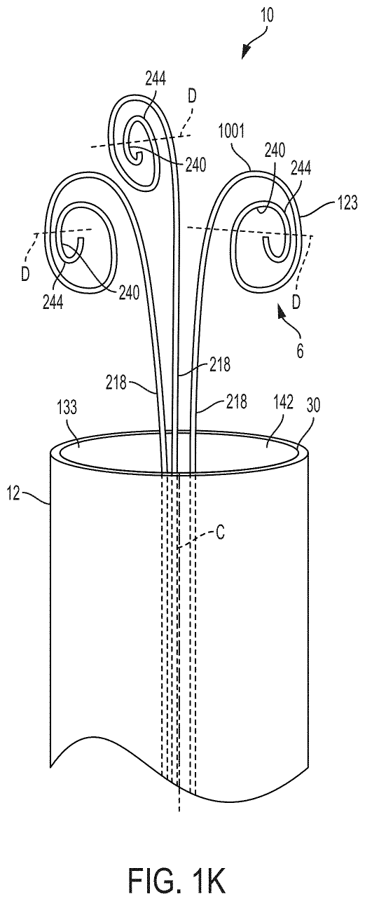

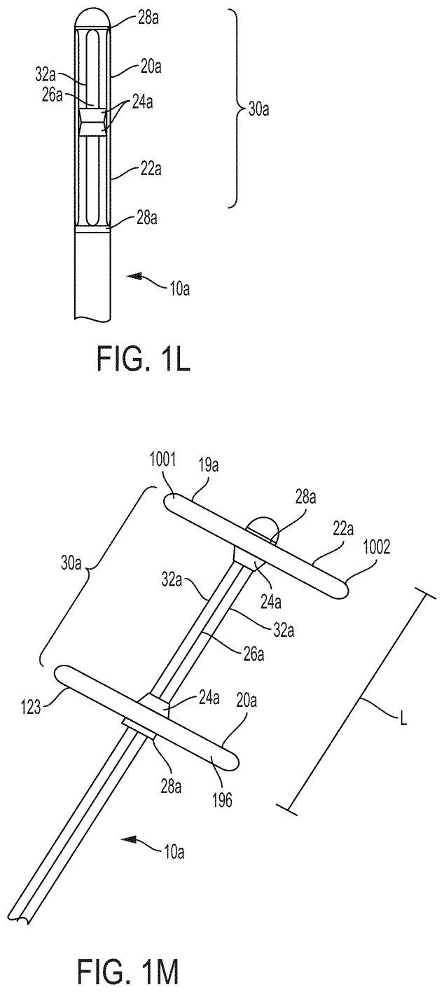

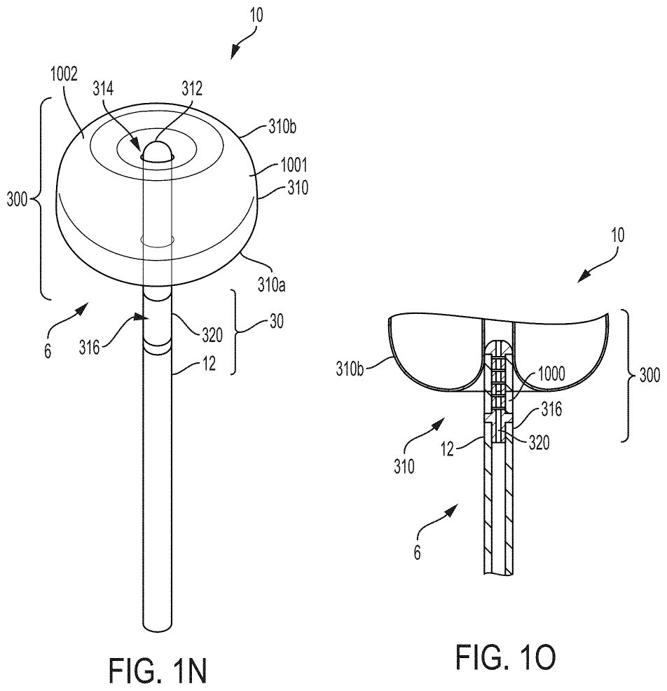

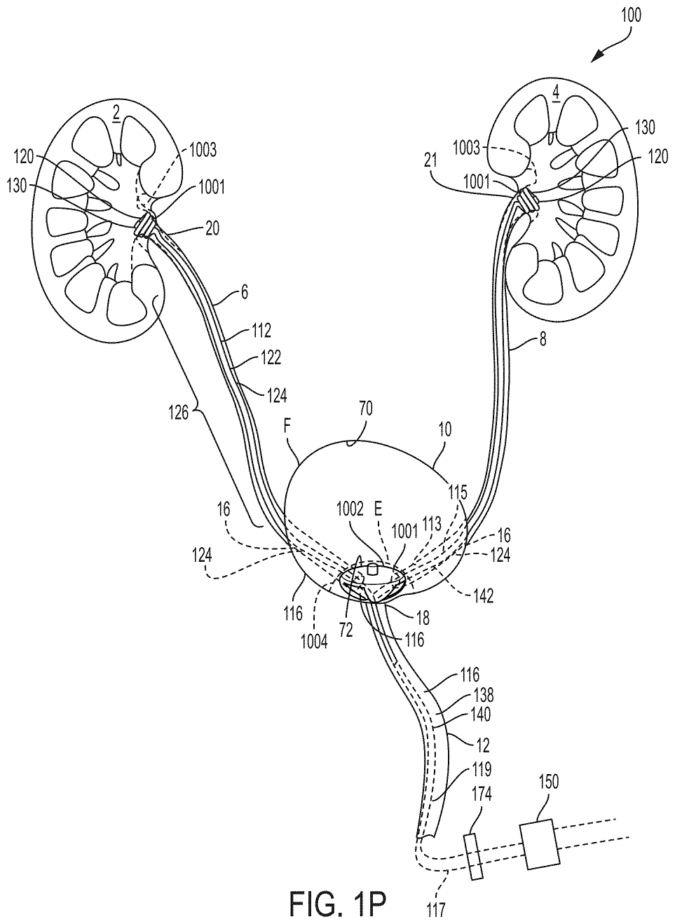

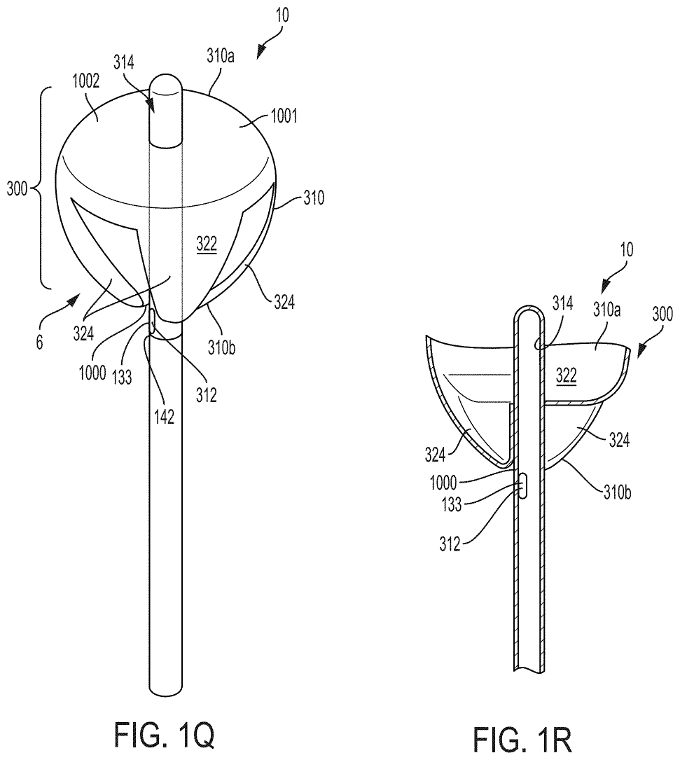

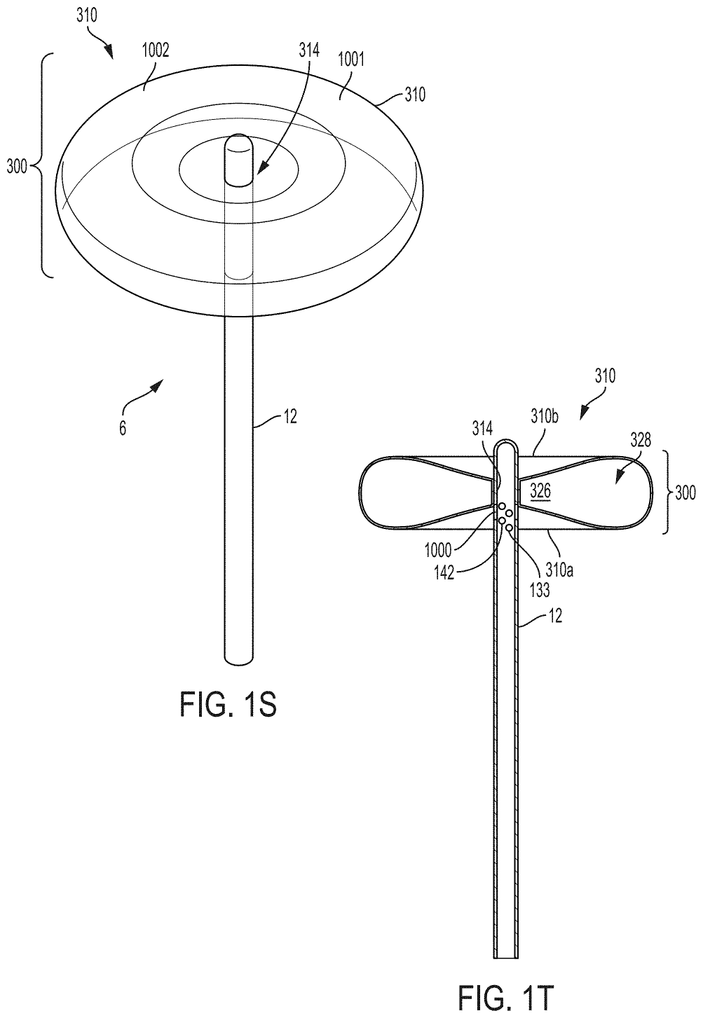

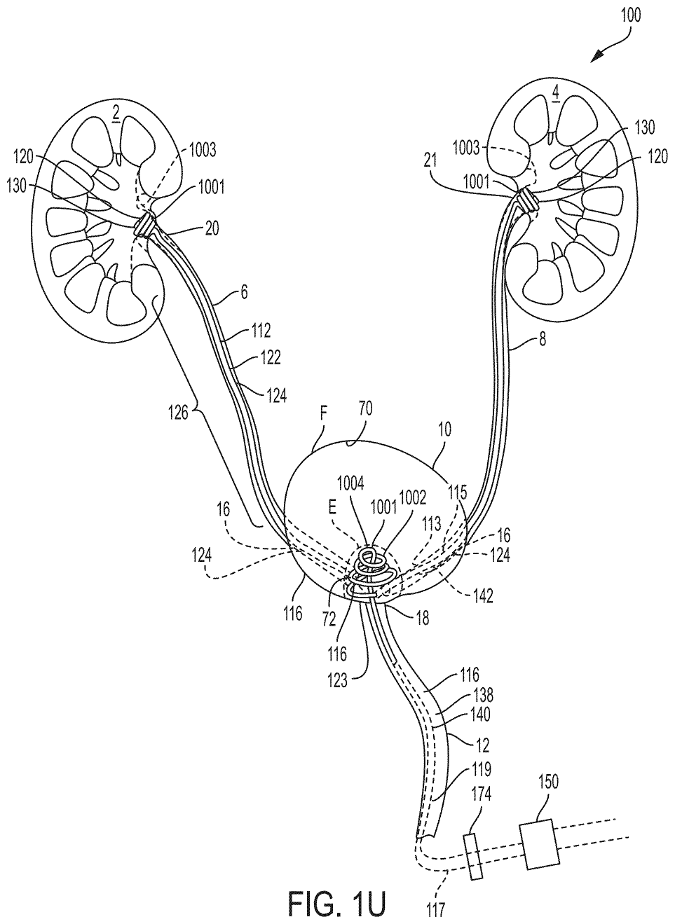

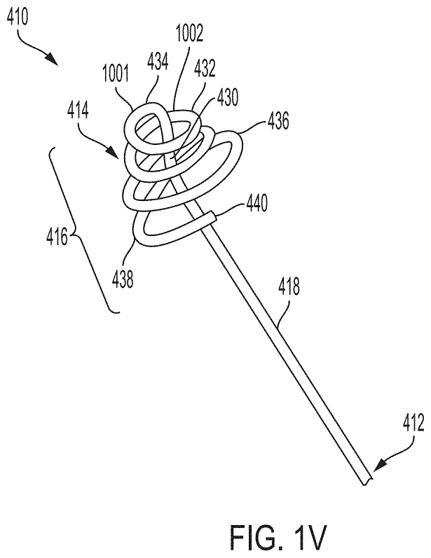

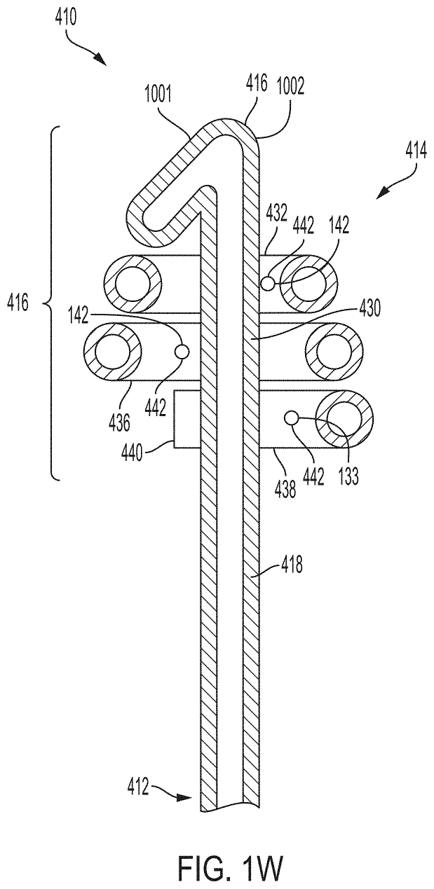

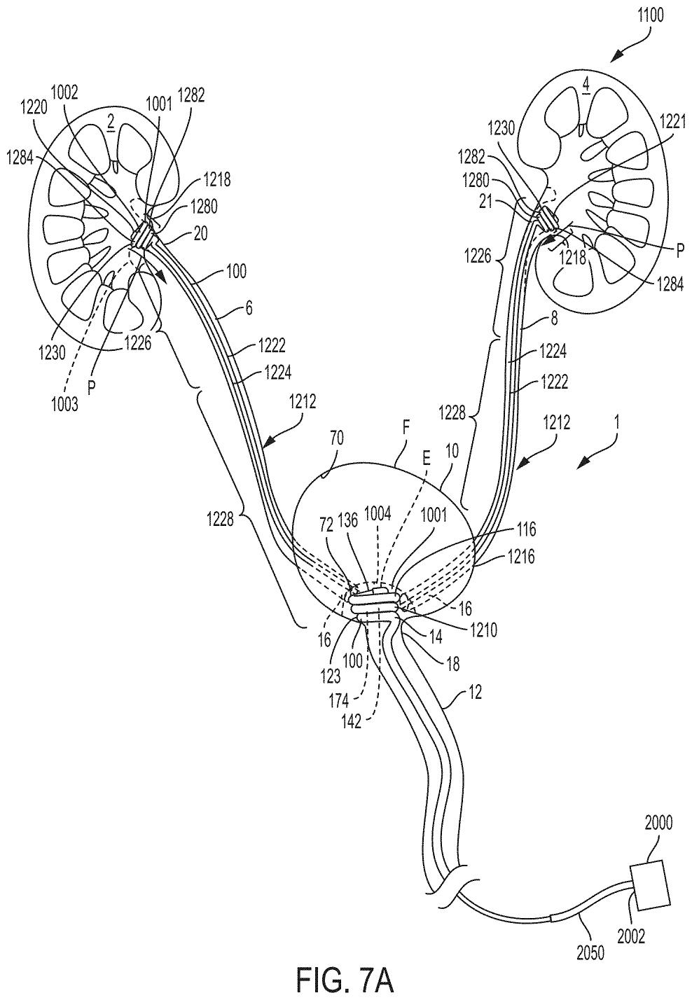

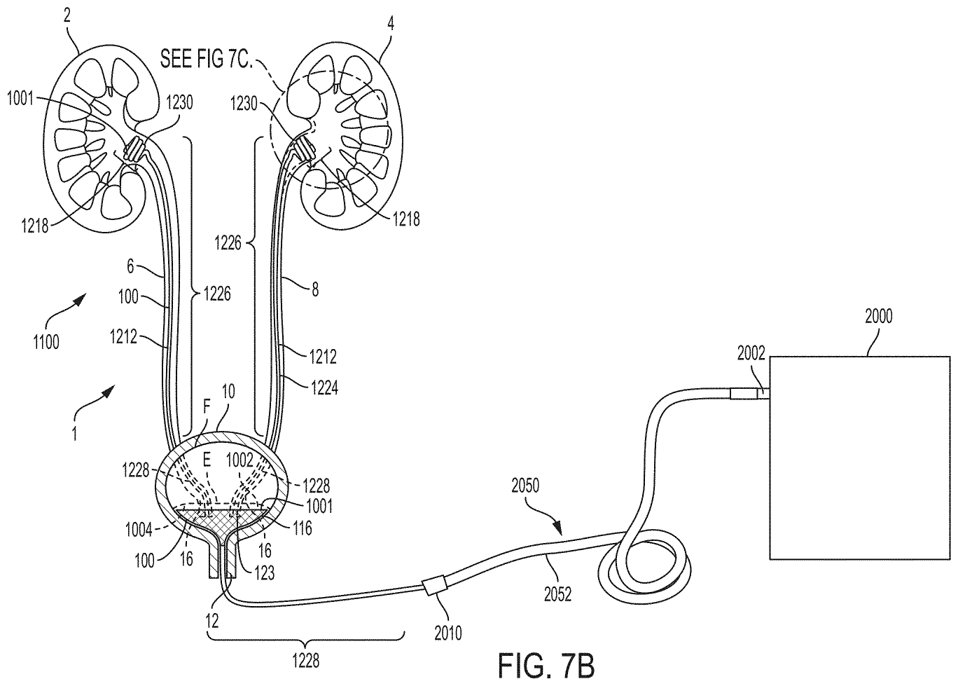

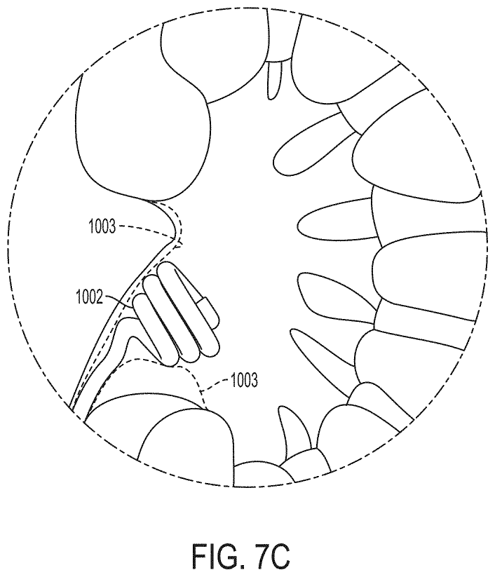

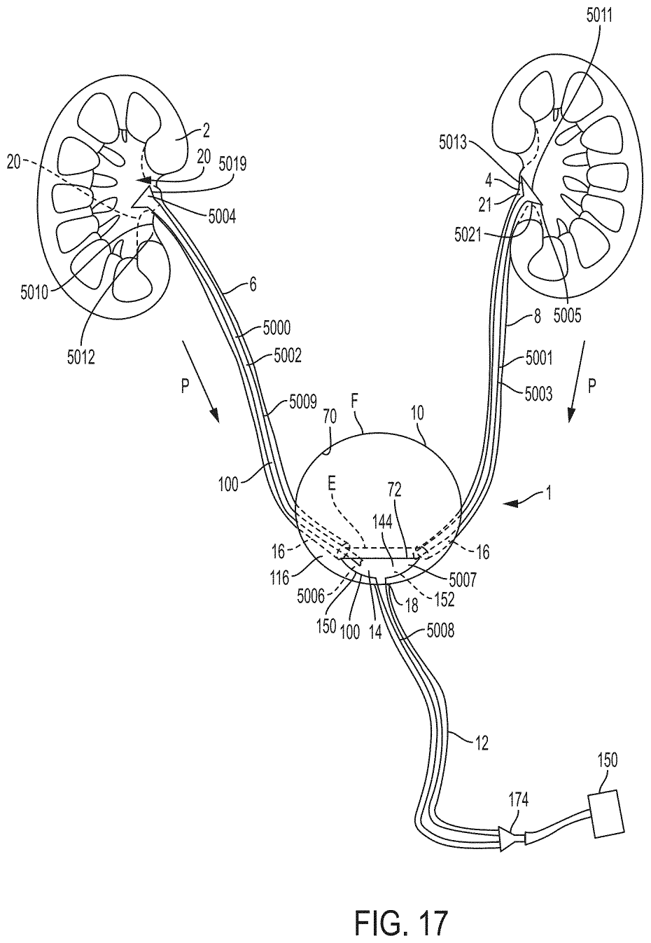

With reference to FIGS. 1A-1C, 1F, 1P, 1U, 2A, 2B, 7A, 7B, 17, and 44, the urinary tract, indicated generally at 1, comprises a patient's right kidney 2 and left kidney 4. As discussed above, the kidneys 2, 4 are responsible for blood filtration and clearance of waste compounds from the body through urine. Urine produced by the right kidney 2 and the left kidney 4 is drained into a patient's bladder 10 through tubules, namely a right ureter 6 and a left ureter 8. For example, urine may be conducted through the ureters 6, 8 by peristalsis of the ureter walls, as well as by gravity. The ureters 6, 8 enter the bladder 10 through a ureter orifice or opening 16. The bladder 10 is a flexible and substantially hollow structure adapted to collect urine until the urine is excreted from the body. The bladder 10 is transitionable from an empty position (signified by reference line E) to a full position (signified by reference line F). When the bladder is in the empty position E, the bladder superior wall 70 can be positioned adjacent to and/or conform to the outer periphery 72, 1002 or protective surface area 1001 of the distal end 136 of the bladder catheter 56, 116, shown for example in FIGS. 1A and 1B as mesh 57, in FIGS. 1C, 1U and 7A as coil 1210, in FIG. 1F as a basket shaped structure or support cap 212 of a bladder superior wall support 210, in FIG. 1P as an annular balloon 310, and in FIG. 17 as funnel 116. Normally, when the bladder 10 reaches a substantially full state, urine is permitted to drain from the bladder 10 to a urethra 12 through a urethral sphincter or opening 18 located at a lower portion of the bladder 10. Contraction of the bladder 10 can be responsive to stresses and pressure exerted on a trigone region 14 of the bladder 10, which is the triangular region extending between the ureteral openings 16 and the urethral opening 18. The trigone region 14 is sensitive to stress and pressure, such that as the bladder 10 begins to fill, pressure on the trigone region 14 increases. When a threshold pressure on the trigone region 14 is exceeded, the bladder 10 begins to contract to expel collected urine through the urethra 12.

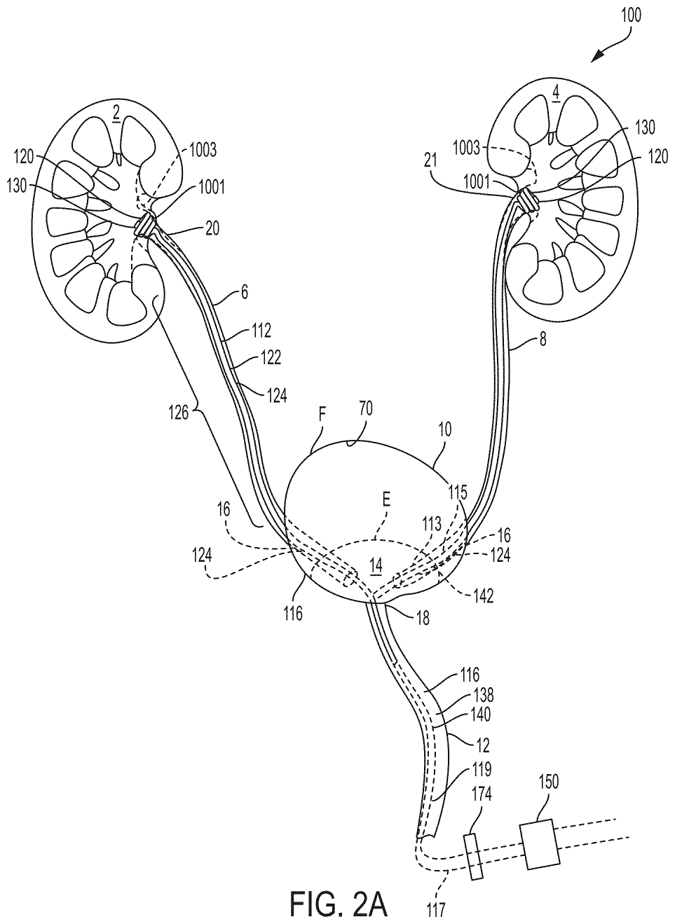

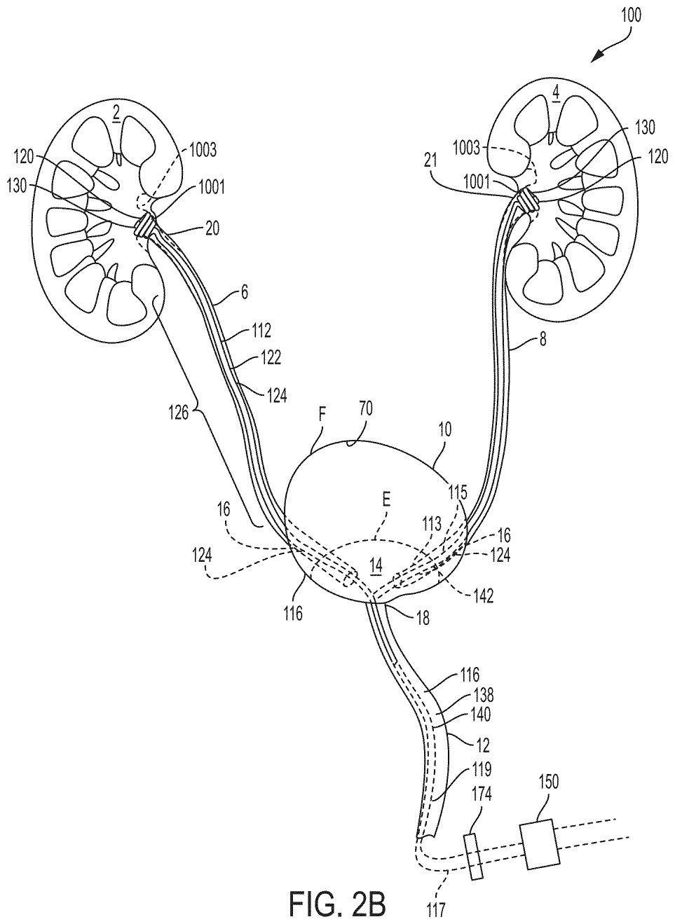

Similarly, as shown in FIGS. 1A, 1B, 1C, 1F, 1P, 1U, 2A and 2B, for example, the outer periphery 72, 1002 or protective surface area 1001 of the ureteral catheters 112, 114 of the present invention can support tissue 1003 of the ureter and/or kidney to maintain patency of fluid flow between the kidney and the bladder of the patient.

In some examples, methods and systems 50, 100, as shown for example in FIGS. 1A, 1B, 1C, 1F, 1P, 1U, 2A, 2B, 7, 17, and 44, are provided for removing fluid (such as urine) from a patient, the method comprising: deploying a ureteral stent 52, 54 (shown in FIG. 1A) or ureteral catheter 112, 114 (shown in FIGS. 1B, 1C, 1F, 1P, 1U, 2A, 2B, 7, 17, and 44) into a ureter 6, 8 of a patient to maintain patency of fluid flow between a kidney 2, 4 and a bladder 10 of the patient; and/or deploying a bladder catheter 56, 116 into the bladder 10 of the patient, wherein the bladder catheter 56, 116 comprises a distal end 136 configured to be positioned in a patient's bladder 10, a drainage lumen portion 140 having a proximal end 117, and a sidewall 119 extending therebetween; and applying negative pressure to the proximal end 117 of the bladder catheter 56, 116 and/or ureteral catheter(s) 112, 114 to induce negative pressure in a portion of the urinary tract of the patient to remove fluid from the patient. In some examples, the method further comprises deploying a second ureteral stent or second ureteral catheter into a second ureter or kidney of the patient to maintain patency of fluid flow between a second kidney and the bladder of the patient, as shown in FIGS. 1A, 1B, 1C, 1F, 1P, 1U, 2A, 2B, 7, 17, and 44. Specific characteristics of exemplary ureteral stents or ureteral catheters of the present invention are described in detail herein.

In some non-limiting examples, the ureteral or bladder catheter 56, 112, 114, 116, 312, 412, 512, 812, 1212, 5000, 5001 comprises (a) a proximal portion 117, 128, 1228, 5006, 5007, 5017 and (b) a distal portion 118, 318, 1218, 5004, 5005, the distal portion comprising a retention portion 130, 330, 410, 500, 1230, 1330, 2230, 3230, 4230, 5012, 5013 that comprises at least one protected drainage hole(s), port(s) or perforation(s) 133, 533, 1233 and is configured to establish an outer periphery 1002 or protective surface area 1001 that inhibits urothelial tissue, such as mucosal tissue 1003 of the ureter and/or kidney and bladder tissue 1004, from occluding the at least one protected drainage hole(s), port(s) or perforation(s) 133, 533, 1233 upon application of negative pressure through the catheter.

### Exemplary Ureteral Catheters

As shown in FIGS. 2A, 7, 17, and 44, examples of systems 100 including ureteral catheters 112, 114 configured to be positioned within the urinary tract of a patient are illustrated. For example, distal ends 120, 121, 1220, 5019, 5021 of the ureteral catheters 112, 114 can be configured to be deployed in at least one of the patient's ureters 2, 4; renal pelvis 20, 21 area of the kidneys 6, 8; or the kidneys 6, 8.

In some examples, suitable ureteral catheters are disclosed in U.S. Pat. No. 9,744,331, US Patent Application Publication No. US 2017/0021128 A1, U.S. patent application Ser. No. 15/687,064, and U.S. patent application Ser. No. 15/687,083, each of which is incorporated by reference herein.

In some examples, the system 100 can comprise two separate ureteral catheters, such as a first catheter 112 disposed in or adjacent to the renal pelvis 20 of the right kidney 2 and a second catheter 114 disposed in or adjacent to the renal pelvis 21 of the left kidney 4. The catheters 112, 114 can be separate for their entire lengths, or can be held in proximity to one another by a clip, ring, clamp, or other type of connection mechanism (e.g., connector) to facilitate placement or removal of the catheters 112, 114. As shown in FIGS. 2A, 7, 17, 27, and 44, the proximal end 113, 115 of each catheter 112, 114 is positioned within the bladder 10, or at the proximal end of the ureter near the bladder 10, such that the fluid or urine drains into the bladder. In some examples, the proximal end 113, 115 of each catheter 112, 114 can be in fluid communication with the distal portion or end 136 of a bladder catheter 56, 116. In some examples, catheters 112, 114 can merge or be connected together within the bladder to form a single drainage lumen that drains into the bladder 10.

As shown in FIG. 2A, in some examples, the proximal end 113, 115 of one or both of the catheters 112, 114 can be positioned within the urethra 12 and optionally connected to additional drainage tubing to drain fluid to the outside of the body of the patient. As shown in FIG. 2B, in some examples, the proximal end 113, 115 of one or both of the catheters 112, 114 can be positioned to extend from the urethra 12 outside of the body of the patient.

In other examples, the catheters 112, 114 can be inserted through or enclosed within another catheter, tube, or sheath along portions or segments thereof to facilitate insertion and retraction of the catheters 112, 114 from the patient's body. For example, a bladder catheter 116 can be inserted over and/or along the same guidewire as the ureteral catheters 112, 114, or within the same tubing used to insert the ureteral catheters 112, 114.

With reference to FIGS. 1B, 1C, 1F, 1P, 1U, 2A, 2B, 7, 8A, and 8B, an exemplary ureteral catheter 112, 1212, 5000 can comprise at least one elongated body or tube 122, 1222, 5009 the interior of which defines or comprises one or more drainage channel(s) or lumen(s), such as drainage lumen 124, 1224, 5002. The tube 122, 1222, 5009 size can range from about 1 Fr to about 9 Fr (French catheter scale). In some examples, the tube 122, 1222, 5009 can have an external diameter ranging from about 0.33 to about 3 mm, and an internal diameter ranging from about 0.165 to about 2.39 mm. In one example, the tube 122 is 6 Fr and has an outer diameter of 2.0±0.1 mm. The length of the tube 122, 1222, 5009 can range from about 30 cm to about 120 cm depending on the age (e.g., pediatric or adult) and gender of the patient.

The tube 122, 1222, 5009 can be formed from a flexible and/or deformable material to facilitate advancing and/or positioning the tube 122, 1222, 5009 in the bladder 10 and ureters 6, 8 (shown in FIGS. 2 and 7). The catheter material should be flexible and soft enough to avoid or reduce irritation of the renal pelvis and ureter, but should be rigid enough that the tube 122, 1222, 5009 does not collapse when the renal pelvis or other portions of the urinary tract exert pressure on the exterior of the tube 122, 1222, 5009, or when the renal pelvis and/or ureter are drawn against the tube 122, 1222, 5009 during inducement of negative pressure. For example, the tube 122, 1222, 5009 or drainage lumen can be formed, at least in part, from one or more materials including copper, silver, gold, nickel-titanium alloy, stainless steel, titanium, and/or polymer such as biocompatible polymer(s), polyurethane, polyvinyl chloride, polytetrafluoroethylene (PTFE), latex, silicone coated latex, silicone, polyglycolide or poly(glycolic acid) (PGA), Polylactide (PLA), Poly(lactide-co-glycolide), Polyhydroxyalkanoates, Polycaprolactone and/or Poly(propylene fumarate). In one example, the tube 122, 1222, 5009 is formed from a thermoplastic polyurethane. The tube 122, 1222, 5009 can also include or be impregnated with one or more of copper, silver, gold, nickel-titanium alloy, stainless steel, and titanium. In some examples, the tube 122, 1222, 5009 is impregnated with or formed from a material viewable by fluoroscopic imaging. For example, the biocompatible polymer which forms the tube 122, 1222, 5009 can be impregnated with barium sulfate or a similar radiopaque material. As such, the structure and position of the tube 122, 1222, 5009 is visible to fluoroscopy.

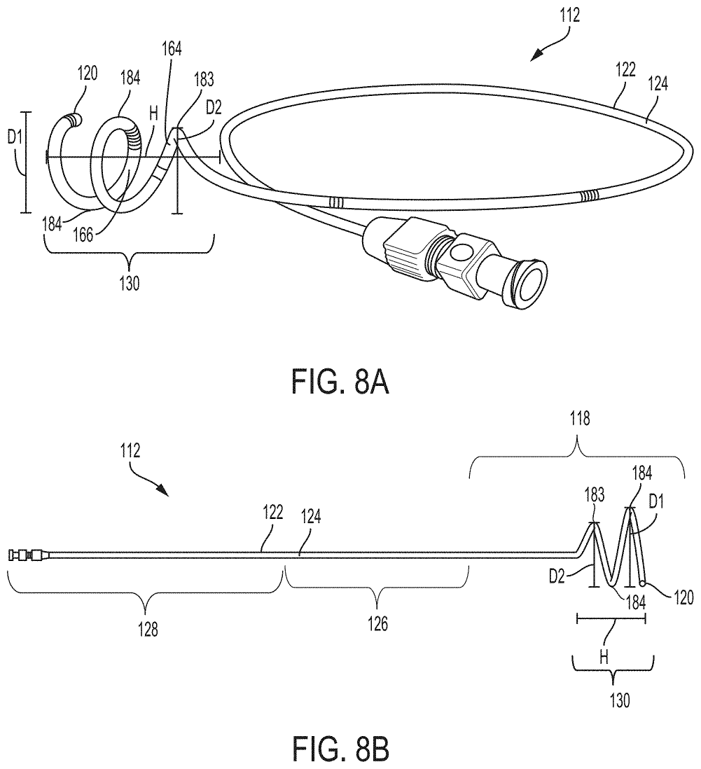

In some examples, as shown in FIG. 8B, for example, the tube 122 can comprise: a distal portion 118 (e.g., a portion of the tube 122 configured to be positioned in the ureter 6, 8 and renal pelvis 20, 21); a middle portion 126 (e.g., a portion of the tube 122 configured to extend from the distal portion 118 through the ureteral openings 16 into the patient's bladder 10 and urethra 12); and a proximal portion 128 (e.g., a portion of the tube 122 extending into the bladder 10, or urethra 12, or extending from the urethra 12 outside of the body of the patient). In one example, the combined length of the proximal portion 128 and the middle portion 126 of the tube 122 is about 54±2 cm. In some examples, the tube 122 terminates in the bladder 10. In that case, fluid drains from the proximal end of the ureteral catheter 112, 114 and is directed from the body through the additional indwelling bladder catheter. In other examples, the tube 122 terminates in the urethra 12, e.g., a bladder catheter is not required. In other examples, the tube extends from the urethra 12 outside of the body of the patient, e.g., a bladder catheter is not required.

### Exemplary Ureteral Retention Portions

Any of the retention portions disclosed herein can be formed from the same material as the drainage lumen discussed above, and can be unitary with or connected to the drainage lumen, or the retention portion can be formed from a different material, such as those that are discussed above for the drainage lumen, and connected thereto. For example, the retention portion can be formed from any of the aforementioned materials, for example a polymer such as polyurethane, flexible polyvinyl chloride, polytetrafluoroethylene (PTFE), latex, silicone, silicon, polyglycolide or poly(glycolic acid) (PGA), Polylactide (PLA), Poly(lactide-co-glycolide), Polyhydroxyalkanoates. Polycaprolactone and/or Poly(propylene fumarate).

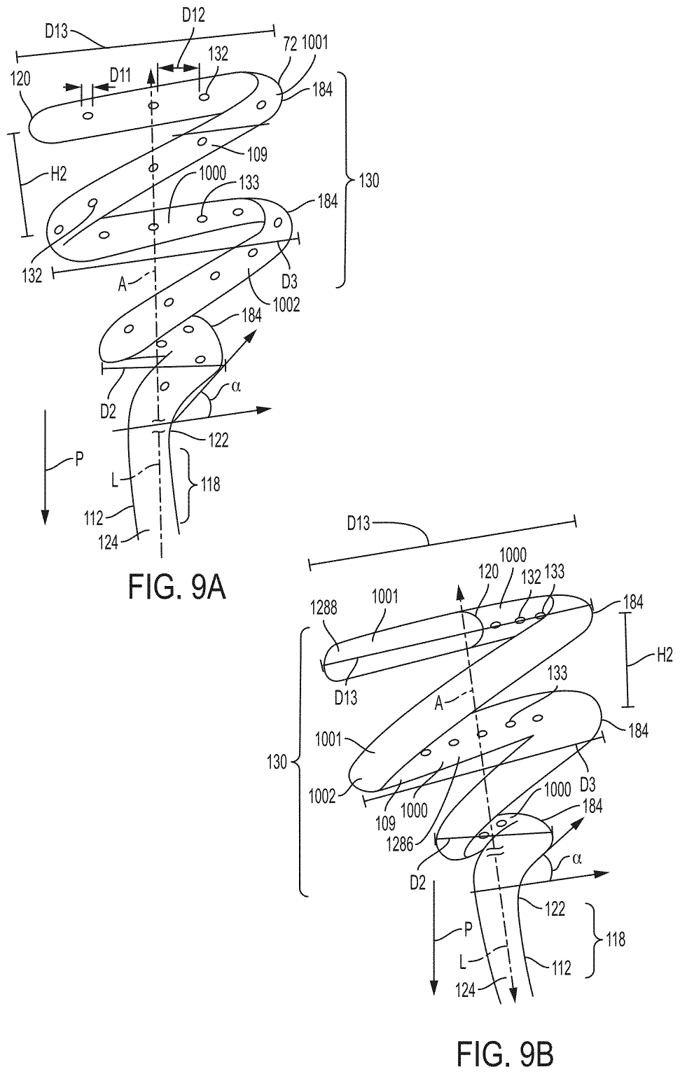

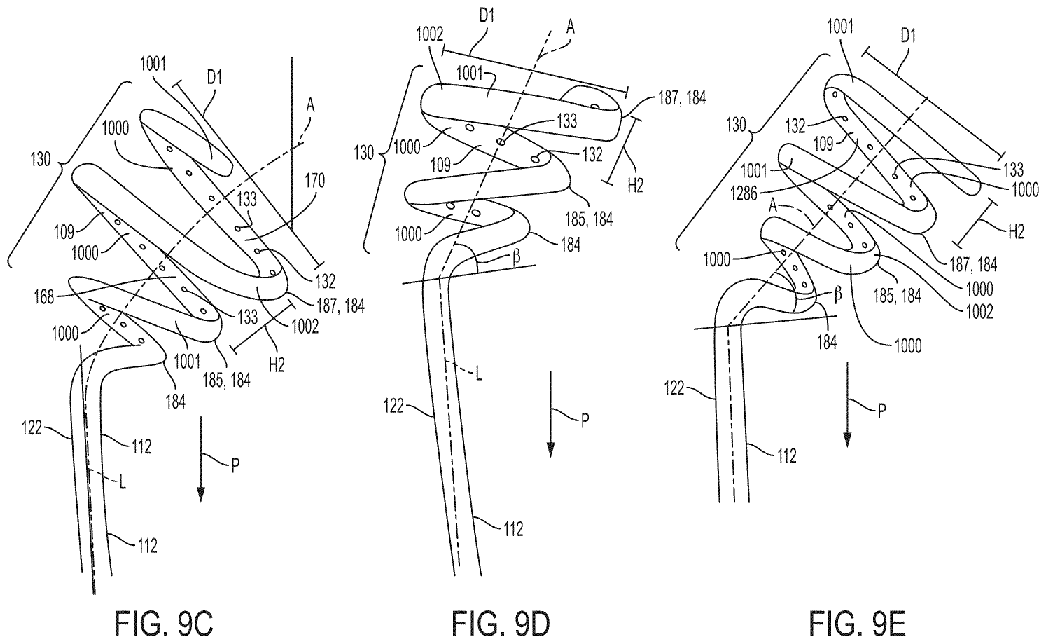

Generally, and as shown for example in FIGS. 2A-C, 8A, and 8B, the distal portion 118 of the ureteral catheter 112 comprises a retention portion 130 for maintaining the distal end 120 of the catheter 112 at a desired fluid collection position proximate to or within the renal pelvis 20, 21 of the kidney 2, 4. In some examples, the retention portion 130 is configured to be flexible and bendable to permit positioning of the retention portion 130 in the ureter and/or renal pelvis. The retention portion 130 is desirably sufficiently bendable to absorb forces exerted on the catheter 112 and to prevent such forces from being translated to the ureters. For example, if the retention portion 130 is pulled in the proximal direction P (shown in FIG. 9A) toward the patient's bladder, the retention portion 130 can be sufficiently flexible to begin to unwind or be straightened so that it can be drawn through the ureter. Similarly, when the retention portion 130 can be reinserted into the renal pelvis or other suitable region within the ureter, it can be biased to return to its deployed configuration.

In some examples, the retention portion 130 is integral with the tube 122. In that case, the retention portion 130 can be formed by imparting a bend or curl to the catheter body 122 that is sized and shaped to retain the catheter at a desired fluid collection location. Suitable bends or coils can include a pigtail coil, corkscrew coil, and/or helical coil, such as are shown in FIGS. 1, 2A, 7A, and 8A-10G. For example, the retention portion 130 can comprise one or more radially and longitudinally extending helical coils configured to contact and passively retain the catheter 112 within the ureter 6, 8 proximate to or within the renal pelvis 20, 21, as shown for example in FIGS. 2A, 7A, and 8A-10G. In other examples, the retention portion 130 is formed from a radially flared or tapered portion of the catheter body 122. For example, the retention portion 130 can further comprise a fluid collecting portion, as shown in FIGS. 17-41C, such as a tapered or funnel-shaped inner surface 186. In other examples, the retention portion 130 can comprise a separate element connected to and extending from the catheter body or tube 122.

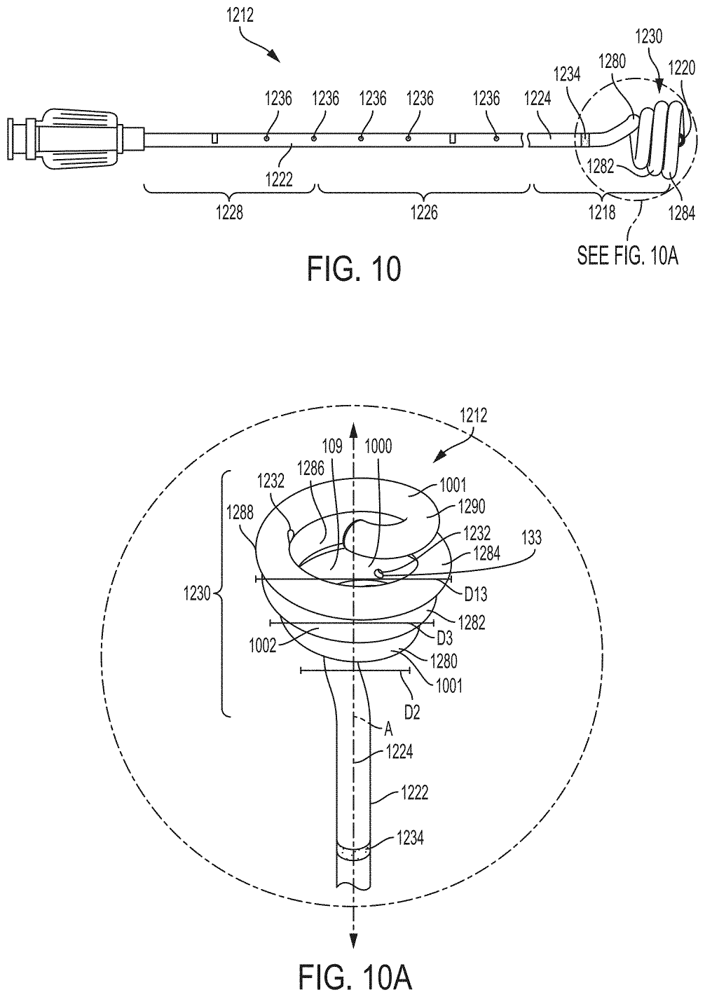

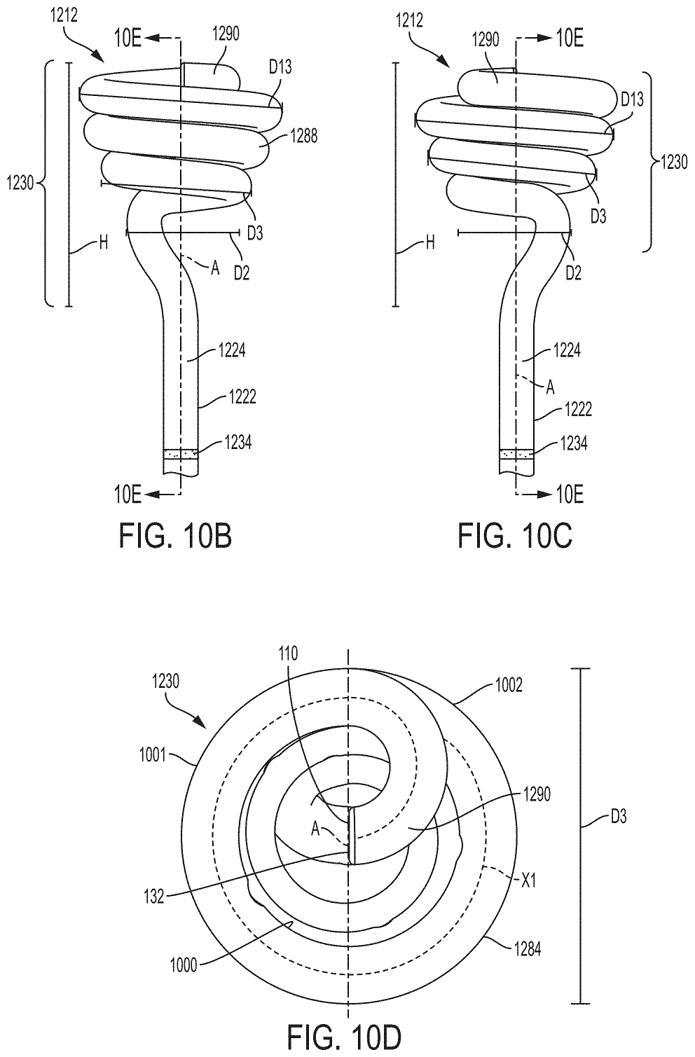

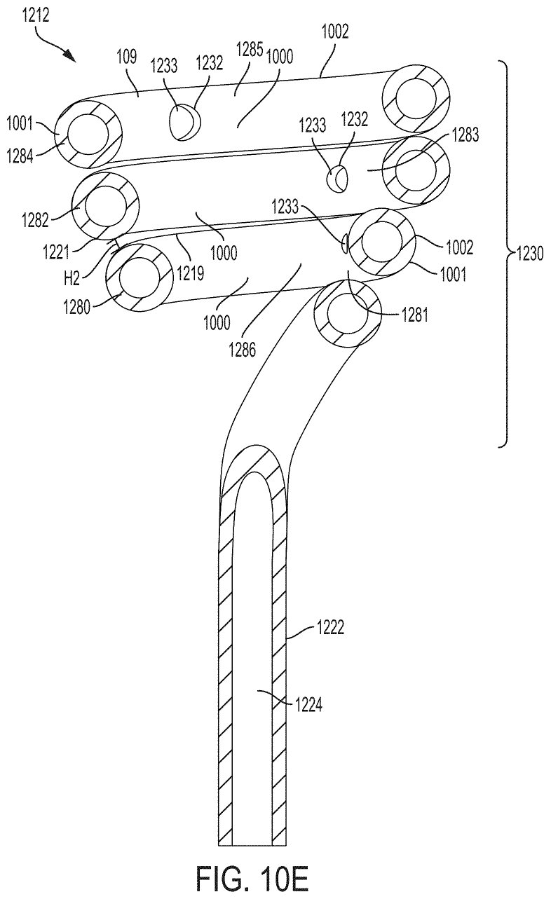

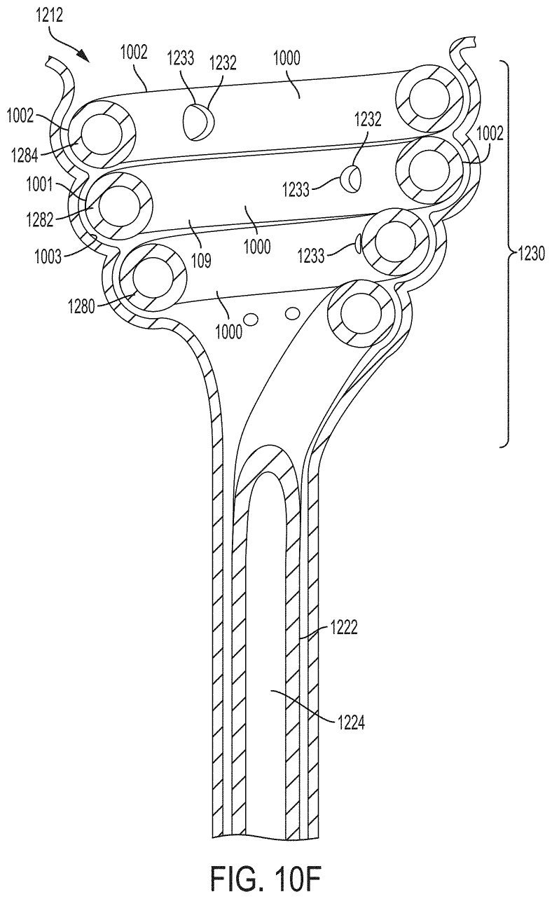

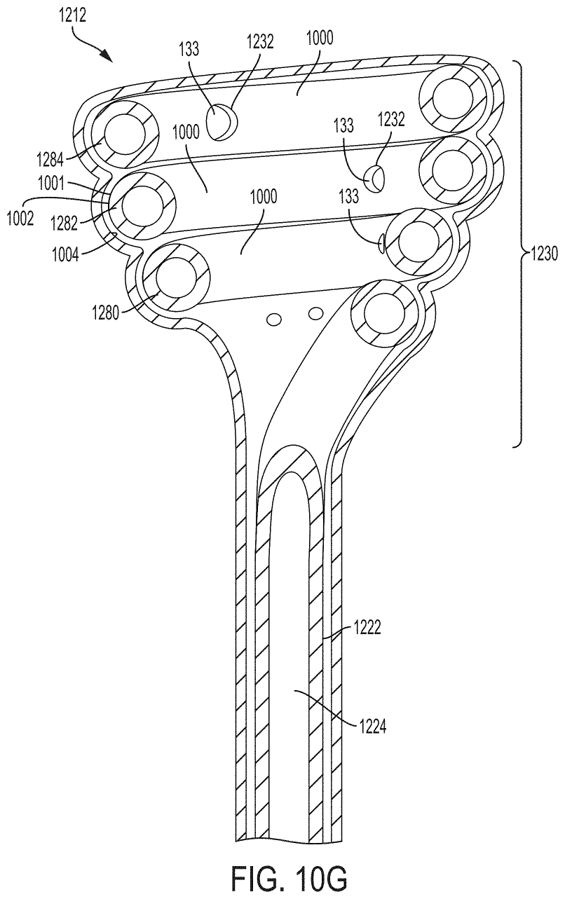

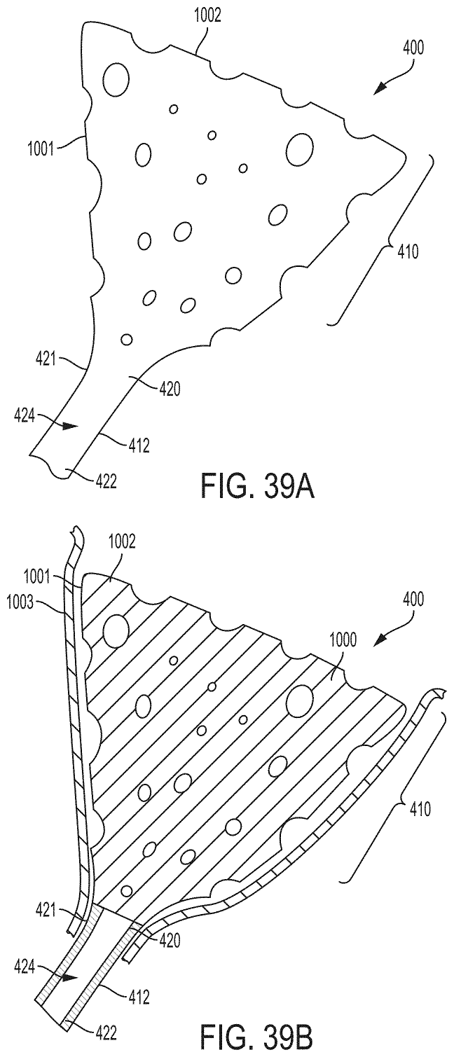

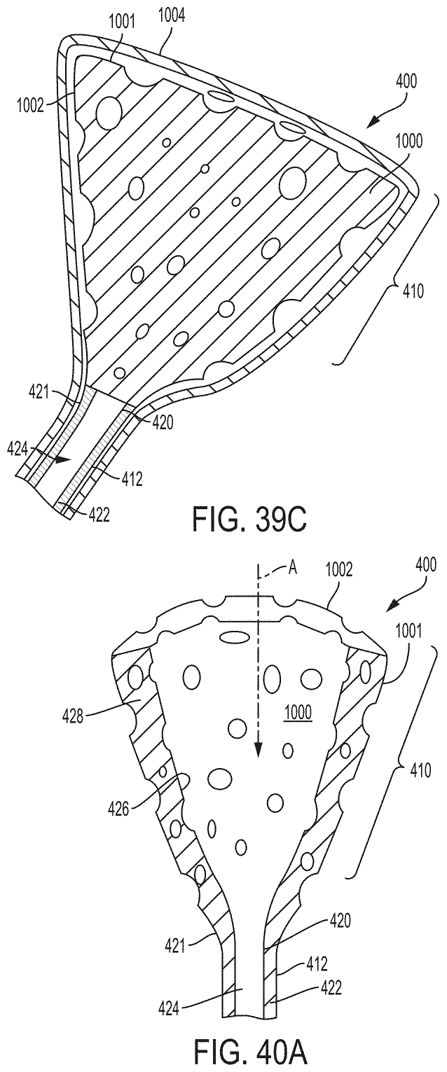

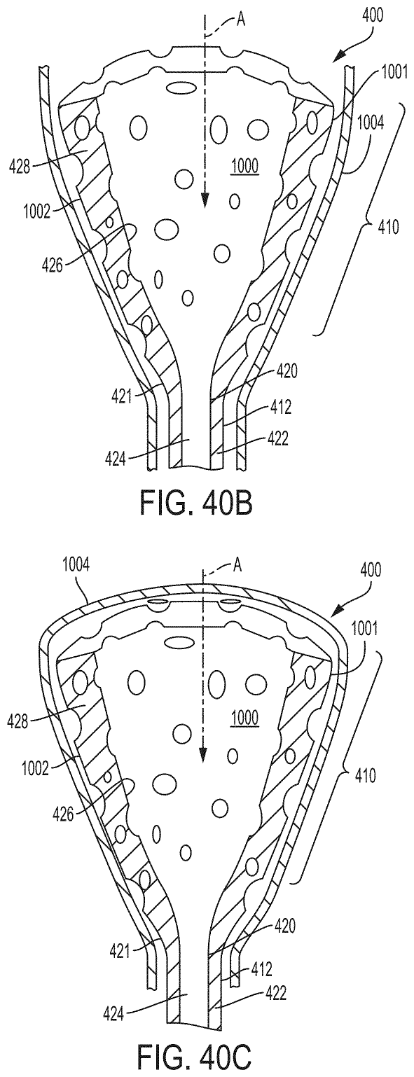

In some examples, the retention portion 130 can further comprise one or more perforated sections, such as drainage holes, perforations or ports 132, 1232 (shown, for example, in FIGS. 9A-9E, 10A, 10E, 11-14, 27, 32A, 32B, 33, 34, and 39-41A-C). A drainage port 132 can be located, for example, at the open distal end 120, 121 of the tube 122, as shown in FIG. 10D. In other examples, perforated sections and/or drainage ports 132, 1232 are disposed along the sidewall 109 of the distal portion 118 of the catheter tube 122, as shown in FIGS. 9A-9E, 10A, 10E, 11-14, 27, 32A, 32B, 33, 34, and 41A-C, or within the material of the retention portion, such as the sponge material of FIGS. 39 and 40. The drainage ports or holes 132, 1232 can be used for assisting in fluid collection by which fluid can flow into the drainage lumen for removal from the patient's body. In other examples, the retention portion 130 is solely a retention structure and fluid collection and/or imparting negative pressure is provided by structures at other locations on the catheter tube 122.

In some examples, such as are shown in FIGS. 9B-E, 10D-G, 18B, 18C-E, 20, 22A-35, 37B, 38A, 39B, 40A-41C, at least a portion of, most, or all of the drainage holes, ports or perforations 132, 1232 are positioned in the ureteral catheter 112, 114 or bladder catheter 116 in protected surface areas or inner surface areas 1000, such that tissue 1004, 1003 from the bladder or kidney does not directly contact or partially or fully occlude the protected drainage holes, ports or perforations 133. For example, as shown in FIGS. 2A-2C, 7A, 7B, 10F, 17, 18D, 24B, 29C, 39B, 40B, and 41B, when negative pressure is induced in the ureter and/or renal pelvis, a portion of the mucosal tissue 1003 of the ureter and/or kidney may be drawn against the outer periphery 72, 1002 or protective surface areas 1001 or outer regions of the retention portion 130 and may partially or fully occlude some drainage holes, ports or perforations 134 positioned on the outer periphery 72, 1002 or protective surface areas 1001 of the retention portion 130. Similarly, as shown in FIGS. 2A-2C, 7A, 7B, 10G, 17, 18E, 24C, 39C, 40C, and 41C, when negative pressure is induced in the bladder, a portion of the bladder tissue 1004, such as the transitional epithelial tissue lining, lamina propria connective tissue, muscularis propria and/or fatty connective tissue, may be drawn against the outer periphery 72, 1002 or protective surface areas 1001 or outer regions of the retention portion 130 and may partially or fully occlude some drainage holes, ports or perforations 134 positioned on the outer periphery 1002 or protective surface areas 1001 or outer regions of the retention portion 130.

At least a portion of protected drainage ports 133 located on the protected surface areas or inner surface areas 1000 of the retention portion 130 would not be partially or fully occluded when such tissues 1003, 1004 contact the outer periphery 72, 1002 or protective surface areas 1001 or outer regions of the retention portion 130. Further, risk of injury to the tissues 1003, 1004 from pinching or contact with the drainage ports 133 can be reduced or ameliorated. The configuration of the outer periphery 72, 1002 or protective surface areas 1001 or outer regions of the retention portion 130 depends upon the overall configuration of the retention portion 130. Generally, the outer periphery 72, 1002 or protective surface areas 1001 or outer regions of the retention portion 130 contacts and supports the bladder 1004 or kidney tissue 1003, and thereby inhibits occlusion or blockage of the protected drainage holes, ports or perforations 133.

For example, as shown in FIG. 10E-G, there is shown an exemplary retention portion 1230 comprising a plurality of helical coils 1280, 1282, 1284. The outer periphery 1002 or protective surface areas 1001 or outer regions of the helical coils 1280, 1282, 1284 contact and support the bladder tissue 1004 or kidney tissue 1003 to inhibit occlusion or blockage of protected drainage holes, ports or perforations 1233 positioned in protected surface areas or inner surface areas 1000 of the helical coils 1280, 1282, 1284. The outer periphery 1002 or protective surface areas 1001 or outer regions of the helical coils 1280, 1282, 1284 provides protection for the protected drainage holes, ports or perforations 1233. In FIG. 10F, the kidney tissue 1003 is shown surrounding and contacting at least a portion of the outer periphery 1002 or protective surface areas 1001 or outer regions of the helical coils 1280, 1282, 1284, which inhibits contact of the kidney tissue 1003 with the protected surface areas or inner surface areas 1000 of the helical coils 1280, 1282, 1284, and thereby inhibits partial or full blockage of the protected drainage holes, ports or perforations 1233 by the kidney tissue 1003. In FIG. 10G, the bladder tissue 1004 is shown surrounding and contacting at least a portion of the outer periphery 1002 or protective surface areas 1001 or outer regions of the helical coils 1280, 1282, 1284, which inhibits contact of the bladder tissue 1004 with the protected surface areas or inner surface areas 1000 of the helical coils 1280, 1282, 1284, and thereby inhibits partial or full blockage of the protected drainage holes, ports or perforations 1233 by the bladder tissue 1004.

Similarly, other examples of configurations of bladder and/or ureteral retention portions shown in FIGS. 1, 2A, 7A, 17, 18A, 18B, 18C, 19, 20, 21, 22A, 22B, 23A, 23B, 24, 25, 26, 27, 28A, 28B, 29A, 29B, 30, 31, 32A, 32B, 33, 34, 35A, 35B, 36, 37A, 37B, 38A, 38B, 39, 40, and 41 provide an outer periphery 1002 or protective surface areas 1001 or outer regions which can contact and support the bladder tissue 1004 or kidney tissue 1003 to inhibit occlusion or blockage of protected drainage holes, ports or perforations 133, 1233 positioned in protected surface areas or inner surface areas 1000 of the retention portions. Each of these examples will be discussed further below.

Referring now to FIGS. 8A, 8B, and 9A-9E, exemplary retention portions 130 for ureteral catheters or bladder catheters comprising a plurality of helical coils, such as one or more full coils 184 and one or more half or partial coils 183, are illustrated. The retention portion 130 is capable of moving between a contracted position and the deployed position with the plurality of helical coils. For example, a substantially straight guidewire can be inserted through the retention portion 130 to maintain the retention portion 130 in a substantially straight contracted position. When the guidewire is removed, the retention portion 130 can transition to its coiled configuration. In some examples, the coils 183, 184 extend radially and longitudinally from the distal portion 118 of the tube 122. With specific reference to FIGS. 8A and 8B, in an exemplary embodiment, the retention portion 130 comprises two full coils 184 and one half coil 183. For example, as shown in FIGS. 8A and 8B, the outer diameter of the full coils 184, shown by line D1, can be about 18±2 mm, the half coil 183 diameter D2 can be about 14 mm±2 mm, and the coiled retention portion 130 can have a height H of about 16±2 mm.

The retention portion 130 can further comprise the one or more drainage holes 132, 1232 (shown in FIGS. 9A-9E, 10A and 10E, for example) configured to draw fluid into an interior of the catheter tube 122. In some examples, the retention portion 130 can comprise two, three, four, five, six, seven, eight or more drainage holes 132, 1232, plus an additional hole 110 at the distal tip or end 120 of the retention portion. In some examples, the diameter of each of the drainage holes 132, 1232 (shown in FIGS. 9A-9E, 10A and 10E, for example) can range from about 0.7 mm to 0.9 mm and, preferably, is about 0.83±0.01 mm. In some examples, the diameter of the additional hole 110 at the distal tip or end of the retention portion 130 (shown in FIGS. 9A-9E, 10A and 10E, for example) can range from about 0.165 mm to about 2.39 mm, or about 0.7 to about 0.97 mm. The distance between adjacent drainage holes 132, specifically the linear distance between the closest outer edges of adjacent drainage holes 132, 1232 when the coils are straightened, can be about 15 mm±2.5 mm, or about 22.5±2.5 mm or more.

As shown in FIGS. 9A-9E, in another exemplary embodiment, the distal portion 118 of the drainage lumen 124 proximal to the retention portion 130 defines a straight or curvilinear central axis L. In some examples, at least a half or first coil 183 and a full or second coil 184 of the retention portion 130 extend about an axis A of the retention portion 130. The first coil 183 initiates or begins at a point where the tube 122 is bent at an angle α ranging from about 15 degrees to about 75 degrees from the central axis L, as indicated by angle α, and preferably about 45 degrees. As shown in FIGS. 9A and 9B, prior to insertion in the body, the axis A can be coextensive with the longitudinal central axis L. In other examples, as shown in FIGS. 9C-9E, prior to insertion in the body, the axis A extends from and is curved or angled, for example at angle (3, relative to the central longitudinal axis L.

In some examples, multiple coils 184 can have the same or different inner and/or outer diameter D and height H2 between adjacent coils 184. In that case, the outer diameter D1 of each of the coils 184 may range from about 10 mm to about 30 mm. The height H2 between each of the adjacent coils 184 may range from about 3 mm to about 10 mm.

In other examples, the retention portion 130 is configured to be inserted in the tapered portion of the renal pelvis. For example, the outer diameter D1 of the coils 184 can increase toward the distal end 120 of the tube 122, resulting in a helical structure having a tapered or partially tapered configuration. For example, the distal or maximum outer diameter D of the tapered helical portion ranges from about 10 mm to about 30 mm, which corresponds to the dimensions of the renal pelvis, and the outer diameter D1 of each adjacent coil can decrease closer to the proximal end 128 of the retention portion 130. The overall height H of the retention portion 130 can range from about 10 mm to about 30 mm.

In some examples, the outer diameter D1 of each coil 184 and/or height H2 between each of the coils 184 can vary in a regular or irregular fashion. For example, the outer diameter D1 of coils or height H2 between adjacent coils can increase or decrease by a regular amount (e.g., about 10% to about 25% between adjacent coils 184). For example, for a retention portion 130 having three coils (as shown, for example, in FIGS. 9A and 9B) an outer diameter D2 of a proximal-most coil or first coil 183 can be about 6 mm to 18 mm, an outer diameter D3 of a middle coil or second coil 185 can be about 8 mm to about 24 mm, and an outer diameter D13 of a distal-most or third coil 187 can be between about 10 mm and about 30 mm.

The retention portion 130 can further comprise the drainage perforations, holes or ports 132 disposed on or through the sidewall 109 of the catheter tube 122 on, or adjacent to, the retention portion 130 to permit urine waste to flow from the outside of the catheter tube 122 to the inside drainage lumen 124 of the catheter tube 122. The position and size of the drainage ports 132 can vary depending upon the desired flow rate and configuration of the retention portion 130. The diameter D11 of each of the drainage ports 132 can range independently from about 0.005 mm to about 1.0 mm. The spacing D12 between the closest edge of each of the drainage ports 132 can range independently from about 1.5 mm to about 5 mm. The drainage ports 132 can be spaced in any arrangement, for example, random, linear or offset. In some examples, the drainage ports 132 can be non-circular, and can have a surface area of about 0.00002 to 0.79 mm².

In some examples, as shown in FIG. 9A, the drainage ports 132 are located around the entire outer periphery 72, 1002 or protective surface area 1001 of the sidewall 109 of the catheter tube 122 to increase an amount of fluid that can be drawn into the drainage lumen 124 (shown in FIGS. 2, 9A, and 9B). In other examples, as shown in FIGS. 9B-9E and 10-10E, the drainage holes, ports or perforations 132 can be disposed essentially only or only on the protected surface areas or inner surface areas 1000 or radially inwardly facing side 1286 of the coils 184 to prevent occlusion or blockage of the drainage ports 132, 1232 and the outwardly facing side 1288 of the coils may be essentially free of drainage ports 132, 1232 or free of drainage ports 132, 1232. The outer periphery 72, 189, 1002 or protective surface area 1001 or outer regions 192 of the helical coils 183, 184, 1280, 1282, 1284 can contact and support the bladder tissue 1004 or kidney tissue 1003 to inhibit occlusion or blockage of protected drainage holes, ports or perforations 133, 1233 positioned in protected surface areas or inner surface areas 1000 of the helical coils 183, 184, 1280, 1282, 1284. For example, when negative pressure is induced in the ureter and/or renal pelvis, mucosal tissue of the ureter and/or kidney may be drawn against the retention portion 130 and may occlude some drainage ports 134 on the outer periphery 72, 189, 1002 of the retention portion 130. Drainage ports 133, 1233 located on the radially inward side 1286 or protected surface areas or inner surface areas 1000 of the retention structure would not be appreciably occluded when such tissues 1003, 1004 contact the outer periphery 72, 189, 1002 or protective surface area 1001 or outer regions of the retention portion 130. Further, risk of injury to the tissues from pinching or contact with the drainage ports 132, 133, 1233, or protected drainage holes, ports or perforations 133, 1233 can be reduced or ameliorated.

With reference to FIGS. 9C and 9D, other examples of ureteral catheters 112 having a retention portion 130 comprising a plurality of coils 184 are illustrated. As shown in FIG. 9C, the retention portion 130 comprises three coils 184 extending about the axis A. The axis A is a curved arc extending from the central longitudinal axis L of the portion of the drainage lumen 181 proximal to the retention portion 130. The curvature imparted to the retention portion 130 can be selected to correspond to the curvature of the renal pelvis, which comprises a cornucopia-shaped cavity.

As shown in FIG. 9D, in another exemplary embodiment, the retention portion 130 can comprise two coils 184 extending about an angled axis A. The angled axis A extends at an angle from a central longitudinal axis L, and is angled, as shown by angle (3, relative to an axis generally perpendicular to the central axis L of the portion of the drainage lumen. The angle (3 can range from about 15 to about 75 degrees (e.g., about 105 to about 165 degrees relative to the central longitudinal axis L of the drainage lumen portion of the catheter 112).

FIG. 9E shows another example of a ureteral catheter 112. The retention portion comprises three helical coils 184 extending about an axis A. The axis A is angled, as shown by angle β, relative to the horizontal. As in the previously-described examples, the angle β can range from about 15 to about 75 degrees (e.g., about 105 to about 165 degrees relative to the central longitudinal axis L of the drainage lumen portion of the catheter 112).

In some examples shown in FIGS. 10-10E, the retention portion 1230 is integral with the tube 1222. In other examples, the retention portion 1230 can comprise a separate tubular member connected to and extending from the tube or drainage lumen 1224.

In some examples, the retention portion comprises a plurality of radially extending coils 184. The coils 184 are configured in the shape of a funnel, and thereby form a funnel support. Some examples of coil funnel supports are shown in FIGS. 2A-C, 7A, 7B, 8A, and 8A-10E.

In some examples, the at least one sidewall 119 of the funnel support comprises at least a first coil 183 having a first diameter and a second coil 184 having a second diameter, the first diameter being less than the second diameter, wherein the maximum distance between a portion of a sidewall of the first coil and a portion of an adjacent sidewall of the second coil ranges from about 0 mm to about 10 mm. In some examples, the first diameter of the first coil 183 ranges from about 1 mm to about 10 mm and the second diameter of the second coil 184 ranges from about 5 mm to about 25 mm. In some examples, the diameter of the coils increases toward a distal end of the drainage lumen, resulting in a helical structure having a tapered or partially tapered configuration. In some embodiments, the second coil 184 is closer to an end of the distal portion 118 of the drainage lumen 124 than is the first coil 183. In some examples, the second coil 184 is closer to an end of the proximal portion 128 of the drainage lumen 124 than is the first coil 183.

In some examples, the at least one sidewall 119 of the funnel support comprises an inwardly facing side 1286 and an outwardly facing side 1288, the inwardly facing side 1286 comprising at least one opening 133, 1233 for permitting fluid flow into the drainage lumen, the outwardly facing side 1288 being essentially free of or free of openings, as discussed below. In some examples, the at least one opening 133, 1233 has an area ranging from about 0.002 mm² to about 100 mm².

In some examples, the first coil 1280 comprises a sidewall 119 comprising a radially inwardly facing side 1286 and a radially outwardly facing side 1288, the radially inwardly facing side 1286 of the first coil 1280 comprising at least one opening 1233 for permitting fluid flow into the drainage lumen.

In some examples, the first coil 1280 comprises a sidewall 119 comprising a radially inwardly facing side 1286 and a radially outwardly facing side 1288, the radially inwardly facing side 1286 of the first coil 1280 comprising at least two openings 1233 for permitting fluid flow into the drainage lumen 1224.

In some examples, the first coil 1280 comprises a sidewall 119 comprising a radially inwardly facing side 1286 and a radially outwardly facing side 1288, the radially outwardly facing side 1288 of the first coil 1280 being essentially free of or free of one or more openings 1232.

In some examples, the first coil 1280 comprises a sidewall 119 comprising a radially inwardly facing side 1286 and a radially outwardly facing side 1288, the radially inwardly facing side 1286 of the first coil 1280 comprising at least one opening 1233 for permitting fluid flow into the drainage lumen 1224 and the radially outwardly facing side 1288 being essentially free of or free of one or more openings 1232.

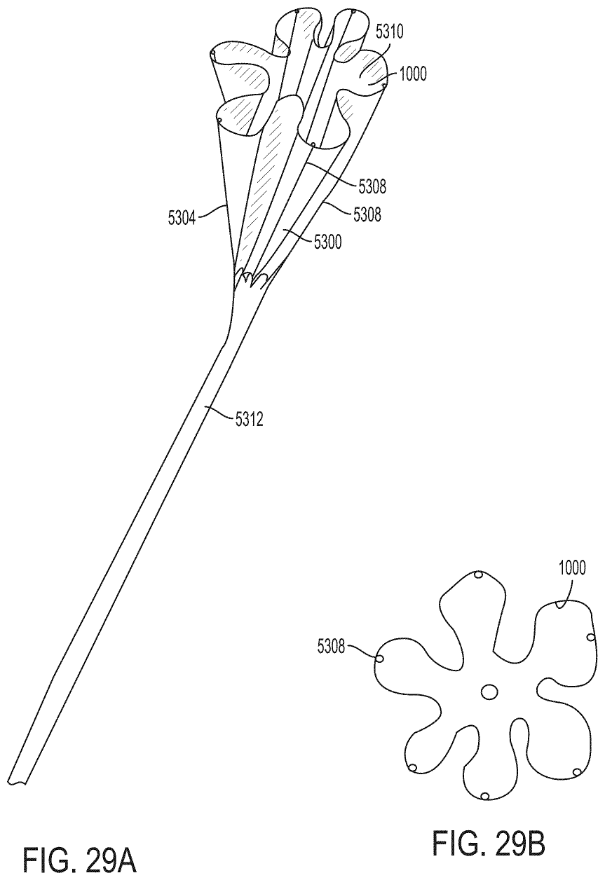

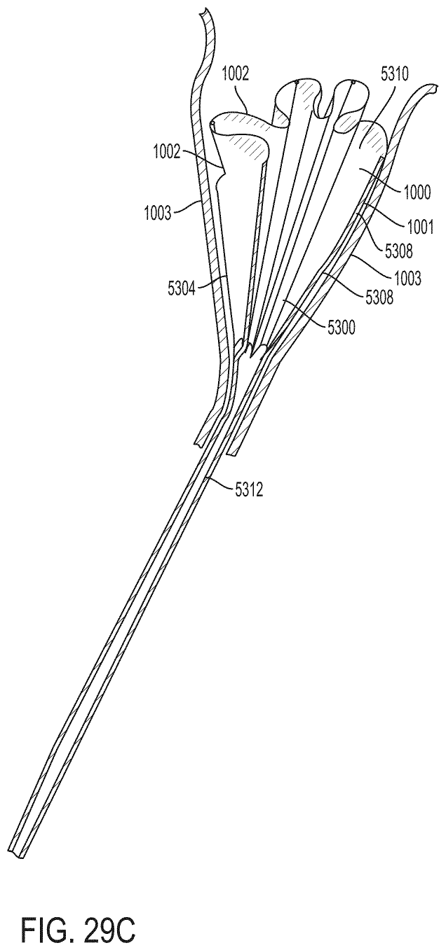

Referring now to FIGS. 10-10E, in some examples, the distal portion 1218 comprises an open distal end 1220 for drawing fluid into the drainage lumen 1224. The distal portion 1218 of the ureteral catheter 1212 further comprises a retention portion 1230 for maintaining the distal portion 1218 of the drainage lumen or tube 1222 in the ureter and/or kidney. In some examples, the retention portion 1230 comprises a plurality of radially extending coils 1280, 1282, 1284. The retention portion 1230 can be flexible and bendable to permit positioning of the retention portion 1230 in the ureter, renal pelvis, and/or kidney. For example, the retention portion 1230 is desirably sufficiently bendable to absorb forces exerted on the catheter 1212 and to prevent such forces from being translated to the ureters. Further, if the retention portion 1230 is pulled in the proximal direction P (shown in FIGS. 9A-9E) toward the patient's bladder 10, the retention portion 1230 can be sufficiently flexible to begin to unwind or be straightened so that it can be drawn through the ureter 6, 8. In some examples, the retention portion 1230 is integral with the tube 1222. In other examples, the retention portion 1230 can comprise a separate tubular member connected to and extending from the tube or drainage lumen 1224. In some examples, the catheter 1212 comprises a radiopaque band 1234 (shown in FIG. 29) positioned on the tube 1222 at a proximal end of the retention portion 1230. The radiopaque band 1234 is visible by fluoroscopic imaging during deployment of the catheter 1212. In particular, a user can monitor advancement of the band 1234 through the urinary tract by fluoroscopy to determine when the retention portion 1230 is in the renal pelvis and ready for deployment.

In some examples, the retention portion 1230 comprises perforations, drainage ports, or openings 1232 in a sidewall of the tube 1222. As described herein, a position and size of the openings 1232 can vary depending upon a desired volumetric flow rate for each opening and size constraints of the retention portion 1230. In some examples, a diameter D11 of each of the openings 1232 can range independently from about 0.05 mm to about 2.5 mm and have an area of about 0.002 mm² to about 5 mm². Openings 1232 can be positioned extending along on a sidewall 119 of the tube 1222 in any direction desired, such as longitudinal and/or axial. In some examples, spacing between the closest adjacent edge of each of the openings 1232 can range from about 1.5 mm to about 15 mm. Fluid passes through one or more of the perforations, drainage ports, or openings 1232 and into the drainage lumen 1234. Desirably, the openings 1232 are positioned so that they are not occluded by tissues 1003 of the ureters 6, 8 or kidney when negative pressure is applied to the drainage lumen 1224. For example, as described herein, openings 1233 can be positioned on interior portions or protected surfaces areas 1000 of coils or other structures of the retention portion 1230 to avoid occlusion of the openings 1232, 1233. In some examples, the middle portion 1226 and proximal portion 1228 of the tube 1222 can be essentially free of or free from perforations, ports, openings or openings to avoid occlusion of openings along those portions of the tube 1222. In some examples, a portion 1226, 1228 which is essentially free from perforations or openings includes substantially fewer openings 1232 than other portions such as distal portion 1218 of the tube 1222. For example, a total area of openings 1232 of the distal portion 1218 may be greater than or substantially greater than a total area of openings of the middle portion 1226 and/or the proximal portion 1228 of the tube 1222.

### Helical Coil Retention Portion

Referring now to FIGS. 10A-10E, an exemplary retention portion 1230 comprises helical coils 1280, 1282, 1284. In some examples, the retention portion 1230 comprises a first or half coil 1280 and two full coils, such as a second coil 1282 and a third coil 1284. As shown in FIGS. 10A-10D, in some examples, the first coil 1280 comprises a half coil extending from 0 degrees to 180 degrees around a curvilinear central axis A of the retention portion 1230. In some examples, as shown the curvilinear central axis A is substantially straight and co-extensive with a curvilinear central axis of the tube 1222. In other examples, the curvilinear central axis A of the retention portion 1230 can be curved giving the retention portion 1230, for example, a cornucopia shape. The first coil 1280 can have a diameter D1 of about 1 mm to 20 mm and preferably about 8 mm to 10 mm. The second coil 1282 can be a full coil extending from 180 degrees to 540 degrees along the retention portion 1230 having a diameter D2 of about 5 mm to 50 mm, preferably about 10 mm to 20 mm, and more preferably about 14 mm±2 mm. The third coil 1284 can be a full coil extending between 540 degrees and 900 degrees and having a diameter D3 of between 5 mm and 60 mm, preferably about 10 mm to 30 mm, and more preferably about 18 mm±2 mm. In other examples, multiple coils 1282, 1284 can have the same inner and/or outer diameter. For example, an outer diameter of the full coils 1282, 1284, can each be about 18±2 mm.

In some examples, an overall height H of the retention portion 1230 ranges from about 10 mm to about 30 mm and, preferably about 18±2 mm. A height H2 of a gap between adjacent coils 1284, namely between the sidewall 1219 of the tube 1222 of the first coil 1280 and the adjacent sidewall 1221 of the tube 122 of the second coil 1282 is less than 3.0 mm, preferably between about 0.25 mm and 2.5 mm, and more preferably between about 0.5 mm and 2.0 mm.

The retention portion 1230 can further comprise a distal-most curved portion 1290. For example, the distal most portion 1290 of the retention portion 1230, which includes the open distal end 1220 of the tube 1222, can be bent inwardly relative to a curvature of the third coil 1284. For example, a curvilinear central axis X1 (shown in FIG. 10D) of the distal-most portion 1290 can extend from the distal end 1220 of the tube 1222 towards the curvilinear central axis A of the retention portion 1230.

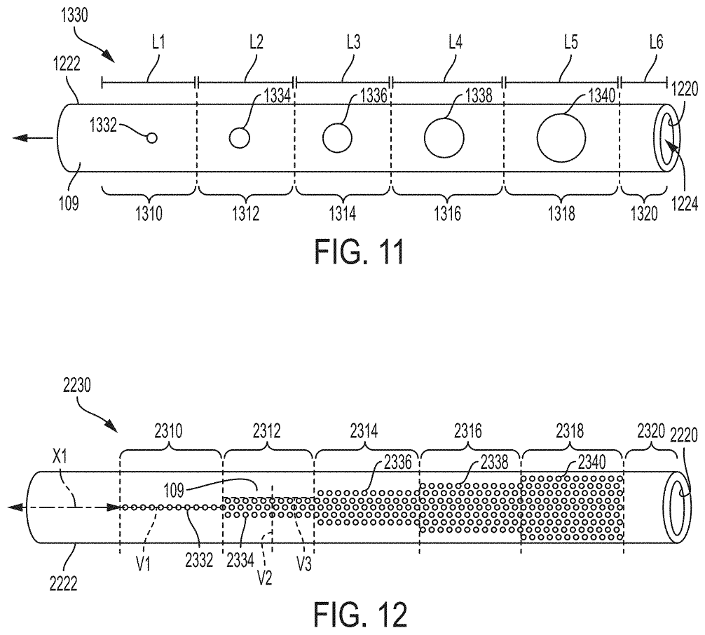

The retention portion 1230 is capable of moving between a contracted position, in which the retention portion 1230 is straight for insertion into the patient's urinary tract, and the deployed position, in which the retention portion 1230 comprises the helical coils 1280, 1282, 1284. Generally, the tube 1222 is naturally biased toward the coiled configuration. For example, an uncoiled or substantially straight guidewire can be inserted through the retention portion 1230 to maintain the retention portion 1230 in its straight contracted position, as shown for example in FIGS. 11-14. When the guidewire is removed, the retention portion 1230 naturally transitions to its coiled position.

In some examples, the openings 1232, 1233 are disposed essentially only or only on a radially inwardly facing side 1286 or protected surface area or inner surface area 1000 of the coils 1280, 1282, 1284 to prevent occlusion or blockage of the openings 1232, 1233. A radially outwardly facing side 1288 of the coils 1280, 1282, 1284 may be essentially free of the openings 1232. In similar examples, a total area of openings 1232, 1233 on the inwardly facing side 1286 of the retention portion 1230 can be substantially greater than a total area of openings 1232 on the radially outwardly facing side 1288 of the retention portion 1230. Accordingly, when negative pressure is induced in the ureter and/or renal pelvis, mucosal tissue of the ureter and/or kidney may be drawn against the retention portion 1230 and may occlude some openings 1232 on the outer periphery 1002 or protective surface area 1001 of the retention portion 1230. However, openings 1232 located on the radially inward side 1286 or protected surface area or inner surface area 1000 of the retention portion 1230 are not appreciably occluded when such tissues contacts the outer periphery 1002 or protective surface area 1001 of the retention portion 1230. Therefore, risk of injury to the tissues from pinching or contact with the drainage openings 1232 can be reduced or eliminated.

### Hole or Opening Distribution Examples

In some examples, the first coil 1280 can be free or essentially free from openings 1232. For example, a total area of openings 1232 on the first coil 1280 can be less than or substantially less than a total area of openings 1232 of the full coils 1282, 1284. Examples of various arrangements of openings or openings 1232, which could be used for a coiled retention portion (such as coiled retention portion 1230 shown in FIGS. 10A-10E), are illustrated in FIGS. 11-14. As shown in FIGS. 11-14, a retention portion 1330 is depicted in its uncoiled or straight position, as occurs when a guidewire is inserted through the drainage lumen.

An exemplary retention portion 1330 is illustrated in FIG. 11. In order to more clearly describe positioning of openings of the retention portion 1330, the retention portion 1330 is referred to herein as being divided into a plurality of sections or perforated sections, such as a proximal-most or first section 1310, a second section 1312, a third section 1314, a fourth section 1316, a fifth section 1318, and a distal-most or sixth section 1320. One of ordinary skill in the art would understand that fewer or additional sections can be included, if desired. As used herein, “section” refers to a discrete length of the tube 1322 within the retention portion 1330. In some examples, sections are equal in length. In other examples, some sections can have the same length, and other sections can have a different length. In other examples, each section has a different length. For example, each of sections 1310, 1312, 1314, 1316, 1318 and 1320 can have a length L1-L6, respectively, ranging from about 5 mm to about 35 mm, and preferably from about 5 mm to 15 mm.

In some examples, each section 1310, 1312, 1314, 1316, 1318 and 1320 comprises one or more openings 1332. In some examples, each section each comprises a single opening 1332. In other examples, the first section 1310 includes a single opening 1332 and other sections comprise multiple openings 1332. In other examples, different sections comprise one or more openings 1332, each of the opening(s) having a different shape or different total area.

In some examples, such as the retention portion 1230 shown in FIGS. 10A-10E, the first or half coil 1280, which extends from 0 to about 180 degrees of the retention portion 1230 can be free from or essentially free from openings. The second coil 1282 can include the first section 1310 extending between about 180 and 360 degrees. The second coil 1282 can also include the second and third sections 1312, 1314 positioned between about 360 degrees and 540 degrees of the retention portion 1230. The third coil 1284 can include the fourth and fifth sections 1316, 1318 positioned between about 540 degrees and 900 degrees of the retention portion 1230.

In some examples, the openings 1332 can be sized such that a total area of openings of the first section 1310 is less than a total area of openings of the adjacent second section 1312. In a similar manner, if the retention portion 1330 further comprises a third section 1314, then openings of a third section 1314 can have a total area that is greater than the total area of the openings of the first section 1310 or the second section 1312. Openings of the forth 1316, fifth 1318, and sixth 1320 sections may also have a gradually increasing total area and/or number of openings to improve fluid flow through the tube 1222.

As shown in FIG. 11, the retention portion 1230 of the tube includes five sections 1310, 1312, 1314, 1316, 1318, each of which includes a single opening 1332, 1334, 1336, 1338, 1340. The retention portion 1330 also includes a sixth section 1320 which includes the open distal end 1220 of the tube 1222. In this example, the opening 1332 of the first section 1310 has the smallest total area. For example, a total area of the opening 1332 of the first section can range from about 0.002 mm² and about 2.5 mm², or about 0.01 mm² and 1.0 mm², or about 0.1 mm² and 0.5 mm². In one example, the opening 1332 is about 55 mm from the distal end 1220 of the catheter, has a diameter of 0.48 mm, and an area of 0.18 mm². In this example, a total area of openings 1334 of the second section 1312 is greater than the total area of openings 1232 of the first section 1310 and can range in size from about 0.01 mm² to about 1.0 mm². The third 1336, fourth 1338, and fifth 1350 openings can also range in size from about 0.01 mm² to about 1.0 mm². In one example, the second opening 1334 is about 45 mm from the distal end of the catheter 1220, has a diameter of about 0.58 mm, and an area of about 0.27 mm². The third opening 1336 can be about 35 mm from the distal end of the catheter 1220 and have a diameter of about 0.66 mm. The fourth opening 1338 can be about 25 mm from the distal end 1220 and have a diameter of about 0.76 mm. The fifth opening 1340 can be about 15 mm from the distal end 1220 of the catheter and have a diameter of about 0.889 mm. In some examples, the open distal end 1220 of the tube 1222 has the largest opening having an area ranging from about 0.5 mm² to about 5.0 mm² or more. In one example, the open distal end 1220 has a diameter of about 0.97 mm and an area of about 0.74 mm².

As described herein, openings 1332 1334, 1336, 1338, 1340 can be positioned and sized so that a volumetric flow rate of fluid passing through the first opening 1332 more closely corresponds to a volumetric flow rate of openings of more distal sections, when negative pressure is applied to the drainage lumen 1224 of the catheter 1212, for example from the proximal portion 1228 of the drainage lumen 1224. As described above, if each opening were the same area, then, when negative pressure is applied to the drainage lumen 1224, the volumetric flow rate of fluid passing through the proximal-most of first opening 1332 would be substantially greater than a volumetric flow rate of fluid passing through openings 1334 closer to the distal end 1220 of the retention portion 1330. While not intending to be bound by any theory, it is believed that when negative pressure is applied, the pressure differential between the interior of the drainage lumen 1224 and external to the drainage lumen 1224 is greater in the region of the proximal-most opening and decreases at each opening moving towards the distal end of the tube. For example, sizes and positions of the openings 1332 1334, 1336, 1338, 1340 can be selected so that a volumetric flow rate for fluid which flows into openings 1334 of the second section 1312 is at least about 30% of a volumetric flow rate of fluid which flows into the opening(s) 1332 of the first section 1310. In other examples, a volumetric flow rate for fluid flowing into the proximal-most or first section 1310 is less than about 60% of a total volumetric flow rate for fluid flowing through the proximal portion of the drainage lumen 1224. In other examples, a volumetric flow rate for fluid flowing into openings 1332, 1334 of the two proximal-most sections (e.g., the first section 1310 and the second section 1312) can be less than about 90% of a volumetric flow rate of fluid flowing through the proximal portion of the drainage lumen 1224 when a negative pressure, for example a negative pressure of about −45 mmHg, is applied to the proximal end of the drainage lumen.

As will be appreciated by one of ordinary skill in the art, volumetric flow rate and distribution for a catheter or tube comprising a plurality of openings or perforations can be directly measured or calculated in a variety of different ways. As used herein, “volumetric flow rate” means actual measurement of the volumetric flow rate downstream and adjacent to each opening or using a method for “Calculated Volumetric Flow Rate” described below.

For example, actual measurement of the dispersed fluid volume over time can be used to determine the volumetric flow rate through each opening 1332, 1334, 1336, 1338, 1340. In one exemplary experimental arrangement, a multi-chamber vessel comprising individual chambers sized to receive sections 1310, 1312, 1314, 1316, 1318, 1320 of the retention portion 1330 could be sealed around and enclose the retention portion 1330. Each opening 1332, 1334, 1336, 1338, 1340 could be sealed in one of the chambers. An amount of fluid volume drawn from the respective chamber into the tube 3222 through each opening 1332, 1334, 1336, 1338, 1340 could be measured to determine an amount of fluid volume drawn into each opening over time when a negative pressure is applied. The cumulative amount of fluid volume collected in the tube 3222 by a negative pressure pump system would be equivalent to the sum of fluid drawn into each opening 1332, 1334, 1336, 1338, 1340.

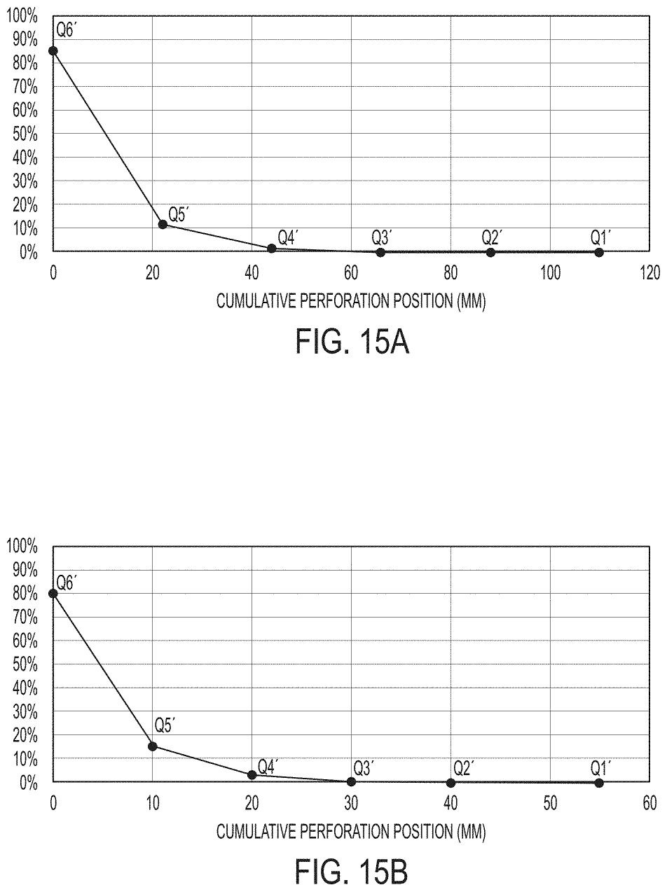

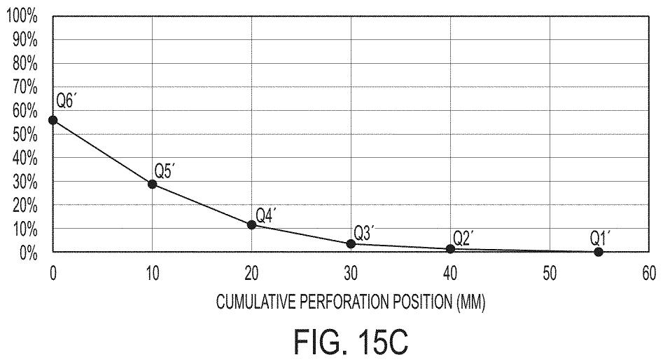

Alternatively, volumetric fluid flow rate through different openings 1332 1334, 1336, 1338, 1340 can be calculated mathematically using equations for modeling fluid flow through a tubular body. For example, volumetric flow rate of fluid passing through openings 1332 1334, 1336, 1338, 1340 and into the drainage lumen 1224 can be calculated based on a mass transfer shell balance evaluation, as described in detail below in connection with the Mathematical Examples and FIGS. 15A-15C. Steps for deriving mass balance equations and for calculating a flow distribution between or volumetric flow rates for the openings 1332 1334, 1336, 1338, 1340 are also described in detail below in connection with FIGS. 15A-15C.

Another exemplary retention portion 2230 with openings 2332, 2334, 2336, 2338, 2340 is illustrated in FIG. 12. As shown in FIG. 12, the retention portion 2230 comprises numerous smaller perforations or openings 2332, 2334, 2336, 2338, 2340. Each of the openings 2332, 2334, 2336, 2338, 2340 can have a substantially identical cross-sectional area or one or more openings 2332, 2334, 2336, 2338, 2340 can have different cross-sectional areas. As shown in FIG. 12, the retention portion 2330 comprises six sections 2310, 2312, 2314, 2316, 2318, 2320, such as are described above, wherein each section comprises a plurality of the openings 2332, 2334, 2336, 2338, 2340. In the example shown in FIG. 12, a number of openings 2332, 2334, 2336, 2338, 2340 per section increases towards the distal end 2220 of the tube 2222, such that a total area of openings 1332 in each section increases compared to a proximally adjacent section.

As shown in FIG. 12, openings 2332 of the first section 2310 are arranged along a first virtual line V1, which is substantially parallel to a central axis X1 of the retention portion 2230. Openings 2334, 2336, 2338, 2340 of the second 2312, third 2314, fourth 2316, and fifth 2318 sections, respectively, are positioned on the sidewall of the tube 2222 in a gradually increasing number of rows, such that openings 2334, 2336, 2338, 2340 of these sections also line up around a circumference of the tube 2222. For example, some of the openings 2334 of the second section 2312 are positioned such that a second virtual line V2 extending around a circumference of the sidewall of the tube 2222 contacts at least a portion of multiple openings 2334. For example, the second section 2312 can comprise two or more rows of perforations or openings 2334, in which each opening 2334 has an equal or different cross-sectional area. Further, in some examples, at least one of the rows of the second section 2312 can be aligned along a third virtual line V3, which is parallel with the central axis X1 of the tube 2222, but is not co-extensive with the first virtual line V1. In a similar manner, the third section 2314 can comprise five rows of perforations or openings 2336, in which each opening 2336 has an equal or different cross-sectional area; the fourth section 2316 can comprise seven rows of perforations or openings 2338; and the fifth section 2318 can comprise nine rows of perforations or openings 2340. As in previous examples, the sixth section 2320 comprises a single opening, namely the open distal end 2220 of the tube 2222. In the example of FIG. 12, each of the openings has the same area, although the area of one or more openings can be different if desired.

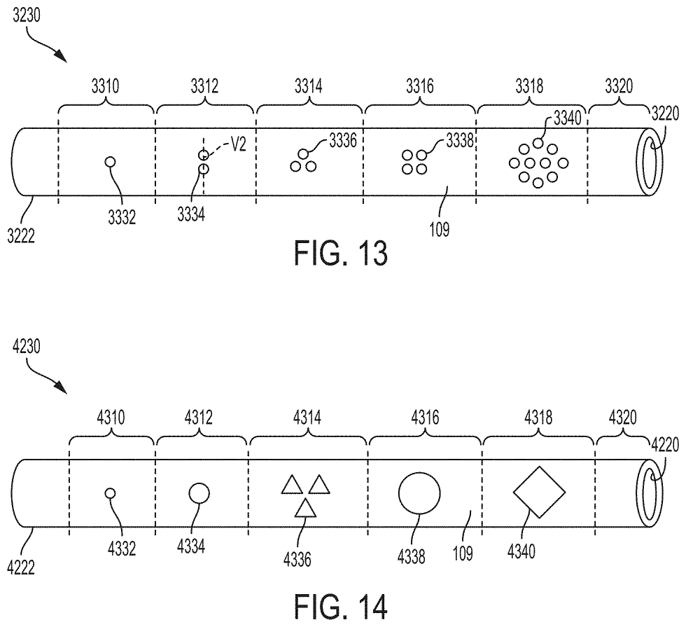

Another exemplary retention portion 3230 with openings 3332, 3334, 3336, 3338, 3340 is illustrated in FIG. 13. The retention portion 3230 of FIG. 13 includes a plurality of similarly sized perforations or openings 3332, 3334, 3336, 3338, 3340. As in previous examples, the retention portion 3230 can be divided into six sections 3310, 3312, 3314, 3316, 3318, 3320, each of which comprises at least one opening. The proximal-most or first section 3310 includes one opening 3332. The second section 3312 includes two openings 3334 aligned along the virtual line V2 extending around a circumference of the sidewall of the tube 3222. The third section 3314 comprises a grouping of three openings 3336, positioned at vertices of a virtual triangle. The fourth section 3316 comprises a grouping of four openings 3338 positioned at corners of a virtual square. The fifth section 3318 comprises ten openings 3340 positioned to form a diamond shape on the sidewall of the tube 3222. As in previous examples, the sixth section 3320 comprises a single opening, namely the open distal end 3220 of the tube 3222. The area of each opening can range from about 0.001 mm² and about 2.5 mm². In the example of FIG. 13, each of the openings has the same area, although the area of one or more openings can be different if desired.

Another exemplary retention portion 4230 with openings 4332, 4334, 4336, 4338, 4340 is illustrated in FIG. 14. The openings 4332 4334, 4336, 4338, 4340 of the retention portion 4330 have different shapes and sizes. For example, the first section 4310 includes a single circular opening 4332. The second section 4312 has a circular opening 4334 with a larger cross sectional area than the opening 4332 of the first section 4310. The third section 4314 comprises three triangular shaped openings 4336. The fourth section 4316 comprises a large circular opening 4338. The fifth section 4318 comprises a diamond shaped opening 4340. As in previous examples, the sixth section 4320 comprises the open distal end 4220 of the tube 4222. FIG. 14 illustrates one example of an arrangement of different shapes of openings in each section. It is understood that the shape of each opening in each section can be independently selected, for example the first section 4310 can have one or more diamond-shaped openings or other shapes. The area of each opening can be the same or different and can range from about 0.001 mm² and about 2.5 mm².

### Examples

Calculation of Volumetric Flow Rate and Percentage of Flow Distribution

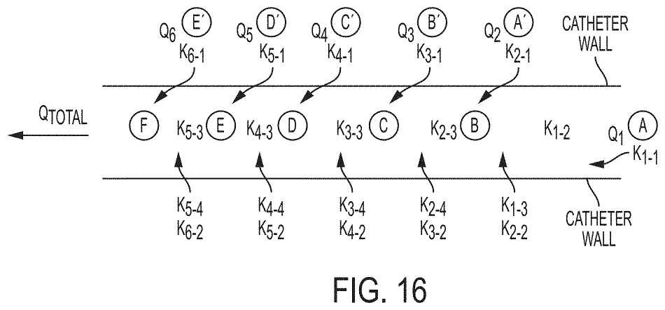

Having described various arrangements of openings for retention portions of the ureteral catheter 1212, a method for determining the Calculated Percentage of Flow Distribution and Calculated Volumetric Flow Rate through the catheter will now be described in detail. A schematic drawing of an exemplary catheter with sidewall openings showing a position of portions of the tube or drainage lumen used in the following calculations is shown in FIG. 16. Calculated Percentage of Flow Distribution refers to a percentage of total fluid flowing through proximal portions of the drainage lumen which entered the drainage lumen through different openings or sections of the retention portion. Calculated Volumetric Flow rate refers to fluid flow per unit time through different portions of the drainage lumen or openings of the retention portion. For example, a volumetric flow rate for a proximal portion of the drainage lumen describes a rate of flow for a total amount of fluid passing through the catheter. A volumetric flow rate for an opening refers to a volume of fluid which passes through the opening and into the drainage lumen per unit time. In Tables 3-5 below flow is described as a percentage of total fluid flow or of a total volumetric flow rate for a proximal portion of the drainage lumen. For example, an opening having a flow distribution of 100% means that all fluid entering the drainage lumen passed through the opening. An opening having a distribution of 0% would indicate that none of the fluid in the drainage lumen entered the drainage lumen through that opening.

These volumetric flow rate calculations were used to determine and model fluid flow through the retention portion 1230 of the ureter catheter 1212 shown in FIGS. 7A and 10-10E. Further, these calculations show that adjusting the area of openings and linear distribution of openings along the retention portion effects a distribution of fluid flow through different openings. For example, reducing the area of the proximal-most opening decreases the proportion of fluid drawn into the catheter through the proximal most opening and increases the proportion of fluid drawn into more distal openings of the retention portion.

For the following calculations, a tube length of 86 cm having an inner diameter of 0.97 mm and an end hole inner diameter of 0.97 mm was used. Density of urine was 1.03 g/mL and had a coefficient of friction μ of 8.02×10-3 Pa·S (8.02×10-3 kg/s·m) at 37° C. The urine volumetric flow rate passing through the catheter was 2.7 ml per minute (Q_{Total}) as determined by experimental measurement.

Calculated Volumetric Flow Rate is determined by a volumetric mass balance equation in which a sum total of volumetric flow through all perforations or openings 1232 of the five sections of the retention portion (referred to herein as volumetric flow Q₂ to Q₆) and through the open distal end 1220 (referred to herein as volumetric flow Q₁) equals the total volumetric flow (Q_{Total}) exiting the proximal end of the tube 1222 at a distance of 10 cm to 60 cm away from the last proximal opening, as shown in Equation 2.

Q_{Total}=Q₁+Q₂+Q₃+Q₄+Q₅+Q₆ (Equation 2)

A Modified Loss Coefficient (K′) for each of the sections is based on three types of loss coefficients within the catheter model, namely: an Inlet Loss Coefficient taking into account a pressure loss resulting at a pipe inlet (e.g., the openings and open distal end of the tube 1222); a Friction Loss Coefficient which takes into account pressure loss resulting from friction between the fluid and pipe wall; and a Flow Junction Loss Coefficient taking into account pressure loss resulting from the interaction of two flows coming together.

The Inlet Loss Coefficient is dependent on a shape of the orifice or opening. For example, a tapered or nozzle shaped orifice would increase flow rate into the drainage lumen 1224. In a similar manner, a sharp-edged orifice would have different flow properties than an orifice with less defined edges. For purposes of the following calculations, it is assumed that the openings 1232 are side orifice openings and the open distal end 1220 of the tube 1222 is a sharp-edged opening. The cross sectional area of each opening is considered constant through the tube sidewall.

The Friction Loss Coefficient approximates pressure loss resulting from friction between the fluid and the adjacent inner wall of the tube 1222. Friction loss is defined according to the following equations:

$$\begin{matrix}Re = \frac{ρ U D}{μ} & ( Equation 3.1 ) \\ f = \frac{{c}_{f}}{Re} & ( Equation 3.2 ) \\ {K}_{1 - 2} = {K}_{2 - 3} = {K}_{3 - 3} = {K}_{4 - 3} = {K}_{5 - 3} = {K}_{f} = f \frac{L}{D} & ( Equation 3.3 )\end{matrix}$$

The Flow Junction Loss Coefficients are derived from loss coefficients for combining flow at a branch angle of 90 degrees. Values for the loss coefficients were obtained from Charts 13.10 and 13.11 of Miller D S, Internal Flow Systems, 1990, incorporated by reference herein. The charts use the ratio of the inlet orifice area (referred to as A1 in the charts) to the pipe cross-sectional area (referred to as A3 in the charts) and the ratio of the inlet orifice volumetric flow rate (Q1 in the charts) to the resulting combined pipe volumetric flow rate (Q3 in the charts). For example, for an area ratio of 0.6 between an area of the opening and an area of the drainage lumen, the following Flow Junction Loss Coefficients (K₁₃ and K₂₃) would be used.

| | Flow Ratio (Q₁/Q₃) | | | | | | | | | |
|---|---|---|---|---|---|---|---|---|---|---|
| | 0.1 | 0.2 | 0.3 | 0.4 | 0.5 | 0.6 | 0.7 | 0.8 | 0.9 | 1.0 |
| K₁₃ | −0.58 | −0.04 | 0.11 | 0.45 | 0.75 | 1.13 | 1.48 | 1.81 | 2.16 | 2.56 |
| K₂₃ | 0.15 | 0.27 | 0.39 | 0.48 | 0.56 | 0.63 | 0.69 | 0.72 | 0.74 | 0.76 |

To calculate the Total Manifold Loss Coefficient (K), it is necessary to separate the model into so-called “reference stations” and progressively work through and balance the pressure and flow distributions of the two paths (e.g., flow through the opening and flow through the drainage lumen of the tube) to reach each station starting from the distal tip to the most proximal “Station”. A graphical representation of the different stations used for this calculation is shown in FIG. 16. For example, a most-distal “Station” A is the distal open end 1220 of the tube 122. A second Station A′ is the distal most opening on the sidewall of the tube 122 (e.g., the opening(s) of the fifth section 1318 in FIGS. 11-14). The next station B is for flow through the drainage lumen 1224 just proximal to the A′ opening.

To calculate loss between Station A (the distal opening) and Station B for fluid entering through the open distal end of the tube 1222 (Path 1), the modified loss coefficient (K′) is equal to:

$$\begin{matrix}{K}^{′} = Inlet Loss + Friction Loss + Flow Junction Loss & ( Equation 4.1 ) \\ {K}_{B}^{′} = {K}_{1 - 1} \times {( \frac{{A}_{Pipe}}{{A}_{1}} \times {Q}_{1} )}^{2} + {K}_{1 - 2} \times {Q}_{1}^{2} + {K}_{1 - 3} \times {( {Q}_{1} + {Q}_{2} )}^{2} & ( Equation 4.2 )\end{matrix}$$

In a similar manner, a second path to Station B is through the opening(s) 1334 of the fifth section 1318 (shown in FIGS. 11-14) of the retention portion 1330. A modified loss calculation for Path 2 is calculated as follows:

$$\begin{matrix}{K}^{′} = Inlet Loss + Flow Junction Loss & ( Equation 5.1 ) \\ {K}_{B}^{′} = {K}_{2 - 1} \times {( \frac{{A}_{Pipe}}{{A}_{2}} \times {Q}_{2} )}^{2} + {K}_{2 - 2} \times {( {Q}_{1} + {Q}_{2} )}^{2} & ( Equation 5.2 )\end{matrix}$$

The modified loss coefficients of both Path 1 and Path 2 must equate to ensure the volumetric flow rates (Q₁ and Q₂) reflect the balanced distribution within the manifold at Station B. The volumetric flow rates are adjusted until equal modified loss coefficients for both paths is achieved. The volumetric flow rates can be adjusted because they represent a fractional portion of a total volumetric flow rate (Q′_{Total}), which is assumed to be unity for the purpose of this step-by-step solution. Upon equating the two modified loss coefficients, one can then proceed to equating the two paths to reach station C (the fourth section 1316 in FIGS. 11-14).

Loss coefficients between Station B (flow through drainage lumen in the fifth section 1318) and Station C (flow through lumen in the fourth section 1316) are calculated in a similar manner as shown by Equations 5.1 and 5.2). For example, for Path 1 (Station B to Station C), the modified loss coefficient (K′) for the opening(s) of the fourth section 1316 is defined as:

K′=Loss to Station B+Friction Loss+Flow Junction Loss (Equation 6.1)

K′_{C}=K′_{B}+K₂₋₃×(Q₁+Q₂)²+K₂₋₄×(Q₁+Q₂+Q₃)² (Equation 6.2)

For Path 2 (Station B to C), the modified loss coefficient (K′) based on the flow area of the opening(s) of the fourth section 1316 are defined as:

$$\begin{matrix} {K}^{′} = Inlet Loss + Flow Junction Loss & ( Equation 7.1 ) \\ {K}_{C}^{′} = {K}_{3 - 1} \times {( \frac{{A}_{Pipe}}{{A}_{3}} \times {Q}_{3} )}^{2} + {K}_{3 - 2} \times {( {Q}_{1} + {Q}_{2} + {Q}_{3} )}^{2} & ( Equation 7.2 )\end{matrix}$$

As with the previous stations, the modified loss coefficients of both Path 1 and Path 2 must equate to ensure the volumetric flow rates (Q₁, Q₂, and Q₃) reflect the balanced distribution within the manifold up to Station C. Upon equating the two modified loss coefficients, one can then proceed to equating the two paths to reach Station D, Station E and Station F. The step-by-step solution process proceeds through each station as demonstrated until calculating the modified loss coefficient for the final station, Station F in this case. The Total Loss Coefficient (K) for the manifold can then be calculated using an actual Q_{Total} (volumetric flow rate through a proximal portion of the drainage lumen) determined through experimental measurement.

$$K = \frac{{K}_{F}^{′}}{{Q}_{T o t a l}} \qquad ( Equation 8 )$$

The fractional volumetric flow rates calculated through the step-by-step exercise can then be multiplied by the actual total volumetric flow rate (Q_{Total}) to determine the flow through each opening 1232 (shown in FIGS. 10-10E) and open distal end 1220.

### Examples

Examples are provided below and shown in Tables 3-5 and FIGS. 15A-15C for the calculated volumetric flow rates.

### Example 1

Example 1 illustrates a distribution of fluid flow for a retention member tube with different sized openings, which corresponds to the embodiment of the retention member 1330 shown in FIG. 11. As shown in Table 3, the proximal most opening (Q6) had a diameter of 0.48 mm, the distal-most opening (Q5) on the sidewall of the tube had a diameter of 0.88 mm, and the open distal end (Q6) of the tube had a diameter of 0.97 mm. Each of the openings was circular.

The Percentage of Flow Distribution and Calculated Volumetric Flow Rate were determined as follows.

### Path to Station B Through Distal End of Tube (Path 1)

| f | 8.4 = C_{f}/Re (C_{f} = 64 for circular cross-section) |
|---|---|
| K_{INLET} | 0.16 (Contraction coefficient. for sharp edged orifice |
| | entering pipe) |
| K_{ORIFICE} | 2.8 (Contraction coefficient. for sharp edged orifice w/no |
| | outlet pipe) |
| K_{FRICTION}= | f*(L/D) (Dependent on the length between orifices) |
| Part 1-1= | Inlet loss coef × (A_{T}/A₁ × Q′1)² |
| Part 1-2= | Catheter friction loss × Q′₁² |
| Part 1-3= | Through flow junction loss to station 2 × (Q′₁ + Q′₂)² |
| A₂/A_{T}= | 0.82 |
| Q′₂/(CQ′₁ + | 0.83 |
| Q′₂)= | |
| K₁₋₃= | 0.61 (From Miller, see table above) |
| Part 1-1= | 0.0000 |
| Part 1-2= | 0.0376 |
| Part 1-3= | 0.0065 |
| K′= | 0.0442 |

### Path to Station B Through Sidewall Opening (Path 2)

| Part 2-1= | Orifice loss coef × (A_{T}/A₂ × Q′₂)² |
|---|---|
| Part 2-2= | Branch flow junction loss to station 2 × |
| | (Q′₁ + Q′₂)² |
| A₂/A_{T}= | 0.82 |
| Q′₂/(Q′₁ + Q′₂)= | 0.83 |
| K₂₋₂= | 1.3 (From Chart 13.10 of Miller) |
| Part 2-1= | 0.0306 |
| Part 2-2= | 0.0138 |
| K′= | 0.0444 |

Path to Station C from Station B (Path 1+Path 2)

| Part 2-3= | Catheter friction loss × (Q′₁ + Q′₂)² |
|---|---|
| Part 2-4= | Through flow junction loss to station 3 × |
| | (Q′₁ + Q′₂ + Q′₃)² |
| A₃/A_{T}= | 0.61 |
| Q′₃/(Q′₁ + Q′₂ + Q′₃)= | 0.76 |
| K₂₋₄= | 0.71 (From Chart 13.11 of Miller) |
| Loss coefficient to | |
| Station 2= | 0.044 |
| Part 2-3= | 0.921 |
| Part 2-4= | 0.130 |
| K′= | 1.095 |

### Path to Station C Through Sidewall Opening (Path 3)

| Part 3-1= | Orifice loss coef × (A_{T}/A₃ × Q′₃)² |
|---|---|
| Part 3-2= | Branch flow junction loss to station 3 × |
| | (Q′₁ + Q′₂ + Q′₃)² |
| A₃/A_{T}= | 0.61 |
| Q′₃/(Q′₁ + Q′₂ + Q′₃)= | 0.76 |
| K₃₋₂= | 1.7 (From Chart 13.10 of Miller) |
| Part 3-1= | 0.785 |
| Part 3-2= | 0.311 |
| K′= | 1.096 |

Path to Station D from Station C (Path 1+Path 2+Path 3)

| Part 3-3= | Catheter friction loss × (Q′1 + Q′2 + Q′3)² |
|---|---|
| Part 3-4= | Through flow junction loss to station 4 × |
| | (Q′1 + Q′2 + Q′3 + Q′4)² |
| A₄/A_{T}= | 0.46 |
| Q′₄/(Q′₁ + Q′₂ + Q′₃ + Q′₄)= | 0.70 |
| K₃₋₄= | 0.77 (From Chart 13.11 of Miller) |
| Loss coefficient to Station 3= | 1.10 |
| Part 3-3= | 15.90 |
| Part 3-4= | 1.62 |
| K′= | 18.62 |

### Path to Station D Through Sidewall Opening (Path 4)

| Part 4-1= | Orifice loss coef × (A_{T}/A₄ × Q′₄)² |
|---|---|
| Part 4-2= | Branch flow junction loss to station 4 × |
| | (Q′₁ + Q′₂ + Q′₃ + Q′₄)² |
| A₄/A_{T}= | 0.46 |
| Q′₄/(Q′₁ + Q′₂ + Q′₃ + Q′₄)= | 0.70 |
| K₄₋₂= | 2.4 (From Chart 13.10 of Miller) |
| Part 4-1= | 13.59 |
| Part 4-2= | 5.04 |
| K′= | 18.62 |

Path to Station E from Station D (Path 1+Path 2+Path 3+Path 4)

| Part 4-3= | Catheter friction loss × (Q′₁ + Q′₂ + Q′₃ + Q′₄)² |
|---|---|
| Part 4-4= | Through flow junction loss to station 5 × |
| | (Q′₁ + Q′₂ + Q′₃ + Q′₄ + Q′₅)² |
| A₅/A_{T}= | 0.36 |
| Q′₅/(Q′₁ + Q′₂ + Q′₃ + | 0.65 |
| Q′₄ + Q′₅)= | |
| K₃₋₄= | 0.78 (From Chart 13.11 of Miller) |
| Loss coefficient to | 18.6 |
| Station 4= | |
| Part 4-3= | 182.3 |
| Part 4-4= | 13.3 |
| K′= | 214.2 |

### Path to Station E Through Sidewall Opening (Path 5)

| Part 5-1= | Orifice loss coef × (A_{T}/A₅ × Q′₅)² |
|---|---|
| Part 5-2= | Branch flow junction loss to station 5 × |
| | (Q′₁ + Q′₂ + Q′₃ + Q′₄ + Q′₅)² |
| A₅/A_{T}= | 0.36 |
| Q′₅/(Q′₁ + Q′₂ + Q′₃ + | 0.65 |
| Q′₄ + Q′₅)= | |
| K₄₋₂= | 3.3 (From Chart 13.10 of Miller) |
| Part 5-1= | 157.8 |
| Part 5-2= | 56.4 |
| K′= | 214.2 |

Path to Station F from Station E (Through Paths 1-5)

| Part 5-3= | Catheter friction loss × (Q′₁ + Q′₂ + Q′₃ + |
|---|---|
| | Q′₄ + Q′₅)² |
| Part 5-4= | Through flow junction loss to station 6 × |
| | (Q′₁ + Q′₂ + Q′₃ + Q′₄ + Q′₅ + Q′₆)² |
| A₆/A_{T}= | 0.24 |
| Q′₆/(Q′₁ + Q′₂ + Q′₃ + Q′₄ + | 0.56 |
| Q′₅ + Q′₆)= | |
| K₃₋₄= | 0.77 (From Chart 13.11 of Miller) |
| Loss coefficient to | 214.2 |
| Station 5= | |
| Part 5-3= | 1482.9 |
| Part 5-4= | 68.3 |
| K′= | 1765.4 |

### Path to Station F Through Sidewall Opening (Path 6)

| Part 6-1= | Orifice loss coef × (A_{T}/A₆ × Q′₆)² |
|---|---|
| Part 6-2= | Branch flow junction loss to station 6 × |
| | (Q′₁ + Q′₂ + Q′₃ + Q′₄ + Q′₅ + Q′₆)² |
| A₆/A_{T}= | 0.24 |
| Q′₆/(Q′₁ + Q′₂ + Q′₃ +Q′₄ + | |
| Q′₅ + Q′₆)= | |
| | 0.56 |
| K₄₋₂= | 5.2 (From Chart 13.10 of Miller) |
| Part 6-1= | 1304.3 |
| Part 6-2= | 461.2 |
| K′= | 1765.5 |

In order to calculate flow distribution for each “Station” or opening, the calculated K′ values were multiplied by actual total volumetric flow rate (Q_{Total}) to determine the flow through each perforation and distal end hole. Alternatively, calculated results could be presented as a percentage of total flow or a flow distribution as shown in Table 3. As shown in Table 3 and in FIG. 15C, the Percentage of Flow Distribution (% Flow Distribution) through the proximal most opening (Q6) was 56.1%. Flow through the two proximal-most openings (Q6 and Q5) was 84.6%.

TABLE 3

| | | | | Cumulative |
|---|---|---|---|---|
| | % Flow | Diameter | Length | Length |
| Position | Distribution | (mm) | (mm) | (mm) |
| Q₆′ | 56.1% | 0.48 | 0 | 0 |
| (proximal) | | | | |
| Q₅′ | 28.5% | 0.58 | 10 | 10 |
| Q₄′ | 10.8% | 0.66 | 10 | 20 |
| Q₃′ | 3.5% | 0.76 | 10 | 30 |
| Q₂′ | 0.9% | 0.88 | 10 | 40 |
| Q₁′ (distal) | 0.2% | 0.97 | 15 | 55 |
| Q_{TOTAL} | 100% | | | |

As demonstrated in Example 1, the increasing diameters of perforations going from the proximal to distal regions of the retention portion of the tube results in more evenly distributed flow across the entire retention portion.

### Example 2

In Example 2, each opening has the same diameter and area. As shown in Table 4 and FIG. 15A, in that case, flow distribution through the proximal-most opening is 86.2% of total flow through the tube. Flow distribution through the second opening is 11.9%. Therefore, in this example, it was calculated that 98.1% of fluid passing through the drainage lumen entered the lumen through the two proximal-most openings. Compared to Example 1, Example 2 has increased flow through the proximal end of the tube. Therefore, Example 1 provides a wider flow distribution in which a greater percentage of fluid enters the drainage lumen through openings other than the proximal-most opening. As such, fluid can be more efficiently collected through multiple openings reducing fluid backup and improving distribution of negative pressure through the renal pelvis and/or kidneys.

TABLE 4

| | | | | Cumulative |
|---|---|---|---|---|
| | % Flow | Diameter | Length | Length |
| Position | Distribution | (mm) | (mm) | (mm) |
| Q₆′ | 86.2% | 0.88 | 0 | 0 |
| (proximal) | | | | |
| Q₅′ | 11.9% | 0.88 | 22 | 22 |
| Q₄′ | 1.6% | 0.88 | 22 | 44 |
| Q₃′ | 0.2% | 0.88 | 22 | 66 |
| Q₂′ | 0.03% | 0.88 | 22 | 88 |
| Q₁′ (distal) | 0.01% | 0.97 | 22 | 110 |
| Q_{TOTAL} | 100% | | | |

### Example 3

Example 2 also illustrates flow distribution for openings having the same diameter. However, as shown in Table 5, the openings are closer together (10 mm vs. 22 mm). As shown in Table 5 and FIG. 15B, 80.9% of fluid passing through the drainage lumen entered the drainage lumen through the proximal most opening (Q6). 96.3% of fluid in the drainage lumen entered the drainage lumen through the two proximal-most openings (Q5 and Q6).

TABLE 5

| | | | | Cumulative |
|---|---|---|---|---|
| | % Flow | Diameter | Length | Length |
| Position | Distribution | (mm) | (mm) | (mm) |
| Q6′ | 80.9% | 0.88 | 0 | 0 |
| (proximal) | | | | |
| Q₅′ | 15.4% | 0.88 | 10 | 10 |
| Q₄′ | 2.9% | 0.88 | 10 | 20 |
| Q₃′ | 0.6% | 0.88 | 10 | 30 |
| Q₂′ | 0.1% | 0.88 | 10 | 40 |
| Q₁′ (distal) | 0.02% | 0.97 | 15 | 55 |
| Q_{TOTAL} | 100% | | | |

Referring generally now to FIGS. 17-41C, and more specifically to FIG. 17, there are shown two exemplary ureteral catheters 5000, 5001 positioned within the urinary tract of a patient, and a bladder catheter 116. The ureteral catheter 5000, 5001 comprises: a drainage lumen 5002, 5003 for draining fluid such as urine from at least one of a patient's kidney 2, 4, renal pelvis 20, 21 or in the ureter 6, 8 adjacent to the renal pelvis 20, 21. The drainage lumen 5002, 5003 comprises a distal portion 5004, 5005 configured to be positioned in a patient's kidney 2, 4, renal pelvis 20, 21 and/or in the ureter 6, 8 adjacent to the renal pelvis 20, 21 and a proximal portion 5006, 5007 through which fluid 5008 is drained to the bladder 10 or outside of the body of the patient, as shown in FIGS. 2B and 2C.

In some examples, the distal portion 5004, 5005 comprises an open distal end 5010, 5011 for drawing fluid into the drainage lumen 5002, 5003. The distal portion 5004, 5005 of the ureteral catheter 5000, 5001 further comprises a retention portion 5012, 5013 for maintaining the distal portion 5004, 5005 of the drainage lumen or tube 5002, 5003 in the ureter and/or kidney. The retention portion 5012, 5013 can be flexible and/or bendable to permit positioning of the retention portion 5012, 5013 in the ureter, renal pelvis, and/or kidney. For example, the retention portion 5012, 5013 is desirably sufficiently bendable to absorb forces exerted on the catheter 5000, 5001 and to prevent such forces from being translated to the ureters. Further, if the retention portion 5012, 5013 is pulled in the proximal direction P (shown in FIG. 17) toward the patient's bladder 10, the retention portion 5012, 5013 can be sufficiently flexible to begin to unwind, straightened or collapsed so that it can be drawn through the ureter 6, 8.

In some examples, the retention portion comprises a funnel support. Non-limiting examples of different shapes of funnel supports are shown in FIGS. 7A, 7B, 17, and 18A-41C, which are discussed in detail below. Generally, the funnel support comprises at least one sidewall. The at least one sidewall of the funnel support comprises a first diameter and a second diameter, the first diameter being less than the second diameter. The second diameter of the funnel support is closer to an end of the distal portion of the drainage lumen than the first diameter.

The proximal portion of the drainage lumen or drainage tube is essentially free of or free of openings. While not intending to be bound by any theory, it is believed that when negative pressure is applied at the proximal end of the proximal portion of the drainage lumen, that openings in the proximal portion of the drainage lumen or drainage tube may be undesirable as such openings may diminish the negative pressure at the distal portion of the ureteral catheter and thereby diminish the draw or flow of fluid or urine from the kidney and renal pelvis of the kidney. It is desirable that the flow of fluid from the ureter and/or kidney is not prevented by occlusion of the ureter and/or kidney by the catheter. Also, while not intending to be bound by any theory, it is believed that when negative pressure is applied at the proximal end of the proximal portion of the drainage lumen, ureter tissue may be drawn against or into openings along the proximal portion of the drainage lumen, which may irritate the tissues.

Some examples of ureteral catheters comprising a retention portion comprising a funnel support according to the present invention are shown in FIGS. 7A, 7B, 17, and 18A-41C. In FIGS. 7A-10E, the funnel support is formed by a coil of tubing. In FIGS. 17-41C, other examples of the funnel support are shown. Each of these funnel supports according to the present invention will be discussed in detail below.

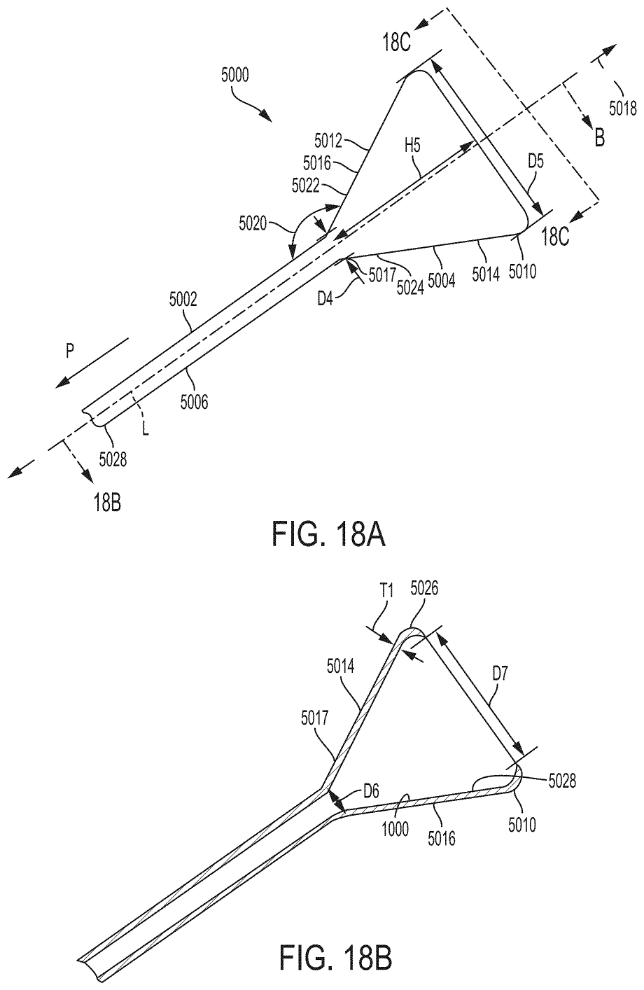

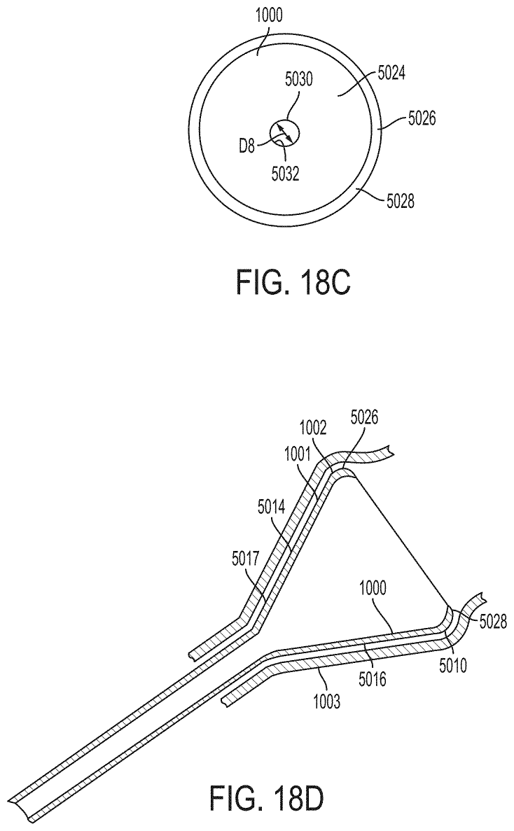

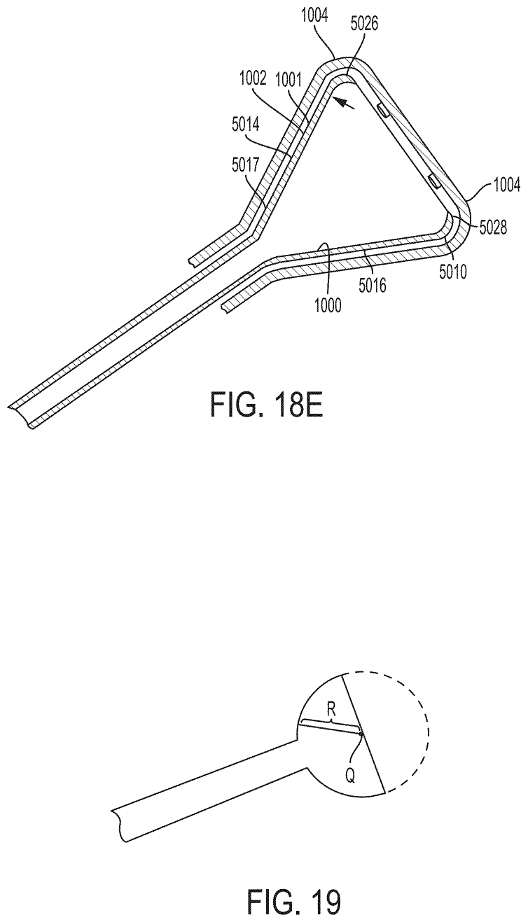

Referring now to FIGS. 18A-D, in some examples, there is shown a distal portion 5004 of the ureteral catheter, indicated generally as 5000. The distal portion 5004 comprises a retention portion 5012 comprising a funnel-shaped support 5014. The funnel-shaped support 5014 comprises at least one sidewall 5016. As shown in FIGS. 18C and 18D, the outer periphery 1002 or protective surface area 1001 comprises the outer surface or outer wall 5022 of the funnel-shaped support 5014. The one or more drainage holes, ports or perforations, or interior opening 5030, are disposed on the protected surface areas or inner surface areas 1000 of the funnel-shaped support 5014. As shown in FIGS. 18C and 18D, there is a single drainage hole 5030 at the base portion 5024 of the funnel-shaped support, although multiple holes can be present.

The at least one sidewall 5016 of the funnel support 5014 comprises a first (outer) diameter D4 and a second (outer) diameter D5, the first outer diameter D4 being less than the second outer diameter D5. The second outer diameter D5 of the funnel support 5014 is closer to the distal end 5010 of the distal portion 5004 of the drainage lumen 5002 than is the first outer diameter D4. In some examples the first outer diameter D4 can range from about 0.33 mm to 4 mm (about 1 Fr to about 12 Fr (French catheter scale)), or about 2.0±0.1 mm. In some examples, the second outer diameter D5 is greater than first outer diameter D4 and can range from about 1 mm to about 60 mm, or about 10 mm to 30 mm, or about 18 mm±2 mm.

In some examples, the at least one sidewall 5016 of the funnel support 5014 can further comprise a third diameter D7 (shown in FIG. 18B), the third diameter D7 being less than the second outer diameter D5. The third diameter D7 of the funnel support 5014 is closer to the distal end 5010 of the distal portion 5004 of the drainage lumen 5002 than is the second diameter D5. The third diameter D7 is discussed in greater detail below regarding the lip. In some examples, the third diameter D7 can range from about 0.99 mm to about 59 mm, or about 5 mm to about 25 mm.

The at least one sidewall 5016 of the funnel support 5014 comprises a first (inner) diameter D6. The first inner diameter D6 is closer to the proximal end 5017 of the funnel support 5014 than is the third diameter D7. The first inner diameter D6 is less than the third diameter D7. In some examples the first inner diameter D6 can range from about 0.05 mm to 3.9 mm, or about 1.25±0.75 mm.

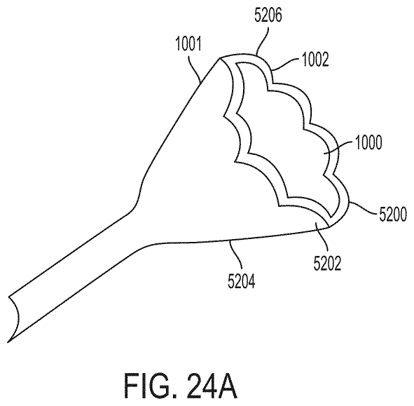

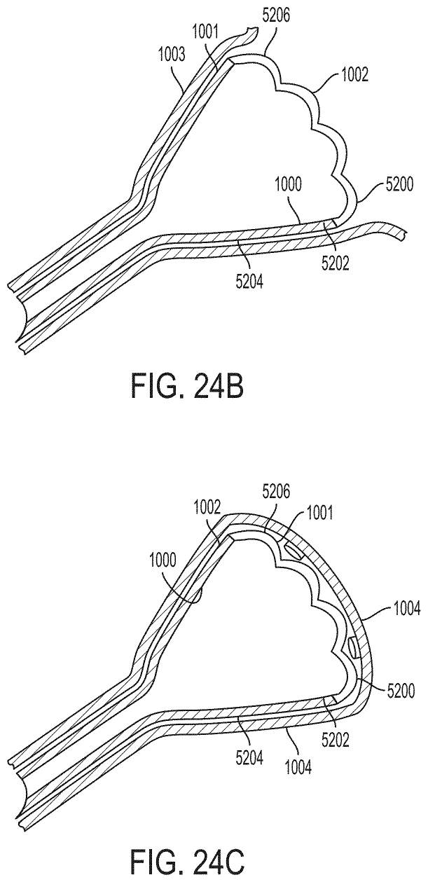

In some examples, an overall height H5 of the sidewall 5016 along a central axis 5018 of the retention portion 5012 can range from about 1 mm to about 25 mm. In some examples, the height H5 of the sidewall can vary at different portions of the sidewall, for example if the sidewall has an undulating edge or rounded edges such as is shown in FIG. 24. In some examples, the undulation can range from about 0.01 mm to about 5 mm or more, if desired.

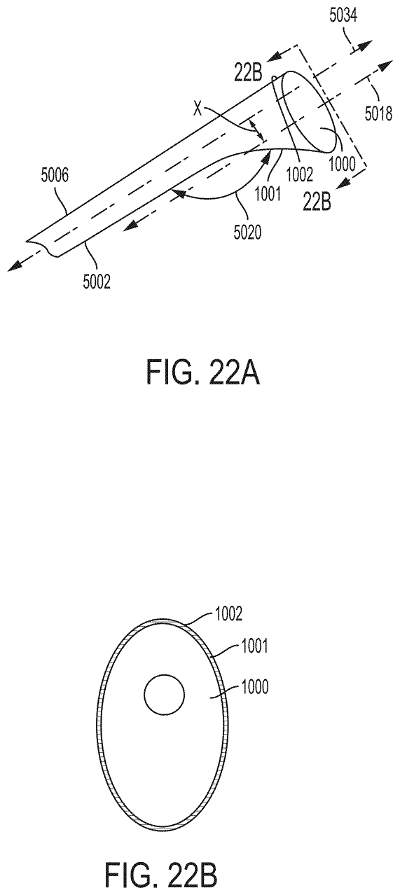

In some examples, as shown in FIGS. 7A-10E, and 17-41C, the funnel support 5014 can have a generally conical shape. In some examples, the angle 5020 between the outer wall 5022 near the proximal end 5017 of the funnel support 5014 and the drainage lumen 5002 adjacent to the base portion 5024 of the funnel support 5014 can range from about 100 degrees to about 180 degrees, or about 100 degrees to about 160 degrees, or about 120 degrees to about 130 degrees. The angle 5020 may vary at different positions about the circumference of the funnel support 5014, such as is shown in FIG. 22A, in which the angle 5020 ranges from about 140 degrees to about 180 degrees.

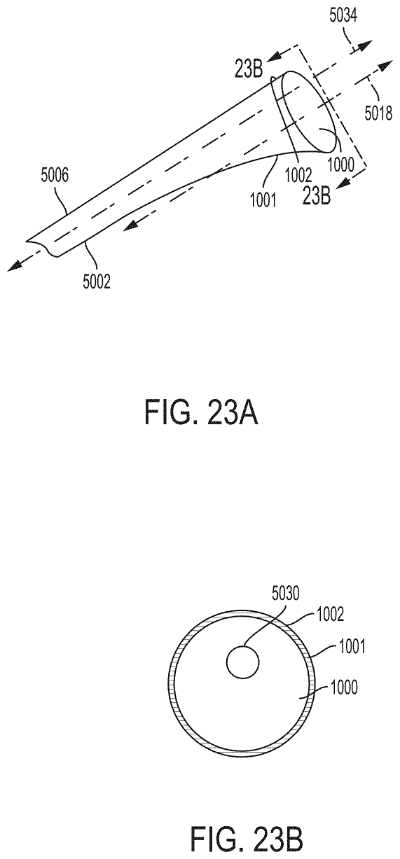

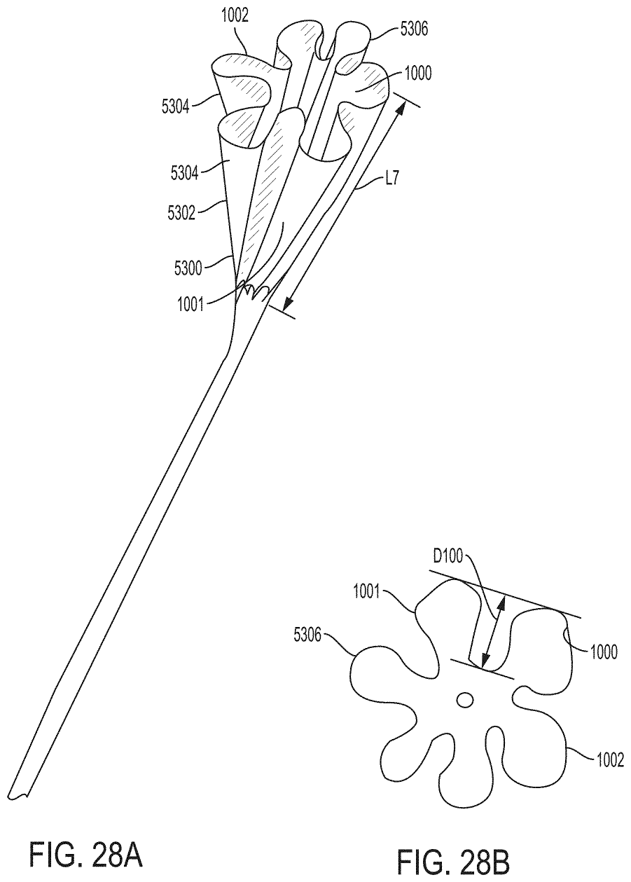

In some examples, the edge or lip 5026 of the distal end 5010 of the at least one sidewall 5016 can be rounded, square, or any shape desired. The shape defined by the edge 5026 can be, for example, circular (as shown in FIGS. 18C and 23B), elliptical (as shown in FIG. 22B), lobes (as shown in FIGS. 28B, 29B and 31), square, rectangular, or any shape desired.

Referring now to FIGS. 28A-31, there is shown a funnel support 5300 wherein the at least one sidewall 5302 comprises a plurality of lobe-shaped longitudinal folds 5304 along the length L7 of the sidewall 5302. The outer periphery 1002 or protective surface area 1001 comprises the outer surface or outer wall 5032 of the funnel-shaped support 5300. The one or more drainage holes, ports or perforations, or interior opening, are disposed on the protected surface areas or inner surface areas 1000 of the funnel-shaped support 5300. As shown in FIG. 28B, there is a single drainage hole at the base portion of the funnel-shaped support, although multiple holes can be present.

The number of folds 5304 can range from 2 to about 20, or about 6, as shown. In this example, the folds 5304 can be formed from one or more flexible materials, such as silicone, polymer, solid material, fabric, or a permeable mesh to provide the desired lobe shape. The folds 5304 can have a generally rounded shape as shown in cross-sectional view 51B. The depth D100 of each fold 5304 at the distal end 5306 of the funnel support 5300 can be the same or vary, and can range from about 0.5 mm to about 5 mm.

Referring now to FIGS. 29A and 29B, one or more folds 5304 can comprise at least one longitudinal support member 5308. The longitudinal support member(s) 5308 can span the entire length L7 or a portion of the length L7 of the funnel support 5300. The longitudinal support members 5308 can be formed from a flexible yet partially rigid material, such as a temperature sensitive shape memory material, for example nitinol. The thickness of the longitudinal support members 5308 can range from about 0.01 mm to about 1 mm, as desired. In some examples, the nitinol frame can be covered with a suitable waterproof material such as silicon to form a tapered portion or funnel. In that case, fluid is permitted to flow down the inner surface 5310 of the funnel support 5300 and into the drainage lumen 5312. In other examples, the folds 5304 are formed from various rigid or partially rigid sheets or materials bended or molded to form a funnel-shaped retention portion.

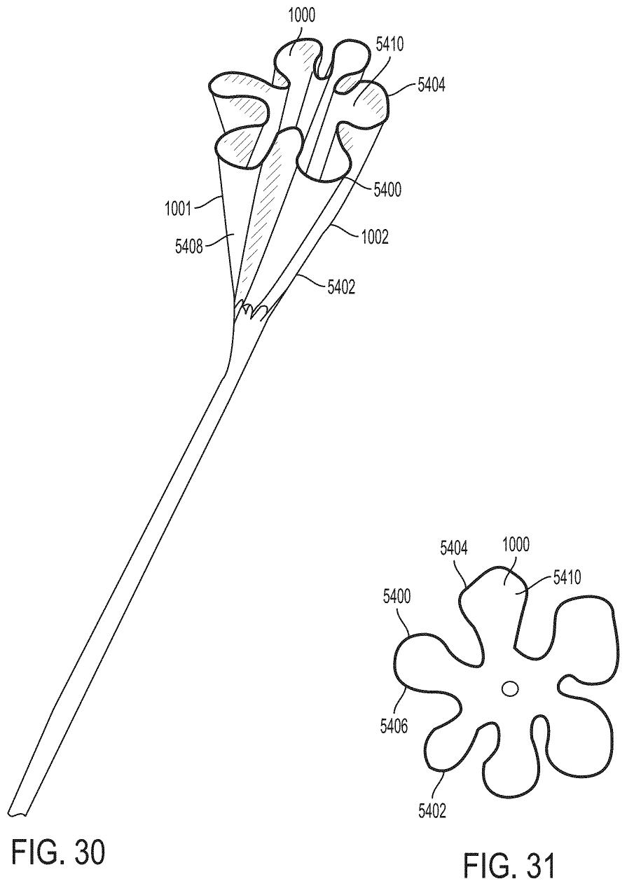

Referring now to FIGS. 30 and 31, the distal end or edge 5400 of the folds 5402 can comprise at least one edge support member 5404. The edge support member(s) 5404 can span the entire circumference 5406 or one or more portions of the circumference 5406 of the distal edge 5400 of the funnel support 5408. The edge support member(s) 5404 can be formed from a flexible yet partially rigid material, such as a temperature sensitive shape memory material, for example nitinol. The thickness of the edge support member(s) 5404 can range from about 0.01 mm to about 1 mm, as desired.

In some examples, such as are shown in FIGS. 18A-C, the distal end 5010 of the drainage lumen 5002 (or funnel support 5014) can have an inwardly facing lip 5026 oriented towards the center of the funnel support 5014, for example of about 0.01 mm to about 1 mm, to inhibit irritating the kidney tissue. Thus, the funnel support 5014 can comprise a third diameter D7 less than the second diameter D5, the third diameter D7 being closer to an end 5010 of the distal portion 5004 of the drainage lumen 5002 than the second diameter D5. The outer surface 5028 of the lip 5026 can be rounded, a square edge, or any shape desired. The lip 5026 may assist in providing additional support to the renal pelvis and internal kidney tissues.

Referring now to FIGS. 24A-C, in some examples, the edge 5200 of the distal end 5202 of the at least one sidewall 5204 can be shaped. For example, the edge 5200 can comprise a plurality of generally rounded edges 5206 or scallops, for example about 4 to about 20 or more rounded edges. The rounded edges 5206 can provide more surface area than a straight edge to help support the tissue of the renal pelvis or kidney and inhibit occlusion. The edge 5200 can have any shape desired, but preferably is essentially free of or free of sharp edges to avoid injuring tissue.

In some examples, such as are shown in FIGS. 18A-C and 22A-23B, the funnel support 5014 comprises a base portion 5024 adjacent to the distal portion 5004 of the drainage lumen 5002. The base portion 5024 comprises at least one interior opening 5030 aligned with an interior lumen 5032 of the drainage lumen 5002 of the proximal portion 5006 of the drainage lumen 5002 for permitting fluid flow into the interior lumen 5032 of the proximal portion 5006 of the drainage lumen 5002. In some examples, the cross-section of the opening 5030 is circular, although the shape may vary, such as ellipsoid, triangular, square, etc.

In some examples, such as is shown in FIGS. 22A-23B, a central axis 5018 of the funnel support 5014 is offset with respect to a central axis 5034 of the proximal portion 5006 of the drainage lumen 5002. The offset distance X from the central axis 5018 of the funnel support 5014 with respect to the central axis 5034 of the proximal portion 5006 can range from about 0.1 mm to about 5 mm.

The at least one interior opening 5030 of the base portion 5024 has a diameter D8 (shown, for example, in FIGS. 18C and 23B) ranging from about 0.05 mm to about 4 mm. In some examples, the diameter D8 of the interior opening 5030 of the base portion 5024 is about equal to the first inner diameter D6 of the adjacent proximal portion 5006 of the drainage lumen.

In some examples, the ratio of the height H5 of the at least one sidewall 5016 funnel support 5014 to the second outer diameter D5 of the at least one sidewall 5016 of the funnel support 5014 ranges from about 1:25 to about 5:1.

In some examples, the at least one interior opening 5030 of the base portion 5024 has a diameter D8 ranging from about 0.05 mm to about 4 mm, the height H5 of the at least one sidewall 5016 of the funnel support 5014 ranges from about 1 mm to about 25 mm, and the second outer diameter D5 of the funnel support 5014 ranges from about 5 mm to about 25 mm.

In some embodiments, the thickness T1 (shown in FIG. 18B, for example) of the at least one sidewall 5016 of the funnel support 5014 can range from about 0.01 mm to about 1.9 mm, or about 0.5 mm to about 1 mm. The thickness T1 can be generally uniform throughout the at least one sidewall 5016, or it may vary as desired. For example, the thickness T1 of the at least one sidewall 5016 can be less or greater near the distal end 5010 of the distal portion 5004 of the drainage lumen 5002 than at the base portion 5024 of the funnel support 5014.

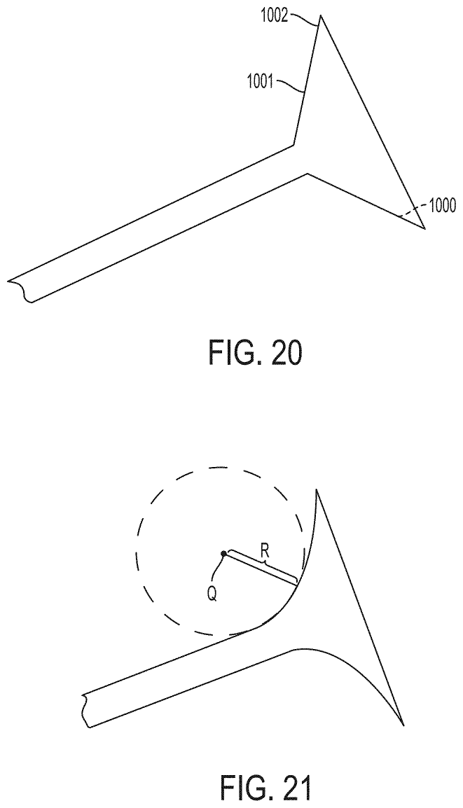

Referring now to FIGS. 18A-21, along the length of the at least one sidewall 5016, the sidewall 5016 can be straight (as shown in FIGS. 18A and 20), convex (as shown in FIG. 19), concave (as shown in FIG. 21), or any combination thereof. As shown in FIGS. 19 and 21, the curvature of the sidewall 5016 can be approximated from the radius of curvature R from the point Q such that a circle centered at Q meets the curve and has the same slope and curvature as the curve. In some examples, the radius of curvature ranges from about 2 mm to about 12 mm. In some examples, the funnel support 5014 has a generally hemispherical shape, as shown in FIG. 19.

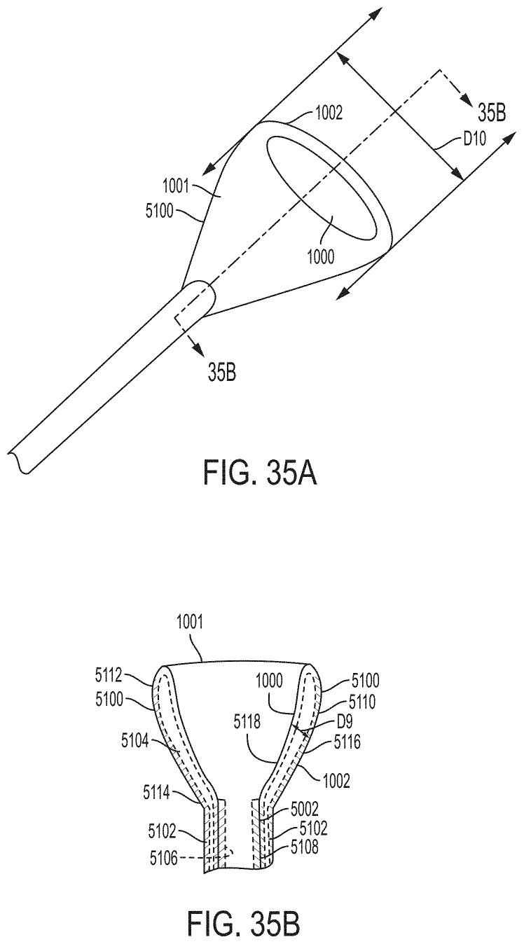

In some examples, the at least one sidewall 5016 of the funnel support 5014 is formed from a balloon 5100, for example as shown in FIGS. 35A, 35 B, 38A and 38B. The balloon 5100 can have any shape that provides a funnel support to inhibit occlusion of the ureter, renal pelvis, and/or the rest of the kidney. As shown in FIGS. 35A and 35B, the balloon 5100 has the shape of a funnel. The balloon can be inflated after insertion or deflated before removal by adding or removing gas or air through the gas port(s) 5102. The gas port(s) 5102 can simply be contiguous with the interior 5104 of the balloon 5100, e.g., the balloon 5100 can be adjacent to the interior 5106 or encase the exterior 5108 of an adjacent portion of the proximal portion 5006 of the drainage lumen 5002. The diameter D9 of the sidewall 5110 of the balloon 5100 can range from about 1 mm to about 3 mm, and can vary along its length such that the sidewall has a uniform diameter, tapers toward the distal end 5112 or tapers toward the proximal end 5114 of the funnel support 5116. The outer diameter D10 of the distal end 5112 of the funnel support 5116 can range from about 5 mm to about 25 mm.

In some examples, the at least one sidewall 5016 of the funnel support 5014 is continuous along the height H5 of the at least one sidewall 5016, for example as shown in FIGS. 18A, 19, 20, and 21. In some examples, the at least one sidewall 5016 of the funnel support 5014 comprises a solid wall, for example the sidewall 5016 is not permeable through the sidewall after 24 hours of contact with a fluid such as urine on one side.

In some examples, the at least one sidewall of the funnel support is discontinuous along the height or the body of the at least one sidewall. As used herein, “discontinuous” means that the at least one sidewall comprises at least one opening for permitting the flow of fluid or urine therethrough into the drainage lumen, for example by gravity or negative pressure. In some examples, the opening can be a conventional opening through the sidewall, or openings within a mesh material, or openings within a permeable fabric. The cross-sectional shape of the opening can be circular or non-circular, such as rectangular, square, triangular, polygonal, ellipsoid, as desired. In some examples, an “opening” is a gap between adjacent coils in a retention portion of a catheter comprising a coiled tube or conduit.

As used herein, “opening” or “hole” means a continuous void space or channel through the sidewall from the outside to the inside of the sidewall, or vice versa. In some examples, each of the at least one opening(s) can have an area which can be the same or different and can range from about 0.002 mm² to about 100 mm², or about 0.002 mm² to about 10 mm². As used herein, the “area” or “surface area” or “cross-sectional area” of an opening means the smallest or minimum planar area defined by a perimeter of the opening. For example, if the opening is circular and has a diameter of about 0.36 mm (area of 0.1 mm²) at the outside of the sidewall, but a diameter of only 0.05 mm (area of 0.002 mm²) at some point within the sidewall or on the opposite side of the sidewall, then the “area” would be 0.002 mm² since that is the minimum or smallest planar area for flow through the opening in the sidewall. If the opening is square or rectangular, the “area” would be the length times the width of the planar area. For any other shapes, the “area” can be determined by conventional mathematical calculations well known to those skilled in the art. For example, the “area” of an irregular shaped opening is found by fitting shapes to fill the planar area of the opening, calculating the area of each shape and adding together the area of each shape.

In some examples, at least a portion of the sidewall comprises at least one (one or more) openings. Generally, the central axis of the opening(s) can be generally perpendicular to the planar outer surface of the sidewall, or the opening(s) can be angled with respect to the planar outer surface of the sidewalls. The dimensions of the bore of the opening may be uniform throughout its depth, or the width may vary along the depth, either increasing, decreasing, or alternating in width through the opening from the exterior surface of the sidewall to the interior surface of the sidewall.

Referring now to FIGS. 9A-9E, 10A, 10E, 11-14, 27, 32A, 32B, 33 and 34, in some examples at least a portion of the sidewall comprises at least one (one or more) openings. The opening(s) can be positioned anywhere along the sidewall. For example, the openings can be uniformly positioned throughout the sidewall, or positioned in specified regions of the sidewall, such as closer to the distal end of the sidewall or closer to the proximal end of the sidewall, or in vertical or horizontal or random groupings along the length or circumference of the sidewall. While not intending to be bound by any theory, it is believed that, when negative pressure is applied at the proximal end of the proximal portion of the drainage lumen, openings in the proximal portion of the funnel support that are directly adjacent to the ureter, renal pelvis and/or other kidney tissue may be undesirable as such openings may diminish the negative pressure at the distal portion of the ureteral catheter and thereby diminish the draw or flow of fluid or urine from the kidney and renal pelvis of the kidney, as well as perhaps irritate the tissue.

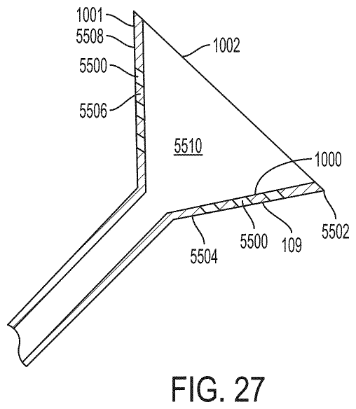

The number of openings can vary from 1 to 1000 or more, as desired. For example, in FIG. 27, six openings (three on each side) are shown. As discussed above, in some examples, each of the at least one opening(s) can have an area which can be the same or different and can range from about 0.002 mm² to about 50 mm², or about 0.002 mm² to about 10 mm².

In some examples, as shown in FIG. 27, the openings 5500 can be positioned closer the distal end 5502 of the sidewall 5504. In some examples, the opening(s) are positioned in the distal half 5506 of the sidewall towards the distal end 5502. In some examples, the openings 5500 are evenly distributed around the circumference of the distal half 5506 or even closer to the distal end 5502 of the sidewall 5504.

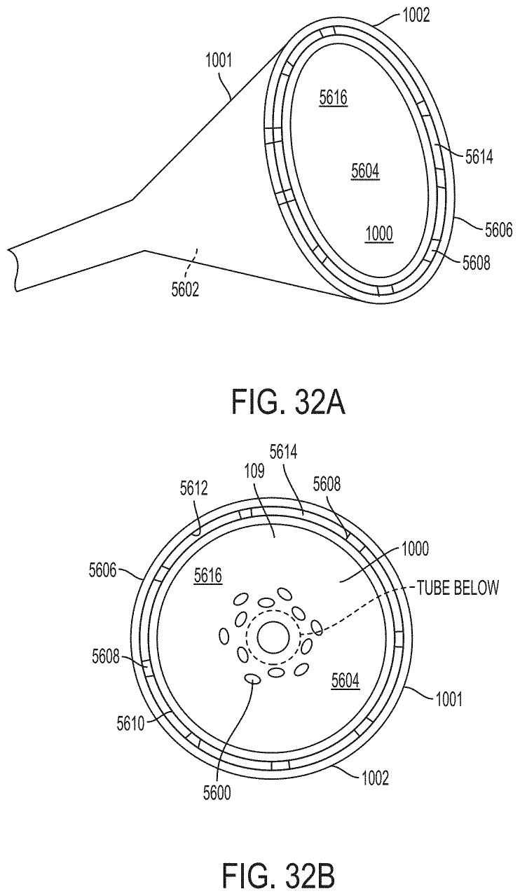

In contrast, in FIG. 32B, the openings 5600 are positioned near the proximal end 5602 of an inner sidewall 5604, and do not directly contact the tissue since there is an outer sidewall 5606 between the opening 5600 and the tissue. Alternatively or additionally, one or more opening(s) 5600 can be positioned near the distal end of the inner sidewall, as desired. The inner sidewall 5604 and outer sidewall 5606 can be connected by one or more supports 5608 or ridges connecting the outside 5610 of the inner sidewall 5604 to the inside 5612 of the outer sidewall 5606.

In some non-limiting examples, such as are shown in FIGS. 9A-9E, 10A, 10D-10G, 18B, 18D, 18E, 20, 22A, 22B, 23A, 23B, 24A-24C, 25, 26, 27, 28A, 28B, 29A-29C, 30, 31, 32A, 32B, 33, 34, 35A, 35B, 37B, 38A, 39B, 39C, 40A-40C, and 41A-41C, a protected surface area(s) or inner surface area(s) 1000 can be established by a variety of different shapes or materials. Non-limiting examples of protected surface areas or inner surface areas 1000 can comprise, for example, the interior portions 152, 5028, 5118, 5310, 5410, 5510, 5616, 5710, 5814, 6004 of a funnel 150, 5014, 5116, 5300, 5408, 5508, 5614, 5702, 5802, 6000, the interior portions 164, 166, 168, 170, 338, 1281, 1283, 1285 of a coil 183, 184, 185, 187, 334, 1280, 1282, 1284, the interior portions 5902, 6003 of a porous material 5900, 6002, the interior portions 162, 5710, 5814 of a mesh 57, 5704, 5804, or the interior portions 536 of a cage 530 with protected drainage holes 533.

In some non-limiting examples, at least one protected drainage hole(s), port(s) or perforation(s) 133, 1233 are disposed on the protected surface area 1000. Upon application of negative pressure therapy through the catheter, the urothelial or mucosal tissue 1003, 1004 conforms or collapses onto the outer periphery 189, 1002 or protective surface area 1001 of the retention portion 130, 330, 410, 500, 1230, 1330, 2230, 3230, 4230, 5012, 5013 of the catheter and is thereby prevented or inhibited from occluding one or more of the protected drainage holes, ports or perforations 133, 1233 disposed on the protected surface area or inner surface area 1000, and thereby a patent fluid column or flow is established, maintained, or enhanced between the renal pelvis and calyces and the drainage lumen 124, 324, 424, 524, 1224, 5002, 5003, 5312, 5708, 5808.

In some examples, the retention portion 130, 330, 410, 500, 1230, 1330, 2230, 3230, 4230, 5012, 5013 comprises one or more helical coils having outwardly facing sides 1288 and inwardly facing sides 1286, and wherein the outer periphery 1002 or protective surface area 1001 comprises the outwardly facing sides 1288 of the one or more helical coils, and the at least one protected drainage hole(s), port(s) or perforation(s) 133, 1233 are disposed on the inwardly facing sides 1286 (protected surface area or inner surface area 1000) of the one or more helical coils.

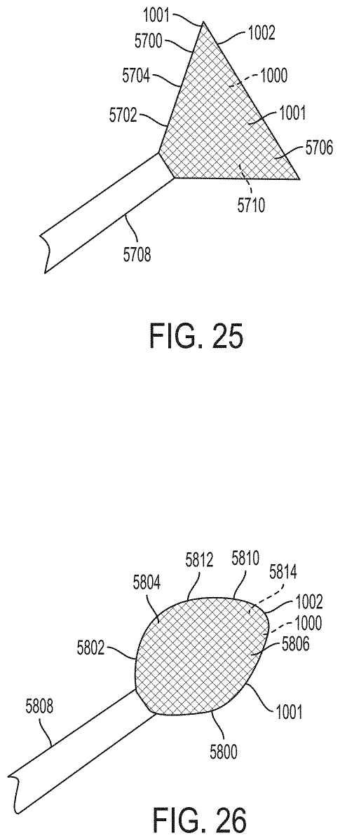

For example, a funnel shape, as shown in FIG. 25, can create a sidewall 5700 that conforms to the natural anatomical shape of the renal pelvis preventing the urothelium from constricting the fluid column. The interior 5710 of the funnel support 5702 provides a protected surface area 1000 having openings 5706 therethrough which provide a passageway through which a fluid column can flow from the calyces into the drainage lumen 5708. Similarly, the mesh form of FIG. 26 can also create a protected surface area 1000, such as interior 5814 of the mesh 5804, between the calyces and the drainage lumen 5808 of the catheter. The mesh 5704, 5804 comprises a plurality of openings 5706, 5806 therethrough for permitting fluid flow into the drainage lumen 5708, 5808. In some examples, the maximum area of an opening can be less than about 100 mm², or less than about 1 mm², or about 0.002 mm² to about 1 mm², or about 0.002 mm² to about 0.05 mm². The mesh 5704, 5804 can be formed from any suitable metallic or polymeric material such as are discussed above.

In some examples, the funnel support further comprises a cover portion over the distal end of the funnel support. This cover portion can be formed as an integral part of the funnel support or connected to the distal end of the funnel support. For example, as shown in FIG. 26, the funnel support 5802 comprises a cover portion 5810 across the distal end 5812 of the funnel support 5802 and projecting from the distal end 5812 of the funnel support 5802. The cover portion 5810 can have any shape desired, such as flat, convex, concave, undulating, and combinations thereof. The cover portion 5810 can be formed from mesh or any polymeric solid material as discussed above. The cover portion 5810 can provide an outer periphery 1002 or protective surface area 1001 to assist in supporting the pliant tissue in the kidney region to facilitate urine production.

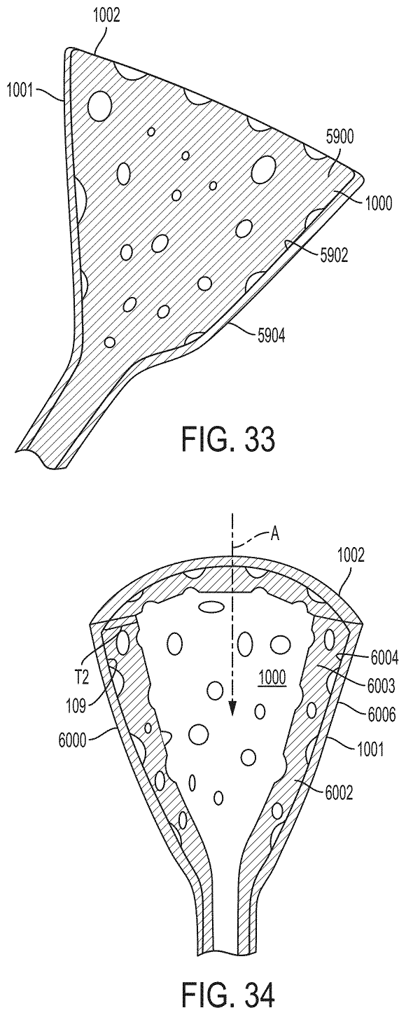

In some examples, the funnel support comprises a porous material, for example as shown in FIGS. 39A-40C. FIGS. 39A-40C and suitable porous materials are discussed in detail below. Briefly, in FIGS. 39 and 40, the porous material itself is the funnel support. In FIG. 39, the funnel support is a wedge of porous material. In FIG. 40, the porous material is in the shape of a funnel. In some examples, such as FIG. 33, the porous material 5900 is positioned within the interior 5902 of the sidewall 5904. In some examples, such as FIG. 34, the funnel support 6000 comprises a porous liner 6002 positioned adjacent to the interior 6004 of the sidewall 6006. The thickness T2 of the porous liner 6002 can range from about 0.5 mm to about 12.5 mm, for example. The area of the openings within the porous material can be about 0.002 mm² to about 100 mm², or less.

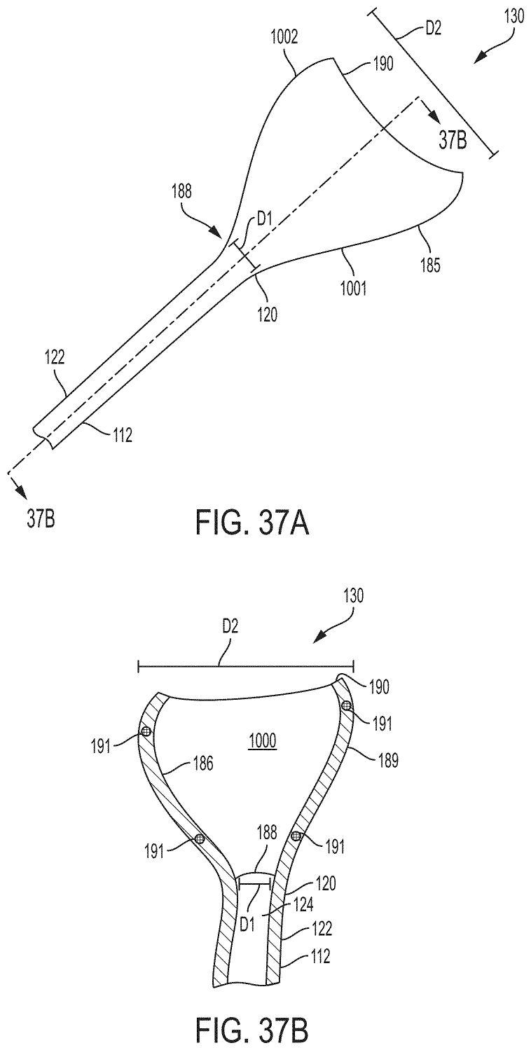

Referring now to FIGS. 37A and 37B, for example, a retention portion 130 of a ureteral catheter 112 comprises a catheter tube 122 having a widened and/or tapered distal end portion which, in some examples, is configured to be positioned in the patient's renal pelvis and/or kidney. For example, the retention portion 130 can be a funnel-shaped structure comprising an outer surface 185 configured to be positioned against the ureter and/or kidney wall and comprising an inner surface 186 configured to direct fluid toward a drainage lumen 124 of the catheter 112. The retention portion can be configured into a funnel-shaped support having an outer surface 185 and an inner surface 186, and wherein the outer periphery 189 or protective surface area 1001 comprises the outer surface 185 of the funnel-shaped support, and the one or more drainage holes, ports or perforations 133, 1233 are disposed on the inner surface 186 at the base of the funnel-shaped support. In another example shown in FIGS. 32A and 32B, the retention portion can be configured into a funnel-shaped support 5614 having an outer surface and an inner surface 5616, and wherein the outer periphery 1002 or protective surface area 1001 comprises the outer surface of the outer sidewall 5606. The protected surface area 1000 can comprise the inner sidewall 5604 of the inner funnel and the one or more drainage holes, ports or perforations 5600 can be disposed on the inner sidewall 5604 of the funnel-shaped support.

Referring to FIGS. 37A and 37B, the retention portion 130 can comprise a proximal end 188 adjacent to the distal end of the drainage lumen 124 and having a first diameter D1 and a distal end 190 having a second diameter D2 that is greater than the first diameter D1 when the retention portion 130 is in its deployed position. In some examples, the retention portion 130 is transitionable from a collapsed or compressed position to the deployed position. For example, the retention portion 130 can be biased radially outward such that when the retention portion 130 is advanced to its fluid collecting position, the retention portion 130 (e.g., the funnel portion) expands radially outward to the deployed state.

The retention portion 130 of the ureteral catheter 112 can be made from a variety of suitable materials that are capable of transitioning from the collapsed state to the deployed state. In one example, the retention portion 130 comprises a framework of tines or elongated members formed from a temperature sensitive shape memory material, such as nitinol. In some examples, the nitinol frame can be covered with a suitable waterproof material such as silicon to form a tapered portion or funnel. In that case, fluid is permitted to flow down the inner surface 186 of the retention portion 130 and into the drainage lumen 124. In other examples, the retention portion 130 is formed from various rigid or partially rigid sheets or materials bended or molded to form a funnel-shaped retention portion as illustrated in FIGS. 37A and 37B.

In some examples, the retention portion of the ureteral catheter 112 can include one or more mechanical stimulation devices 191 for providing stimulation to nerves and muscle fibers in adjacent tissues of the ureter(s) and renal pelvis. For example, the mechanical stimulation devices 191 can include linear or annular actuators embedded in or mounted adjacent to portions of the sidewall of the catheter tube 122 and configured to emit low levels of vibration. In some examples, mechanical stimulation can be provided to portions of the ureters and/or renal pelvis to supplement or modify therapeutic effects obtained by application of negative pressure. While not intending to be bound by theory, it is believed that such stimulation affects adjacent tissues by, for example, stimulating nerves and/or actuating peristaltic muscles associated with the ureter(s) and/or renal pelvis. Stimulation of nerves and activation of muscles may produce changes in pressure gradients or pressure levels in surrounding tissues and organs which may contribute to or, in some cases, enhance therapeutic benefits of negative pressure therapy.

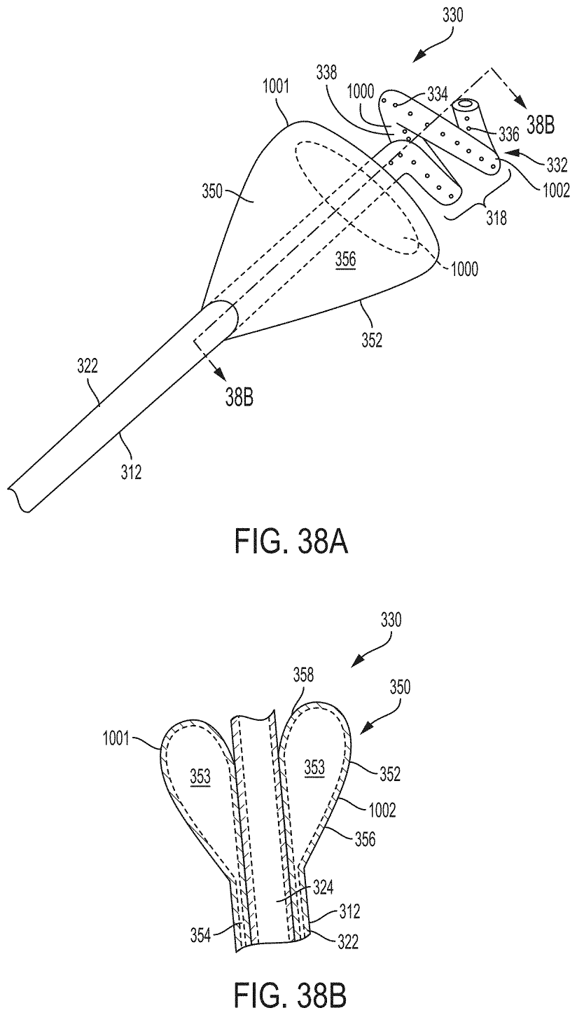

With reference to FIGS. 38A and 38B, according to another example, a retention portion 330 of a ureteral catheter 312 comprises a catheter tube 322 having a distal portion 318 formed in a helical structure 332 and an inflatable element or balloon 350 positioned proximal to the helical structure 332 to provide an additional degree of retention in the renal pelvis and/or fluid collection location. A balloon 350 can be inflated to pressure sufficient to retain the balloon in the renal pelvis or ureter, but low enough to avoid distending or damaging these structures. Suitable inflation pressures are known to those skilled in the art and are readily discernible by trial and error. As in previously-described examples, the helical structure 332 can be imparted by bending the catheter tube 322 to form one or more coils 334. The coils 334 can have a constant or variable diameter and height as described above. The catheter tube 322 further comprises a plurality of drainage ports 336 disposed on the sidewall of the catheter tube 322 to allow urine to be drawn into the drainage lumen 324 of the catheter tube 322 and to be directed from the body through the drainage lumen 324, for example on the inwardly facing and/or outwardly facing sides of the coil 334.

As shown in FIG. 38B, the inflatable element or balloon 350 can comprise an annular balloon-like structure having, for example, a generally heart-shaped cross section and comprising a surface or cover 352 defining a cavity 353. The cavity 353 is in fluid communication with an inflation lumen 354 extending parallel to the drainage lumen 324 defined by the catheter tube 322. The balloon 350 can be configured to be inserted in the tapered portion of the renal pelvis and inflated such that an outer surface 356 thereof contacts and rests against an inner surface of the ureter and/or renal pelvis. The inflatable element or balloon 350 can comprise a tapered inner surface 358 extending longitudinally and radially inward towards the catheter tube 322. The inner surface 358 can be configured to direct urine toward the catheter tube 322 to be drawn into the drainage lumen 324. The inner surface 358 can also be positioned to prevent fluid from pooling in the ureter, such as around the periphery of the inflatable element or balloon 350. The inflatable retention portion or balloon 350 is desirably sized to fit within the renal pelvis and can have a diameter ranging from about 10 mm to about 30 mm.

With reference to FIGS. 39A-40C, in some examples, an assembly 400 including a ureteral catheter 412 comprising a retention portion 410 is illustrated. The retention portion 410 is formed from a porous and/or sponge-like material that is attached to a distal end 421 of a catheter tube 422. The porous material can be configured to channel and/or absorb urine and direct the urine toward a drainage lumen 424 of the catheter tube 422. The retention portion 410 can be configured into a funnel-shaped support having an outer surface and an inner surface, and wherein the outer periphery 1002 or protective surface area 1001 comprises the outer surface of the funnel-shaped support, and the one or more drainage holes, ports or perforations in the porous material can be disposed within the porous material or on the inner surface 426 of the funnel-shaped support.

As shown in FIG. 40, the retention portion 410 can be a porous wedge shaped-structure configured for insertion and retention in the patient's renal pelvis. The porous material comprises a plurality of holes and/or channels. Fluid can be drawn through the channels and holes, for example, by gravity or upon inducement of negative pressure through the catheter 412. For example, fluid can enter the wedge-shaped retention portion 410 through the holes and/or channels and is drawn toward a distal opening 420 of the drainage lumen 424, for example, by capillary action, peristalsis, or as a result of the inducement of negative pressure in the holes and/or channels. In other examples, as shown in FIG. 40, the retention portion 410 comprises a hollow, funnel structure formed from the porous sponge-like material. As shown by arrow A, fluid is directed down an inner surface 426 of the funnel structure into the drainage lumen 424 defined by the catheter tube 422. Also, fluid can enter the funnel structure of the retention portion 410 through holes and channels in the porous sponge-like material of a sidewall 428. For example, suitable porous materials can include open-celled polyurethane foams, such as polyurethane ether. Suitable porous materials can also include laminates of woven or non-woven layers comprising, for example, polyurethane, silicone, polyvinyl alcohol, cotton, or polyester, with or without antimicrobial additives such as silver, and with or without additives for modifying material properties such as hydrogels, hydrocolloids, acrylic, or silicone.

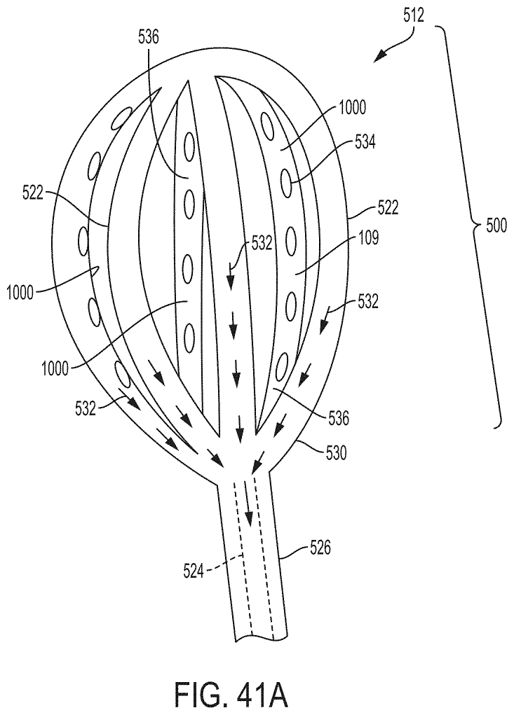

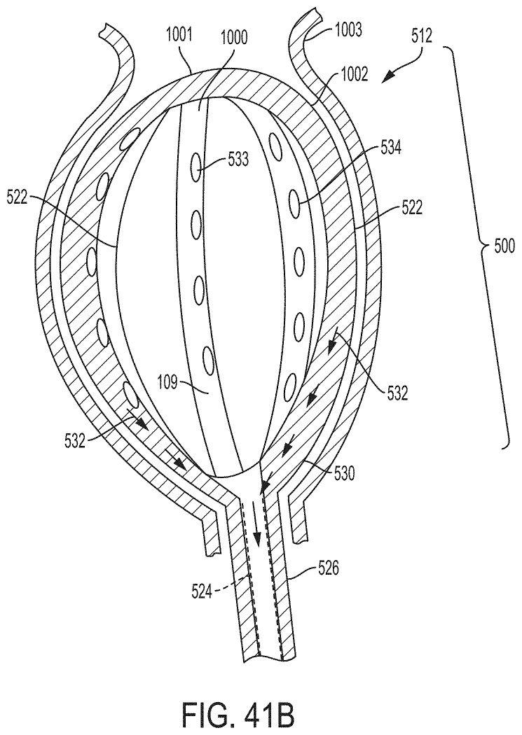

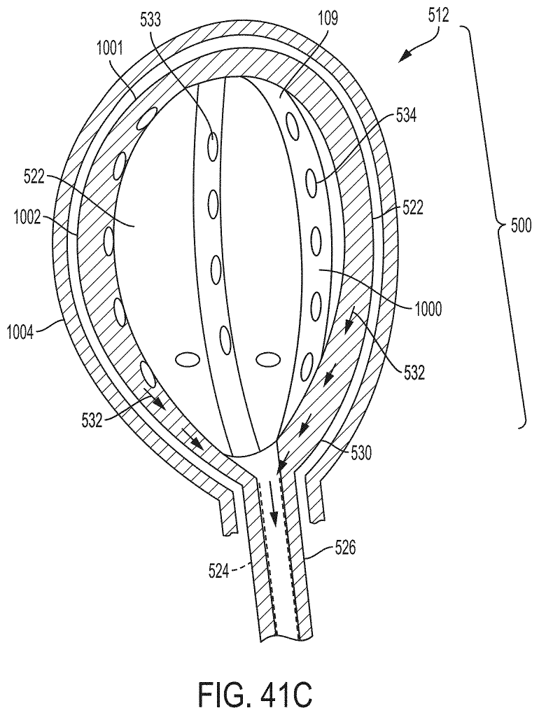

With reference to FIG. 41, according to another example, a retention portion 500 of a ureteral catheter 512 comprises an expandable cage 530. The expandable cage 530 comprises one or more longitudinally and radially extending hollow tubes 522. For example, the tubes 522 can be formed from an elastic, shape memory material such as nitinol. The cage 530 is configured to transition from a contracted state, for insertion through the patient's urinary tract, to a deployed state for positioning in the patient's ureters and/or kidney. The hollow tubes 522 comprise a plurality of drainage ports 534 which can be positioned on the tubes, for example, on radially inward facing sides thereof. The ports 534 are configured to permit fluid to flow or be drawn through the ports 534 and into the respective tubes 522. The fluid drains through the hollow tubes 522 into a drainage lumen 524 defined by a catheter body 526 of the ureteral catheter 512. For example, fluid can flow along the path indicated by the arrows 532 in FIG. 41. In some examples, when negative pressure is induced in the renal pelvis, kidneys, and/or ureters, portions of the ureter wall and/or renal pelvis may be drawn against the outward facing surfaces of the hollow tubes 522. The drainage ports 534 are positioned and configured so as not to be appreciably occluded by ureteral structures upon application of negative pressure to the ureters and/or kidney.

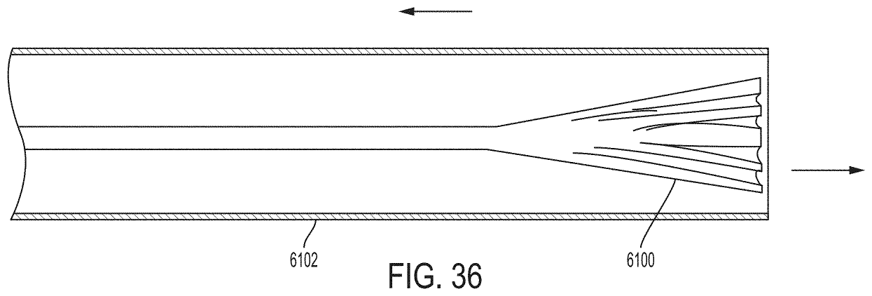

In some examples, the ureteral catheter comprising a funnel support can be deployed into a patient's urinary tract, and more specifically in the renal pelvis region/kidney using a conduit through the urethra and into the bladder. The funnel support 6100 is in a collapsed state (shown in FIG. 36) and sheathed in a ureteral sheath 6102. To deploy the ureteral catheter, the medical professional would insert a cystoscope into the urethra to provide a channel for tools to enter the bladder. The ureteral orifice would be visualized and guide wire would be inserted through the cystoscope and ureter until the tip of the guide wire reaches the renal pelvis. The cystoscope likely would be removed, and a “pusher tube” would be fed over the guide wire up to the renal pelvis. The guidewire would be removed while the “pusher tube” stays in place to act as deployment sheath. The ureteral catheter would be inserted through the pusher tube/sheath and the catheter tip would be actuated once it extends beyond the end of the pusher tube/sheath. The funnel support would expand radially to assume the deployed position.

### Exemplary Ureteral Stents

Referring now to FIG. 1A, in some examples, the ureteral stent 52, 54 comprises an elongated body comprising a proximal end 62, a distal end 58, a longitudinal axis, and at least one drainage channel that extends along the longitudinal axis from the proximal end to the distal end to maintain patency of fluid flow between a kidney and a bladder of the patient. In some examples, the ureteral stent further comprises a pigtail coil or loop(s) on at least one of the proximal end or the distal end. In some examples, the body of the ureteral stent further comprises at least one perforation on a sidewall thereof. In other examples, the body of the ureteral stent is essentially free of or free of perforation(s) on a sidewall thereof.

Some examples of ureteral stents 52, 54 that can be useful in the present systems and methods include CONTOUR™ ureteral stents, CONTOUR VL™ ureteral stents, POLARIS™ Loop ureteral stents, POLARIS™ Ultra ureteral stents, PERCUFLEX™ ureteral stents, PERCUFLEX™ Plus ureteral stents, STRETCH™ VL Flexima ureteral stents, each of which are commercially available from Boston Scientific Corporation of Natick, Mass. See “Ureteral Stent Portfolio”, a publication of Boston Scientific Corp., (July 2010), hereby incorporated by reference herein. The CONTOUR™ and CONTOUR VL™ ureteral stents are constructed with soft Percuflex™ Material that becomes soft at body temperature and is designed for a 365-day indwelling time. Variable length coils on distal and proximal ends allow for one stent to fit various ureteral lengths. The fixed length stent can be 6F-8F with lengths ranging from 20 cm-30 cm, and the variable length stent can be 4.8F-7F with lengths of 22-30 cm. Other examples of suitable ureteral stents include INLAY® ureteral stents, INLAY® OPTIMA® ureteral stents, BARDEX®double pigtail ureteral stents, and FLUORO-4™ silicone ureteral stent, each of which are commercially available from C. R. Bard, Inc. of Murray Hill, N.J. See “Ureteral Stents”, http://www.bardmedical.com/products/kidney-stone-management/ureteral-stents/(Jan. 21, 2018), hereby incorporated by reference herein.

The stents 52, 54 can be deployed in one or both of the patient's kidneys or kidney area (renal pelvis or ureters adjacent to the renal pelvis), as desired. Typically, these stents are deployed by inserting a stent having a nitinol wire therethrough through the urethra and bladder up to the kidney, then withdrawing the nitinol wire from the stent, which permits the stent to assume a deployed configuration. Many of the above stents have a planar loop 58, 60 on the distal end (to be deployed in the kidney), and some also have a planar loop 62, 64 on the proximal end of the stent which is deployed in the bladder. When the nitinol wire is removed, the stent assumes the pre-stressed planar loop shape at the distal and/or proximal ends. To remove the stent, a nitinol wire is inserted to straighten the stent and the stent is withdrawn from the ureter and urethra.

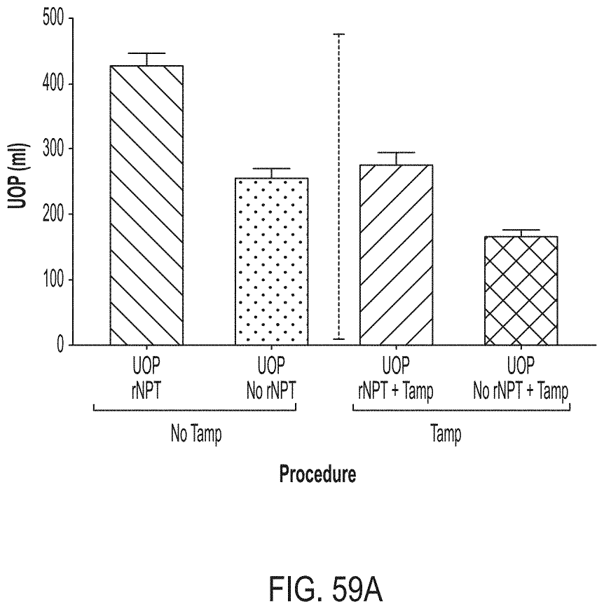

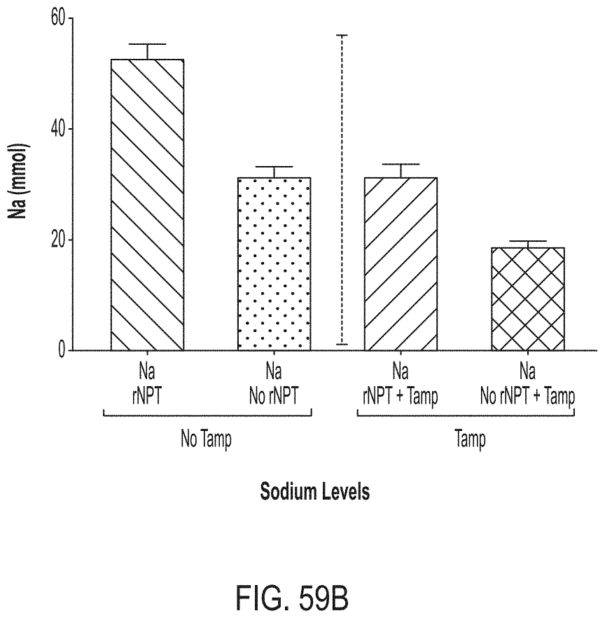

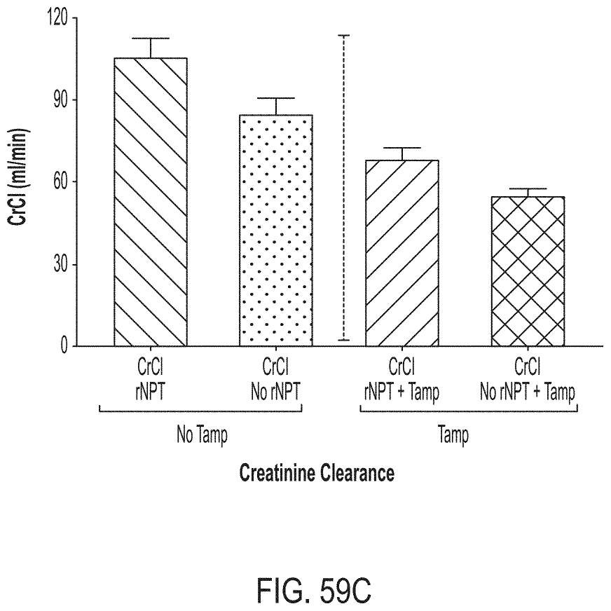

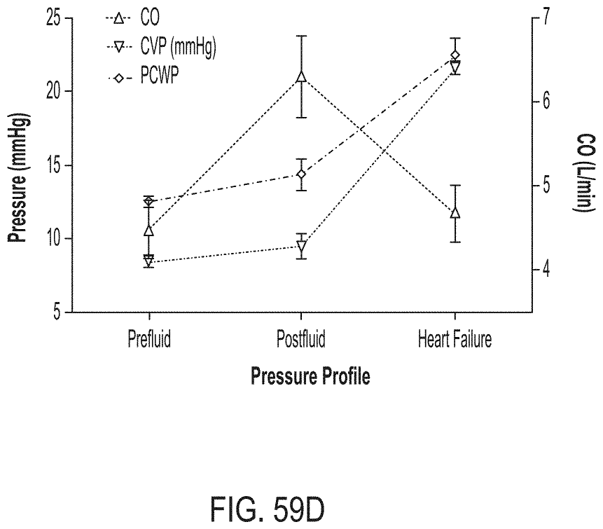

Other examples of suitable ureteral stents 52, 54 are disclosed in PCT Patent Application Publication WO 2017/019974, which is incorporated by reference herein. In some examples, as shown, for example, in FIGS. 1-7 of WO 2017/019974 and in FIG. 3 herein (same as FIG. 1 of WO 2017/019974), the ureteral stent 100 can comprise: an elongated body 101 comprising a proximal end 102, a distal end 104, a longitudinal axis 106, an outer surface 108, and an inner surface 110, wherein the inner surface 110 defines a transformable bore 111 that extends along the longitudinal axis 106 from the proximal end 102 to the distal end 104; and at least two fins 112 projecting radially away from the outer surface 108 of the body 101; wherein the transformable bore 111 comprises: (a) a default orientation 113A (shown on the left in FIG. 59) comprising an open bore 114 defining a longitudinally open channel 116; and (b) a second orientation 113B (shown on the right in FIG. 59) comprising an at least essentially closed bore 118 or closed bore defining a longitudinally essentially closed drainage channel 120 along the longitudinal axis 106 of the elongated body 101, wherein the transformable bore 111 is moveable from the default orientation 113A to the second orientation 113B upon radial compression forces 122 being applied to at least a portion of the outer surface 108 of the body 101.

In some examples, as shown in FIG. 3, the drainage channel 120 of the ureteral stent 100 has a diameter D which is reduced upon the transformable bore 111 moving from the default orientation 113A to the second orientation 113B, wherein the diameter is reducible up to the point above where urine flow through the transformable bore 111 would be reduced. In some examples, the diameter D is reduced by up to about 40% upon the transformable bore 111 moving from the default orientation 113A to the second orientation 113B. In some examples, the diameter D in the default orientation 113A can range from about 0.75 to about 5.5 mm, or about 1.3 mm or about 1.4 mm. In some examples, the diameter D in the second orientation 113B can range from about 0.4 to about 4 mm, or about 0.9 mm.

In some examples, one or more fins 112 comprise a flexible material that is soft to medium soft based on the Shore hardness scale. In some examples, the body 101 comprises a flexible material that is medium hard to hard based on the Shore hardness scale. In some examples, one or more fins have a durometer between about 15 A to about 40 A. In some examples, the body 101 has a durometer between about 80 A to about 90 A. In some examples, one or more fins 112 and the body 101 comprise a flexible material that is medium soft to medium hard based on the Shore hardness scale, for example having a durometer between about 40 A to about 70 A.

In some examples, one or more fins 112 and the body 101 comprise a flexible material that is medium hard to hard based on the Shore hardness scale, for example having a durometer between about 85 A to about 90 A.

In some examples, the default orientation 113A and the second orientation 113B support fluid or urine flow around the outer surface 108 of the stent 100 in addition to through the transformable bore 111.

In some examples, one or more fins 112 extend longitudinally from the proximal end 102 to the distal end 104. In some examples, the stent has two, three or four fins.

In some examples, the outer surface 108 of the body has an outer diameter in the default orientation 113A ranging from about 0.8 mm to about 6 mm, or about 3 mm. In some examples, the outer surface 108 of the body has an outer diameter in the second orientation 113B ranging from about 0.5 mm to about 4.5 mm, or about 1 mm. In some examples, one or more fins have a width or tip ranging from about 0.25 mm to about 1.5 mm, or about 1 mm, projecting from the outer surface 108 of the body in a direction generally perpendicular to the longitudinal axis.

In some examples, the radial compression forces are provided by at least one of normal ureter physiology, abnormal ureter physiology, or application of any external force. In some examples, the ureteral stent 100 purposefully adapts to a dynamic ureteral environment, the ureteral stent 100 comprising: an elongated body 101 comprising a proximal end 102, a distal end 104, a longitudinal axis 106, an outer surface 108, and an inner surface 110, wherein the inner surface 110 defines a transformable bore 111 that extends along the longitudinal axis 106 from the proximal end 102 to the distal end 104; wherein the transformable bore 111 comprises: (a) a default orientation 113A comprising an open bore 114 defining a longitudinally open channel 116; and (b) a second orientation 113B comprising an at least essentially closed bore 118 defining a longitudinally essentially closed channel 120, wherein the transformable bore is moveable from the default orientation 113A to the second orientation 113B upon radial compression forces 122 being applied to at least a portion of the outer surface 108 of the body 101, wherein the inner surface 110 of the body 101 has a diameter D which is reduced upon the transformable bore 111 moving from the default orientation 113A to the second orientation 113B, wherein the diameter is reducible up to the point above where fluid flow through the transformable bore 111 would be reduced. In some examples, the diameter D is reduced by up to about 40% upon the transformable bore 111 moving from the default orientation 113A to the second orientation 113B.

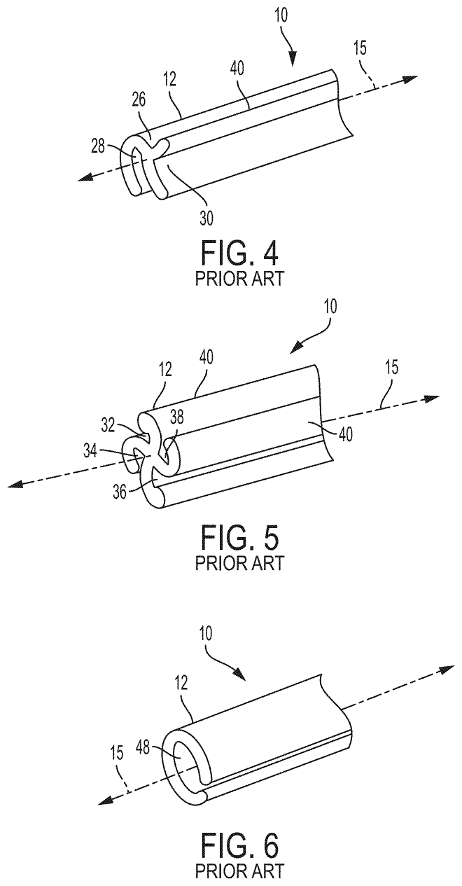

Other examples of suitable ureteral stents are disclosed in US Patent Application Publication US 2002/0183853 A1, which is incorporated by reference herein. In some examples, as shown, for example, in FIGS. 4, 5 and 7 of US 2002/0183853 A1 and in FIGS. 4-6 herein (same as FIGS. 1 of 4, 5 and 7 of US 2002/0183853 A1), the ureteral stent comprises an elongated, body 10 comprising a proximal end 12, a distal end 14 (not shown), a longitudinal axis 15, and at least one drainage channel (for example, 26, 28, 30 in FIGS. 4; 32, 34, 36 and 38 in FIG. 5; and 48 in FIG. 6) that extends along the longitudinal axis 15 from the proximal end 12 to the distal end 14 to maintain patency of fluid flow between a kidney and a bladder of the patient. In some examples, the at least one drainage channel is partially open along at least a longitudinal portion thereof. In some examples, the at least one drainage channel is closed along at least a longitudinal portion thereof. In some examples, the at least one drainage channel is closed along the longitudinal length thereof. In some examples, the ureteral stent is radially compressible. In some examples, the ureteral stent is radially compressible to narrow the at least one drainage channel. In some examples, the elongated body 10 comprises at least one external fin 40 along the longitudinal axis 15 of the elongated body 10. In some examples, the elongated body comprises one to four drainage channels. The diameter of the drainage channel can be the same as described above.

Coated and/or Impregnated Catheter or Stent Device

In some examples, at least a portion or all of the device, such as any of the catheter(s) and/or the stent(s) described herein, can be coated and/or impregnated with at least one of the coating/impregnant material(s) described herein. Referring generally to FIGS. 1A-1W, 2A-14, 17-41C, 52A-54, and 56-57, portions or all of any of the devices described herein, generally designated collectively as 7010, such as a catheter or stent, can be coated and/or impregnated with at least one of the coating/impregnant materials described herein.

In some examples, the device 7010 can be configured to facilitate insertion and/or removal of the coated device 7010 within a urinary tract 1 of the patient and/or, once inserted, the at least one coating(s) and/or impregnation(s) 7022 can improve function of the device 7010. The device 7010 can be configured for insertion in one or more of a bladder, ureter, renal pelvis, and/or kidney of a patient. The device 7010 can be deployed to maintain an end 7044 or retention portion 7020 of the device 7010 at a desired position within the urinary tract 1. The device 7010 can be sized to fit securely at a desired position within the urinary tract 1, as described in detail herein. The device 7010 can be narrow enough in a retracted state so that the coated device 7010 can be easily inserted and removed. The device 7010 can have any of the configurations described herein, for example a catheter or stent. Non-limiting examples of suitable catheters and stents are disclosed herein and are shown in FIGS. 1A-1W, 2A-14, 17-41C, 52A-54, and 56-57. In some examples, the retention portion 7020 of a suitable catheter comprises a funnel, coil, balloon, cage, sponge, and/or combinations thereof.

The device 7010 to be coated and/or impregnated can be formed from or comprise at least one device material(s) comprising at least one of copper, silver, gold, nickel-titanium alloy, stainless steel, titanium, and/or biocompatible polymer(s), such as polyurethane, polyvinyl chloride, polytetrafluoroethylene (PTFE), latex, silicone coated latex, silicone, polyglycolide or poly(glycolic acid) (PGA), Polylactide (PLA), Poly(lactide-co-glycolide), Polyhydroxyalkanoates, Polycaprolactone and/or Poly(propylene fumarate), as discussed in detail above.

In some examples, the at least one coating(s) and/or impregnation(s) 7022 can be present on and/or in at least the outer periphery 72, 1002 or the protective surface area 1001, 7038 of the device 7010 that inhibits mucosal tissue 1003 from occluding the at least one protected drainage hole(s), port(s) or perforation(s) 7036 upon application of negative pressure through the catheter. In some examples, the at least one coating(s) and/or impregnation(s) 7022 can be present on and/or in any portion(s) of the device 7010, and/or on or in the entire device 7010. In some examples, the at least one coating(s) and/or impregnation(s) 7022 can be present on and/or in at least a portion(s) of the retention portion 7020, or on and/or in all of the retention portion 7020. In some examples, the at least one coating(s) and/or impregnation(s) 7022 can be present on and/or in at least a portion(s) of a surface of the device, or on and/or in the entire surface of the device. In some examples, the at least one coating(s) and/or impregnation(s) 7022 can be present on and/or in at least an outer surface 7028 of the device, or on and/or in the entire outer surface 7028 of the device. In some examples, the at least one coating(s) and/or impregnation(s) 7022 can be present on and/or in other portions of the device 7010, such as portions or all of the delivery catheters of any of the above described catheter assemblies. In some examples, the at least one coating(s) and/or impregnation(s) 7022 are formed from one or more flexible coating materials, which do not appreciably or substantially affect a flexibility of the coated and/or impregnated device 7010.

In some examples, the at least one coating(s) 7022 can comprise one or more coatings, for example one to ten coatings, or two to four coatings. In some examples, the material(s) from which the device 7010 is formed (device material(s) discussed herein) can be coated with at least one of the coating/impregnant material(s) discussed herein. The coating(s) 7022 may be applied or formed in layers, with the understanding that it is possible that components of one coating layer may migrate into one or more adjacent or proximate layers, and or into the surface or within the device 7010.

In some examples, the material(s) from which the device 7010 is formed (device material(s) discussed herein) can be impregnated with at least one of the coating/impregnant material(s) discussed herein. As used herein, “impregnated” means that at least a portion of the coating/impregnant material(s) discussed herein permeate beneath an outer surface of and/or within at least a portion of the device material(s) from which the device 7010 is formed. In some examples, different coating/impregnant material(s) and/or different amounts of respective coating/impregnant material(s) discussed herein can be used to impregnate different portions or regions of the device 7010. For example, the retention portion 7020 can be impregnated with at least one of the coating/impregnant material(s) described herein, such as at least one lubricant material(s) and/or at least one antimicrobial material(s), while the drainage tube is impregnated only with at least one antimicrobial material(s). Portions or all of the device 7010 can be both impregnated and/or coated, as desired. In some examples, there can be layers of different impregnants at different depths within the device 7010.

The at least one coating/impregnant material(s) (which can be used as a coating material(s) and/or impregnant material(s), referred to as “coating/impregnant material(s)” for brevity) comprises at least one (one or more) of lubricant(s), antimicrobial material(s), pH buffer(s) or anti-inflammatory material(s). In some examples, the at least one coating(s) and/or impregnation(s) 7022 can be used to improve short term or long term performance of the device 7010, reduce pain during insertion/removal of the device 7010 into the urinary tract, and/or mitigate risks associated with prolonged use of an indwelling device.

For example, at least a portion of the device 7010 can be coated and/or impregnated with at least one coating/impregnant material(s) comprising at least one lubricant(s). The at least one coating(s) and/or impregnation(s) 7022 comprising the at least one lubricant(s) can, for example, have a lower coefficient of friction than the uncoated/unimpregnated device, function as a lubricant, and/or become lubricated or slippery in the presence of fluid such as moisture or urine. The presence of a lubricant in the at least one coating(s) and/or impregnation(s) 7022 may make the device 7010 easier to deploy and remove. Generally, in some examples, the at least one coating(s) and/or impregnation(s) 7022 can comprise materials configured to address issues and sources of discomfort associated with indwelling catheters.

Alternatively or additionally, the device 7010 can be coated and/or impregnated with at least one of antimicrobial material(s), pH buffer(s) and/or anti-inflammatory material(s). The at least one of antimicrobial material(s), pH buffer(s) and/or anti-inflammatory material(s) may mitigate risks associated with prolonged use of indwelling catheters, such as tissue ingrowth through portions of the device, foreign body reactions caused when portions of the device contact surrounding fluid and/or tissues, infection to tissues surrounding the device, and/or formation of encrustations on portions of the device. Encrustations can be caused by, for example, protein adsorption and/or buildup of minerals or urine crystals.

In some examples, the at least one coating(s) and/or impregnation(s) 7022 comprises at least one lubricant. In some examples, outer surface(s) or layer(s) of the at least one coating(s) and/or impregnation(s) 7022 comprise at least one lubricant. Lubricious coating(s)/impregnant(s) can be described in terms of their degree of lubricity or kinetic coefficient of friction, or the amount of friction reduction provided compared to an uncoated device, or a device comprising one or more coating(s)/impregnant(s) having an outer layer(s) having a kinetic coefficient of friction which is greater than the kinetic coefficient of friction of a lubricant coated comparable device. The kinetic coefficient of friction can be determined using ASTM Method D1894-14 (March 2014). A rigid mandrel can be inserted through the inner lumen of the stent/catheter section being tested, which is sized to minimize the amount of open space inside the stent/catheter inner lumen and any potential resultant constriction of the inner lumen when the sled is dragged along the material. Alternatively, the catheter tube can be slit and opened into a flattened sheet for testing.

In some examples, the lubricant(s) can comprise at least one hydrophilic lubricant material. Exemplary hydrophilic lubricant materials comprise at least one (one or more) of polyethylene glycol, polyvinylpyrrolidone, polytetrafluoroethylene, polyvinyl alcohol, polyacrylamide, polymethacrylate, as well as other acrylic polymers or copolymers of the above-listed materials, or polyelectrolytes. An exemplary hydrophilic coating material/impregnant is ComfortCoat® polyelectrolyte-containing hydrophilic coating which is available from Koninklijke D S M N. V. Examples of suitable hydrophilic coating/impregnant material(s) comprising polyelectrolyte(s) are disclosed in U.S. Pat. No. 8,512,795, which is incorporated by reference herein.

In some examples, the at least one coating(s)/impregnation(s) 7022 can comprise at least one material(s) which is not hydrophilic. For example, one or more layers of the at least one coating(s) and/or impregnation(s) 7022 can be comprise or be formed from polytetrafluoroethylene (e.g., Teflon), siloxane(s), silicone or polysiloxane(s), or other slippery and/or low friction materials.

In some examples, the lubricant can comprise at least one polymer material(s), such as at least partially cross-linked polymer material(s) (e.g., a gel or hydrogel). In some examples, the at least one polymer material(s) readily takes up or entraps fluid or liquid. Gels or hydrogels are systems that comprise three-dimensional, physically or chemically bonded polymer networks that entrap fluid or liquid, such as water, in intermolecular space. As known in the art, a gel or hydrogel can refer to an at least partially cross-linked material comprising a substantial liquid portion, but which exhibits little or no flow when in a steady state. By weight, a gel is generally mostly liquid, but may behave like a solid due to the cross-linked structure. Due to their ability to accommodate high water content, porosity, and soft consistency, hydrogels closely simulate natural living tissue, Gels and hydrogels may be chemically stable or they may degrade and eventually disintegrate and dissolve. In some examples, the at least one lubricant is biocompatible.

For example, useful gels or hydrogels can comprise one or more of polyethylene glycol, polyvinylpyrrolidone, polytetrafluoroethylene, polyvinyl alcohol, polyacrylamide, polymethacrylate, and/or hydrogels comprising polyacrylic acid (PAA) and/or disulphide-crosslinked (poly(oligo(ethyleneoxide) monomethyl ether methacrylate)) (POEMA). As a result of taking up or entrapping fluid (such as moisture), some hydrophilic materials can become gel-like, slick, and/or smooth.

Accordingly, when in the presence of fluid (such as moisture and/or urine), the hydrophilic material of the lubricant can provide increased lubricity between the stent or catheter device 7010 and adjacent portions of the urinary tract of the patient.

Combinations or mixtures of hydrophilic lubricant material(s), non-hydrophilic lubricant material(s) and/or polymer lubricant material(s) can be used in the same coating/impregnant or different coating(s)/impregnant(s), or layers thereof, as desired. In some examples, the concentration of the at least one lubricant(s) in the at least one coating(s) and/or impregnation(s) 7022 prior to drying or curing can range from about 0.1 to about 99.9 weight percent or 100 weight percent based upon the total weight of the coating/impregnant material(s) composition, or about 20 to 100 weight percent, or about 50 to about 100 weight percent. In some examples, the concentration of the at least one lubricant(s) in the at least one coating(s) and/or impregnation(s) 7022 after drying or curing can range from about 0.1 to about 99.9 weight percent or 100 weight percent based upon the total weight of the dried or cured coating/impregnation, or about 20 to 100 weight percent, or about 50 to about 100 weight percent.

In some examples, the at least one coating(s) and/or impregnation(s) 7022 can comprise at least one antimicrobial material(s), for example to inhibit tissue growth and/or to prevent infection. For example, any of the at least one coating(s) and/or impregnation(s) 7022, such as the outermost layer 7024 and/or any of the sublayer(s) 7026, can comprise the at least one antimicrobial material itself, or one or more material(s) comprising the at least one antimicrobial material, for example a polymer matrix formed from a suitable biocompatible material impregnated with antimicrobial material(s). Alternatively or additionally, the sublayer(s) 7026 can comprise a liposome-coating or similar material, and can be configured to deliver bacteriophages or drug therapies. An antimicrobial material can refer to, for example, at least one of antiseptic material(s), antiviral material(s), antibacterial material(s), antifungal material(s), and/or an antibiotic material(s), such as antibiotic medication or therapeutic agent. Examples of suitable antibacterial, antifungal, and/or antiseptic agents and materials can comprise chlorhexidine, silver ions, nitric oxide, bacteriophages, sirolimus, and/or sulfonamides. Some antibacterial materials, such as sirolimus, may also act as an immunosuppressant to reduce a foreign body response induced by an indwelling catheter. Examples of suitable antibiotic materials that can be included in at least one coating(s) and/or impregnation(s) 7022 can comprise amdinocillin, levofloxacin, penicillin, tetracyclines, sparfloxacin, and/or vancomycin. Doses or concentrations of such antimicrobial medications can be selected to avoid or reduce occurrence of infection, such as are known to those skilled in the art, such as about 1 to about 100 mcg/cm³. The antimicrobial and/or antibacterial materials of the coating(s) can also comprise materials such as heparin, phosphorylcholine, silicone dioxide, and/or diamond-like carbon, to inhibit any of protein adsorption, biofilm formation, mineral and/or crystal buildup, and similar risk factors. Other suitable antimicrobial materials, which provide useful functional properties for the coating(s), can comprise other antimicrobial peptides, caspofungin, chitosan, parylenes, as well as organosilanes and other materials that impart mechanical antimicrobial properties.

In some examples, the concentration of the at least one antimicrobial material(s) in the at least one coating(s) and/or impregnation(s) 7022 prior to drying or curing can range from about 0.1 to about 99.9 weight percent or 100 weight percent based upon the total weight of the coating/impregnant material(s) composition, or about 20 to 100 weight percent, or about 50 to about 100 weight percent. In some examples, the concentration of the at least one antimicrobial material(s) in the at least one coating(s) and/or impregnation(s) 7022 after drying or curing can range from about 0.1 to about 99.9 weight percent or 100 weight percent based upon the total weight of the dried or cured coating/impregnation, or about 20 to 100 weight percent, or about 50 to about 100 weight percent.

In some examples, the at least one coating(s) and/or impregnation(s) 7022 comprising the antimicrobial material(s) should provide suitable protection for the coated device 7010 for the entire usable life of the device 7010, although the time period in which antimicrobial properties are present may be shorter. Accordingly, the at least one coating(s) and/or impregnation(s) 7022 should be thick enough and contain enough antimicrobial material to continue to exhibit antimicrobial properties for the usable life of the coated device 7010, which can be from about 1 day to about one year, or from about 10 days to about 180 days, or from about 30 days to about 90 days.

In some examples, alternatively or additionally, the at least one coating(s) and/or impregnation(s) 7022 can comprise at least one pH buffering material(s) to buffer the pH of the fluid in the urinary tract, such as urine. Such a buffering material(s), for example, may reduce or eliminate encrustations, which may adhere to surfaces of the coated device 7010. A pH buffering material is believed to reduce encrustations by inhibiting or preventing formation of urine crystals, which often adhere to indwelling structures positioned within the urinary tract. For example, in the presence of organisms capable of producing the urease enzyme, local increases in ammonium concentration and pH can result in the formation of crystals of at least one of calcium phosphate, magnesium phosphate, or magnesium ammonium phosphate, while crystals of urates and oxalates are more commonly seen with decreased pH level. The pH of urine can range from about 4.5 to about 8.0, typically about 6.0.

When the pH of the fluid in the urinary tract rises above a predetermined value, such as 6.0 or 7.0, for example, the at least one coating(s) and/or impregnation(s) 7022 can release a portion or all of the at least one buffering material into the fluid. Alternatively or additionally, when the pH of the fluid in the urinary tract falls below a predetermined value, such as 5.5 or 6.0, for example, the at least one coating(s) and/or impregnation(s) 7022 can release a portion or all of the at least one buffering material into the fluid. Alternatively or additionally, the at least one coating(s) and/or impregnation(s) 7022 can release a portion or all of the at least one buffering material into the fluid when the concentration of at least one of calcium, magnesium, phosphorous, oxalates or uric acid reaches a predetermined value. For example in patients with elevated levels of at least one of calcium, magnesium, phosphorous, oxalates or uric acid in their fluid or urine, examples of suitable predetermined values of analytes at which the at least one coating(s) and/or impregnation(s) 7022 can release a portion or all of the at least one buffering material into the fluid are: for calcium at least about 15 mg/deciliter (dl), for magnesium at least about 9 mg/dl, for phosphorous at least about 60 mg/dl, for oxalates at least about 1.5 mg/dl, and for uric acid at least about 36 mg/dl. Calcium specifically is commonly referenced as a ratio to urinary creatinine, i.e., normal value would be urine calcium:urine creatinine <0.14. The levels of these analytes in fluid or urine can be determined using one or more of colorimetric analysis, spectrometry or microscopy methods of the fluid or urine samples. “Normal” values and reference ranges are often provided in units of ‘mg/24 hrs’ since the excretion is highly driven by dietary intake and so would be expected to be variable over time. Excretion of these analytes can also be significantly impacted by the use of certain medications, such as diuretics. A device could intrinsically “sense” and react to analyte levels by releasing one or more buffer agents as a result of the binding of analytes to at least a portion of or a component of a coating layer. Predetermined thresholds can be set for specific analytes in order to determine binding affinities of a coating layer so that different levels of binding would trigger release of varying amounts of the one or more buffer agents, as desired.

Examples of suitable pH buffering material(s) can comprise, for example, an acid salt impregnated in a dissolvable polymer material layer. As will be appreciated by those skilled in the art, as the acid salt dissolves in the presence of bodily fluid or moisture, an acid solution is produced. Desirably, the produced acidic solution inhibits formation of the encrustations, but is not so acidic as to damage body tissues. Examples of suitable acid salts that can be used as a suitable pH buffer layer can comprise weakly acidic salts, such as sodium citrate, sodium acetate, and/or sodium bicarbonate. In some examples, the pH buffering material(s) can be dispersed in a hydrogel, colloid and/or copolymer matrix, such as methacrylic acid and methyl methacrylate copolymer, and dispersed or layered with high affinity calcium- or phosphate-binding agent(s), such as ethylene glycol tetraacetic acid (EGTA).

In some examples, the concentration of the at least one pH buffering material(s) in the at least one coating(s) and/or impregnation(s) 7022 prior to drying or curing can range from about 0.1 to about 99.9 weight percent or 100 weight percent based upon the total weight of the coating/impregnant material(s) composition, or about 20 to 100 weight percent, or about 50 to about 100 weight percent. In some examples, the concentration of the at least one pH buffering material(s) in the at least one coating(s) and/or impregnation(s) 7022 after drying or curing can range from about 0.1 to about 99.9 weight percent or 100 weight percent based upon the total weight of the dried or cured coating/impregnation, or about 20 to 100 weight percent, or about 50 to about 100 weight percent.

In some examples, the at least one coating(s) and/or impregnation(s) 7022 can comprise outermost layer(s) 7024 and a single or multiple sublayer(s) 7026 (e.g., comprising either an antimicrobial sublayer or a pH buffering sublayer). In other examples, the sublayer(s) 7026 of the at least one coating(s) and/or impregnation(s) 7022 can comprise, for example, a first sublayer 7030 applied to the outer surface 7028 of the device 7010 comprising a pH buffering material, for example for reducing encrustation of urine crystals, and a second sublayer 7032 covering at least a portion of the first sublayer 7030. The second sublayer 7032 can comprise the antimicrobial material(s), for example. Alternatively, the first sublayer 7030 can comprise the antimicrobial material(s) and the second sublayer 7032 can comprise the pH buffering material(s).

In some examples, alternatively or additionally, the at least one coating(s) and/or impregnation(s) 7022 can comprise at least one anti-inflammatory material(s). Following insertion into the body, proteins and other biomolecules in the body, such as in the blood plasma and biological fluids, absorb onto the surface of the biomaterial of the device or implant. Nonspecific biomolecule and protein adsorption and subsequent leukocyte adhesion, known as “biofouling” may result. Subsequent inflammatory reactions can result, such as biomaterial-mediated inflammation, which is a complex reaction of protein adsorption, leukocyte recruitment/activation, secretion of inflammatory mediators, and fibrous encapsulation of part or all of the device or implant. Reducing the inflammatory response, for example by reducing the protein binding and ability of the immune response to propagate, can prevent or reduce possible injury to the urinary tract tissues by contact with the device in the absence or presence of negative pressure.

Non-limiting examples of suitable anti-inflammatory material(s) comprise anti-inflammatory agent(s) and non-fouling surface treatment material(s). Examples of suitable anti-inflammatory agent(s) comprise at least one of Dexamethasone (DEX), Heparin or Alpha-melanocyte-stimulating hormone α-MSH). Examples of suitable non-fouling surface treatment material(s) comprise at least one of polyethylene glycol-containing polymers, poly(2-hydroxyethyl methacrylate), poly(N-isopropyl acrylamide), poly(acrylamide), phosphoryl choline-based polymers, mannitol, oligomaltose, and taurine groups.

In some examples, the concentration of the at least one anti-inflammatory material(s) in the at least one coating(s) and/or impregnation(s) 7022 prior to drying or curing can range from about 0.1 to about 99.9 weight percent or 100 weight percent based upon the total weight of the coating/impregnant material(s) composition, or about 20 to 100 weight percent, or about 50 to about 100 weight percent. In some examples, the concentration of the at least one anti-inflammatory material(s) in the at least one coating(s) and/or impregnation(s) 7022 after drying or curing can range from about 0.1 to about 99.9 weight percent or 100 weight percent based upon the total weight of the dried or cured coating/impregnation, or about 20 to 100 weight percent, or about 50 to about 100 weight percent.

In some examples, the at least one coating(s) and/or impregnation(s) 7022 can comprise the outermost layer(s) 7024 and a single or multiple sublayer(s) 7026 comprising the at least one anti-inflammatory material(s). In some examples, the outermost layer(s) 7024 can comprise at least one pH buffer material, and one or more sublayer(s) 7026 can comprise the at least one anti-inflammatory material(s).

In some examples, an overall thickness of the at least one coating(s) and/or impregnation(s) 7022, or depth of impregnation into the device material(s), can range from about 0.001 micrometer (about 1 nanometer) to about 10.0 millimeters, or about 0.001 micrometer to about 5 mm, or about 0.001 mm to about 5.0 mm, or about 0.01 mm to about 1.0 mm, or about 0.001 micrometer to about 0.2 mm, after application and drying and/or curing of the coating(s)/impregnant(s). In some examples, each coating/impregnant layer within multiple coating(s)/impregnant(s) layers can have a thickness ranging from about 0.001 micrometer to about 10.0 millimeters, or about 0.001 micrometer to about 5.0 mm, or about 0.001 micrometer to about 500 micrometers, after application and drying or curing of the coating(s)/impregnant(s) layer.

In some examples, an overall thickness of the at least one hydrated or swelled coating(s) and/or impregation(s) 7022, or depth of impregnation into the device material(s), can range from about 0.1 micrometer to about 25.0 millimeters, or about 0.1 micrometer to about 500 micrometers, or about 20 micrometers±20%.

In some examples, the density of the coating/impregnant material(s) composition can range from about 0.1 to about 200 mg/microliter prior to drying or curing, or about 1 mg/microliter. The coating/impregnant material(s) to be applied to the device 7010, prior to drying or curing, can further comprise at least one carrier or adjuvant, such as water, alcohol(s), silica oils such as polydimethylsiloxane(s), and/or polymeric matrix materials such as hydrogels, for example comprising polyacrylic acid (PAA) and/or disulphide-crosslinked (poly(oligo(ethyleneoxide) monomethyl ether methacrylate)) (POEMA).

In some examples, the concentration of at least one of lubricant(s), antimicrobial material(s), pH buffer(s) or anti-inflammatory material(s) in the coating/impregnant material(s) composition prior to drying or curing can range from about 0.1 to about 99.9 weight percent or 100 weight percent based upon the total weight of the coating/impregnant composition used for the respective layer. In some examples, the coating/impregnant material(s) composition can be applied to the device 7010, prior to drying or curing, in an amount ranging from at about 0.001 mg/cm² to about 5 mg/cm², or about 2 mg/cm²±50%, per layer. In some examples, for coating/impregnant material(s) composition comprising at least one antimicrobial(s), the coating(s)/impregnant(s) can be applied to the device 7010, prior to drying or curing, in an amount ranging from at about 0.001 mg/cm² to about 5 mg/cm², or about 2 mg/cm²±50%, or about 0.005 mg/cm² to about 0.025 mg/cm² per layer.

In some examples, an outermost coating/impregnant layer(s) 7024 comprises the at least one lubricant. In some examples, the at least one lubricant (such as a hydrophilic material, gel or hydrogel) is configured to remain or to at least partially or fully dissipate when exposed to fluid (such as moisture and/or urine), such as occurs when the catheter device 7010 is deployed in the patient's urinary tract 1, to reveal or uncover other materials of the at least one coating(s) and/or impregnation(s) 7022 7022 or the outer surface 7028 of the catheter device 7010 beneath the lubricant coating. For example, as the lubricant dissipates, one or more sublayer(s) or underlying layers positioned below the outermost layer(s) 7024 may be exposed. The lubricant of the outermost layer(s) 7024 can be configured to dissipate into surrounding fluid or tissue within a desired time period following implantation. Since the lubricant can be primarily intended to facilitate insertion and positioning of the coated device 7010, a portion or all of the outermost layer 7024 may dissipate within a rather short period of time following insertion in the urinary tract 1. For example, the outermost layer 7024 may be configured to entirely, substantially or partially dissipate within 6 hours to 10 days, or 12 hours to 5 days, or 1 day to 3 days, following insertion and/or placement within the urinary tract. As used herein, a material, such as a portion or all of the outermost layer 7024, substantially dissipates when at least about 90%, or at least about 95%, or about 95%, or about 98%, of the outermost layer 7024 has released from the surface of the catheter 7010, or coating beneath the outermost layer, and been absorbed into surrounding fluid and/or tissues 1003, 1004, and/or expelled from the patient's body. In some examples, an outermost layer 7024 which dissipates within 1 day to 10 days can have a total thickness, prior to or when hydrated or activated, ranging from 0.01 micrometer to 5.0 millimeters, or 0.001 mm to 2.5 mm, or 0.01 mm to 1.0 mm. In some embodiments, a thickness of the outermost layer 7024 may be largely dependent on how long the outermost layer 7024 should remain in place when within the urinary tract before dissolving to expose sublayer(s) 7026 of the at least one coating(s) and/or impregnation(s) 7022 and/or the outer surface 7028 of the catheter device 7010.

Upon dissipation of the outermost layer 7024, material of one or more sublayer(s) 7026 positioned below the outermost layer(s) 7024 may remain in place to provide a particular property or function for an extended period of time or may be configured to release into surrounding fluid and/or tissue to, for example, provide a desired therapeutic or beneficial effect for the surrounding fluid and/or tissue. For example, the material of the one or more sublayer(s) 7026 can be configured for slow release into surrounding tissue over a period ranging from about 1 day to about one year, or about 30 days to about 180 days, or about 45 days to about 90 days. In some examples, a rate of dissipation for the sublayer(s) 7026 is dependent on a thickness of the outermost layer 7024. For example, the sublayer(s) 7026 can have a total thickness ranging from about 0.01 micrometer to 5.0 millimeter, or about 0.01 mm to 4.0 mm, or about 0.1 mm to 3.0 mm. In some examples, the thickness of the sublayer(s) 7026 can be selected so that it can remain, or dissolve and release materials, for improving function of the catheter device 7010 for the entire useful life or time that the device 7010 is within the urinary tract.

In other examples, the outermost layer 7024 can be configured to remain adhered to the coated device 7010, and in some examples to maintain its beneficial properties, throughout some or all of the time period in which the coated device 7010 is within the urinary tract. For example, the outermost layer(s) 7024 may remain in place for a period of up to 10 days, 45 days, 90 days, or up to, at least, one year, when within a patient's urinary tract 1. In order to maintain beneficial or hydrophilic properties for up to at least one year, the outermost layer 7024 may be as thin as or thinner than 0.01 mm, or may be thicker than 5.0 mm, possibly up to 10.0 mm thick. Alternatively or additionally, the outermost layer 7024 can be formed from a material that does not dissolve or degrade, or only degrades slowly when within the urinary tract 1. For example, certain slippery or low friction non-hydrophilic materials, such as polytetrafluoroethene (PTFE) (e.g., Teflon), may remain in place without dissolving for extended periods of time.

When configured to maintain properties, such as hydrophilic properties, for an extended duration, the outermost layer 7024 can be configured for time-dependent permeability or release, such that bulk material of the sublayer(s) 7026 can pass through the outermost layer(s) 7024 and to surrounding fluid and/or tissue. For example, the outermost layer 7024 can comprise structures and/or void spaces for permitting moisture or fluid to penetrate through the outermost layer 7024 and to the one or more sublayer(s) 7026. In order to obtain such permeability, the outermost layer(s) 7024 can comprise a composite material wherein bulk hydrophilicity is maintained, while the at least one contributing material of the outermost layer 7024 provides properties, such as selective diffusibility, solubility, and/or porosity (e.g., microporosity, mesoporosity, or macroporosity). According to International Union of Pure and Applied Chemistry (IUPAC) nomenclature, microporosity, mesoporosity, and macroporosity describe materials exhibiting pores with diameters of less than 2.0 nanometers, between 2.0 and 50 nanometers, and greater than 50 nanometers, respectively. Processes that may be used to form porous materials can comprise, for example, phase separation, gas foaming, and soft and hard templating techniques, as well as other selective and additive manufacturing methods.

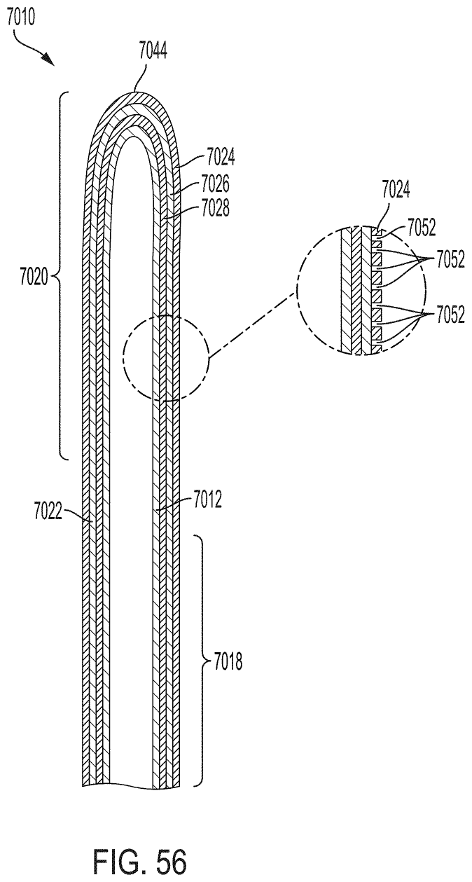

Alternatively or additionally, as shown schematically in FIG. 56, the outermost layer 7024 can comprise at least one opening(s), hole(s), space(s), and/or micro-channel(s) 7052 extending through the outermost layer 7024 to the one or more sublayer(s) 7026. The at least one opening(s), hole(s), space(s), and/or micro-channel(s) 7052 can be inherent, naturally occurring, or created (man-made) in the material of the outermost layer 7024. For example, the outermost layer 7024 may be naturally porous. In some examples, at least one opening(s), hole(s), space(s), and/or micro-channel(s) can be formed by any suitable process including, for example, pressing a pin or puncture needle through the cured outermost layer 7024. In other examples, the at least one opening(s), hole(s), space(s), and/or micro-channel(s) 7052 can be formed by etching or dissolving portions of the outermost layer 7024. The at least one opening(s), hole(s), space(s), and/or micro-channel(s) 7052 can be configured such that fluid, such as moisture, passes through the outermost layer 7024 to the sublayer(s) 7026, and dissolved material from the sublayer(s) 7026 passes through the at least one opening(s), hole(s), space(s), and/or micro-channel(s) 7052 of the outermost layer 7024 to the surrounding fluid and/or tissue. In some examples, the at least one opening(s), hole(s), space(s), and/or micro-channel(s) 7052 initially extend through the entire outermost layer 7024, such that fluid can penetrate to the one or more sublayer(s) 7026 as soon as the device 7010 is positioned in the urinary tract. In other examples, the at least one opening(s), hole(s), space(s), and/or micro-channel(s) 7052 may extend partially through the outermost layer 7024. In that case, fluid, such as moisture or urine, may collect in the at least one opening(s), hole(s), space(s), and/or micro-channel(s) 7052, causing portions of the at least one opening(s), hole(s), space(s), and/or micro-channel(s) 7052 to dissolve during an initial period following insertion into the urinary tract. Over time, the at least one opening(s), hole(s), space(s), and/or micro-channel(s) 7052 dissolve through the remaining portion of the outermost layer(s) 7024, eventually contacting and exposing portions of the one or more sublayer(s) 7026. In this way, release of the functional material(s) of the sublayer(s) 7026 is delayed until a period of time after the device 7010 is inserted into the urinary tract. The at least one opening(s), hole(s), space(s), and/or micro-channel(s) 7052 can be of any size and number sufficient for permitting fluid, such as moisture, to pass to contact the one or more sublayer(s) 7026 and for permitting dissolved material of the sublayer(s) 7026 to pass through the at least one opening(s), hole(s), space(s), and/or micro-channel(s) 7052 to surrounding body fluid and tissues. For example, the at least one opening(s), hole(s), space(s), and/or micro-channel(s) 7052 can have a cross-sectional area of about 0.01 micrometer to about 1.0 milimeter², or about 0.1 mm² to about 0.5 mm², or about 0.2 mm² to about 0.4 mm². The at least one opening(s), hole(s), space(s), and/or micro-channel(s) 7052 can be formed in or on the outermost layer 7024 in a variety of configurations and arrangements. For example, the at least one opening(s), hole(s), space(s), and/or micro-channel(s) 7052 can be a plurality of openings having any configuration desired, for example, substantially circular, elliptical, or any shape in cross section. In other examples, the at least one opening(s), hole(s), space(s), and/or micro-channel(s) 7052 can be troughs or burrows extending in any direction (e.g., axially and/or or circumferentially) along a surface of or within the outermost layer(s) 7024.

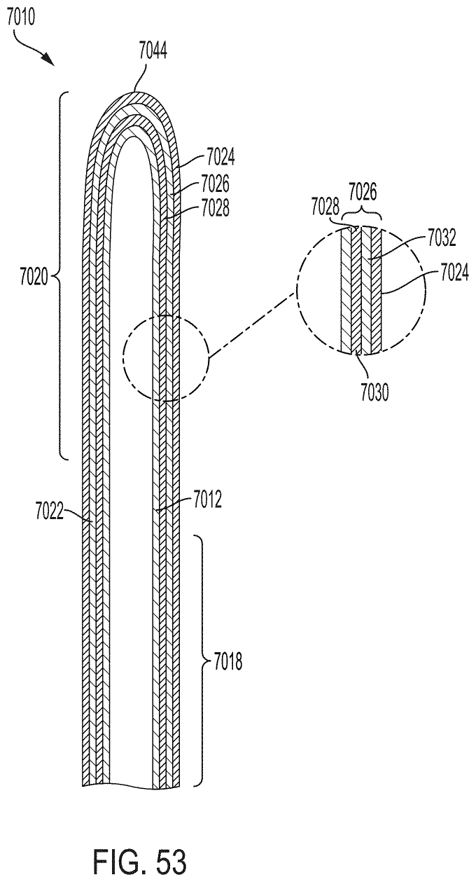

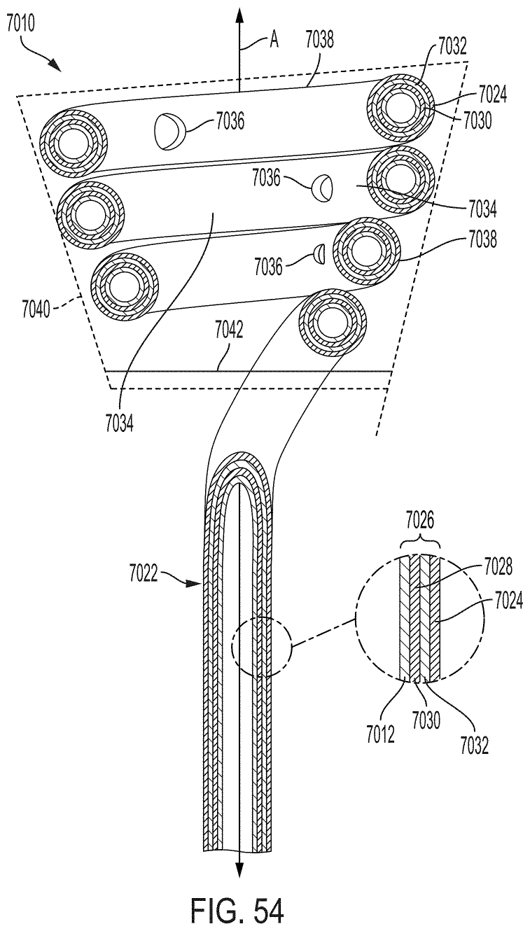

As shown in FIGS. 53 and 54, in some examples, the sublayer(s) 7026 are positioned between an outer surface 7028 of the device 7010 (such as elongated tube 7012) and the outermost layer(s) 7024. The sublayer(s) 7026 can be configured to improve long-term performance of the coated device 7010, for example, by addressing one or more of the above-described issues associated with prolonged use of the device 7010, such as an indwelling catheter. For example, the one or more sublayer(s) 7026 can improve long-term performance of the coated device 7010 by one or more of: inhibiting tissue ingrowth; mitigating a foreign body reaction for tissues surrounding the deployed coated device 7010; reducing infection of tissues surrounding the coated device 7010; and/or reducing encrustation of urine crystals onto the coated device 7010.

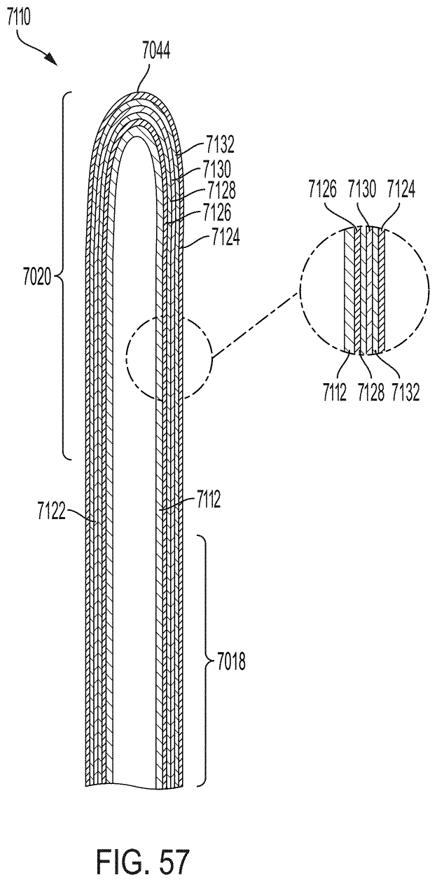

In some examples, the at least one coating(s) and/or impregnation(s) 7022 are intended to contact portions of fluid and/or tissue 1003, 1004 surrounding the coated device 7010, which can be brought into contact with the device 7010 by natural forces or applied negative pressure. Accordingly, the at least one coating(s) and/or impregnation(s) 7022 may need only to be applied to at least a portion or all of the outer periphery 72, 1002, or the outwardly facing side, or the protective surface(s) 1001, 7038, of at least a portion of, such as the retention portion 7020, or all of the device 7010. In some instances, as previously described, the inner periphery, inwardly facing side, or protected surface area 1000, 7034 of the retention portion 7020, which includes at least one protected drainage hole(s), port(s) or perforation(s) 7036, can be substantially free of or free from the at least one coating(s) and/or impregnation(s) 7022. Alternatively, as described in connection with FIG. 57, both the protected surface 1000, 7034 and the protective surface 1001, 7038 of the device 7010 may be coated with the at least one coating(s) and/or impregnation(s) 7022, for example to provide one or more of the aforementioned benefits of the coating(s) and/or facilitate manufacturability (e.g., deposition of the coating onto the device 7010).

The at least one coating(s) and/or impregnation(s) 7022 described herein can be adapted for use with any or all of the devices 7010, such as stents, ureteral catheters and/or bladder catheters, described herein. For example, the at least one coating(s) and/or impregnation(s) 7022 can be applied to a device 7010 comprising a distal portion 7018 comprising an expandable retention portion 7020 which, when deployed at a desired location within the kidney and/or renal pelvis, defines a three-dimensional shape 7040 sized and positioned to maintain patency of fluid flow between the kidney and a proximal portion 7014 and/or proximal end 7016 of the device 7010, such that at least a portion of the fluid flows through the expandable retention portion 7020. In that case, the at least one coating(s) and/or impregnation(s) 7022 may be applied to portions of the retention portion 7020 that contact a surface of the three dimensional shape 7040. Further, as in previously described examples, in order to match a size and shape of the renal pelvis, an area of two-dimensional slices 7042 of the three-dimensional shape 7040 defined by the deployed expandable retention portion 7020 in a plane transverse to a central axis A of the expandable retention portion 7020 can increase towards a distal end 7044 of the expandable retention portion 7020.

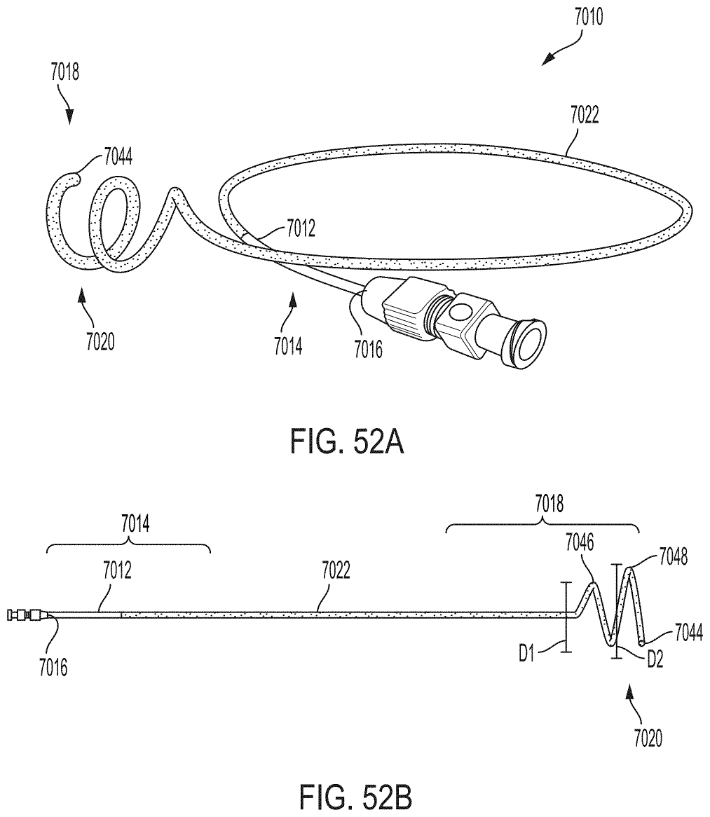

In some examples, the retention portion 7020 comprises a coiled retention portion extending radially from the renal pelvis to the kidney. The coiled retention portion 7020 can comprise at least a first coil 7046 (shown in FIG. 52B) having a first diameter D1 and at least a second coil 7048 having a second diameter D2. The first diameter D1 can be less than the second diameter D2 to correspond to a size and shape of the renal pelvis. In some examples, the at least one coating(s) and/or impregnation(s) 7022 need only be applied to the outer or protective surfaces 1001, 7038 of the coil(s) 7046, 7048 since only such outer or protective surfaces 1001, 7038 are contacted by body tissues. The protected surfaces 1000, 7034 of the coils 7046, 7048 may not be coated by the at least one coating(s) and/or impregnation(s) 7022.

In some examples, at least a portion or all of both of the protective surfaces 1001, 7038 and the protected surfaces 1000, 7034 of the device 7010, such as coil(s) 7046, 7048 can be coated by the at least one coating(s) and/or impregnation(s) 7022. For example, applying the at least one coating(s) and/or impregnation(s) 7022 to all surfaces of the device 7010 or elongated tube 7012 may be easier for manufacturing or production. In some examples, the device 7010, for example the entire elongated tube 7012, may be coated by a hydrophilic or outermost layer(s) 7024, since the elongated tube 7012 or device 7010 may be in a substantially linear (e.g., uncoiled) configuration during insertion through the patient's urinary tract. The sublayers 7030, 7032, which may help to improve long term performance of the device 7010, need only be applied to portions of the device 7010 or elongated tube 7012 likely to be contacted by bodily fluid or tissues (e.g., outwardly facing portions of the tube 7012).

### Multi-Layer Coating(s)/Impregnant(s) for Sequential Functionality

In other examples, as shown in FIG. 57, a coated device 7110 comprises one or more coating(s) and/or impregnation(s) 7122 having multiple or different functionalities. The coating(s) and/or impregnation(s) 7122 can be applied to at least a portion or all of the device 7010. As in prior examples, the coating(s) and/or impregnation(s) 7122 can comprise one or more outermost layer(s) 7124 and one or more innermost layer(s) 7126. The coating(s) and/or impregnation(s) 7122 can further comprise multiple sublayers 7128, 7130, 7132 positioned between the one or more outermost layer(s) 7124 and the one or more innermost layer(s) 7126. The multiple sublayers 7128, 7130, 7132 can be formed from different materials and can each address different issue(s) of indwelling catheters and/or provide different functional improvements for the device 7110. The one or more outermost layer(s) 7124 and multiple sublayers 7128, 7130, 7132 may be configured to dissipate sequentially, such that the coating(s) and/or impregnation(s) provides a first property or functionality for a predetermined period, a second property for a predetermined second period, and a third property for a predetermined third period. For example, the one or more coating(s) and/or impregnation(s) 7122 may be configured such that dissipation of outermost layer(s) 7124 and sublayers 7128, 7130, 7132 over time results in periodic and/or intermittent effects including, but not limited to, periods of antimicrobial and/or antibacterial effects cycled with intermittent periods of drug delivery. In some instances, drug delivery could be controlled to occur at pre-specified times, such as limiting drug delivery to a 12-hour release, or to a 24-hour release, or to a 48-hour release, following insertion of the coated device 7110 into the urinary tract. Similarly, another 12-hour release, 24-hour release, or 48 hour release of drug may be provided prior to removal of the coated device 7110.

The outermost layer(s) 7124 can be substantially similar in thickness and material properties to the outermost layers previously described. For example, the outermost layer(s) 7124 can provide a lubricious outer surface configured to make insertion and placement of the device 7110 in the urinary tract easier than when no lubricated coating is present. The outermost layer(s) 7124 can be configured to remain or can dissipate shortly after being implanted in the body, such as within from 1 day to 10 days of implantation.

The multiple sublayers 7128, 7130, 7132 can be selected such that the sublayers remain for a predetermined period and dissipate over the lifespan or intended duration of the catheter device 7110 in the urinary tract. For example, for a catheter designed to be present in the urinary tract for a period of ten to twenty days, each of five sublayers may be configured to dissipate or dissolve in about two to four days. As discussed above, in another example, a layer comprising a therapeutic agent may dissipate within 12 hours, 24 hours, or 48 hours of insertion. Sublayers containing other materials, such as antimicrobial materials and/or pH buffering materials, may dissipate over a longer period of time, such as over periods of 1 day to 10 days, or 2 days to 8 days, or 3 days to 5 days.

In some examples, the multiple sublayers 7128, 7130, 7132 comprise a first sublayer 7128, positioned below the outermost layer(s) 7124. The first sublayer 7128 can be configured to begin to dissipate into surrounding tissue when contacted by moisture, as occurs once portions of the outermost layer(s) 7124 dissipate. As in previous examples, material of the first sublayer 7128 can be configured to address issues with indwelling catheters and/or improve functional properties of the coating(s) 7122. For example, the first sublayer 7128 can comprise an antimicrobial layer that provides protection from ingress of microbes into or onto the coating(s) 7122 for a predetermined time period, such as a few days following implantation.

Over the course of the few hours and/or days following insertion, the first sublayer 7128 dissipates, exposing the second sublayer 7130 to fluid or moisture. The second sublayer 7130 can comprise one or more coating(s) and/or impregnation(s) material(s) for providing another property for improving a function of the device 7110. For example, the second sublayer 7130 can comprise a dose of the therapeutic agent, such as a dose of an antibiotic. The second sublayer 7130 can be configured to deliver the dose of the therapeutic agent over either a short period of time (e.g., a few hours or one day) or for slow release of the therapeutic agent over a slightly longer time period (e.g., from one day to ten days, or from 2 days to 8 days, or from 3 days to 5 days). Once the therapeutic agent is released and the second sublayer 7130 dissipates into surrounding fluid or tissues, a third sublayer 7132 can be exposed to moisture or fluid of the urinary tract. The third sublayer 7132 may comprise material(s) with additional or different functional properties. For example, the third sublayer 7132 can be a pH buffering layer for reducing or eliminating a presence of encrustations on the device 7110. In other examples, the third sublayer 7132 could be another antimicrobial and/or antibacterial layer. The third sublayer 7132 can be configured to remain in place for a number of hours or days, as was the case with previous sublayers or layers.

The device 7110 can further comprise one or more additional sublayer(s) 7026 including materials with different properties for addressing issues of indwelling catheters and/or for improving a function of the coating(s) 7122 and coated device 7110. For example, the coating(s) 7122 could comprise a number of therapeutic layers including a dose of an antibiotic agent positioned between sublayer(s) 7026 comprising antimicrobial materials. Accordingly, the coating(s) 7122 can provide intermittent antibiotic doses separated by time periods in which no antibiotic is being delivered, thereby reducing a risk that antibiotic concentration would increase above suitable levels.

In some examples, the coating(s) 7122 also comprise the innermost layer 7126 positioned between the outer surface 7124 of the device 7110 or elongated tube 7112 and an innermost sublayer 7128. The innermost layer 7126 can be similar in size and material composition to the outermost layers 7124 described herein. For example, the innermost layer 7126 may comprise any of the coating materials discussed above, such as a hydrophilic material that becomes lubricated when exposed to moisture. In some examples, the innermost layer 7126 can be exposed shortly before removal of the device 7110. Once exposed to fluid or moisture, the innermost layer 7126 can be configured to become slippery and lubricious, which assists in removal of the device 7110 through the urinary tract. For example, when the innermost layer 7126 becomes lubricated, the elongated tube 7112 of the device 7110 can slide more easily through body tissues, facilitating removal of the device 7110.

Method of Making a Coating and/or Coated Catheter

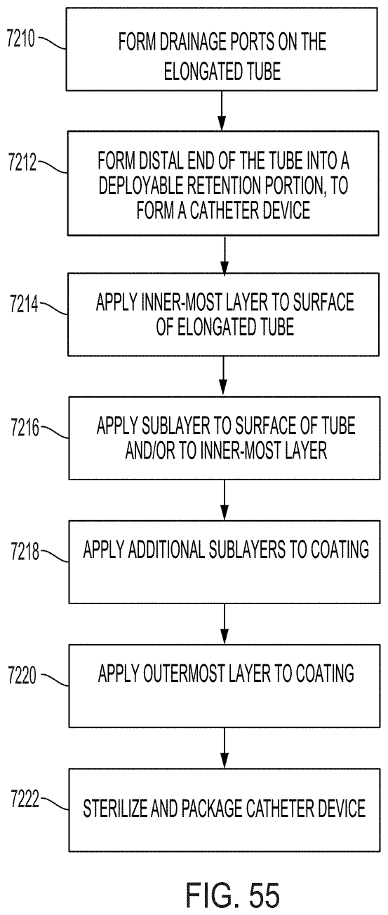

A method of making a coated and/or impregnated device 7010, such as a coated and/or impregnated catheter and/or stent device, is shown in FIG. 55. The method comprises steps 7210 to 7222, many of which can be performed in any order. For example, the at least one coating(s) and/or impregnation(s) 7022 could be applied directly to the device 7010, prior to forming other portions of a device 7010. Alternatively, as shown in FIG. 55, the at least one coating(s) and/or impregnation(s) 7022 can be applied to a device 7010 as a final step prior to packaging of the device 7010. Also, some of the steps shown in FIG. 55 are optional and need not be performed to make the device 7010 and/or the at least one coating(s) and/or impregnation(s) 7022. For ease of discussion, the method is discussed below with reference to preparing a device comprising a coil from an elongated tube, although any of the other device embodiments disclosed herein, such as a funnel or sponge, can be prepared in a similar manner.

As shown in FIG. 55, in some examples, the method initially comprises forming at least one opening(s), hole(s), space(s), and/or micro-channel(s) 7036 on portions of device 7210, such as the elongated tube as shown at 7210. For example, at least one opening(s), hole(s), space(s), and/or micro-channel(s) 7036 can be formed by puncturing portions of the elongated tube or device 7210 using a needle, drill, or similar tool. The opening(s), hole(s), space(s), and/or micro-channel(s) can be equidistantly spaced along a length of the device 7210 or elongated tube. Alternatively, the at least one opening(s), hole(s), space(s), and/or micro-channel(s) may be spaced more closely together near the distal end of the device 7210 or elongated tube to improve distribution of negative pressure applied through the device 7210 or tube. The at least one opening(s), hole(s), space(s), and/or micro-channel(s) can be the same or different sizes, as described herein. Alternatively, the at least one opening(s), hole(s), space(s), and/or micro-channel(s) near the distal end of the tube may be larger to improve negative pressure distribution, as described herein.

In some examples, the method can further comprise forming a retention portion 7020 on a distal portion 7018 of an elongated tube 7012, as shown at 7212. As in previously described examples, the retention portion 7020 can be configured to transition between a contracted state and an expanded or deployed state. In the deployed state, the retention portion 7020 can comprise an inwardly facing side or protected surface 7034 and an outwardly facing side or protective surface 1001, 7038. Desirably, the at least one opening(s), hole(s), space(s), and/or micro-channel(s) 7036 are positioned on the inwardly facing side or protected surface 7034 of the retention portion 7020, when deployed. The outwardly facing side or protective surface 1001, 7038, when deployed, can be substantially free from drainage ports, openings, holes, and perforations 7036.

The retention portion 7020 can be formed using a variety of techniques. For coiled retention portions, the elongated tube 7012 may be wrapped around a template or mandrel for an extended period of time to impart a desired curvature for the coils. For other types of expandable retention portions, such as expandable cages or frames, forming the retention portion can comprise bending or machining members, such as metal tines, into a desired shape and attaching the cage or frame to the distal portion of the elongated tube. Similarly, a balloon-shaped retention portion could also be attached to the distal portion of the elongated tube.

The at least one coating(s) and/or impregnation(s) 7022 can be applied to the device 7010 at any time during formation of the device, as shown at steps 7214 to 7220. At 7214, optionally, the method of forming the at least one coating(s) and/or impregnation(s) 7022 initially comprises applying a lubricious innermost layer 7126 to a surface 7028 of the device 7010, such as to a surface 7028 of an elongated tube 7012 of the catheter and/or stent device. For example, the innermost layer 7126 may be applied to distal portions of the device 7010 or elongated tube, such as portions of the device 7010 or tube configured to be deployed within the urinary tract. At 7216, in some examples, the method further comprises applying one or more sublayer(s) 7026. The sublayer(s) 7026 cover at least a portion of the innermost layer 7126, if present. If an innermost layer 7126 is not present, the sublayer(s) 7026 can be applied directly to portions of the device 7010, such as directly to an outer surface 7028 of the elongated tube. As previously described, the sublayer(s) 7026 can be configured to address issues related to indwelling catheters and/or to improve long-term performance of the device.

The at least one coating(s) and/or impregnation(s) 7022 can be applied using conventional coating techniques and processes. For example, the at least one coating(s) and/or impregnation(s) 7022 may be applied to the device 7010 by spraying portions of the device 7010 with the coating/impregnant material(s) for each of the layer(s), followed by allowing the material to dry, cure or harden. In other examples, the coating/impregnant material(s) for each of the layer(s) can be dripped or painted onto the surface(s) of the device 7010. In other examples, portions of the device 7010 can be dipped or immersed in a bath of the coating/impregnant material(s) for each of the layer(s), sequentially for multiple coatings. In other examples, the coating(s)/impregnant(s) material(s) can be applied by dispersion polymerization, suspension polymerization, bulk polymerization, atom transfer radical polymerization (ATRP), chemical vapor deposition polymerization, radical emulsion polymerization, polycondensation reaction, supercritical fluid methods, self-assembled colloidal crystal template methods, sacrificed polymer template methods, high internal phase emulsion template methods, and combinations of any of the methods discussed herein.

As discussed previously, the at least one coating(s) and/or impregnation(s) 7022 are desirably formed from one or more materials that improve long-term performance of the coating and/or of the device 7010. For example, the at least one coating(s) and/or impregnation(s) 7022 may be configured to improve long term performance of an indwelling device 7010 by one or more of: inhibiting tissue ingrowth; mitigating a foreign body reaction for tissues surrounding the deployed device; reducing infection of tissues surrounding the deployed device; and/or reducing encrustation of urine crystals on the device.

In some examples, as shown at 7218, one or more additional sublayer(s) 7026 can be applied to the surface of an initially applied sublayer. For example, as described above, a first sublayer 7030 can be a pH buffering sublayer formed from a pH buffering material. A subsequent sublayer can be an antibacterial sublayer, such as a sublayer comprising an antimicrobial and/or antibiotic material.

Following formation of the sublayer(s) 7026, at 7220, one or more outermost layer(s) 7024 are applied covering portion(s) of the sublayer(s) 7026. As previously described, in some examples the outermost layer(s) 7024 can comprise a hydrophilic lubricant material, such as a hydrophilic material that becomes gel-like and increases lubricity in the presence of moisture. Other low-friction and/or slick materials can also be used, as described herein.

Once the at least one coating(s) and/or impregnation(s) 7022 and drainage and retention portion(s) 7020 have been formed on the device 7010, the device 7010 can be substantially ready to be delivered to a user. In order to provide the device 7010 to the user, at 7222, the device can be sterilized and placed in appropriate packaging. The packaged device 7010, such as a catheter or stent, can be then be shipped to the user along with use instructions and any other required information.

### Systems for Inducing Negative Pressure

In some examples, a system for inducing negative pressure in a portion of a urinary tract of a patient or for removing fluid from the urinary tract of a patient is provided, comprising: a ureteral stent or ureteral catheter for maintaining patency of fluid flow between at least one of a kidney and a bladder of the patient; a bladder catheter comprising a drainage lumen for draining fluid from the bladder of the patient; and a pump in fluid communication with a distal end of the drainage lumen, the pump comprising a controller configured to actuate the pump to apply negative pressure to the proximal end of the catheter to induce negative pressure in a portion of the urinary tract of the patient to remove fluid from the urinary tract of the patient.

In some examples, a system for inducing negative pressure in a portion of a urinary tract of a patient is provided, the system comprising: (a) a ureteral catheter comprising a distal portion for insertion within the patient's kidney and a proximal portion; (b) a bladder catheter comprising a distal portion for insertion within the patient's bladder and a proximal portion for application of negative pressure, the proximal portion extending outside of the patient's body; and (c) a pump external to the patient's body for application of negative pressure through both the bladder catheter and the ureteral catheter, which in turn causes fluid from the kidney to be drawn into the ureteral catheter, through both the ureteral catheter and the bladder catheter, and then outside the patient's body.

In some examples, a system for inducing negative pressure in a portion of a urinary tract of a patient is provided, the system comprising: (a) at least one ureteral catheter, the at least one ureteral catheter comprising a distal portion for insertion within the patient's kidney and a proximal portion; (b) a bladder catheter comprising a distal portion for insertion within the patient's bladder and a proximal portion for receiving negative pressure from a negative pressure source, wherein at least one of the at least one ureteral catheter(s) or the bladder catheter comprises (a) a proximal portion; and (b) a distal portion, the distal portion comprising a retention portion that comprises at least one protected drainage hole(s), port(s) or perforation(s) and is configured to establish an outer periphery or protective surface area that inhibits mucosal tissue from occluding the at least one protected drainage hole(s), port(s) or perforation(s) upon application of negative pressure through the catheter; and (c) a negative pressure source for application of negative pressure through both the bladder catheter and the ureteral catheter(s), which in turn causes fluid from the kidney to be drawn into the ureteral catheter(s), through both the ureteral catheter(s) and the bladder catheter, and then outside of the patient's body.

In some examples, a system for inducing negative pressure in a portion of a urinary tract of a patient, the system comprising: (a) at least one ureteral catheter, the at least one ureteral catheter comprising a distal portion for insertion within the patient's kidney and a proximal portion; (b) a bladder catheter comprising a distal portion for insertion within the patient's bladder and a proximal portion for receiving a pressure differential, wherein the pressure differential causes fluid from the kidney to be drawn into the ureteral catheter(s), through both the ureteral catheter(s) and the bladder catheter, and then outside of the patient's body, the pressure differential being applied to increase, decrease and/or maintain fluid flow there through, wherein at least one of the at least one ureteral catheter(s) or the bladder catheter comprises (a) a proximal portion; and (b) a distal portion, the distal portion comprising a retention portion that comprises at least one protected drainage hole(s), port(s) or perforation(s) and is configured to establish an outer periphery or protective surface area that inhibits mucosal tissue from occluding the at least one protected drainage hole(s), port(s) or perforation(s) upon application of differential pressure through the catheter.

With reference to FIGS. 1A, 1B, 1C, 1F, 1P, 1U, 2A, 2B, 7A and 7B, an exemplary system 1100 for inducing negative pressure in a urinary tract of a patient for increasing renal perfusion is illustrated. The system 1100 comprises one or two ureteral catheters 1212 (or alternatively ureteral stents shown in FIG. 1A) connected to a fluid pump 2000 for generating the negative pressure. More specifically, the patient's urinary tract comprises the patient's right kidney 2 and left kidney 4. The kidneys 2, 4 are responsible for blood filtration and clearance of waste compounds from the body through urine. Urine or fluid produced by the right kidney 2 and the left kidney 4 is drained into a patient's bladder 10 through tubules, namely a right ureter 6 and a left ureter 8, which are connected to the kidneys at the renal pelvis 20, 21. Urine may be conducted through the ureters 6, 8 by peristalsis of the ureter walls, as well as by gravity. The ureters 6, 8 enter the bladder 10 through a ureter orifice or opening 16. The bladder 10 is a flexible and substantially hollow structure adapted to collect urine until the urine is excreted from the body. The bladder 10 is transitionable from an empty position (signified by reference line E) to a full position (signified by reference line F). Normally, when the bladder 10 reaches a substantially full state, fluid or urine is permitted to drain from the bladder 10 to a urethra 12 through a urethral sphincter or opening 18 located at a lower portion of the bladder 10. Contraction of the bladder 10 can be responsive to stresses and pressure exerted on a trigone region 14 of the bladder 10, which is the triangular region extending between the ureteral openings 16 and the urethral opening 18. The trigone region 14 is sensitive to stress and pressure, such that as the bladder 10 begins to fill, pressure on the trigone region 14 increases. When a threshold pressure on the trigone region 14 is exceeded, the bladder 10 begins to contract to expel collected urine through the urethra 12.

As shown in FIGS. 1, 2A, 7A and 7B, distal portions of the ureteral catheter(s) are deployed in the renal pelvis 20, 21 near the kidneys 2, 4. Proximal portions of the one or more of the catheter(s) 1212 empty into the bladder, into the urethra or outside of the body. In some examples, the proximal portion 1216 of the ureteral catheter 1212 is in fluid communication with the distal portion or end 136 of the bladder catheter 56, 116. A proximal portion 1216 of the bladder catheter 56, 116 is connected to a source of negative pressure, such as a fluid pump 2000. The shape and size of the connector can be selected based on the type of pump 2000 being used. In some examples, the connector can be manufactured with a distinctive configuration so that it can only be connected to a particular pump type, which is deemed to be safe for inducing negative pressure in a patient's bladder, ureter, or kidneys. In other examples, as described herein, the connector can be a more generic configuration adapted for attachment to a variety of different types of fluid pumps. System 1100 is but one example of a negative pressure system for inducing negative pressure that can be used with the bladder catheters disclosed herein.

Referring now to FIGS. 1A, 1B, 1C, 1F, 1P, 1U, 2A, 2B 7A, 7B, 17, in some examples the system 50, 100 comprises a bladder catheter 116. The distal ends 120, 121 of the ureteral catheters 112, 114 can drain directly into the bladder, and the fluid can drain through the bladder catheter 116, and optionally along the sides of the bladder catheter tube.

### Exemplary Bladder Catheters

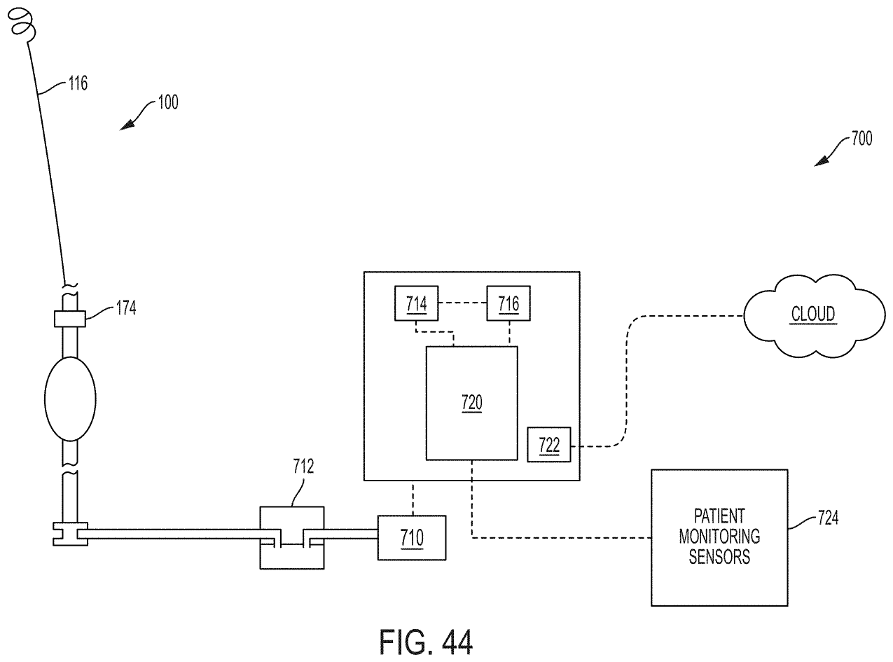

Any of the ureteral catheters disclosed herein can be used as bladder catheters useful in the present methods and systems. In some examples, the bladder catheter 116 comprises a retention portion 123 or deployable seal and/or anchor 136 for anchoring, retaining, and/or providing passive fixation for indwelling portions of the urine collection assembly 100 and, in some examples, to prevent premature and/or untended removal of assembly components during use. The retention portion 123 or anchor 136 is configured to be located adjacent to the lower wall of the patient's bladder 10 (shown in FIGS. 1A, 1B, 1C, 1F, 1P, 1U, 2A, 2B, 7A, 7B, 17) to prevent patient motion and/or forces applied to indwelling catheters 112, 114, 116 from translating to the ureters. The bladder catheter 116 comprises an interior of which defines a drainage lumen 140 configured to conduct urine from the bladder 10 to an external urine collection container 712 (shown in FIG. 44). In some examples, the bladder catheter 116 tube size can range from about 8 Fr to about 24 Fr. In some examples, the bladder catheter 116 can have an external tube diameter ranging from about 2.7 to about 8 mm. In some examples, the bladder catheter 116 can have an internal diameter ranging from about 2.16 to about 10 mm. The bladder catheter 116 can be available in different lengths to accommodate anatomical differences for gender and/or patient size. For example, the average female urethra length is only a few inches, so the length of a tube 138 can be rather short. The average urethra length for males is longer due to the penis and can be variable. It is possible that woman can use bladder catheters 116 with longer length tubes 138 provided that the excess tubing does not increase difficulty in manipulating and/or preventing contamination of sterile portions of the catheter 116. In some examples, a sterile and indwelling portion of the bladder catheter 116 can range from about 1 inch to 3 inches (for women) to about 20 inches for men. The total length of the bladder catheter 116 including sterile and non-sterile portions can be from one to several feet.

In some examples, such as are shown in FIGS. 1A, 1B, 1C, 1F, 1P, 1U, 2A, 2B, 7A and 7B, the distal portion 136 of the bladder catheter 56, 116 comprises a retention portion 123 that includes one or more drainage holes, ports or perforations 142 and is configured to establish an outer periphery 1002 or protective surface area 1001 that inhibits mucosal tissue from occluding the one or more drainage holes, ports or perforations 142 upon the application of negative pressure by the pump 710, 2000.

In some examples in which the retention portion 123 comprises a tube 138, the tube 138 can comprise one or more drainage holes, ports or perforations 142 configured to be positioned in the bladder 10 for drawing urine into the drainage lumen 140. For example, fluid or urine that flows into the patient's bladder 10 from the ureteral catheters 112, 114 is expelled from the bladder 10 through the ports 142 and drainage lumen 140. The drainage lumen 140 may be pressurized to a negative pressure to assist in fluid collection. In some examples, such as are shown in FIGS. 1A, 1B, 1C, 1F, 1P, 1U, 2A, 2B, 7A and 7B, the one or more drainage holes, ports or perforations 142, 172 of the bladder catheter 56, 116, like the ureteral catheters discussed above, are disposed on a protected surface area or inner surface area 1000 of the retention portion 123, and wherein, upon application of negative pressure, the mucosal tissue 1003, 1004 conforms or collapses onto the outer periphery 1002 or protective surface area 1001 of the retention portion 173 of the bladder catheter 56, 116 and is thereby prevented or inhibited from occluding the one or more of the protected drainage holes, ports or perforations 172 of the bladder catheter 56, 116

With specific reference to FIGS. 1A, 1B, 1C, 1F, 1P, 1U, 2A, 2B, 7A and 7B, the retention portion 123 or deployable seal and/or anchor 136 is disposed at or adjacent to a distal end 148 of the bladder catheter 116. The retention portion 123 or deployable anchor 136 is configured to transition between a contracted state for insertion into the bladder 10 through the urethra 12 and urethral opening 18 and a deployed state. The retention portion 123 or deployable anchor 136 is configured to be deployed in and seated adjacent to a lower portion of the bladder 10 and/or against the urethral opening 18. For example, the retention portion 123 or deployable anchor 136 can be positioned adjacent to the urethral opening 18 to enhance suction of a negative pressure applied to the bladder 10 or to partially, substantially, or entirely seal the bladder 10 to ensure that urine in the bladder 10 is directed through the drainage lumen 140 and to prevent leakage to the urethra 12. For a bladder catheter 116 including an 8 Fr to 24 Fr elongated tube 138, the retention portion 123 or deployable anchor 136 can have a diameter of about 10 mm to about 100 mm) in the deployed state.

### Exemplary Bladder Anchor Structures

Any of the ureteral catheters disclosed herein can be used as bladder catheters useful in the present methods and systems. For example, the bladder catheter can comprise a mesh as a bladder anchor, such as is shown in FIGS. 1A, 1B and 7B. In another example, the bladder catheter 116 can comprise a coil 36, 38, 40, 183, 184, 185, 334, 1210 as a bladder anchor, such as is shown in FIGS. 1C-1W and 7A. In another example, the bladder catheter 116 can comprise a mesh funnel 57 as a bladder anchor, such as is shown in FIG. 7B. In another example, the bladder catheter 116 can comprise a funnel 150 as a bladder anchor, such as is shown in FIG. 17. Regardless of the embodiment selected, the retention portion 123 creates an outer periphery 1002 or protective surface area 1001 to prevent the tissues 1003, 1004 from contracting or collapsing into the fluid column under negative pressure.

In some examples, the retention portion 123 comprises a coiled retention portion similar to the retention portions of the ureteral catheters described in connection with FIGS. 2A and 7A-14. In some examples such as are shown in FIGS. 1C-1E, 1U-1W, the coiled retention portion 123 can comprise a plurality of helical coils 36, 38, 40 or 438, 436, 432 arranged such that an outer periphery 1002 or outer region of the helical coils 36, 38, 40 or 438, 436, 432 contacts and supports bladder tissue 1004 to inhibit occlusion or blockage of protected drainage holes, ports or perforations 172 positioned in protected surface areas or inner surface areas of the helical coils 36, 38, 40 or 438, 436, 432.

The coiled retention portion 123 can comprise at least the first coil 36, 438 having an outer diameter D1 (see FIG. 1E), at least a second coil 38, 436 having an outer diameter D2, and at least a third coil 40, 432 having an outer diameter D3. The diameter D3 of the distal-most or third coil 40, 432 can be smaller than a diameter of either the first coil 36, 438 or the second coil 38, 436. Accordingly, a diameter of the coils 36, 38, 40 or 438, 436, 432, and/or a step distance or height between adjacent coils 36, 38, 40 or 438, 436, 432 can vary in a regular or irregular manner. In some examples, the plurality of coils 36, 38, 40 or 438, 436, 432 can form a tapered or reverse pyramid shape in which D1>D2>D3. In some examples, the coiled retention portion 123 can comprise a plurality of similarly sized coils or, for example, can include a plurality of proximal similarly sized coils and a distal-most coil having a smaller diameter than other coils of the plurality of coils. The diameter of the coils 36, 38, 40 or 438, 436, 432 and distance or height between adjacent coils is selected so that the retention portion 123 remains in the bladder for a desired period of time, such as hours, days or up to about 6 months. The coiled retention portion 123 can be large enough so that it remains in the bladder 10 and does not pass into the urethra until the catheter is ready to be removed from the bladder 10. For example, the outer diameter D1 of the proximal most or first coil 36 438 can range from about 2 mm to 80 mm. The outer diameter D2 of the second coil 38, 436 can range from about 2 mm to 60 mm. The distal-most or third coil 40, 432 can have an outer diameter D3 ranging from about 1 mm to 45 mm. The diameter of the coil tube can range from about 0.33 mm to 9.24 mm (about 1 Fr to about 28 Fr (French catheter scale).

The configurations, sizes and positions of the holes, ports or perforations 142, 172 can be any of the configurations, sizes and positions discussed above for the ureteral or other catheters. In some examples, holes, ports or perforations 142 are present on the outer periphery 1002 or protective surface area 1001 and protected holes, ports or perforations 172 are present on the protected surface areas or inner surface areas 1000. In some examples, the outer periphery 1002 or protective surface area 1001 is essentially free of or free of holes, ports or perforations 142, and the protected holes, ports or perforations 172 are present on the protected surface areas or inner surface areas 1000.

The retention portion 416 shown in FIGS. 1U-1W is a coiled retention portion comprising a plurality of coils wrapped around a substantially linear or straight portion 430 of the elongated tube 418. In some examples, the coiled retention portion 416 comprises a straight portion 430 and a distal-most coil 432 formed from a bend 434 of from about 90 degrees to 180 degrees in the elongated tube 418. The retention portion 416 further comprises one or more additional coils, such as a second or middle coil 436 and a third or proximal most coil 438, which are wrapped around the straight portion 430. The elongated tube 418 can further comprise a distal end 440 after the proximal most coil 438. The distal end 440 can be closed or can be open to receive urine or fluid from the bladder 10.

An area of two-dimensional slices 34 (shown in FIG. 1E) of the three-dimensional shape 32 defined by the deployed expandable retention portion 123 in a plane transverse to a central axis A of the expandable retention portion 16 can decrease towards the distal end 22 of the expanded or deployed retention portion 123, giving the retention portion 123 a pyramid or reversed conical shape. In some examples, a maximum cross-sectional area of the three-dimensional shape 32 defined by the deployed or expanded retention portion 123 in a plane transverse to the central axis A of the deployed or expanded retention portion 132 can range from about 100 mm² to 1500 mm², or about 750 mm².

Other examples of a catheter device 10 are shown in FIGS. 1F-1J. The retention portion 123 of the catheter device 10 comprises a basket shaped structure or support cap 212 of a bladder superior wall support 210 or outer periphery 1002, configured to be disposed within a distal portion of the tube 12 in a retracted position and to extend from the distal end of the tube 12 in a deployed position. The bladder superior wall support 210 comprises a support cap 212 configured to support a superior wall or bladder tissue 1004 and a plurality of support members, such as legs 214, connected to a proximal surface of the support cap 212. The legs 214 can be positioned so that the cap 212 is spaced apart from an open distal end of the drainage tube 12. For example, the legs 214 can be configured to maintain a gap, cavity, or space of distance D1 between an open distal end 30 of the tube 12 and the support cap 212. The distance D1 can range from about 1 mm to about 40 mm, or about 5 mm to about 40 mm. The height D2 of the bladder superior wall support 210 or retention portion can range from about 25 mm to about 75 mm, or about 40 mm. The maximum diameter of the support cap 212 can range from about 25 mm to about 60 mm in the deployed state, and preferably range from about 35 mm and 45 mm.

In some examples, the legs 214 comprise flexible tines, which can be formed from a flexible or shape memory material, such as a nickel titanium. The number of legs can range from about 3 to about 8. The length of each leg can range from about 25 mm to about 100 mm, or longer if the deployment mechanism is external to the patient's body. The width and/or thickness, e.g., diameter, of each leg can range from about 0.003 inches to about 0.035 inches.

In some examples, the support cap 212 can be a flexible cover 216 mounted to and supported by the legs 214. The flexible cover 216 can be formed from a flexible, soft and/or resilient material, such as silicone or Teflon®, for preventing fluid from passing through the cover 216, a porous material, or combinations thereof. In some examples, the flexible material is formed from a material which does not appreciably abrade, irritate, or damage the mucosal lining of the bladder wall or the urethra when positioned adjacent to the mucosal lining, such as silicone or Teflon® materials or porous materials. The thickness of the cover 216 can range from about 0.05 mm to about 0.5 mm. In some examples, the flexible cover 216 and legs 214 are sufficiently structurally rigid so that the cover 216 and legs 214 maintain their form when contacted by the superior wall or bladder tissue 1004. Accordingly, the legs 214 and flexible cover 216 prevent the bladder from collapsing and occluding perforations on the retention portion 6 and/or an open distal end 30 of the tube 12. Also, the legs 214 and flexible cover 216 effectively keep the trigone region and ureteral orifices open so that negative pressure can draw urine into the bladder and drainage tube 12. As discussed herein, if the bladder were permitted to collapse too far, flaps of tissue would extend over the ureter openings, thereby preventing negative pressure from being transmitted to the ureteral catheter(s), ureteral stent(s) and/or ureters and thereby inhibit drawing of urine into the bladder.

In some examples, the catheter device 10 further comprises a drainage tube 218. As shown in FIGS. 1G-1J, the drainage tube 218 can comprise an open distal end 220 positioned adjacent to or extending from the open distal end 30 of the tube 12. In some examples, the open distal end 220 of the drainage tube 218 is the only opening for drawing urine from the bladder into the interior of the drainage tube 218. In other examples, a distal portion of the drainage tube 218 may comprise perforations (not shown in FIGS. 1G-1I) or holes, ports or perforations 174 on a sidewall 222 thereon, as shown in FIG. 1J. The holes, ports or perforations 174 can provide additional spaces for drawing urine into the interior of the drainage tube 218, thereby ensuring that fluid collection can continue even if the open distal end 220 of the drainage tube 218 is occluded. Also, holes, ports or perforations 174 can increase surface area available for drawing fluid into the drainage tube 218, thereby increasing efficiency and/or fluid collection yield.

In some examples, a distal most portion of the support cap 212 can comprise a sponge or pad 224, such as a gel pad. The pad 224 can be positioned to contact and press against the superior bladder wall or bladder tissue 1004 for the purpose of preventing drainage, aspiration, or other trauma to the bladder 10 during negative pressure treatment.

With reference to FIG. 1J, the bladder superior wall support 210 comprises a support cap 212 and a plurality of legs 214. As in previously described examples, the bladder superior wall support 210 is capable of being moved between a retracted position, in which the support 210 is at least partially retracted in a conduit or tube 12, and a deployed position to support the superior wall of the bladder. In some examples, the catheter device 10 also includes a drainage tube 218 extending from the open distal end 30 of the conduit or tube 12. Unlike in the previously-described examples, the support cap 212 shown in FIG. 4 comprises an inflatable balloon 226. The inflatable balloon 226 can be a substantially semi-spherical and can comprise a curved distal surface 228 configured to contact and support at least a portion of the superior bladder wall or bladder tissue 1004 when deployed.

In some examples, the drainage tube 218 comprises a perforated portion 230 extending between the open distal end 30 of the tube 12 and the support structure 212. The perforated portion 230 is positioned to draw fluid into an interior of the drainage tube 218 so that it can be removed from the bladder 100. Desirably, the perforated portion 230 is positioned so as not to be occluded either by the deployed support cap 212 or the bladder wall when negative pressure is applied thereto. The drainage tube 218 can comprise or be positioned adjacent to an inflation lumen 232 for providing fluid or gas to an interior 234 of the balloon 226 for inflating the balloon 226 from its contracted position to the deployed position. For example, as shown in FIG. 1J, the inflation lumen 232 can be disposed within the drainage tube 218.

With reference to FIG. 1K, an exemplary retention portion 6, 123 of a urine collection catheter device 10 including multiple coiled drainage lumens, generally denoted as lumens 218, is illustrated. The retention portion 6 comprises the tube 12 having a distal open end 30. The drainage lumens 218 are positioned partially within the tube 12. In a deployed position, the draining lumens 218 are configured to extend from the open distal end 30 of the tube 12 and to conform to a coiled orientation. The drainage lumens 218 can be separate for the entire length of the catheter device 10, or may empty into a single drainage lumen defined by the tube 12. In some examples, as shown in FIG. 6, the drainage lumens 218 can be pigtail coils having one or more coils 244. Unlike in the previously described example, the pigtail coils 244 are coiled about an axis that is not coextensive with an axis C of an uncoiled portion of the tube. Instead, as shown in FIG. 6, the pigtail coils can be coiled about an axis D that is approximately perpendicular to the axis C of the tube 12. In some examples, the drainage lumens 218 can comprise holes, ports or perforations (not shown in FIG. 1K), similar to perforations 132, 133 in FIG. 9A or 9B, for drawing fluid from the bladder into an interior of the drainage lumens 218. In some examples, the perforations can be positioned on a radially inwardly facing side 240 and/or outwardly facing side of the coiled portions of the drainage lumens. As previously described, perforations positioned on radially inwardly facing sides of the drainage lumens 218 or tube 12 are less likely to be occluded by the bladder walls during application of negative pressure to the bladder. Urine can also be drawn directly into one or more drainage lumens defined by the tube 12. For example, rather than being drawing into the drainage lumen(s) 218 through the perforations 230, urine can be drawn directly through the open distal end 30 and into a drainage lumen defined by the tube 12.

With reference to FIGS. 1L and 1M, another example of a retention portion 123 is shown. A fluid receiving portion or distal end portion 30a of the catheter device 10a is shown in a contracted position in FIG. 1L, and in a deployed position in FIG. 1M. The distal end 30a includes opposing bladder wall supports 19a, 19b for supporting the superior and inferior bladder walls 1004. For example, the distal end portion 30a can comprise a proximal sheath 20a and a distal sheath 22a. Each sheath 20a, 22a extends between a slidable ring or collar 24a and stationary or mounted ring or collar 28a. The sheaths 20a, 22a are formed from a flexible, non-porous material, such as silicon or any of the materials discussed herein. The sheaths 20a, 22a are held together by one or more flexible wires or cables 26a. The sheaths 20a, 22a can also be connected by one or more rigid members, such as supports 32a. In some examples, the supports 32a can be tines formed from a flexible, shape-memory material, such as nickel titanium. The supports 32a are positioned to provide support for the proximal sheath 20a and to prevent the distal end 30a from collapsing when it is in the deployed position. In the contracted position, the collars 24a, 28a are positioned apart from one another, such that the sheaths 20a, 22a are stretched or folded against the cable 26a and supports 32a. In the deployed position, the slidable collars 24a are moved toward the stationary collars 28a, allowing the sheaths 20a, 22a to unfold from the central cables 26a and to form a substantially flat disk-shaped structure.

In use, the distal end 30a of the catheter device 10a is inserted into the bladder of a patient in the contracted position. Once inserted in the bladder, the distal sheath 22a is released by sliding the slidable collar 24a in a distal direction toward the stationary collar 28a. Once the distal sheath 22a is deployed, the proximal sheath 20a is released or deployed in a similar manner by sliding the slidable collar 24a in the proximal direction toward the respective stationary collar 28a. At this point, the proximal sheath 20a is floating within the bladder, and is not positioned or sealed against the inferior wall of the bladder. Pressure against the distal sheath 22a caused by collapsing of the bladder is transferred to the proximal sheath 20a through the supports 32a and causes the proximal sheath 20a to move toward the desired position adjacent to the opening of the urethra. Once the proximal sheath 20a is in place, a seal over the urethra opening may be created. The proximal sheath 20a assists in maintaining a negative pressure within the bladder and prevents air and/or urine from exiting the bladder through the urethra.

With reference to FIGS. 1N-1T, retention portions 123 comprising an inflatable support cap, such as an annular balloon 310, positioned to contact the superior wall of the bladder 10 to prevent the bladder 10 from contracting and occluding either fluid port(s) 312 of the catheter device 10 or the ureteral openings of the bladder. In some examples, a distal end portion 30 of the tube 12 extends through a central opening 314 of the balloon 310. The distal end portion 30 of the tube 12 can also contact the superior bladder wall.

Referring now to FIGS. 1N and 10, in some examples, the tube 12 comprises a fluid access portion 316 positioned proximal to the balloon 310 and extending through a sidewall of the tube 12. The fluid access portion 316 can comprise a filter 318 (shown in FIG. 1O) disposed about a central lumen of the tube 12. In some examples, a sponge material 320 can be positioned over the filter 318 for increased absorbance of fluid within the bladder. For example, the sponge material 320 can be injection molded over the filter 318. In use, urine is absorbed by the sponge material 320 and, upon application of negative pressure through the tube 12, passes through the filter 318 and into the central lumen of the tube 12.

Referring now to FIGS. 1P-1R, in another example, the support cap, such as the annular balloon 310, comprises a substantially bulbous distal portion 322 configured to contact and support the superior bladder wall. The balloon 310 further comprises a plurality of proximally extending lobes 324. For example, the balloon 310 can comprise three lobes 324 spaced equidistantly around a portion of the tube 12 proximal to the balloon 310. As shown in FIG. 1R, the fluid ports 312 can be positioned between adjacent lobes 324. In this configuration, the lobes 324 and bulbous distal portion 322 contact the bladder wall, which prevents the bladder wall from blocking or occluding the fluid ports 312.

Referring now to FIGS. 1S and 1T, in another example, the annular balloon 310 is provided with a flattened and elongated shape. For example, the annular balloon 310 can have a substantially teardrop shaped radial cross section as shown in FIG. 1T, with a narrower portion 326 thereof positioned adjacent to the tube 12 and the enlarged or bulbous portion 328 positioned on the radially outwardly facing side thereof. The flatted annular balloon 310 is configured to span and optionally seal the periphery of the trigone region of the bladder such that when deployed in the bladder, the outer circumference of the balloon 310 extends radially beyond the ureteral openings. For example, when positioned in the patient's bladder, the central opening 314 of the balloon 310 can be configured to be positioned above the trigone region. Fluid port(s) 312 can be positioned proximal to the central portion balloon 310, as shown in FIG. 1T. Desirably, the fluid port(s) 312 are positioned between the central opening 314 of the balloon and the trigone region. When the bladder contracts from application of negative pressure, the bladder wall is supported by the outer circumference of the balloon 310 to avoid blocking the ureter openings. Accordingly, in this configuration, the balloon 310 contacts and prevents the bladder wall from blocking or occluding the fluid ports 312. In a similar manner, as discussed herein, the balloon 310 keeps the trigone region open so that urine can be drawn from the ureters into the bladder through the ureteral openings.

With reference to FIG. 41, in another example of a bladder catheter, an expandable cage 530 can anchor the bladder catheter in the bladder. The expandable cage 530 comprises a plurality of flexible members or tines extending longitudinally and radially outward from a catheter body of a bladder catheter which, in some examples, can be similar to those discussed above with respect to the retention portion of the ureteral catheter of FIG. 41. The members can be formed from a suitable elastic and shape memory material such as nitinol. In a deployed position, the members or tines are imparted with a sufficient curvature to define a spherical or ellipsoid central cavity. The cage is attached to an open distal open end of the catheter tube or body, to allow access to a drainage lumen defined by the tube or body. The cage is sized for positioning within the lower portion of the bladder and can define a diameter and length ranging from 1.0 cm to 2.3 cm, and preferably about 1.9 cm (0.75 in).

In some examples, the cage further comprises a shield or cover over distal portions of the cage to prevent or reduce the likelihood that tissue, namely, the distal wall of the bladder, will be caught or pinched as a result of contact with the cage or member. More specifically, as the bladder contracts, the inner distal wall of the bladder comes into contact with the distal side of the cage. The cover prevents the tissue from being pinched or caught, may reduce patient discomfort, and protect the device during use. The cover can be formed at least in part from a porous and/or permeable biocompatible material, such as a woven polymer mesh. In some examples, the cover encloses all or substantially all of the cavity. In some examples, the cover covers only about the distal ⅔, about the distal half, or about the distal third portion or any amount, of the cage 210.

The cage and cover are transitionable from a contracted position, in which the members are contracted tightly together around a central portion and/or around the bladder catheter 116 to permit insertion through a catheter or sheath to the deployed position. For example, in the case of a cage constructed from a shape memory material, the cage can be configured to transition to the deployed position when it is warmed to a sufficient temperature, such as body temperature (e.g., 37° C.). In the deployed position, the cage has a diameter D that is preferably wider than the urethral opening, and prevents patient motion from translating through the ureteral catheters 112, 114 to the ureters. The open arrangement of the members 212 or tines does not obstruct or occlude the distal opening 248 and/or drainage ports of the bladder catheter 216, making manipulation of the catheters 112, 114 easier to perform.

It is understood that any of the above-described bladder catheters may also be useful as ureteral catheters. The bladder catheter is connected to the vacuum source, such as pump assembly 710 by, for example, flexible tubing 166 defining a fluid flow path.

Exemplary Fluid Sensors

With reference again to FIGS. 1A, 1B, 1C, 1F, 1P, 1U, 2A, 2B, in some examples, the system or assembly 100, 700, 1100 further comprises one or more sensors 174 for monitoring physical parameters or fluid characteristics of fluid or urine being collected from the ureters 6, 8 and/or bladder 10. The one or more physiological sensors 174 associated with the patient can be configured to provide information representative of at least one physical parameter to a controller. As discussed herein in connection with FIG. 44, information obtained from the sensors 174 can be transmitted to a central data collection module or processor and used, for example, to control operation of an external device, such as the pump 710 (shown in FIG. 44). The sensors 174 can be integrally formed with one or more of the catheters 112, 114, 116 such as, for example, embedded in a wall of the catheter body or tube and in fluid communication with drainage lumens 124, 140. In other examples, one or more of the sensors 174 can be positioned in a fluid collection container 712 (shown in FIG. 44) or in internal circuitry of an external device, such as the pump 710.

Exemplary sensors 174 that can be used with the urine collection assembly 100 can comprise one or more of the following sensor types. For example, the catheter assembly 100 can comprise a conductance sensor or electrode that samples conductivity of urine. The normal conductance of human urine is about 5-10 mS/m. Urine having a conductance outside of the expected range can indicate that the patient is experiencing a physiological problem, which requires further treatment or analysis. The catheter assembly 100 can also comprise a flow meter for measuring a flow rate of urine through the catheter(s) 112, 114, 116. Flow rate can be used to determine a total volume of fluid excreted from the body. The catheter(s) 112, 114, 116 can also comprise a thermometer for measuring urine temperature. Urine temperature can be used to collaborate the conductance sensor. Urine temperature can also be used for monitoring purposes, as urine temperature outside of a physiologically normal range can be indicative of certain physiological conditions. In some examples, the sensors 174 can be urine analyte sensors configured to measure a concentration of creatinine and/or proteins in urine. For example, various conductivity sensors and optical spectrometry sensors may be used for determining analyte concentration in urine. Sensors based on color change reagent test strips may also be used for this purpose.

### Method of Insertion of a System

Having described the system 100 comprising the ureteral catheter(s) and/or ureteral stent(s) and bladder catheter, some examples of methods for insertion and deployment of the ureteral stent(s) or ureteral catheter(s) and bladder catheter will now be discussed in detail.

In some examples, a method for inducing negative pressure in a portion of a urinary tract of a patient is provided, the method comprising: deploying a ureteral catheter into a ureter of a patient to maintain patency of fluid flow between a kidney and a bladder of the patient, the ureteral catheter comprising a distal portion for insertion within the patient's kidney and a proximal portion; deploying a bladder catheter into the bladder of the patient, wherein the bladder catheter comprises a distal portion for insertion within the patient's bladder and a proximal portion for application of negative pressure, the proximal portion extending outside of the patient's body; and applying negative pressure to the proximal end of the bladder catheter to induce negative pressure in a portion of the urinary tract of the patient to remove fluid from the patient. In some examples, at least one of the ureteral catheter or the bladder catheter comprises (a) a proximal portion; and (b) a distal portion, the distal portion comprising a retention portion that comprises at least one protected drainage hole(s), port(s) or perforation(s) and is configured to establish an outer periphery or protective surface area that inhibits mucosal tissue from occluding the at least one protected drainage hole(s), port(s) or perforation(s) upon application of negative pressure through the catheter.

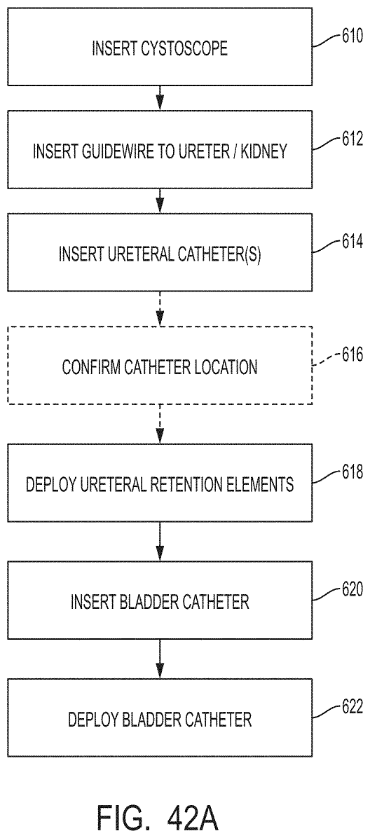

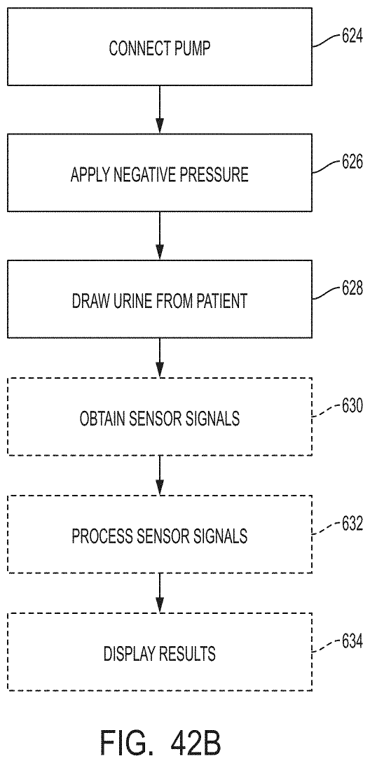

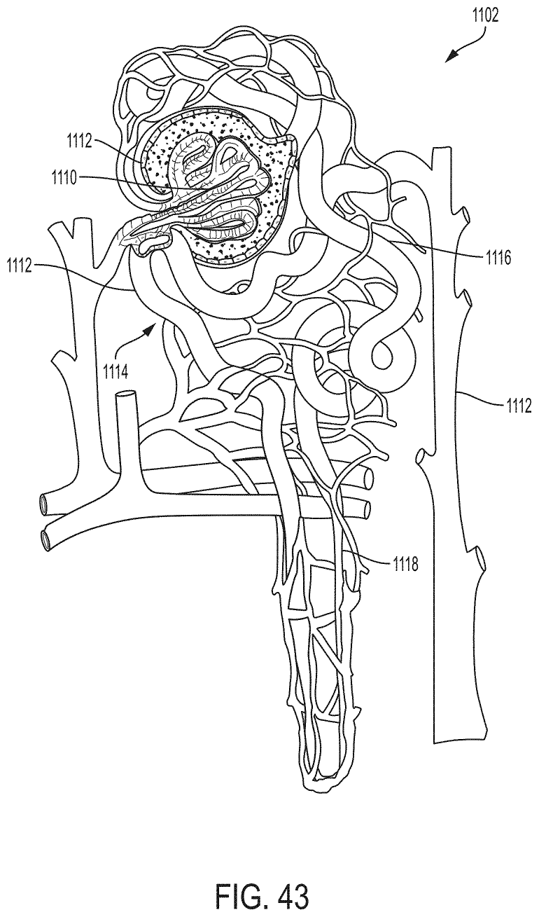

With reference to FIG. 42A, an example of steps for positioning a system in a patient's body and, optionally, for inducing negative pressure in a patient's urinary tract, such as bladder, ureter and/or kidneys are illustrated. As shown at box 610, a medical professional or caregiver inserts a flexible or rigid cystoscope through the patient's urethra and into the bladder to obtain visualization of the ureteral orifices or openings. Once suitable visualization is obtained, as shown at box 612, a guidewire is advanced through the urethra, bladder, ureteral opening, ureter, and to a desired fluid collection position, such as the renal pelvis of the kidney. Once the guidewire is advanced to the desired fluid collection position, a ureteral stent or ureteral catheter of the present invention (examples of which are discussed in detail above) is inserted over the guidewire to the fluid collection position, as shown at box 614. In some examples, the location of the ureteral stent or ureteral catheter can be confirmed by fluoroscopy, as shown at box 616. Once the position of the distal end of the ureteral stent or ureteral catheter is confirmed, as shown at box 618, the retention portion of the ureteral catheter can be deployed. For example, the guidewire can be removed from the catheter, thereby allowing the distal end and/or retention portion to transition to a deployed position. In some examples, the deployed distal end portion of the catheter does not entirely occlude the ureter and/or renal pelvis, such that urine is permitted to pass outside the catheter and through the ureters into the bladder. Since moving the catheter can exert forces against urinary tract tissues, avoiding complete blockage of the ureters avoids application of force to the ureter sidewalls, which may cause injury.

After the ureteral stent or ureteral catheter is in place and deployed, the same guidewire can be used to position a second ureteral stent or second ureteral catheter in the other ureter and/or kidney using the same insertion and positioning methods described herein. For example, the cystoscope can be used to obtain visualization of the other ureteral opening in the bladder, and the guidewire can be advanced through the visualized ureteral opening to a fluid collection position in the other ureter. A second ureteral stent or second ureteral catheter can be drawn alongside the guidewire and deployed in the manner described herein. Alternatively, the cystoscope and guidewire can be removed from the body. The cystoscope can be reinserted into the bladder over the first ureteral catheter. The cystoscope is used, in the manner described above, to obtain visualization of the ureteral opening and to assist in advancing a second guidewire to the second ureter and/or kidney for positioning of the second ureteral stent or second ureteral catheter. Once the ureteral stents or catheters are in place, in some examples, the guidewire and cystoscope are removed. In other examples, the cystoscope and/or guidewire can remain in the bladder to assist with placement of the bladder catheter.

In some examples, once the ureteral catheters are in place, as shown at box 620, the medical professional, caregiver or patient can insert a distal end of a bladder catheter in a collapsed or contracted state through the urethra of the patient and into the bladder. The bladder catheter can be a bladder catheter of the present invention as discussed in detail above. Once inserted in the bladder, as shown at box 622, an anchor connected to and/or associated with the bladder catheter is expanded to a deployed position. In some examples, the bladder catheter is inserted through the urethra and into the bladder without using a guidewire and/or cystoscope. In other examples, the bladder catheter is inserted over the same guidewire used to position the ureteral stents or catheters.

In some examples, the ureteral stent or ureteral catheter is deployed and remains in the patient's body for at least 24 hours or longer. In some examples, the ureteral stent or ureteral catheter is deployed and remains in the patient's body for at least 30 days or longer. In some examples, the ureteral stent(s) or ureteral catheter(s) can be replaced periodically, for example every week or every month, to extend the length of therapy.

In some examples, the bladder catheter is replaced more often that the ureteral stent or ureteral catheter. In some examples, multiple bladder catheters are placed and removed sequentially during the indwell time for a single ureteral stent or ureteral catheter. For example, a physician, nurse, caregiver or patient can place the bladder catheter(s) in the patient at home or in any healthcare setting. Multiple bladder catheters can be provided to the healthcare professional, patient or caregiver in a kit, optionally with instructions for placement, replacement and optional connection of the bladder catheter(s) to the negative pressure source or drainage to a container, as needed. In some examples, negative pressure is applied each evening for a predetermined number of evenings (such as for 1 to 30 evenings or more). Optionally, the bladder catheter can be replaced each evening before application of negative pressure.

In some examples, the urine is permitted to drain by gravity or peristalsis from the urethra. In other examples, a negative pressure is induced in the bladder catheter to facilitate drainage of the urine. While not intending to be bound by any theory, it is believed that a portion of the negative pressure applied to the proximal end of the bladder catheter is transmitted to the ureter(s), renal pelvis or other portions of the kidney(s) to facilitate drainage of the fluid or urine from the kidney.

With reference to FIG. 42B, steps for using the system for inducement of negative pressure in the ureter(s) and/or kidney(s) are illustrated. As shown at box 624, after the indwelling portions of the ureteral stents or ureteral catheters and bladder catheters are correctly positioned and any anchoring/retention structures, if present, are deployed, the external proximal end of the bladder catheter is connected to a fluid collection or pump assembly. For example, the bladder catheter can be connected to a pump for inducing negative pressure at the patient's bladder, renal pelvis and/or kidney.

Once the bladder catheter and pump assembly are connected, negative pressure is applied to the renal pelvis and/or kidney and/or bladder through the drainage lumen of the bladder catheter, as shown at box 626. The negative pressure is intended to counter congestion mediated interstitial hydrostatic pressures due to elevated intra-abdominal pressure and consequential or elevated renal venous pressure or renal lymphatic pressure. The applied negative pressure is therefore capable of increasing flow of filtrate through the medullary tubules and of decreasing water and sodium re-absorption.

As a result of the applied negative pressure, as shown at box 628, urine is drawn into the bladder catheter at the drainage port(s) at the distal end thereof, through the drainage lumen of the bladder catheter, and to a fluid collection container for disposal. As the urine is being drawn to the collection container, at box 630, optional sensors disposed in the fluid collection system can provide a number of measurements about the urine that can be used to assess physical parameters, such as the volume of urine collected, as well as information about the physical condition of the patient and composition of the urine produced. In some examples, the information obtained by the sensors is processed, as shown at box 632, by a processor associated with the pump and/or with another patient monitoring device and, at box 634, is displayed to the user via a visual display of an associated feedback device.

### Exemplary Fluid Collection System

Having described an exemplary system and method of positioning such a system in the patient's body, with reference to FIG. 44, a system 700 for inducing negative pressure to a patient's bladder, ureter(s), renal pelvis and/or kidney(s) will now be described. The system 700 can comprise the ureteral stent(s) and/or ureteral catheter(s), bladder catheter or the system 100 described hereinabove. As shown in FIG. 44, the bladder catheter 116 of the system 100 is connected to one or more fluid collection containers 712 for collecting urine drawn from the bladder. The fluid collection container 712 connected to the bladder catheter 116 can be in fluid communication with an external fluid pump 710 for generating negative pressure in the bladder, ureter(s) and/or kidney(s) through the bladder catheter 116 and/or ureteral catheter(s) 112, 114. As discussed herein, such negative pressure can be provided for overcoming interstitial pressure and forming urine in the kidney or nephron. In some examples, a connection between the fluid collection container 712 and pump 710 can comprise a fluid lock or fluid barrier to prevent air from entering the bladder, renal pelvis or kidney in case of incidental therapeutic or non-therapeutic pressure changes. For example, inflow and outflow ports of the fluid container can be positioned below a fluid level in the container. Accordingly, air is prevented from entering medical tubing or the catheter through either the inflow or outflow ports of the fluid container 712. As discussed previously, external portions of the tubing extending between the fluid collection container 712 and the pump 710 can include one or more filters to prevent urine and/or particulates from entering the pump 710.

As shown in FIG. 44, the system 700 further comprises a controller 714, such as a microprocessor, electronically coupled to the pump 710 and having or associated with computer readable memory 716. In some examples, the memory 716 comprises instructions that, when executed, cause the controller 714 to receive information from sensors 174 located on or associated with portions of the assembly 100. Information about a condition of the patient can be determined based on information from the sensors 174. Information from the sensors 174 can also be used to determine and implement operating parameters for the pump 710.

In some examples, the controller 714 is incorporated in a separate and remote electronic device in communication with the pump 710, such as a dedicated electronic device, computer, tablet PC, or smart phone. Alternatively, the controller 714 can be included in the pump 710 and, for example, can control both a user interface for manually operating the pump 710, as well as system functions such as receiving and processing information from the sensors 174.

As used herein, the terms “communication” and “communicate” refer to the receipt or transfer of one or more signals, messages, commands, or other type of data. For one unit or component to be in communication with another unit or component means that the one unit or component is able to directly or indirectly receive data from and/or transmit data to the other unit or component. This can refer to a direct or indirect connection that can be wired and/or wireless in nature. Additionally, two units or components can be in communication with each other even though the data transmitted can be modified, processed, routed, and the like, between the first and second unit or component. For example, a first unit can be in communication with a second unit even though the first unit passively receives data, and does not actively transmit data to the second unit. As another example, a first unit can be in communication with a second unit if an intermediary unit processes data from one unit and transmits processed data to the second unit. It will be appreciated that numerous other arrangements are possible.

The controller 714 is configured to receive information from the one or more sensors 174 and to store the information in the associated computer-readable memory 716. For example, the controller 714 can be configured to receive information from the sensor 174 at a predetermined rate, such as once every second, and to determine a conductance based on the received information. In some examples, the algorithm for calculating conductance can also include other sensor measurements, such as urine temperature, to obtain a more robust determination of conductance.

The controller 714 can also be configured to calculate patient physical statistics or diagnostic indicators that illustrate changes in the patient's condition over time. For example, the system 700 can be configured to identify an amount of total sodium excreted. The total sodium excreted may be based, for example, on a combination of flow rate and conductance over a period of time.

With continued reference to FIG. 44, the system 700 can further comprise a feedback device 720, such as a visual display or audio system, for providing information to the user. In some examples, the feedback device 720 can be integrally formed with the pump 710. Alternatively, the feedback device 720 can be a separate dedicated or a multipurpose electronic device, such as a computer, laptop computer, tablet PC, smart phone, or other handheld electronic devices. The feedback device 720 is configured to receive the calculated or determined measurements from the controller 714 and to present the received information to a user via the feedback device 720. For example, the feedback device 720 may be configured to display current negative pressure (in mmHg) being applied to the urinary tract. In other examples, the feedback device 720 is configured to display current flow rate of urine, temperature, current conductance in mS/m of urine, total urine produced during the session, total sodium excreted during the session, other physical parameters, or any combination thereof.

In some examples, the feedback device 720 further comprises a user interface module or component that allows the user to control operation of the pump 710. For example, the user can engage or turn off the pump 710 via the user interface. The user can also adjust pressure applied by the pump 710 to achieve a greater magnitude or rate of sodium excretion and fluid removal.

Optionally, the feedback device 720 and/or pump 710 further comprise a data transmitter 722 for sending information from the device 720 and/or pump 710 to other electronic devices or computer networks. The data transmitter 722 can utilize a short-range or long-range data communications protocol. An example of a short-range data transmission protocol is Bluetooth®. Long-range data transmission networks include, for example, Wi-Fi or cellular networks. The data transmitter 722 can send information to a patient's physician or caregiver to inform the physician or caregiver about the patient's current condition. Alternatively, or in addition, information can be sent from the data transmitter 722 to existing databases or information storage locations, such as, for example, to include the recorded information in a patient's electronic health record (EHR).

With continued reference to FIG. 44, in addition to the urine sensors 174, in some examples, the system 700 can further comprise one or more patient monitoring sensors 724. Patient monitoring sensors 724 can include invasive and non-invasive sensors for measuring information about the patient's physical parameters, such as urine composition, as discussed in detail above, blood composition (e.g., hematocrit ratio, analyte concentration, protein concentration, creatinine concentration) and/or blood flow (e.g., blood pressure, blood flow velocity). Hematocrit is a ratio of the volume of red blood cells to the total volume of blood. Normal hematocrit is about 25% to 40%, and preferably about 35% and 40% (e.g., 35% to 40% red blood cells by volume and 60% to 65% plasma).

Non-invasive patient monitoring sensors 724 can include pulse oximetry sensors, blood pressure sensors, heart rate sensors, and respiration sensors (e.g., a capnography sensor). Invasive patient monitoring sensors 724 can include invasive blood pressure sensors, glucose sensors, blood velocity sensors, hemoglobin sensors, hematocrit sensors, protein sensors, creatinine sensors, and others. In still other examples, sensors may be associated with an extracorporeal blood system or circuit and configured to measure parameters of blood passing through tubing of the extracorporeal system. For example, analyte sensors, such as capacitance sensors or optical spectroscopy sensors, may be associated with tubing of the extracorporeal blood system to measure parameter values of the patient's blood as it passes through the tubing. The patient monitoring sensors 724 can be in wired or wireless communication with the pump 710 and/or controller 714.

In some examples, the controller 714 is configured to cause the pump 710 to provide treatment for a patient based information obtained from the urine analyte sensor 174 and/or patient monitoring sensors 724, such as blood monitoring sensors. For example, pump 710 operating parameters can be adjusted based on changes in the patient's blood hematocrit ratio, blood protein concertation, creatinine concentration, urine output volume, urine protein concentration (e.g., albumin), and other parameters. For example, the controller 714 can be configured to receive information about a blood hematocrit ratio or creatinine concentration of the patient from the patient monitoring sensors 724 and/or analyte sensors 174. The controller 714 can be configured to adjust operating parameters of the pump 710 based on the blood and/or urine measurements. In other examples, hematocrit ratio may be measured from blood samples periodically obtained from the patient. Results of the tests can be manually or automatically provided to the controller 714 for processing and analysis.

As discussed herein, measured hematocrit values for the patient can be compared to predetermined threshold or clinically acceptable values for the general population. Generally, hematocrit levels for females are lower than for males. In other examples, measured hematocrit values can be compared to patient baseline values obtained prior to a surgical procedure. When the measured hematocrit value is increased to within the acceptable range, the pump 710 may be turned off ceasing application of negative pressure to the ureter or kidneys. In a similar manner, the intensity of negative pressure can be adjusted based on measured parameter values. For example, as the patient's measured parameters begin to approach the acceptable range, intensity of negative pressure being applied to the ureter and kidneys can be reduced. In contrast, if an undesirable trend (e.g., a decrease in hematocrit value, urine output rate, and/or creatinine clearance) is identified, the intensity of negative pressure can be increased in order to produce a positive physiological result. For example, the pump 710 may be configured to begin by providing a low level of negative pressure (e.g., ranging from about 0.1 mm Hg to about 10 mm Hg). The negative pressure may be incrementally increased until a positive trend in patient creatinine level is observed. In some examples, the negative pressure can range from about 0.1 mm Hg to about 150 mm Hg gauge pressure measured at the pump output, or, the negative pressure provided by the pump 710 can be about 0.1 to about 150 mm Hg.

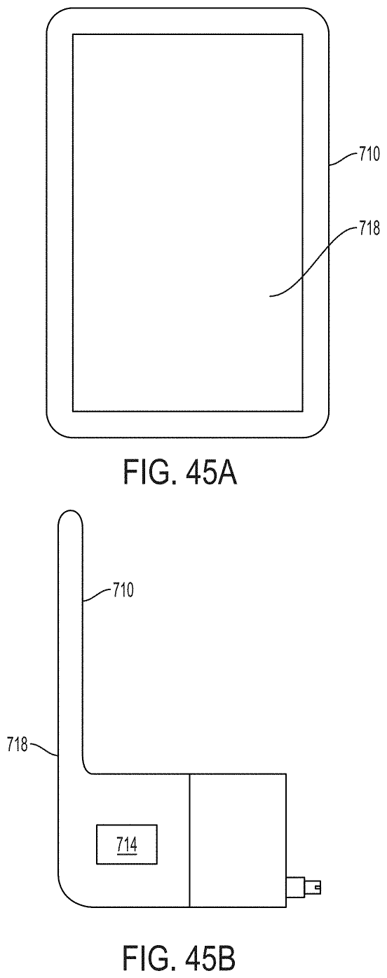

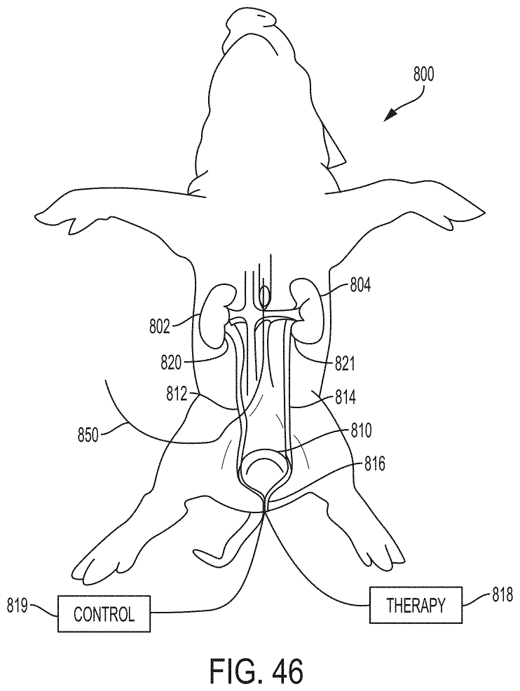

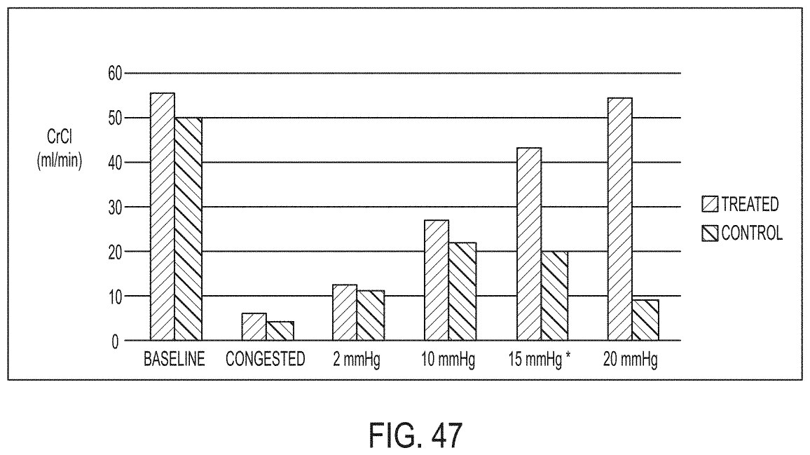

With reference to FIGS. 45A and 45B, an exemplary pump 710 for use with the system is illustrated. In some examples, the pump 710 is a micro-pump configured to draw fluid from the catheter(s) 112, 114 (shown, for example, in FIGS. 1A, 1B, 1C, 1F, 1P, 1U, 2A, 2B) and having a sensitivity or accuracy of about 10 mm Hg or less. Desirably, the pump 710 is capable of providing a range of flow of urine between 0.05 ml/min and 3 ml/min for extended periods of time, for example, for about 8 hours to about 24 hours per day, for one (1) to about 30 days or longer. At 0.2 ml/min, it is anticipated that about 300 mL of urine per day is collected by the system 700. The pump 710 can be configured to provide a negative pressure to the bladder of the patient, the negative pressure ranging from about 0.1 mm Hg to about 150 mm Hg, or about 0.1 mm Hg to about 50 mm Hg, or about 5 mm Hg to about 20 mm Hg (gauge pressure at the pump 710). For example, a micro-pump manufactured by Langer Inc. (Model BT100-2J) can be used with the presently disclosed system 700. Diaphragm aspirator pumps, as well as other types of commercially available pumps, can also be used for this purpose. Peristaltic pumps can also be used with the system 700. In other examples, a piston pump, vacuum bottle, or manual vacuum source can be used for providing negative pressure. In other examples, the system can be connected to a wall suction source, as is available in a hospital, through a vacuum regulator for reducing negative pressure to therapeutically appropriate levels.

In some examples, at least a portion of the pump assembly can be positioned within the patient's urinary tract, for example within the bladder. For example, the pump assembly can comprise a pump module and a control module coupled to the pump module, the control module being configured to direct motion of the pump module. At least one (one or more) of the pump module, the control module, or the power supply may be positioned within the patient's urinary tract. The pump module can comprise at least one pump element positioned within the fluid flow channel to draw fluid through the channel. Some examples of suitable pump assemblies, systems and methods of use are disclosed in U.S. Patent Application No. 62/550,259, entitled “Indwelling Pump for Facilitating Removal of Urine from the Urinary Tract”, filed on Aug. 25, 2017, which is incorporated by reference herein in its entirety.

In some examples, the pump 710 is configured for extended use and, thus, is capable of maintaining precise suction for extended periods of time, for example, for about 8 hours to about 24 hours per day, or for 1 to about 30 days or longer, except for replacement time of bladder catheters. Further, in some examples, the pump 710 is configured to be manually operated and, in that case, includes a control panel 718 that allows a user to set a desired suction value. The pump 710 can also include a controller or processor, which can be the same controller that operates the system 700 or can be a separate processor dedicated for operation of the pump 710. In either case, the processor is configured for both receiving instructions for manual operation of the pump and for automatically operating the pump 710 according to predetermined operating parameters. Alternatively, or in addition, operation of the pump 710 can be controlled by the processor based on feedback received from the plurality of sensors associated with the catheter.

In some examples, the processor is configured to cause the pump 710 to operate intermittently. For example, the pump 710 may be configured to emit pulses of negative pressure followed by periods in which no negative pressure is provided. In other examples, the pump 710 can be configured to alternate between providing negative pressure and positive pressure to produce an alternating flush and pump effect. For example, a positive pressure of about 0.1 mm Hg to about 20 mm Hg, or about 5 mm Hg to about 20 mm Hg can be provided followed by a negative pressure ranging from about 0.1 mm Hg to about 150 mm Hg.

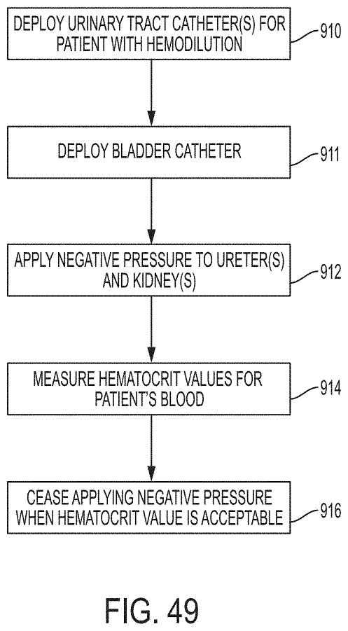

Steps for removing excess fluid from a patient using the devices and systems described herein are illustrated in FIG. 49. As shown in FIG. 49, the treatment method comprises deploying a ureteral stent or a urinary tract catheter, such as a ureteral catheter, in the ureter and/or kidney of a patient such that flow of urine from the ureter and/or kidney, as shown at box 910. The catheter may be placed to avoid occluding the ureter and/or kidney. In some examples, a fluid collecting portion of the stent or catheter may be positioned in the renal pelvis of the patient's kidney. In some examples, a ureteral stent or ureteral catheter may be positioned in each of the patient's kidneys. In other examples, a urine collection catheter may be deployed in the bladder or ureter, as shown in box 911. In some examples, the ureteral catheter comprises one or more of any of the retention portions described herein. For example, the ureteral catheter can comprise a tube defining a drainage lumen comprising a helical retention portion and a plurality of drainage ports. In other examples, the catheter can include a funnel-shaped fluid collection and retention portion or a pigtail coil. Alternatively, a ureteral stent, having, for example, a pigtail coil, can be deployed.

As shown at box 912, the method further comprises applying negative pressure to at least one of the bladder, the ureter and/or kidney through the bladder catheter to induce or facilitate production of fluid or urine in the kidney(s) and to extract the fluid or urine from the patient. Desirably, negative pressure is applied for a period of time sufficient to reduce the patient's blood creatinine levels by a clinically significant amount.

Negative pressure may continue to be applied for a predetermined period of time. For example, a user may be instructed to operate the pump for the duration of a surgical procedure or for a time period selected based on physiological characteristics of the patient. In other examples, patient condition may be monitored to determine when sufficient treatment has been provided. For example, as shown at box 914, the method may further comprise monitoring the patient to determine when to cease applying negative pressure to the patient's bladder, ureter and/or kidneys. In some examples, a patient's hematocrit level is measured. For example, patient monitoring devices may be used to periodically obtain hematocrit values. In other examples, blood samples may be drawn periodically to directly measure hematocrit. In some examples, concentration and/or volume of urine expelled from the body through the bladder catheter may be monitored to determine a rate at which urine is being produced by the kidneys. In a similar manner, expelled urine output may be monitored to determine protein concentration and/or creatinine clearance rate for the patient. Reduced creatinine and protein concentration in urine may be indicative of over-dilution and/or depressed renal function. Measured values can be compared to the predetermined threshold values to assess whether negative pressure therapy is improving patient condition, and should be modified or discontinued. For example, as discussed herein, a desirable range for patient hematocrit may be between 25% and 40%. In other examples, as described herein, patient body weight may be measured and compared to a dry body weight. Changes in measured patient body weight demonstrate that fluid is being removed from the body. As such, a return to dry body weight represents that hemodilution has been appropriately managed and the patient is not over-diluted.

As shown at box 916, a user may cause the pump to cease providing negative pressure therapy when a positive result is identified. In a similar manner, patient blood parameters may be monitored to assess effectiveness of the negative pressure being applied to the patient's kidneys. For example, a capacitance or analyte sensor may be placed in fluid communication with tubing of an extracorporeal blood management system. The sensor may be used to measure information representative of blood protein, oxygen, creatinine, and/or hematocrit levels. Measured blood parameter values may be measured continuously or periodically and compared to various threshold or clinically acceptable values. Negative pressure may continue to be applied to the patient's bladder, kidney or ureter until a measured parameter value falls within a clinically acceptable range. Once a measured values fails within the threshold or clinically acceptable range, as shown at box 916, application of negative pressure may cease.

In some examples, there is provided a method of removing excess fluid from a patient for systemic fluid volume management associated with chronic edematous, hypertension, chronic kidney disease and/or acute heart failure. According to another aspect of the disclosure, a method for removing excess fluid for a patient undergoing a fluid resuscitation procedure, such as coronary graft bypass surgery, by removing excess fluid from the patient is provided. During fluid resuscitation, solutions such as saline solutions and/or starch solutions, are introduced to the patient's bloodstream by a suitable fluid delivery process, such as an intravenous drip. For example, in some surgical procedures, a patient may be supplied with between 5 and 10 times a normal daily intake of fluid. Fluid replacement or fluid resuscitation can be provided to replace bodily fluids lost through sweating, bleeding, dehydration, and similar processes. In the case of a surgical procedure such as coronary graft bypass, fluid resuscitation is provided to help maintain a patient's fluid balance and blood pressure within an appropriate rate. Acute kidney injury (AKI) is a known complication of coronary artery graft bypass surgery. AKI is associated with a prolonged hospital stay and increased morbidity and mortality, even for patients who do not progress to renal failure. See Kim, et al., Relationship between a perioperative intravenous fluid administration strategy and acute kidney injury following off-pump coronary artery bypass surgery: an observational study, Critical Care 19:350 (1995). Introducing fluid to blood also reduces hematocrit levels which has been shown to further increase mortality and morbidity. Research has also demonstrated that introducing saline solution to a patient may depress renal functional and/or inhibit natural fluid management processes. As such, appropriate monitoring and control of renal function may produce improved outcomes and, in particular, may reduce post-operative instances of AKI.

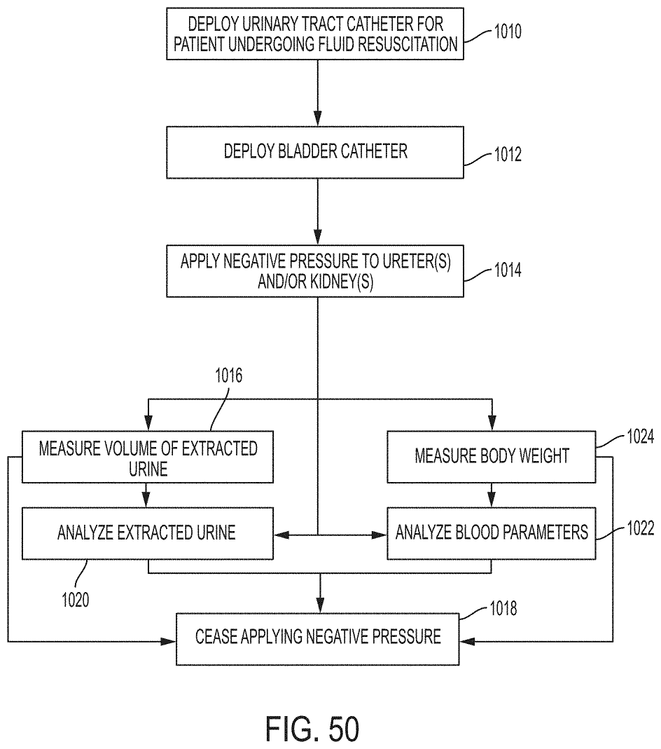

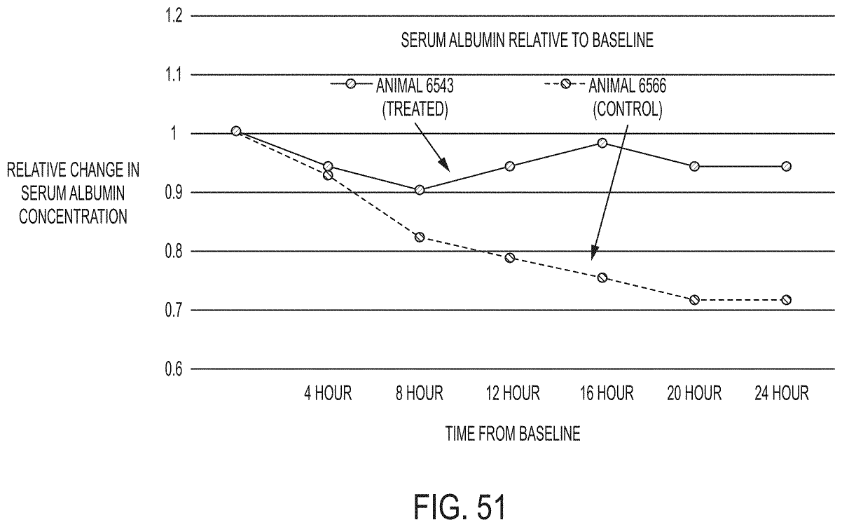

A method of treating a patient for removing excess fluid is illustrated in FIG. 50. As shown at box 1010, the method comprises deploying a ureteral stent or ureteral catheter in the ureter and/or kidney of a patient such that flow of urine from the ureter and/or kidney is not prevented by occlusion of the ureter and/or kidney. For example, a distal end of the ureteral stent or fluid collecting portion of the catheter may be positioned in the renal pelvis. In other examples, the catheter may be deployed in the kidney or ureter. The catheter can comprise one or more of the ureter catheters described herein. For example, the catheter can comprise a tube defining a drainage lumen and comprising a helical retention portion and a plurality of drainage ports. In other examples, the catheter can include a pigtail coil.

As shown at box 1012, a bladder catheter can be deployed in the patient's bladder. For example, the bladder catheter may be positioned to at least partially seal the urethra opening to prevent passage of urine from the body through the urethra. The bladder catheter can, for example, include an anchor for maintaining the distal end of the catheter in the bladder. As described herein, other arrangements of coils and helices, funnel, etc. may be used to obtain proper positioning of the bladder catheter. The bladder catheter can be configured to collect fluid which entered the patient's bladder prior to placement of the ureteral catheter(s), as well as fluid collected from the ureters, ureteral stents, and/or ureteral catheters during treatment. The bladder catheter may also collect urine which flows past the fluid collection portion(s) of the ureteral catheter and enters the bladder. In some examples, a proximal portion of the ureteral catheter may be positioned in a drainage lumen of the bladder catheter. In a similar manner, the bladder catheter may be advanced into the bladder using the same guidewire used for positioning of the ureteral catheter(s). In some examples, negative pressure may be provided to the bladder through the drainage lumen of the bladder catheter. In other examples, negative pressure may only be applied to the bladder catheter(s). In that case, the ureteral catheter drains into the bladder by gravity.

As shown at box 1014, following deployment of the ureteral stents and/or ureteral catheter(s) and the bladder catheter, negative pressure is applied to the bladder, ureter and/or kidney through the bladder catheter. For example, negative pressure can be applied for a period of time sufficient to extract urine comprising a portion of the fluid provided to the patient during the fluid resuscitation procedure. As described herein, negative pressure can be provided by an external pump connected to a proximal end or port of the bladder catheter. The pump can be operated continually or periodically dependent on therapeutic requirements of the patient. In some cases, the pump may alternate between applying negative pressure and positive pressure.

Negative pressure may continue to be applied for a predetermined period of time. For example, a user may be instructed to operate the pump for the duration of a surgical procedure or for a time period selected based on physiological characteristics of the patient. In other examples, patient condition may be monitored to determine when a sufficient amount of fluid has been drawn from the patient. For example, as shown at box 1016, fluid expelled from the body may be collected and a total volume of obtained fluid may be monitored. In that case, the pump can continue to operate until a predetermined fluid volume has been collected from the ureteral and/or bladder catheters. The predetermined fluid volume may be based, for example, on a volume of fluid provided to the patient prior to and during the surgical procedure. As shown at box 1018, application of negative pressure to the bladder, ureter and/or kidneys is stopped when the collected total volume of fluid exceeds the predetermined fluid volume.

In other examples, operation of the pump can be determined based on measured physiological parameters of the patient, such as measured creatinine clearance, blood creatinine level, or hematocrit ratio. For example, as shown at box 1020, urine collected form the patient may be analyzed by one or more sensors associated with the catheter and/or pump. The sensor can be a capacitance sensor, analyte sensor, optical sensor, or similar device configured to measure urine analyte concentration. In a similar manner, as shown at box 1022, a patient's blood creatinine or hematocrit level could be analyzed based on information obtain from the patient monitoring sensors discussed hereinabove. For example, a capacitance sensor may be placed in an existing extracorporeal blood system. Information obtained by the capacitance sensor may be analyzed to determine a patient's hematocrit ratio. The measured hematocrit ratio may be compared to certain expected or therapeutically acceptable values. The pump may continue to apply negative pressure to the patient's ureter and/or kidney until measured values within the therapeutically acceptable range are obtained. Once a therapeutically acceptable value is obtained, application of negative pressure may be stopped as shown at box 1018.

In other examples, as shown at box 2024, patient body weight may be measured to assess whether fluid is being removed from the patient by the applied negative pressure therapy. For example, a patient's measured bodyweight (including fluid introduced during a fluid resuscitation procedure) can be compared to a patient's dry body weight. As used herein, dry weights is defined as normal body weight measured when a patient is not over-diluted. For example, a patient who is not experiencing one or more of: elevated blood pressure, lightheadedness or cramping, swelling of legs, feet, arms, hands, or around the eyes, and who is breathing comfortably, likely does not have excess fluid. A weight measured when the patient is not experiencing such symptoms can be a dry body weight. Patient weight can be measured periodically until the measured weight approaches the dry body weight. When the measured weight approaches (e.g., is within about 2% to 10% of dry body weight), as shown at box 1018, application of negative pressure can be decreased or stopped.

The aforementioned details of treatment using the systems of the present invention can be used to treat a variety of conditions that can benefit from increased urine or fluid output or removal. For example, a method for preserving renal function by application of negative pressure to decrease interstitial pressure within tubules of the medullar region to facilitate urine output and to prevent venous congestion-induced nephron hypoxia in the medulla of the kidney is provided. The method comprises: deploying a ureteral stent or ureteral catheter into a ureter or kidney of a patient to maintain patency of fluid flow between a kidney and a bladder of the patient; deploying a bladder catheter into the bladder of the patient, wherein the bladder catheter comprises a distal end configured to be positioned in a patient's bladder, a drainage lumen portion having a proximal end, and a sidewall extending therebetween; and applying negative pressure to the proximal end of the catheter to induce negative pressure in a portion of the urinary tract of the patient for a predetermined period of time to remove fluid from the urinary tract of the patient.

In another example, a method for treatment of acute kidney injury due to venous congestion is provided. The method comprises: deploying a ureteral stent or ureteral catheter into a ureter or kidney of a patient to maintain patency of fluid flow between a kidney and a bladder of the patient; deploying a bladder catheter into the bladder of the patient, wherein the bladder catheter comprises a distal end configured to be positioned in a patient's bladder, a drainage lumen portion having a proximal end, and a sidewall extending therebetween; and applying negative pressure to the proximal end of the catheter to induce negative pressure in a portion of the urinary tract of the patient for a predetermined period of time to remove fluid from the urinary tract of the patient, thereby reducing venous congestion in the kidney to treat acute kidney injury.

In another example, a method for treatment of New York Heart Association (NYHA) Class III and/or Class IV heart failure through reduction of venous congestion in the kidney(s) is provided. The method comprises: deploying a ureteral stent or ureteral catheter into a ureter or kidney of a patient to maintain patency of fluid flow between a kidney and a bladder of the patient; deploying a bladder catheter into the bladder of the patient, wherein the bladder catheter comprises a distal end configured to be positioned in a patient's bladder, a drainage lumen portion having a proximal end, and a sidewall extending therebetween; and applying negative pressure to the proximal end of the catheter to induce negative pressure in a portion of the urinary tract of the patient for a predetermined period of time to remove fluid from the urinary tract of the patient to treat volume overload in NYHA Class III and/or Class IV heart failure.

In another example, a method for treatment of Stage 4 and/or Stage 5 chronic kidney disease through reduction of venous congestion in the kidney(s) is provided. The method comprises: deploying a ureteral stent or ureteral catheter into a ureter or kidney of a patient to maintain patency of fluid flow between a kidney and a bladder of the patient; deploying a bladder catheter into the bladder of the patient, wherein the bladder catheter comprises a distal end configured to be positioned in a patient's bladder, a drainage lumen portion having a proximal end, and a sidewall extending therebetween; and applying negative pressure to the proximal end of the catheter to induce negative pressure in a portion of the urinary tract of the patient to remove fluid from the urinary tract of the patient to reduce venous congestion in the kidney(s).

In some examples, a kit is provided for removing fluid from the urinary tract of a patient and/or inducing negative pressure in a portion of a urinary tract of a patient. The kit comprises: a ureteral stent or ureteral catheter comprising a drainage channel for facilitating flow of fluid from the ureter and/or kidney through the drainage channel of the ureteral stent or ureteral catheter towards the bladder of the patient; and a pump comprising a controller configured to induce a negative pressure in at least one of the ureter, kidney or bladder of the patient to draw urine through a drainage lumen of a catheter deployed in the patient's bladder. In some examples, the kit further comprises at least one bladder catheter. In some examples, the kit further comprises instructions for one or more of the following: inserting/deploying the ureteral stent(s) and/or ureteral catheter(s), inserting/deploying the bladder catheter, and operating the pump to draw urine through a drainage lumen of the bladder catheter deployed the patient's bladder.

In some examples, another kit comprises: a plurality of disposable bladder catheters, each bladder catheter comprising a drainage lumen portion having a proximal end, a distal end configured to be positioned in a patient's bladder, and a sidewall extending therebetween; and a retention portion extending radially outward from a portion of the distal end of the drainage lumen portion, and being configured to be extended into a deployed position in which a diameter of the retention portion is greater than a diameter of the drainage lumen portion; instructions for inserting/deploying the bladder catheter; and instructions for connecting the proximal end of the bladder catheter to a pump and for operating the pump to draw urine through the drainage lumen of the bladder catheter, for example by applying negative pressure to the proximal end of the bladder catheter.

In some examples, a kit is provided, the kit comprising: a plurality of disposable bladder catheters, each bladder catheter comprising (a) a proximal portion; and (b) a distal portion, the distal portion comprising a retention portion that comprises at least one protected drainage hole(s), port(s) or perforation(s) and is configured to establish an outer periphery or protective surface area that inhibits mucosal tissue from occluding the at least one protected drainage hole(s), port(s) or perforation(s) upon application of negative pressure through the catheter; instructions for deploying the bladder catheter; and instructions for connecting the proximal end of the bladder catheter to a pump and for operating the pump to draw urine through the drainage lumen of the bladder catheter.

### Exemplary Percutaneous Catheters

In some examples, a percutaneous catheter, such as are disclosed in US Patent Application Publication No. 2019/0105465 which is incorporated by reference herein in its entirety, is suitable for use with the methods disclosed herein.

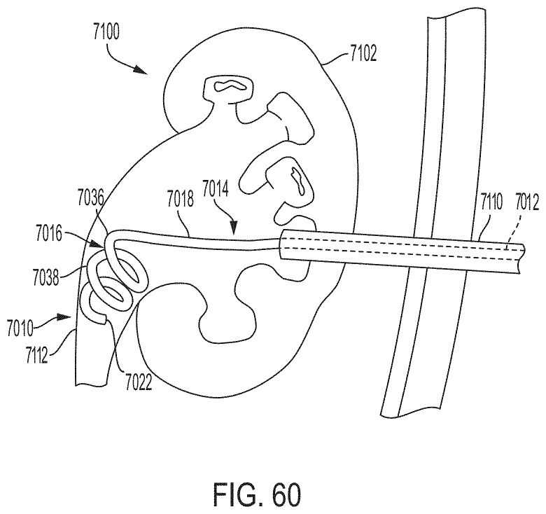

Referring now to FIG. 60, an exemplary percutaneous nephrostomy tube or urinary bypass catheter 7010 will be discussed. It is understood, however, that any of the catheters discussed herein can be used as a percutaneous catheter in a similar manner as described below. The exemplary urinary bypass catheter 7010 is configured to be deployed in a urinary tract 7100 (shown in FIGS. 60, 61, and 62A-62E) of a patient. The catheter 7010 comprises an elongated tube 7018 extending from a proximal end 7020 to a distal end 7022. The elongated tube 7018 comprises a proximal portion 7012 configured to pass into an abdomen of the patient through a percutaneous opening or access site 7110 (shown in FIG. 60) and a distal portion 7014 comprising a retention portion 7016 configured to be deployed in a renal pelvis 7112, kidney 7102 (shown in FIG. 60), and/or bladder, of the patient. The percutaneous access site 7110 can be formed in a conventional manner, such as by inserting a tip of a needle through the skin into the abdomen.

The tube 7018 can be formed from and/or comprise one or more biocompatible polymer(s), such as polyurethane, polyvinyl chloride, polytetrafluoroethylene (PTFE), latex, silicone coated latex, silicone, polyglycolide or poly(glycolic acid) (PGA), Polylactide (PLA), Poly(lactide-co-glycolide), Polyhydroxyalkanoates, Polycaprolactone and/or Poly(propylene fumarate). Portions of the elongated tube 18 can also comprise and/or be impregnated with metal materials, such as copper, silver, gold, nickel-titanium alloy, stainless steel, and/or titanium. The elongated tube 7018 should be of sufficient length to extend from the renal pelvis 7112, through the kidney and percutaneous access site, and to the external fluid collection container. The tube 7018 size can range from about 1 Fr to about 9 Fr (French catheter scale), or about 2 Fr to 8 Fr, or can be about 4 Fr. In some examples, the tube 18 can have an external diameter ranging from about 0.33 mm to about 3.0 mm, or about 0.66 mm to 2.33 mm, or about 1.0 mm to 2.0 mm, and an internal diameter ranging from about 0.165 mm to about 2.40 mm, or about 0.33 mm to 2.0 mm, or about 0.66 mm to about 1.66 mm. In one example, the tube 7018 is 6 Fr and has an outer diameter of 2.0±0.1 mm. A length of the tube 7018 can range from about 30 cm to about 120 cm depending on the age (e.g., pediatric or adult) and size of the patient.

The retention portion 7016 of the bypass catheter 7010 can be integrally formed with the distal portion 7014 of the catheter 7010 or can be a separate structure mounted to the distal end 7022 of the elongated tube 7018 by a conventional fastener or adhesive. Many exemplary retention portions 7016 suitable for retaining the distal end 7022 of the elongated tube 7018 within the renal pelvis 7112 are provided in previous exemplary embodiments of ureteral catheters 7010. For example, retention portions 7016 comprising one or more of coils, funnels, cages, balloons, and/or sponges can be adapted for use with the bypass catheter 7010. In some cases, such retention portions 7016 can be adapted for use with urinary bypass catheters 7010 by, for example, inverting the retention portion(s) 7016 to account for the fact that a urinary bypass catheter 7010 enters the renal pelvis 7112 through the kidney 7102, rather than through the ureters.

Regardless of the embodiment selected, the retention portion 7016 creates an outer periphery or protected surface area to prevent urinary tract tissues from constricting or occluding a fluid column extending between nephrons of the kidney 7102 and a lumen of the elongated tube 7018. In some examples, such a retention portion 7016 could comprise an inwardly facing side or protected surface area 7024 comprising one or more drainage openings, perforations, and/or ports 7026 for receiving fluid, such as urine, produced by the kidneys 7102 and an outwardly facing side or protective surface area 7028, which can be free from or substantially free from the drainage ports 7026. Desirably, the inwardly facing side or protected surface area 7024 and the outwardly facing side or protective surface area 7028 are configured such that, when negative pressure is applied through the elongated tube 7018, the urine is drawn into a lumen of the tube 7018 through the one or more drainage ports 7026, while mucosal tissues, such as tissue of the ureters and/or renal pelvis 7112, are prevented from appreciably occluding the one or more drainage ports 7026. As in previously described ureteral catheters, sizes and spacing between the drainage ports 7026 may vary to achieve different distributions of negative pressure within the renal pelvis 7112 and/or kidney 7102, as are disclosed herein. In some examples, each of the one or more drainage ports 7026 has a diameter of about 0.0005 mm to about 2.0 mm, or about 0.05 mm to 1.5 mm, or about 0.5 mm to about 1.0 mm. In some examples, the drainage ports 7026 can be non-circular, and can have a surface area of about 0.0002 mm² to about 100 mm², or about 0.002 mm² to about 10 mm², or about 0.2 mm² to about 1.0 mm². The drainage ports 7026 can be spaced equidistantly along an axial length of the retention portion 7016. In other examples, drainage ports 7026 nearer to the distal end 7022 of the retention portion 7016 may be spaced more closely together to increase fluid flow through more distal drainage ports 7026, compared to examples where the ports 7026 are evenly spaced.

The proximal portion 7012 of the catheter 7010 generally extends from the kidney 7102 of the patient through the percutaneous access site 7110. The proximal portion 7012 of the catheter 7010 is free from or substantially free from perforations, openings, or drainage ports 7026 to avoid drawing fluids from the abdominal cavity into the elongated tube 7018. Also, the proximal end 7020 of the proximal portion 7012 can be configured to be connected to a fluid collection container and/or pump, as shown in 61. 55.

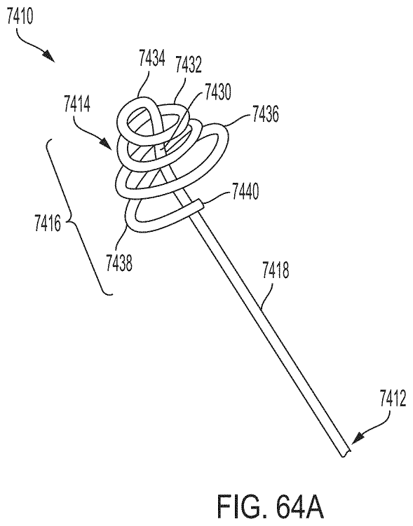

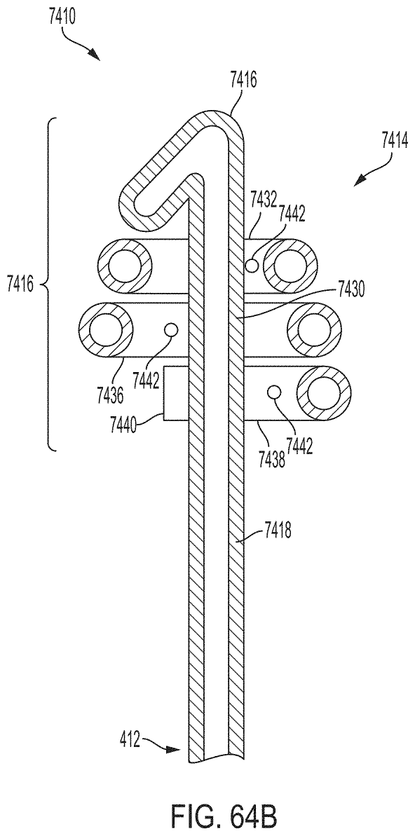

Another example of a ureteral catheter 7410 configured for percutaneous insertion into the renal pelvis of a patient is shown in FIGS. 64A and 64B. As in previous examples, the ureteral catheter 7410 is formed from an elongated tube 7418, and comprises a proximal portion 7412 and a distal portion 7414 comprising a retention portion 7416. The retention portion 7416 is a coiled retention portion comprising a plurality of coils wrapped around a substantially linear or straight segment or portion 7430 of the elongated tube 7418.

The coiled retention portion 416 further comprises a distal-most coil 7432 formed from a bend 7434 of from about 90 degrees to 180 degrees at a distal end of the straight segment or portion 7430 of the retention portion 7416. The retention portion 7416 further comprises one or more additional coils, such as a second or middle coil 7436 and a third or proximal most coil 7438, which are wrapped around the straight portion 7430 of the tube 7418. The elongated tube 7418 further comprises a distal end 7440 following the proximal most coil 7438. The distal end 7440 can be closed or can be open to receive urine from the patient's urinary tract.

As in previous examples, the size and orientation of the coils 7432, 7436, 7438 is selected so that the retention portion 7416 remains in the renal pelvis and does not pass into the ureter or retract back into the kidney. For example, the largest or proximal most coil 7438 can be about 10 mm to 30 mm in diameter, or about 15 mm to 25 mm in diameter, or about 20 mm in diameter. Coils 7436 and 7438 can have a smaller diameter of, for example, 5 mm to 25 mm, or about 10 mm to 20 mm, or about 15 mm. As in previous examples, the coiled retention portion 7416 can have a tapered appearance in which the coils 7432, 7436, 7438 become progressively narrower, giving the retention portion 7416 a reverse pyramid or reverse conical appearance.

Also as in previous examples, the retention portion 7416 further comprises openings or drainage ports 7442 positioned on a radially inward side or protected surface area of the coiled retention portion 7416. Since the coils 7432, 7436, 7438 extend around the straight portion 7430 and prevent tissue of the renal pelvis and/or kidneys from contacting the straight portion 7430, openings or drainage ports 7442 (shown in FIG. 60B) can also be positioned on the straight portion 7430 of the retention portion 7416. The size of the openings or drainage ports can be as described above. As in previous examples, the retention portion 7416 is inserted through the kidney and renal pelvis in a linear orientation over a guidewire. When the guidewire is removed, the retention portion 7416 adopts the coiled or deployed configuration.

Urine Collection System with Percutaneous Catheters

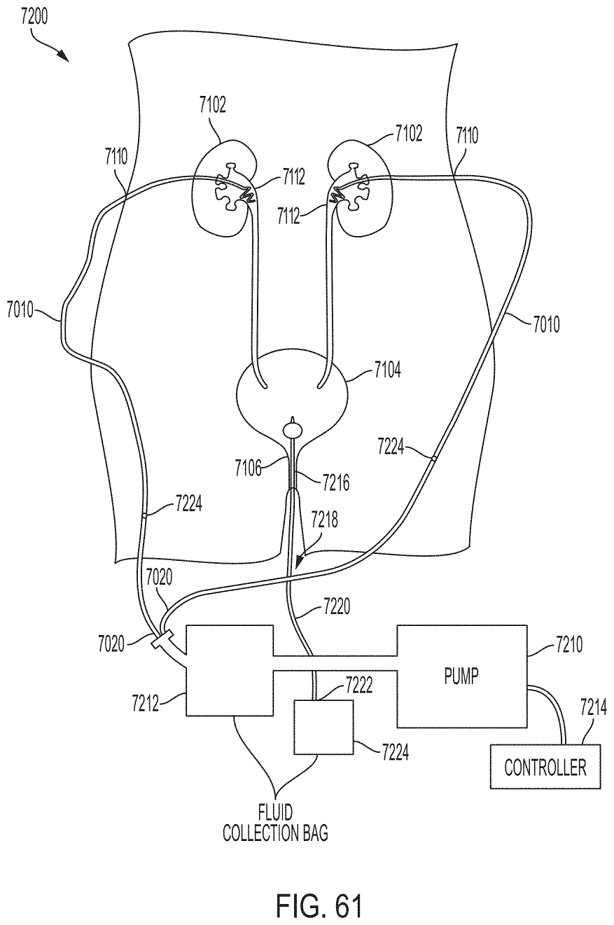

The urinary bypass catheter(s) 7010, 7410 can be used with systems for inducing negative pressure in a portion of a urinary tract 7100 of a patient. As shown in FIG. 61, an exemplary system 7200 comprises urinary bypass catheters 7010 deployed in the respective renal pelvis 7112 of each respective kidney 7102 of the patient. Proximal ends 7020 of the catheters 7010 are connected directly or indirectly to a pump 7210. For example, proximal ends 7020 of the catheters 7010 can be connected to fluid inflow ports of a rigid fluid collection container 7212. The pump 7210 can be connected to another port of the fluid collection container 7212 to induce negative pressure in the fluid collection container 7212 and catheters 7010 connected thereto. The pump 7210 can be similar to pumps in previously described examples and, particularly, can be configured to deliver gentle negative pressure to the urinary tract 7100 of the patient. The pump 7210 can be an external pump. In other examples, the pump 7210 can be an indwelling pump as described, for example, in PCT Appl. No. PCT/IB2018/056444, published as PCT Publication WO 2019/038730, entitled “Indwelling Pump for Facilitating Removal of Urine from the Urinary Tract.” Generally, the applied negative pressure is a gentle negative pressure, such as a negative pressure of less than 50 mm Hg. In other examples, negative pressure can be from 2 mm Hg to 100 mm Hg, or more, depending on therapeutic requirements of particular patients. The pump 7210 desirably has a sensitivity of 10 mmHg or less.

In some examples, the system 7200 further comprises a bladder catheter 7216 deployed within the patient's bladder 7104. The bladder catheter 7216 can be any suitable bladder catheter, as described in previous examples. The bladder catheter 7216 comprises an elongated tube 7218 comprising a proximal portion 7220, which extends through the urethra 7106 and from the patient. A proximal end 7222 of the proximal portion 7220 of the bladder catheter 7216 can be connected to the fluid collection container 7212. In other examples, the bladder catheter 7216 can be connected to a separate fluid collection container 7224, which is not connected to a pump 7210 for inducing negative pressure. In that case, fluid may pass from the patient's bladder 7104 through the bladder catheter 7216 by gravity.

In some examples, the system 7200 further comprises a controller 7214 electrically connected to the pump 7210 configured to actuate the pump 7210 and to control pump operating parameters. As in previous examples, the controller 7214 can be a microprocessor of the pump 7210 or a separate electronic device configured to provide operating instructions and/or operating parameters for the pump 7210. For example, the controller 7214 can be associated with a computer, laptop, tablet, smartphone, or similar electronic device.

The system can further comprise one or more physiological sensors 7226 associated with the patient, fluid collection container 7212, or catheter(s) 710, 7216. The physiological sensors 7226 can be configured to provide information representative of at least one physical parameter of the patient to the controller 7214. In that case, the controller 7214 can be configured to actuate or stop operation of the pump based on the at least one physical parameter.

Deployment

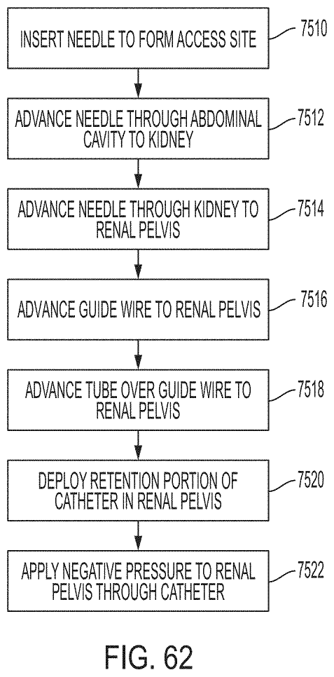

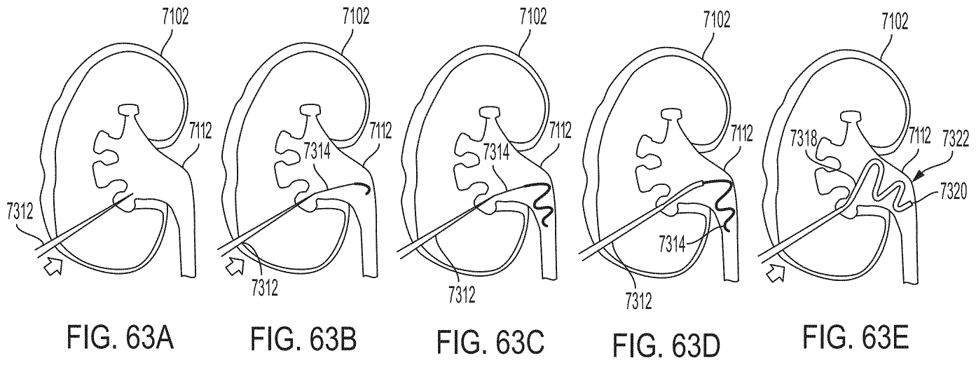

Having described examples of a urinary bypass catheter 7010 and system 7200 for applying negative pressure to a patient, a method for insertion and/or deployment of the urinary bypass catheter will now be described in connection with the flow chart of FIG. 62. Schematic drawings showing different examples of the catheter deployment method are shown in FIGS. 63A-63E. Initially, at box 7510, a needle 7312 (shown in FIGS. 63A-63C) of a conical tip catheter 7310 (shown in FIGS. 63A-63E) is inserted into an abdominal region of the patient, thereby creating a percutaneous access site. The catheter 7310 and needle 7312 should be of sufficient size to permit the urinary bypass catheter to pass through the catheter 7310. For example, the catheter 7310 can be about 3 Fr to about 10 Fr (French catheter scale), or from about 5 Fr to about 8 Fr, or about 6 Fr. In some examples, the catheter 7310 can have an external diameter ranging from about 0.5 mm to about 4 mm, and an internal diameter ranging from about 0.2 mm to about 3.5 mm. The needle 7312 can be from about 10 gauge to 30 gauge, or about 20 gauge to 25 gauge, and can have an outer diameter of from 0.3 mm to 3.5 mm, or about 0.5 mm to 1.0 mm. The needle 7312 can be any suitable length, such as from 10 mm to 50 mm, or about 30 mm.

Once the needle 7312 is inserted through the patient's skin, at 7512, the needle 7312 is advanced through the abdominal cavity and inserted into the kidney 7102. At 7514, the needle 7312 is advanced through the kidney 7102 and into the renal pelvis 7112, as shown in FIG. 63B. Once the needle 7312 is advanced to the renal pelvis 7112, at box 7516, a guidewire 7314 can be advanced to the renal pelvis 7112 through the needle 7312, as shown in FIG. 63C. Once the guidewire 7314 is in position, the needle 7312 can be retracted back through the catheter 7310. Next, at 7518, an elongated tube 7318 of the catheter 7310 can be inserted into the patient's abdominal cavity through the percutaneous access site and advanced to the renal pelvis over a guidewire 7314 and/or needle 7312, as shown in FIG. 63D. At 7520, once a distal end 7320 and retention portion 7322 of the elongated tube 7318 reach the renal pelvis, the retention portion 7322 can transition from its retracted state to the expanded or deployed state, as shown in FIG. 63E. As described herein, desirably, when deployed in the renal pelvis 7112, the retention portion 7322 maintains patency of fluid flow from the kidney 7102 into a lumen extending through at least a portion of the elongated tube 7318.

In some examples, deploying the retention portion 7322 can comprise retracting an outer tube or sheath in a proximal direction away from the retention portion 7322. Once the outer tube or sheath is removed, the retention portion 7322 automatically expands and returns to an unconstrained shape. In other examples, such as when the retention portion 7322 comprises a coiled retention portion, retracting the guidewire 7314 causes the retention portion 7322 to adopt the coiled or deployed configuration. Deploying the retention portion can also comprise, for example, blowing up a balloon or releasing a cage-like structure to protect the distal end of the elongated tube 7318.

In some examples, as shown in box 7522, negative pressure can be applied to the renal pelvis by attaching a proximal end of the elongated tube 7318 directly or indirectly to a fluid pump and actuating the pump to generate the negative pressure. For example, negative pressure can be applied continuously for a predetermined period of time. In other examples, negative pressure can be applied as pressure pulses provided for short duration at predetermined intervals. In some examples, the pump can alternate between providing negative pressure and positive pressure. It is believed that such alternating pressure therapy may further stimulate the kidneys resulting in increased urine production. In other examples, as described in greater detail above, negative pressure may be delivered to the renal pelvis through the elongated tube 7318 due to a pressure distribution or pressure gradient induced in the tube 7318, but without use of a pump or negative pressure source. For example, negative pressure sufficient to draw fluid, such as urine, into the tube 7318 may be created in distal portions of the tube 7318 in response to fluid flow through the tube 7318 by gravity. While not intending to be bound by theory, it is believed that the generated suction force is dependent on a vertical distance between the retention portion of the catheter and the proximal end of the catheter. Accordingly, the created negative pressure can be increased by increasing the vertical distance between the retention portion of the deployed catheter and the fluid collection container and/or proximal end of the catheter.

### Experimental Examples of Inducing Negative Pressure Using Ureteral Catheters

Inducement of negative pressure within the renal pelvis of farm swine was performed for the purpose of evaluating effects of negative pressure therapy on renal congestion in the kidney. An objective of these studies was to demonstrate whether a negative pressure delivered into the renal pelvis significantly increases urine output in a swine model of renal congestion. In Example 1, a pediatric Fogarty catheter, normally used in embolectomy or bronchoscopy applications, was used in the swine model solely for proof of principle for inducement of negative pressure in the renal pelvis. It is not suggested that a Fogarty catheter be used in humans in clinical settings to avoid injury of urinary tract tissues. In Example 2, the ureteral catheter 112 shown in FIGS. 2A and 2B, and including a helical retention portion for mounting or maintaining a distal portion of the catheter in the renal pelvis or kidney, was used.

### Example 1

Method

Four farm swine 800 were used for purposes of evaluating effects of negative pressure therapy on renal congestion in the kidney. As shown in FIG. 21, pediatric Fogarty catheters 812, 814 were inserted to the renal pelvis region 820, 821 of each kidney 802, 804 of the four swine 800. The catheters 812, 814 were deployed within the renal pelvis region by inflating an expandable balloon to a size sufficient to seal the renal pelvis and to maintain the position of the balloon within the renal pelvis. The catheters 812, 814 extend from the renal pelvis 802, 804, through a bladder 810 and urethra 816, and to fluid collection containers external to the swine.

Urine output of two animals was collected for a 15 minute period to establish a baseline for urine output volume and rate. Urine output of the right kidney 802 and the left kidney 804 were measured individually and found to vary considerably. Creatinine clearance values were also determined.

Renal congestion (e.g., congestion or reduced blood flow in the veins of the kidney) was induced in the right kidney 802 and the left kidney 804 of the animal 800 by partially occluding the inferior vena cava (IVC) with an inflatable balloon catheter 850 just above to the renal vein outflow. Pressure sensors were used to measure IVC pressure. Normal IVC pressures were 1-4 mm Hg. By inflating the balloon of the catheter 850 to approximately three quarters of the IVC diameter, the IVC pressures were elevated to between 15-25 mm Hg. Inflation of the balloon to approximately three quarters of IVC diameter resulted in a 50-85% reduction in urine output. Full occlusion generated IVC pressures above 28 mm Hg and was associated with at least a 95% reduction in urine output.

One kidney of each animal 800 was not treated and served as a control (“the control kidney 802”). The ureteral catheter 812 extending from the control kidney was connected to a fluid collection container 819 for determining fluid levels. One kidney (“the treated kidney 804”) of each animal was treated with negative pressure from a negative pressure source (e.g., a therapy pump 818 in combination with a regulator designed to more accurately control the low magnitude of negative pressures) connected to the ureteral catheter 814. The pump 818 was an Air Cadet Vacuum Pump from Cole-Parmer Instrument Company (Model No. EW-07530-85). The pump 818 was connected in series to the regulator. The regulator was an V-800 Series Miniature Precision Vacuum Regulator—1/8 NPT Ports (Model No. V-800-10-W/K), manufactured by Airtrol Components Inc.

The pump 818 was actuated to induce negative pressure within the renal pelvis 820, 821 of the treated kidney according to the following protocol. First, the effect of negative pressure was investigated in the normal state (e.g., without inflating the IVC balloon). Four different pressure levels (−2, −10, −15, and −20 mm Hg) were applied for 15 minutes each and the rate of urine produced and creatinine clearance were determined. Pressure levels were controlled and determined at the regulator. Following the −20 mm Hg therapy, the IVC balloon was inflated to increase the pressure by 15-20 mm Hg. The same four negative pressure levels were applied. Urine output rate and creatinine clearance rate for the congested control kidney 802 and treated kidney 804 were obtained. The animals 800 were subject to congestion by partial occlusion of the IVC for 90 minutes. Treatment was provided for 60 minutes of the 90 minute congestion period.

Following collection of urine output and creatinine clearance data, kidneys from one animal were subjected to gross examination then fixed in a 10% neutral buffered formalin. Following gross examination, histological sections were obtained, examined, and magnified images of the sections were captured. The sections were examined using an upright Olympus BX41 light microscope and images were captured using an Olympus DP25 digital camera. Specifically, photomicrograph images of the sampled tissues were obtained at low magnification (20× original magnification) and high magnification (100× original magnification). The obtained images were subjected to histological evaluation. The purpose of the evaluation was to examine the tissue histologically and to qualitatively characterize congestion and tubular degeneration for the obtained samples.

Surface mapping analysis was also performed on obtained slides of the kidney tissue. Specifically, the samples were stained and analyzed to evaluate differences in size of tubules for treated and untreated kidneys. Image processing techniques calculated a number and/or relative percentage of pixels with different coloration in the stained images. Calculated measurement data was used to determine volumes of different anatomical structures.

Results

Urine Output and Creatinine Clearance

Urine output rates were highly variable. Three sources of variation in urine output rate were observed during the study. The inter-individual and hemodynamic variability were anticipated sources of variability known in the art. A third source of variation in urine output, upon information and belief believed to be previously unknown, was identified in the experiments discussed herein, namely, contralateral intra-individual variability in urine output.

Baseline urine output rates were 0.79 ml/min for one kidney and 1.07 ml/min for the other kidney (e.g., a 26% difference). The urine output rate is a mean rate calculated from urine output rates for each animal.

When congestion was provided by inflating the IVC balloon, the treated kidney urine output dropped from 0.79 ml/min to 0.12 ml/min (15.2% of baseline). In comparison, the control kidney urine output rate during congestion dropped from 1.07 ml/min to 0.09 ml/min (8.4% of baseline). Based on urine output rates, a relative increase in treated kidney urine output compared to control kidney urine output was calculated, according to the following equation:

$$Relative Increase = \frac{Therapy Treated / Baseline Treated}{Therapy Control / Baseline Control}$$

$$\frac{0.12 mL / min / 0.79 mL / min}{0.09 mL / min / 1.07 mL / min} = 180.6 \% Relative Increase$$

Thus, the relative increase in treated kidney urine output rate was 180.6% compared to control. This result shows a greater magnitude of decrease in urine production caused by congestion on the control side when compared to the treatment side. Presenting results as a relative percentage difference in urine output adjusts for differences in urine output between kidneys.

Creatinine clearance measurements for baseline, congested, and treated portions for one of the animals are shown in FIG. 22.

Gross Examination and Histological Evaluation

Based on gross examination of the control kidney (right kidney) and treated kidney (left kidney), it was determined that the control kidney had a uniformly dark red-brown color, which corresponds with more congestion in the control kidney compared to the treated kidney. Qualitative evaluation of the magnified section images also noted increased congestion in the control kidney compared to the treated kidney. Specifically, as shown in Table 1, the treated kidney exhibited lower levels of congestion and tubular degeneration compared to the control kidney. The following qualitative scale was used for evaluation of the obtained slides.

Congestion

| Lesion | Score |
|---|---|
| None: | 0 |
| Mild: | 1 |
| Moderate: | 2 |
| Marked: | 3 |
| Severe: | 4 |

Tubular degeneration

| Lesion | Score |
|---|---|
| None: | 0 |
| Mild: | 1 |
| Moderate: | 2 |
| Marked: | 3 |
| Severe: | 4 |

TABLE 1
TABULATED RESULTS

| | | Histologic lesions | | |
|---|---|---|---|---|
| | | | Tubular | |
| Animal ID/Organ/Gross | Slide | Conges- | hyaline | Granu- |
| lesion | number | tion | casts | lomas |
| 6343/Left Kidney/Normal | R16-513-1 | 1 | 1 | 0 |
| 6343/Left Kidney/Normal with | R16-513-2 | 1 | 1 | 0 |
| hemorrhagic streak | | | | |
| 6343/Right Kidney/Congestion | R16-513-3 | 2 | 2 | 1 |
| 6343/Right Kidney/Congestion | R16-513-4 | 2 | 1 | 1 |

As shown in Table 1, the treated kidney (left kidney) exhibited only mild congestion and tubular degeneration. In contrast, the control kidney (right kidney) exhibited moderate congestion and tubular degeneration. These results were obtained by analysis of the slides discussed below.

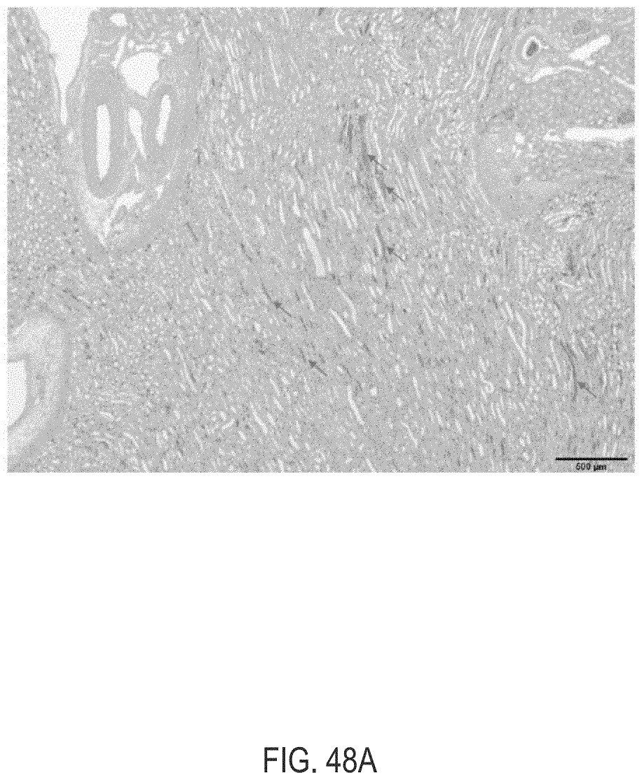

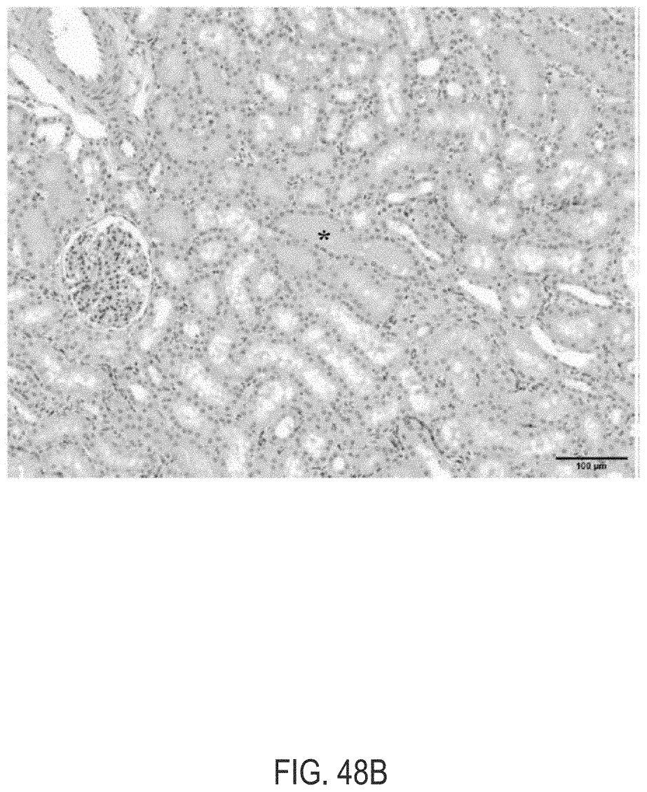

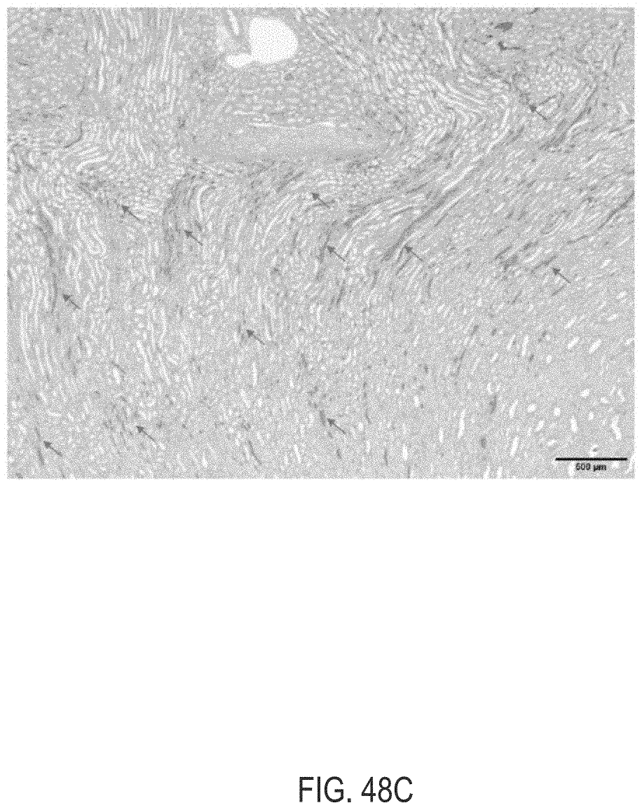

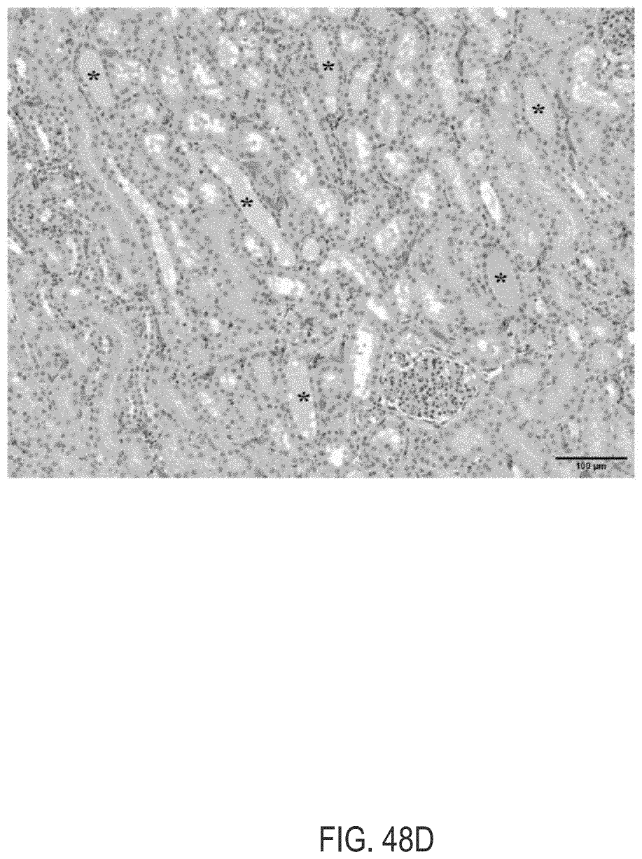

FIGS. 48A and 48B are low and high magnification photomicrographs of the left kidney (treated with negative pressure) of the animal. Based on the histological review, mild congestion in the blood vessels at the corticomedullary junction was identified, as indicated by the arrows. As shown in FIG. 48B, a single tubule with a hyaline cast (as identified by the asterisk) was identified.

FIGS. 48C and 48D are low and high resolution photomicrographs of the control kidney (right kidney). Based on the histological review, moderate congestion in the blood vessel at the corticomedullary junction was identified, as shown by the arrows in FIG. 48C. As shown in FIG. 48D, several tubules with hyaline casts were present in the tissue sample (as identified by asterisks in the image). Presence of a substantial number of hyaline casts is evidence of hypoxia.

Surface mapping analysis provided the following results. The treated kidney was determined to have 1.5 times greater fluid volume in Bowman's space and 2 times greater fluid volume in tubule lumen. Increased fluid volume in Bowman's space and the tubule lumen corresponds to increased urine output. In addition, the treated kidney was determined to have 5 times less blood volume in capillaries compared to the control kidney. The increased volume in the treated kidney appears to be a result of (1) a decrease in individual capillary size compared to the control and (2) an increase in the number of capillaries without visible red blood cells in the treated kidney compared to the control kidney, an indicator of less congestion in the treated organ.

Summary

These results indicate that the control kidney had more congestion and more tubules with intraluminal hyaline casts, which represent protein-rich intraluminal material, compared to the treated kidney. Accordingly, the treated kidney exhibits a lower degree of loss of renal function. While not intending to be bound by theory, it is believed that as severe congestion develops in the kidney, hypoxemia of the organ follows. Hypoxemia interferes with oxidative phosphorylation within the organ (e.g., ATP production). Loss of ATP and/or a decrease in ATP production inhibits the active transport of proteins causing intraluminal protein content to increase, which manifests as hyaline casts. The number of renal tubules with intraluminal hyaline casts correlates with the degree of loss of renal function. Accordingly, the reduced number of tubules in the treated left kidney is believed to be physiologically significant. While not intending to be bound by theory, it is believed that these results show that damage to the kidney can be prevented or inhibited by applying negative pressure to a ureteral catheter inserted into the renal pelvis to facilitate urine output.

### Example 2

Method

Four (4) farm swine (A, B, C, D) were sedated and anesthetized. Vitals for each of the swine were monitored throughout the experiment and cardiac output was measured at the end of each 30-minute phase of the study. Ureteral catheters, such as the ureteral catheter 112 shown in FIGS. 8A and 8B, were deployed in the renal pelvis region of the kidneys of each of the swine. The deployed catheters were a 6 Fr catheter having an outer diameter of 2.0±0.1 mm. The catheters were 54±2 cm in length, not including the distal retention portion. The retention portion was 16±2 mm in length. As shown in the catheter 112 in FIGS. 8A and 8B, the retention portion included two full coils and one proximal half coil. The outer diameter of the full coils, shown by line D1 in FIGS. 2A and 2B, was 18±2 mm. The half coil diameter D2 was about 14 mm. The retention portion of the deployed ureteral catheters included six drainage openings, plus an additional opening at the distal end of the catheter tube. The diameter of each of the drainage openings was 0.83±0.01 mm. The distance between adjacent drainage openings 132, specifically the linear distance between drainage openings when the coils were straightened, was 22.5±2.5 mm.

The ureteral catheters were positioned to extend from the renal pelvis of the swine, through the bladder, and urethra, and to fluid collection containers external to each swine. Following placement of the ureteral catheters, pressure sensors for measuring IVC pressure were placed in the IVC at a position distal to the renal veins. An inflatable balloon catheter, specifically a PTS® percutaneous balloon catheter (30 mm diameter by 5 cm length), manufactured by NuMED Inc. of Hopkinton, N.Y., was expanded in the IVC at a position proximal to the renal veins. A thermodilution catheter, specifically a Swan-Ganz thermodilution pulmonary artery catheter manufactured by Edwards Lifesciences Corp. of Irvine, Calif., was then placed in the pulmonary artery for the purpose of measuring cardiac output.

Initially, baseline urine output was measured for 30 minutes, and blood and urine samples were collected for biochemical analysis. Following the 30-minute baseline period, the balloon catheter was inflated to increase IVC pressure from a baseline pressure of 1-4 mm Hg to an elevated congested pressure of about 20 mm Hg (+/−5 mm Hg). A congestion baseline was then collected for 30 minutes with corresponding blood and urine analysis.

At the end of the congestion period, the elevated congested IVC pressure was maintained and negative pressure diuresis treatment was provided for swine A and swine C. Specifically, the swine (A, C) were treated by applying a negative pressure of −25 mm Hg through the ureteral catheters with a pump. As in previously-discussed examples, the pump was an Air Cadet Vacuum Pump from Cole-Parmer Instrument Company (Model No. EW-07530-85). The pump was connected in series to a regulator. The regulator was a V-800 Series Miniature Precision Vacuum Regulator—1/8 NPT Ports (Model No. V-800-10-W/K), manufactured by Airtrol Components Inc. The swine were observed for 120 minutes, as treatment was provided. Blood and urine collection were performed every 30 minutes, during the treatment period. Two of the swine (B, D) were treated as congested controls (e.g., negative pressure was not applied to the renal pelvis through the ureteral catheters), meaning that the two swine (B, D) did not receive negative pressure diuresis therapy.

Following collection of urine output and creatinine clearance data for the 120-minute treatment period, the animals were sacrificed and kidneys from each animal were subjected to gross examination. Following gross examination, histological sections were obtained and examined, and magnified images of the sections were captured.

Results

Measurements collected during the Baseline, Congestion, and Treatment periods are provided in Table 2. Specifically, urine output, serum creatinine, and urinary creatinine measurements were obtained for each time period. These values allow for the calculation of a measured creatinine clearance as follows:

$$Creatinine Clearance : CrCl = Urine Output ( m l / min ) * \frac{Urinary Creatinine ( mg / dl )}{Serum Creatinine ( mg / dl )}$$

In addition, Neutrophil gelatinase-associated lipocalin (NGAL) values were measured from serum samples obtained for each time period and Kidney Injury Molecule 1 (KIM-1) values were measured from the urine samples obtained for each time period. Qualitative histological findings determined from review of the obtained histological sections are also included in Table 2.

TABLE 2

| Animal | A | | B | | C | | D | |
|---|---|---|---|---|---|---|---|---|
| Treatment assignment | Treatment | | Control | | Treatment | | Control | |
| Baseline: | | | | | | | | |
| Urine output (ml/min) | 3.01 | | 2.63 | | 0.47 | | 0.98 | |
| Serum creatinine (mg/dl) | 0.8 | | 0.9 | | 3.2 | | 1.0 | |
| Creatinine clearance (ml/min) | 261 | | 172 | | 5.4 | | 46.8 | |
| Serum NGAL (ng/ml) | 169 | | * | | 963 | | 99 | |
| Urinary KIM-1 (ng/ml) | 4.11 | | * | | 3.59 | | 1.16 | |
| Congestion: | | | | | | | | |
| Urine output (ml/min) | 0.06 | (2%) | 0.53 | (20%) | 0.12 | (25%) | 0.24 | (25%) |
| Serum creatinine (mg/dl) | 1.2 | (150%) | 1.1 | (122%) | 3.1 | (97%) | 1.2 | (120%) |
| Creatinine clearance (ml/min) | 1.0 | (0.4%) | 30.8 | (18%) | 1.6 | (21%) | 16.2 | (35%) |

| | | | | | | | |
|---|---|---|---|---|---|---|---|
| Serum NGAL (ng/ml) | 102 | (60%) | * | 809 | (84%) | 126 | (127%) |
| Urinary KIM-1 (ng/ml) | 24.3 | (591%) | * | 2.2 | (61%) | 1.39 | (120%) |
| Treatment: | | | | | | | |
| Urine output (ml/min) | 0.54 | (17%) | | 0.47 | (101%) | 0.35 | (36%) |
| Serum creatinine (mg/dl) | 1.3 | (163%) | | 3.1 | (97%) | 1.7 | (170%) |
| Creatinine clearance (ml/min) | 30.6 | (12%) | ** | 18.3 | (341%) | 13.6 | (29%) |
| Serum NGAL (ng/ml) | 197 | (117%) | | 1104 | (115%) | 208 | (209%) |
| Urinary KIM-1 (ng/ml) | 260 | (6326%) | | 28.7 | (799%) | 233 | (20000%) |

Histological findings:

| | | | | |
|---|---|---|---|---|
| Blood volume in capillary space | 2.4% | | 0.9% | 4.0% |
| Hyaline casts | Mild/Mod | ** | None | Mod |
| Degranulation | Mild/Mod | | None | Mod |

Data are raw values (% baseline)
* not measured
** confounded by phenylephrine

Animal A: The animal weighed 50.6 kg and had a baseline urine output rate of 3.01 ml/min, a baseline serum creatinine of 0.8 mg/dl, and a measured CrCl of 261 ml/min. It is noted that these measurements, aside from serum creatinine, were uncharacteristically high relative to other animals studied. Congestion was associated with a 98% reduction in urine output rate (0.06 ml/min) and a >99% reduction in CrCl (1.0 ml/min). Treatment with negative pressure applied through the ureteral catheters was associated with urine output and CrCl of 17% and 12%, respectively, of baseline values, and 9× and >10×, respectively, of congestion values. Levels of NGAL changed throughout the experiment, ranging from 68% of baseline during congestion to 258% of baseline after 90 minutes of therapy. The final value was 130% of baseline. Levels of KIM-1 ωere 6 times and 4 times of baseline for the first two 30-minute windows after baseline assessment, before increasing to 68×, 52×, and 63× of baseline values, respectively, for the last three collection periods. The 2-hour serum creatinine was 1.3 mg/dl. Histological examination revealed an overall congestion level, measured by blood volume in capillary space, of 2.4%. Histological examination also noted several tubules with intraluminal hyaline casts and some degree of tubular epithelial degeneration, a finding consistent with cellular damage.

Animal B: The animal weighed 50.2 kg and had a baseline urine output rate of 2.62 ml/min and a measured CrCl of 172 ml/min (also higher than anticipated). Congestion was associated with an 80% reduction in urine output rate (0.5 ml/min) and an 83% reduction in CrCl (30 ml/min). At 50 minutes into the congestion (20 minutes after the congestion baseline period), the animal experienced an abrupt drop in mean arterial pressure and respiration rate, followed by tachycardia. The anesthesiologist administered a dose of phenylephrine (75 mg) to avert cardiogenic shock. Phenylephrine is indicated for intravenous administration when blood pressure drops below safe levels during anesthesia. However, since the experiment was testing the impact of congestion on renal physiology, administration of phenylephrine confounded the remainder of the experiment.

Animal C: The animal weighed 39.8 kg and had a baseline urine output rate of 0.47 ml/min, a baseline serum creatinine of 3.2 mg/dl, and a measured CrCl of 5.4 ml/min. Congestion was associated with a 75% reduction in urine output (0.12 ml/min) and a 79% reduction in CrCl (1.6 ml/min). It was determined that baseline NGAL levels were >5× the upper limit of normal (ULN). Treatment with negative pressure applied to the renal pelvis through the ureteral catheters was associated with a normalization of urine output (101% of baseline) and a 341% improvement in CrCl (18.2 ml/min). Levels of NGAL changed throughout the experiment, ranging from 84% of baseline during congestion to 47% to 84% of baseline between 30 and 90 minutes. The final value was 115% of baseline. Levels of KIM-1 decreased 40% from baseline within the first 30 minutes of congestion, before increasing to 8.7×, 6.7×, 6.6×, and 8× of baseline values, respectively, for the remaining 30-minute windows. Serum creatinine level at 2 hours was 3.1 mg/dl. Histological examination revealed an overall congestion level, measured by blood volume in capillary space, of 0.9%. The tubules were noted to be histologically normal.

Animal D: The animal weighed 38.2 kg and had a baseline urine output of 0.98 ml/min, a baseline serum creatinine of 1.0 mg/dl, and a measured CrCl of 46.8 ml/min. Congestion was associated with a 75% reduction in urine output rate (0.24 ml/min) and a 65% reduction in Cr Cl (16.2 ml/min). Continued congestion was associated with a 66% to 91% reduction of urine output and 89% to 71% reduction in CrCl. Levels of NGAL changed throughout the experiment, ranging from 127% of baseline during congestion to a final value of 209% of baseline. Levels of KIM-1 remained between 1× and 2× of baseline for the first two 30-minute windows after baseline assessment, before increasing to 190×, 219×, and 201× of baseline values for the last three 30-minute periods. The 2-hour serum creatinine level was 1.7 mg/dl. Histological examination revealed an overall congestion level 2.44× greater than that observed in tissue samples for the treated animals (A, C) with an average capillary size 2.33 times greater than that observed in either of the treated animals. The histological evaluation also noted several tubules with intraluminal hyaline casts as well as tubular epithelial degeneration, indicating substantial cellular damage.

Summary

While not intending to be bound by theory, it is believed that the collected data supports the hypothesis that venous congestion creates a physiologically significant impact on renal function. In particular, it was observed that elevation of the renal vein pressure reduced urine output by 75% to 98% within seconds. The association between elevations in biomarkers of tubular injury and histological damage is consistent with the degree of venous congestion generated, both in terms of magnitude and duration of the injury.

The data also appears to support the hypothesis that venous congestion decreases the filtration gradients in the medullary nephrons by altering the interstitial pressures. The change appears to directly contribute to the hypoxia and cellular injury within medullary nephrons. While this model does not mimic the clinical condition of AKI, it does provide insight into the mechanical sustaining injury.

The data also appears to support the hypothesis that applying negative pressure to the renal pelvis through ureteral catheters can increase urine output in a venous congestion model. In particular, negative pressure treatment was associated with increases in urine output and creatinine clearance that would be clinically significant. Physiologically meaningful decreases in medullary capillary volume and smaller elevations in biomarkers of tubular injury were also observed. Thus, it appears that by increasing urine output rate and decreasing interstitial pressures in medullary nephrons, negative pressure therapy may directly decrease congestion. While not intending to be bound by theory, by decreasing congestion, it may be concluded that negative pressure therapy reduces hypoxia and its downstream effects within the kidney in a venous congestion mediated AKI.

The experimental results appear to support the hypothesis that the degree of congestion, both in terms of the magnitude of pressure and duration, is associated with the degree of cellular injury observed. Specifically, an association between the degree of urine output reduction and the histological damage was observed. For example, treated Swine A, which had a 98% reduction in urine output, experienced more damage than treated Swine C, which had a 75% reduction in urine output. As would be expected, control Swine D, which was subjected to a 75% reduction in urine output without benefit of therapy for two and a half hours, exhibited the most histological damage. These findings are broadly consistent with human data demonstrating an increased risk for AKI onset with greater venous congestion. See e.g., Legrand, M. et al., Association between systemic hemodynamics and septic acute kidney injury in critically ill patients: a retrospective observational study. Critical Care 17:R278-86, 2013.

### Example 3

Method

Inducement of negative pressure within the renal pelvis of farm swine using ureteral catheters was performed for the purpose of evaluating effects of negative pressure therapy on hemodilution of the blood. An objective of these studies was to demonstrate whether a negative pressure delivered into the renal pelvis significantly increases urine output in a swine model of fluid resuscitation.

Two pigs were sedated and anesthetized using ketamine, midazolam, isoflurane and propofol. One animal (#6543) was treated with a ureteral catheter and negative pressure therapy as described herein. The other, which received a Foley type bladder catheter, served as a control (#6566). Following placement of the ureretal catheters, the animals were transferred to a sling and monitored for 24 hours.

Fluid overload was induced in both animals with a constant infusion of saline (125 mL/hour) during the 24 hour follow-up. Urine output volume was measured at 15 minute increments for 24 hours. Blood and urine samples were collected at 4 hour increments. As shown in FIG. 21, a therapy pump 818 was set to induce negative pressure within the renal pelvis 820, 821 (shown in FIG. 21) of both kidneys using a pressure of −45 mmHg (+/−2 mm Hg).

Results

Both animals received 7 L of saline over the 24 hour period. The treated animal produced 4.22 L of urine while the control produced 2.11 L. At the end of 24 hours, the control had retained 4.94 L of the 7 L administered, while the treated animal retained 2.81 L of the 7 L administered. FIG. 26 illustrates the change in serum albumin. The treated animal had a 6% drop in the serum albumin concentration over 24 hours, while the control animal had a 29% drop.

Summary

While not intending to be bound by theory, it is believed that the collected data supports the hypothesis that fluid overload induces clinically significant impact on renal function and, consequently induces hemodilution. In particular, it was observed that administration of large quantities of intravenous saline cannot be effectively removed by even healthy kidneys. The resulting fluid accumulation leads to hemodilution. The data also appears to support the hypothesis that applying negative pressure diuresis therapy using ureteral catheters to fluid overloaded animals can increase urine output, improve net fluid balance and decrease the impact of fluid resuscitation on development of hemodilution.

### Example 4

Method

An animal model of hypervolemic tamponade has been previously reported to replicate volume overload, venous congestion and the wet warm phenotype of acute decompensated heart failure (ADHF). This model also induces renal dysfunction comparable to the cardiorenal syndrome in ADHF. However, there have been no previous reports of animal models addressing the utility of diuretics or renal negative pressure treatment on this syndrome. Rao V, Turner J, Griffen M et al., First-in-Human Experience With Peritoneal Direct Sodium Removal Using a Zero-Sodium Solution: A New Candidate Therapy for Volume Overload, Circulation (2020) 141:1043-1053. Therefore, the prior model was modified to investigate the effects of a renal negative pressure treatment in conjunction with loop diuretics. This study examined the effects these treatments have on congestion via volume overload and the cardiorenal syndrome during ADHF via tamponade in pigs, while also allowing comparison between the standard first-line clinical approach of using only diuretics for treatment versus using a renal negative pressure treatment system in conjunction with diuretics.

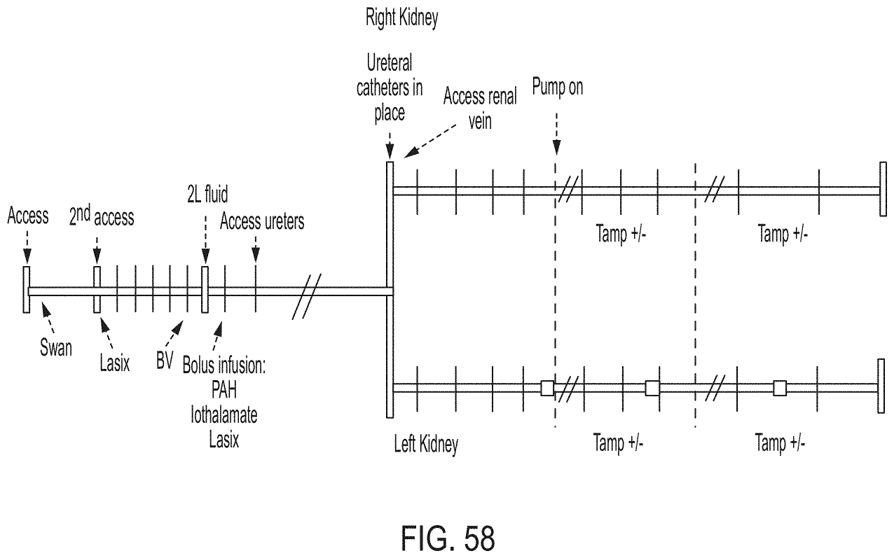

Ten swine were anesthetized followed by insertion of vein and arterial catheters for fluid loading and blood pressure monitoring, respectively. Tamponade was induced via volume loading of the pericardial space with 6% hydroxyethyl starch. This pericardial fluid loading elevates pericardial pressure and creates a fluid compression of the heart to reflect right-sided heart failure. The test procedure is shown in FIG. 58.

Ten ˜80 kg anesthetized pigs underwent left lateral thoracotomy with implantation of an intra-pericardial catheter, a Swan Ganz catheter, and placement of a JuxtaFlow® catheter into the renal pelvis of each kidney. The JuxtaFlow® catheter is a memory polymer catheter which creates a 3-dimensional helix in the renal pelvis upon deployment. High dose of furosemide (400 mg bolus followed by 80 mg/hr infusion) was administered at the beginning of fluid loading. Each animal served as its own control with randomization of the left vs. right kidney to −30 mm Hg renal negative pressure treatment. HF was induced via cardiac tamponade. After administration of 4 liters of IV normal saline, ˜200 ml of 6% hydroxyethyl starch was instilled into the pericardium. Pericardial pressure was increased to a target of 20-22.5 mm Hg and maintained at that level via a combination of IV normal saline and additional pericardial fluid. FIG. 58 illustrates the timeline for the procedure with respect to each animal.

Summary

With saline loading alone (hypervolemia), mild congestion was achieved as noted by an increase in central venous pressure (CVP) to approximately 8 mm Hg. Normal CVP is 0-6 mm Hg. Treatment of this congested state with negative pressure using a system as described herein (−30 mm Hg) resulted in a significant increase in urine output rate (+69%), sodium mass excretion (+67%) and creatinine clearance (+25%) beyond the use of diuretics alone. The results are illustrated in FIG. 59.

During tamponade (fluid loading of the pericardium), significant congestion was noted by an increase of CVP to approximately 20 mm Hg. Despite the continued presence of furosemide, these elevated venous pressures were associated with a significant decrease in urine output (−35%), mass sodium excretion (−40%), and creatinine clearance (−36%) compared to the mild congestion state seen in saline loading. Treatment with negative pressure using a system as described herein (−30 mm Hg) in combination with the administration of furosemide resulted in a significant and unexpected synergistic increase in urine output (+67%) (FIG. 59A), mass sodium excretion (+72%) (FIG. 59B) and creatinine clearance (+26%) (FIG. 59C) beyond the use of diuretics alone.

During furosemide diuresis and prior to induction of heart failure, renal negative pressure treatment resulted in a significant increase in urine output, sodium excretion, and creatinine clearance from the treated kidney compared to the non-treated kidney (p<0.001 for all, FIG. 59). Induction of HF resulted in the target hemodynamic profile with relatively stable cardiac output, but severely elevated filling pressures (Figure). Urine output, sodium excretion, and creatinine clearance significantly decreased during HF (p<0.001 for all, Figure), but were consistently greater in the treated kidney compared to the control kidney (p<0.05 for all, FIG. 59). Notably, urine output (p=0.38) and natriuresis (p=0.91) was the same during HF in the treated kidney as the control kidney prior to HF.

### Example 5: Renal Negative Pressure Treatment with Furosemide

Example 5 evaluates use of Negative Pressure Treatment (rNPT) for improvement of diuresis, natriuresis, and renal function in a congestion heart failure (HF) model.

Method

Ten Yorkshire farm pigs that were from 18-20 weeks of age (˜80 kg) were used to investigate effects of renal Negative Pressure Treatment (rNPT) using the JuxtaFlow® catheter and pump system. As previously discussed, the JuxtaFlow® catheter is a memory polymer catheter which deploys into a 3-dimensional helix when placed in the renal pelvis allowing application of negative pressure to the kidney without causing tissue collapse or obstruction. The JuxtaFlow® catheter is similar or identical to the ureteral catheter 112 described previously and shown, for example, in FIGS. 8A and 8B. The JuxtaFlow® pump is a tightly controlled, self-regulating negative pressure pump system designed for use with the JuxtaFlow® catheter and rNPT. The JuxtaFlow® pump includes features of the pump 710 shown in FIG. 44.

In order to deploy the JuxtaFlow® catheters, after an overnight fast, pigs were anesthetized with a combination of intramuscular ketamine and tiletamine/zolazepam (Telazol), intubated, and maintained on inhaled isoflurane. An intra-pericardial catheter was placed via a left lateral thoracotomy. A Swan-Ganz catheter was placed via a right internal jugular vein cutdown. An arterial line for continuous hemodynamic monitoring was placed in the carotid or femoral artery by either Seldinger technique or arterial cutdown. Large bore central venous access was similarly placed in either the contralateral jugular or a femoral vein for fluid and tracer infusions. To catheterize the ureters, the bladder was retracted caudally through a small suprapubic incision and each ureter was isolated and directly cannulated through a small incision. The JuxtaFlow® catheters were then advanced into the renal pelvis under fluoroscopic guidance. Each kidney was drained through the JuxtaFlow® catheters either passively or under negative pressure provided by the JuxtaFlow® pump for applying rNPT.

Given that the human heart failure use-case for the JuxtaFlow® catheter and system may be in conjunction with intravenous loop diuretic use, two experimental phases were conducted to investigate the effect of rNPT: 1) during maximal furosemide diuresis without heart failure (HF); and 2) in a state of HF characterized by venous congestion and concurrent furosemide diuresis. During the two phases, each animal served as its own control with randomization of either the left or right kidney to rNPT versus No-rNPT. The experiment was started with an equilibration period where intravenous (IV) boluses and continuous infusion of the following agents were initiated and maintained for a period of 2.5 hours: iothalamate (“IOT”, 120 mg bolus with 0.3 mg/min infusion, Guerbet, USA); para-aminohippurate (“PAH”, 800 mg bolus with 8.4 mg/min infusion, MilliporeSigma, USAsupplier); and furosemide (400 mg bolus with infusion at 80 mg/hr). To avoid volume depletion, a 4 Liter IV infusion of normal saline followed by a maintenance IV infusion titrated to match urine output 1:1 (mL) occurred during this equilibrium period.

Experimental Periods

After equilibration of tracers, the right and left kidneys were randomized (−30 mmHg rNPT was applied to one kidney, while the other kidney drained by passive drainage) and the rNPT therapy was started. In order to ensure that the pre-tamponade and tamponade experimental periods had similar background fluid status, a rapid, large-volume, normal saline infusion of 20% to 25% body weight occurred at this point. After 10 minutes of equilibration, the animals underwent two 15 minute “post fluid” clearance periods. Next, cardiac tamponade was induced by pericardial instillation of approximately 200 mL of 6% hydroxyethyl starch. Pericardial hydroxyethyl starch and additional IV normal saline infusion were titrated to maintain a hemodynamic profile sufficient for relative preservation of cardiac output and mean arterial pressure (compared to the baseline pre-fluid readings), while maintaining a central venous pressure of less than 20 mmHg. After stabilization and a 10 minute equilibration, two 15 minute study periods were repeated.

Assays and Calculations

A Randox Imola automated clinical chemistry analyzer was used to measure concentration of urine or serum chemistry parameters. The calibrators, reagents, and urine Level 2 and Level 3 controls were purchased from Randox Laboratories. All assay measurements were carried out in accordance with the manufacturer's instructions (Randox Laboratories, UK). Creatinine measurements were standardized to Isotope Dilution Mass Spectrometry (IDMS) traceable National Institute of Standards and Technology reference material (SRM 967). Urine and plasma iothalamate were measured using Agilent 6490 QTOF equipped with Agilent 1290 UHPLC.

A stock solution of iothalamate was serially diluted in 0.1% formic acid containing deuterated iothalamate to create the calibration curve (1-2000 ng/ml). Plasma samples (100 μL) were deproteinized by adding 300 μL of 100% methanol containing deuterated iothalamate (1000 ng/ml) (Cambridge Isotope Laboratories, Inc), vortexed, and centrifuged at 12,000 rpm for 10 minutes. 200 μL of the supernatant was then transferred to glass sampler vials, and 10 μL of the sample was injected to the UHPLC-MS/MS system. The urine samples were diluted 10-fold with 0.1% formic acid containing the internal standard. 10 μL of the diluted urine sample was injected into the UHPLC-MS/MS system. Separation was achieved using Agilent Zorbax Eclipse plus RP 2.1×50 mm 1.8 μm column with a constant flow rate of 400 μL/min. An instrument-controlled gradient of 0.1% formic acid and 100% methanol were used as Buffer A and Buffer B, respectively. Quantitation was carried out using the Agilent MassHunter Quantitative analysis software. Urine and plasma PAH were measured using the PAH colorimetric assay kit from Abcam, according to manufacturer's recommendation. Urine neutrophil gelatinase-associated lipocalin (NGAL) was measured with porcine NGAL kit from Alpco (Alpco, Salem, N.H.). Urine cGMP concentrations were assayed using a commercially available competitive enzyme-linked immunosorbent assay kit according the manufacturer's guidelines (Parameter cGMP Assay, R&D Systems Inc, Minneapolis, Minn. USA).

Measured creatinine clearance was calculated as Urine creatinine×Volume of urine per minute/Plasma creatinine. Measured GFR was calculated as Urine iothalamate×Volume of urine per minute/Plasma iothalamate. Renal plasma flow was calculated as Urine PAH×Volume of urine per minute/Plasma PAH. Filtration fraction was calculated as GFR/(renal plasma flow/0.9). Fractional excretion of sodium (FENa) was calculated as was calculated as (Naᵤᵣᵢₙₑ/Naₛₑᵣᵤₘ)×(Crₛₑᵣᵤₘ/Crᵤᵣᵢₙₑ)×100%.

Statistical Analysis

Continuous data is shown as mean±standard deviation or median (quartile 1-quartile 3) according to observed distribution. Categorical data is shown as frequency (percentage). Variables with skewed distribution were log transformed to approximate normal distribution. Changes in continuous variables from baseline to post-fluid (No HF) or to HF model of venous congestion were compared with the paired t test. Changes in continuous variables during the experiments were analyzed via linear mixed models accounting for correlations within animals. rNPT and HF models of venous congestion were included as main factors (binary variables) in a full factorial model. Statistical significance was defined as 2-tailed P<0.05. Statistical analysis was performed with IBM SPSS Statistics version 26 (IBM Corp, Armonk, N.Y.) and Stata SE version 16.0 (StataCorp, College Station, Tex.).

### Results

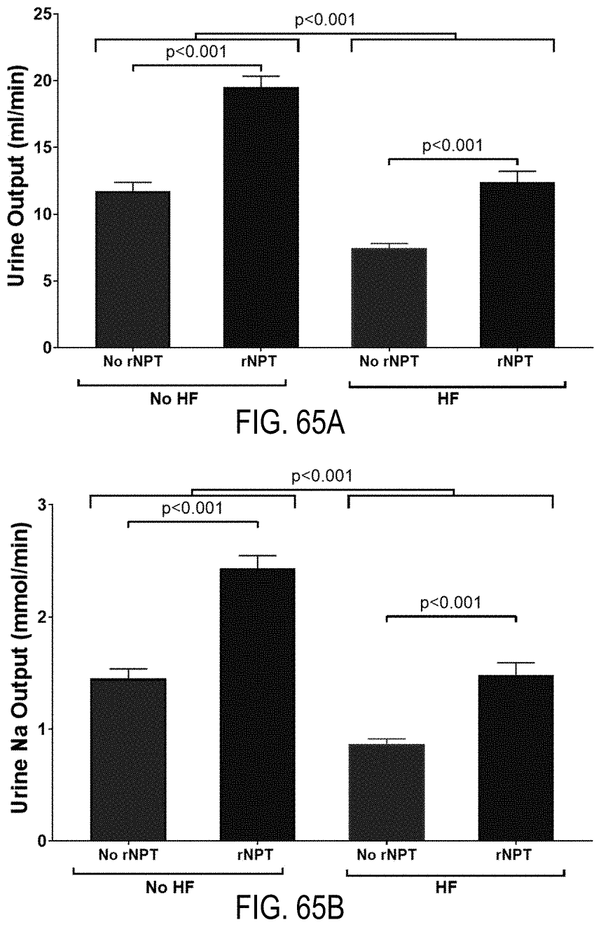

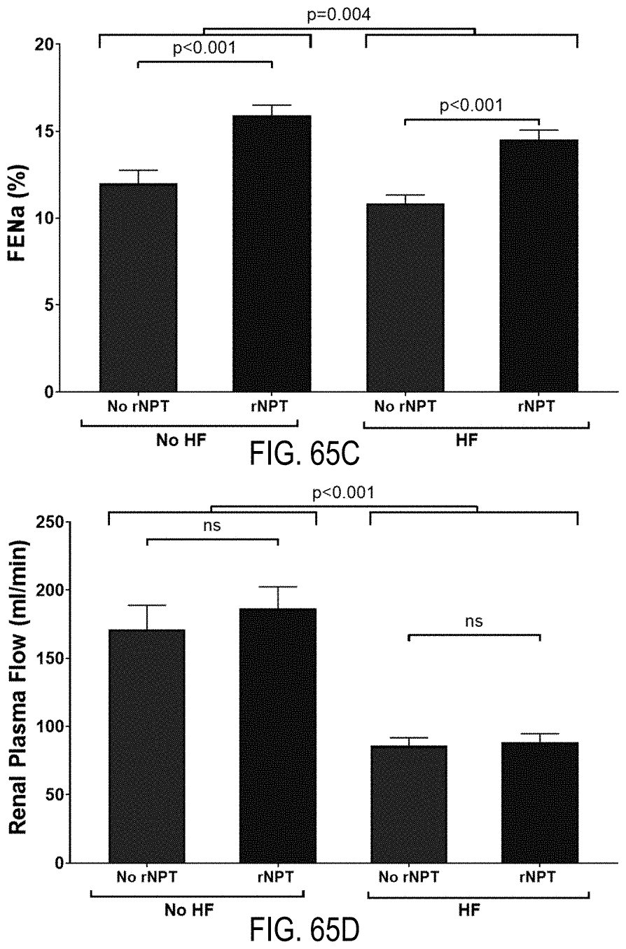

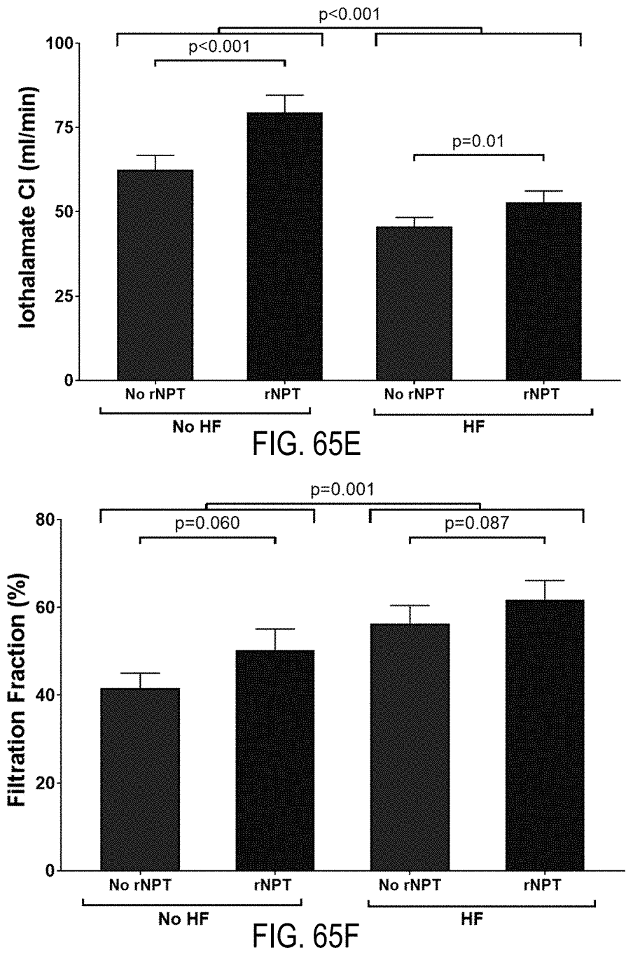

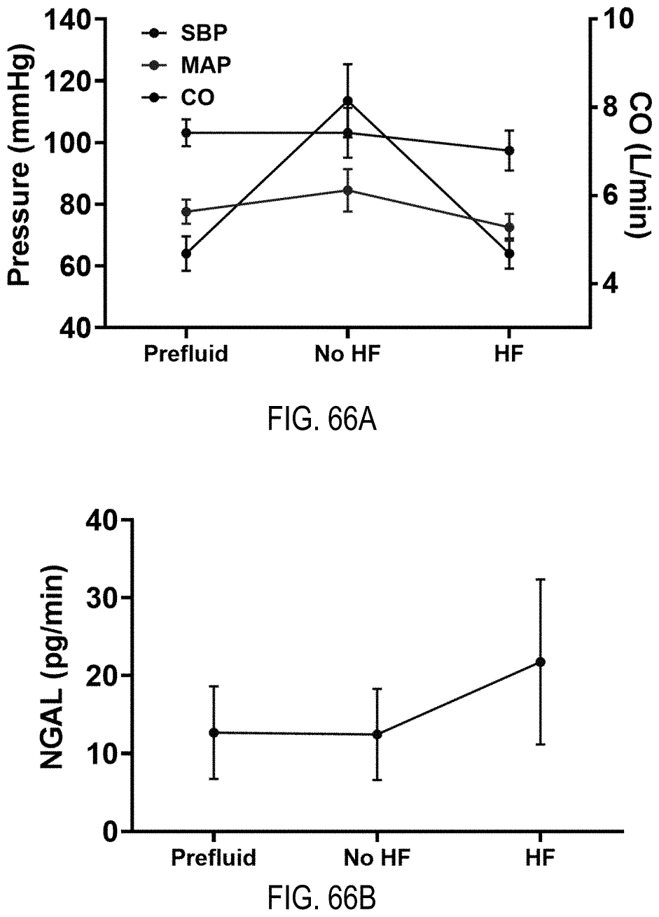

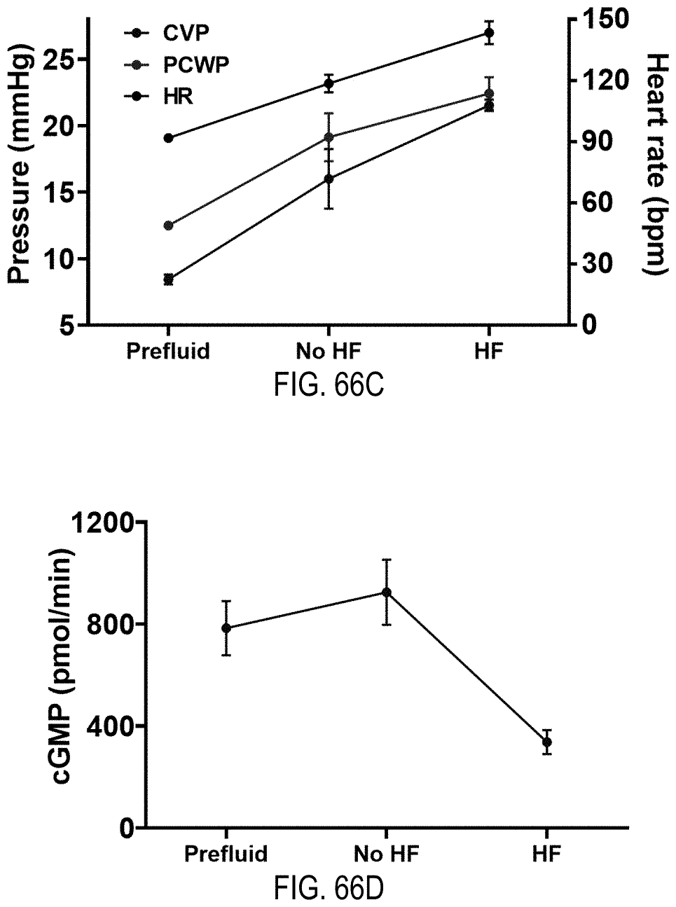

Results for Example 5 are shown in FIGS. 65A-66D. FIGS. 65A-65F are graphs illustrating results for urine output (FIG. 65A), cumulative urine sodium excretion (FIG. 65B), fractional excretion of sodium (FIG. 65C), renal plasma flow (FIG. 65D), glomerular filtration rate measured by iothalamate (IOH) (FIG. 65E), and filtration fraction (FIG. 65F). The graphs are presented as mean±standard error of the mean. Each graph compares a 15-minute baseline period without renal negative pressure therapy to a 15-minute period of renal negative pressure therapy in a non-HF state and in a HF model.

FIGS. 66A-66D are line graphs for hemodynamic variables collected during the experiments of Example 5. The hemodynamic variables are presented as mean±standard error of the mean across the three study periods: 1) before intravenous (IV) fluid administration (Prefluid), 2) after IV fluid administration with no heart failure (No HF), and 3) after induction of a HF model from cardiac tamponade. As shown in FIG. 66A, SBP and MAP (FIG. 66A) were not statistically different among the three periods (p>0.12 for each comparison), while CO increased from the prefluid to the No-HF period (p<0.01). CO was not statistically different in the HF model compared to the Prefluid period (p=0.90).

CVP, PCWP, and HR (FIG. 66C) increased significantly from the Prefluid to the No-HF period, and from the No-HF period to the HF model (p<0.05 for all comparisons). Neutrophil Gelatinase-Associated Lipocalin (NGAL) (FIG. 66B) did not change from the Prefluid to the No-HF period, but tended to increase from the No-HF to the HF period (p=0.053). Cyclic GMP (cGMP) (FIG. 66D) did not change from the Prefluid to the No-HF period, but decreased significantly from the No-HF to the HF period (p<0.001).

As illustrated by these figures, during furosemide diuresis, rNPT substantially increased natriuresis (2.4±0.6 mmol/min vs 1.5±0.5 mmol/min; p<0.001) and diuresis (19.7±4.5 ml/min vs 11.8±3.7 ml/min; p<0.001) compared to control. See FIGS. 65A and 65B. rNPT also increased iothalamate clearance (79±28 ml/min vs 62±23 ml/min; p<0.001) and creatinine clearance (105±38 vs. 85±30, p=0.001). See FIG. 65E. Renal plasma flow (p=0.13) did not differ significantly between rNPT and control. See FIG. 65D. The increased natriuresis with rNPT was not solely driven by increased sodium filtration, because the fractional excretion of sodium (FENa) was also higher with rNPT (15.9%±3.3% vs 12.0%±4.2%, p<0.001). See FIG. 65C.

The figures also show that induction of cardiac tamponade was successful in producing a “warm and wet” HF phenotype with preserved cardiac output and blood pressure, but with severely elevated right sided filling pressures. See FIGS. 66A and 66C. A cardio-renal phenotype also emerged as urine output (37%), renal sodium excretion (40%), measured GFR (27%), and renal plasma flow (50%) all decreased substantially with induction of HF (p<0.001). Furthermore, urine cyclic GMP decreased substantially (p<0.001) and NGAL tended to increase with induction of HF (p=0.053). Filtration fraction increased during induction of HF (42±18% vs 56±21%, p=0.001).

The effect of rNPT on GFR was similar between HF and No-HF periods (i.e., similar increments in GFR with rNPT; p interaction=0.23 for a different effect of rNPT in HF and no HF). rNPT did not significantly change renal plasma flow in either the HF or no HF periods (p=0.47 for the interaction). During HF, rNPT yielded greater urine output (276 ml±113 ml vs 167 ml±55 ml p<0.001) and urine sodium excretion (33.0±14.5 mmol vs 19.5±6.8 mmol; p<0.001) compared to the control kidney. See FIGS. 65A and 65B. FENa was also higher with rNPT (14.5%±3.0% vs 10.9%±2.7%, p<0.001). See FIG. 65C. Renal plasma flow did not change significantly with rNPT in HF (p=0.58). See FIG. 65D. Filtration fraction increased during rNPT (55.6±24.8% vs 49.0±20.8%; p=0.034) with a similar effect between HF and no HF periods (p=0.70 for the interaction). See FIG. 65F. Urine NGAL was similar with and without rNPT (p=0.70). Importantly, during HF, the rNPT kidney had similar urine output (p=0.52) and sodium excretion (p=0.87), and higher FENa (14.5±3.0% vs 12.0±4.2%; p=0.001) compared to the non-rNPT kidney without HF.

Discussion

The foregoing test results of Example 5 demonstrate that negative pressure applied to the renal pelvis during high dose furosemide therapy significantly improves a wide range of cardio-renal parameters, such as increased GFR, increased urine output, and increased sodium output. The mechanism of the increase in cumulative urine sodium excretion was not due purely to an increase in GFR, because both total and fractional sodium excretion increased. Importantly, the benefit appears to be of a clinically significant magnitude as urine output and sodium excretion with rNPT during experimental heart failure was similar to the non-rNPT kidney during the control period, i.e., after the heart failure model was shown to significantly decrease renal function, application of rNPT during heart failure appeared to restore renal function back to substantially normal levels.

As known to those skilled in the art, in ADHF, elevated central venous pressure is transmitted to renal veins. The transmission of central venous pressure to the renal veins increases renal venous pressure and decreases venous compliance, without changing renal arterial resistance or compliance. Renal venous congestion raises intrarenal and tubular pressure in the fixed space of the encapsulated kidney. While not intending to be bound by any theory, since alterations in renal venous blood flow normalize with decongestion, the inventors theorized that interventions to reduce intrarenal pressure may improve diuretic response and, potentially, ADHF outcomes. The experiments of Example 5 show that an intervention with renal negative pressure therapy increased urine output and urine sodium excretion. These findings are consistent with observations in humans with ADHF, where abnormal measurements of renal venous impedance and flow have been shown to be associated with higher sodium avidity, diminished diuretic response, and worsening HF outcomes independent of central venous pressure. In patients with ADHF, the inverse relationship between diuretic response and elevated renal venous impedance is independent of GFR. Similarly, the experiments of Example 5 show that the improved natriuresis with renal negative pressure therapy was not driven by improvement in GFR.

In addition to the beneficial effects in a HF model, renal negative pressure therapy appears to improve natriuresis and GFR during high dose furosemide therapy. This is an unexpected result because one might hypothesize that, in the normal state, increased filtration from rNPT would activate tubular glomerular feedback (TGF), which reduces filtration and brings GFR back to baseline. However, it is known that furosemide can reduce GFR. For example, it has been reported that furosemide administration can acutely increase proximal tubular pressure by ˜10 mmHg to ˜15 mmHg and increase renal interstitial pressure by ˜7 mmHg, both of which are likely contributors to the fall in GFR observed with loop diuretic administration. Thus, even in the absence of HF, furosemide would be expected to reduce GFR and elevate tubular and interstitial pressures, which could theoretically be improved with rNPT. This observation may be expected to have clinical relevance as the therapeutic value of rNPT may extend beyond the often-brief period when patients have intravascular congestion severe enough to negatively impact kidney function.

While not intending to be bound by theory, it is believed that the current observations from the congestion predominate HF model of Example 5 may shed light on the human literature on kidney dysfunction in human HF. The majority of contemporary human studies have not found a meaningful association between cardiac output and kidney function. Thus, the finding of Example 5 showing substantial deterioration in kidney function in an HF model with normal “forward flow” is congruent with conditions described in relevant literature. See e.g., Damman K, Navis G, Smilde T D et al., Decreased cardiac output, venous congestion and the association with renal impairment in patients with cardiac dysfunction, European journal of heart failure (2007) 9:872-878; Uthoff H, Breidthardt T, Klima T et al., Central venous pressure and impaired renal function in patients with acute heart failure, European journal of heart failure (2011) 13:432-9; and Nohria A, Hasselblad V, Stebbins A et al., Cardiorenal interactions: insights from the ESCAPE trial, Journal of the American College of Cardiology (2008) 51:1268-74. Further, these several human studies have noted an association between central venous pressure and renal function. However, it appears that these findings are heterogeneous with studies reporting that decongestion of patients with high CVP is associated with worsening kidney function in some individuals and improved kidney function for others. In Example 5, a large volume of intravenous normal saline substantially increased cardiac filling pressures, but available metrics of renal function were either unaffected or even improved. Upon induction of cardiac tamponade, a substantial reduction in natriuresis, renal plasma flow, GFR, and urinary cGMP was observed. Much like the human literature showing sometimes opposite effects of congestion on renal function and diuresis, the experiments of Example 5 appear to illustrate that the overall balance of natriuretic and anti-natriuretic factors ultimately determine the impact of volume expansion on kidney function.

While interpreting the results in Example 5, it should be considered that although the acute cardiac tamponade model employed sought to provide a relatively stable, predictable, and titratable “warm and wet” HF phenotype, acute tamponade is a rare human HF presentation. Thus, the findings of Example 5 may not extrapolate to acute or chronic decompensated human HF. Although the human-use case for the Juxtaflow® catheter and system may involve high dose loop diuretics, the lack of data in humans on the effect of rNPT in the absence of diuretics is a consideration. While the presumed mechanism underlying the improved renal function with rNPT is reduction of intra-tubular and interstitial pressure, this was not directly measured. Although utilizing the JuxtaFlow® catheter for both negative and atmospheric pressure provided a control for any mechanical effects of instrumenting the renal pelvis, the experiments of Example 5 did not measure delivered pressure at the level of the renal pelvis through the single lumen of the Juxtaflow® catheter. Thus, it was not determined if the actual delivered pressure deviated from −30 mmHg in the rNPT group and 0 mmHg in the non-rNPT group. However, Example 5 provides proof of concept results showing the benefits of rNPT to improve renal function in an acute cardiac tamponade model in pigs.

Example 5 shows that in the setting of high dose loop diuretic therapy in pigs, rNPT with the JuxtaFlow® catheter and pump system resulted in significantly increased diuresis, natriuresis, and mGFR. Importantly, the benefit appeared to be of clinically significant magnitude as urine output and sodium excretion with rNPT during experimental heart failure was similar to the non-rNPT kidney during the control period.

The preceding examples and embodiments of the invention have been described with reference to various examples. Modifications and alterations will occur to others upon reading and understanding the foregoing examples. Accordingly, the foregoing examples are not to be construed as limiting the disclosure.

1. A method for increasing urine output and/or sodium output from a patient having venous congestion, the method comprising:
(a) administering at least one medicament to a patient, wherein the medicament increases urine output and/or sodium output from the patient;
(b) applying negative pressure to a drainage lumen of a urinary catheter such that flow of urine from a ureter and/or kidney of the patient is transported within the drainage lumen to extract urine from the patient;
wherein administering the at least one medicament occurs before, during and/or after applying negative pressure.

2. The method according to claim 1, wherein the at least one medicament is selected from the group consisting of diuretic(s), SGLT-2 inhibitor(s), and combinations thereof.

3. The method according to claim 2, wherein the at least one medicament comprises at least one diuretic.

4. The method according to claim 3, wherein the at least one diuretic is selected from the group consisting of loop diuretic(s), carbonic anhydrase inhibitor(s), potassium-sparing diuretic(s), calcium-sparing diuretic(s), osmotic diuretic(s), thiazide diuretic(s), and combinations thereof.

5. The method according to claim 3, wherein the at least one diuretic comprises at least one loop diuretic.

6. The method according to claim 5, wherein the at least one loop diuretic is selected from the group consisting of bumetanide, ethacrynic acid, torsemide, furosemide, and combinations thereof.

7. The method according to claim 5, wherein the at least one loop diuretic is furosemide.

8. The method according to claim 2, wherein the at least one medicament comprises at least one SGLT-2 inhibitor.

9. The method according to claim 8, wherein the at least one SGLT-2 inhibitor is selected from the group consisting of ertugliflozin, canagliflozin, empagliflozin, dapagliflozin, and combinations thereof.

10. The method according to claim 1, wherein the patient is administered at least two medicaments, wherein each of the at least two medicaments is independently selected from the group consisting diuretic(s), SGLT-2 inhibitor(s), and combinations thereof.

11. The method according to claim 10, wherein the patient is administered at least one diuretic and at least one SGLT-2 inhibitor.

12. The method according to claim 10, wherein the at least one diuretic is furosemide.

13. The method according to claim 1, wherein the patient has fluid overload.

14. The method according to claim 1, wherein the patient has kidney impairment and/or reduced kidney function.

15. The method according to claim 1, wherein the patient has chronic kidney disease or acute kidney injury.

16. The method according to claim 1, wherein the patient has a GFR of 90 or lower, or a GFR of 60 or lower, or GFR of 44 or lower, or GFR of 30 or lower, prior to treatment.

17. The method according to claim 1, wherein the patient has heart failure or acute decompensated heart failure.

18. The method according to claim 1, wherein the patient has sepsis.

19. The method according to claim 1, wherein the urinary catheter further comprises: a proximal portion, a distal portion, and a drainage lumen, the distal portion, the distal portion comprising a retention portion that comprises at least one protected drainage hole(s), port(s) or perforation(s) and is configured to establish an outer periphery or protective surface area that inhibits mucosal tissue from occluding the at least one protected drainage hole(s), port(s) or perforation(s) upon application of negative pressure through the catheter.

20. The method according to claim 19, wherein the proximal portion of the urinary catheter is configured to pass through a percutaneous opening.

21. The method according to claim 19, wherein the at least one protected drainage hole(s), port(s) or perforation(s) are disposed on a protected surface area or inner surface area of the retention portion, and wherein the outer periphery or protective surface area of the retention portion of the catheter is configured to support the mucosal tissue and thereby prevent occlusion of the one or more of the protected drainage holes, ports or perforations upon application of negative pressure through the ureteral catheter.

22. The method according to claim 19, wherein the retention portion comprises one or more helical coils, each coil having an outwardly facing side and an inwardly facing side, and wherein the outer periphery or protective surface area comprises the outwardly facing side(s) of the one or more helical coil(s), and the at least one protected drainage hole(s), port(s) or perforation(s) are disposed on the inwardly facing side(s) of the one or more helical coil(s).

23. The method according to claim 19, wherein the retention portion is configured to be extended into a deployed position in which a diameter of the retention portion is greater than a diameter of the drainage lumen portion.

24. The method according to claim 19, wherein a number of the drainage holes, ports or perforations towards a distal end of the retention portion is greater that a number of the drainage holes, ports or perforations towards a proximal end of the retention portion.

25. The method according to claim 19, wherein a size of one or more of the drainage holes, ports or perforations towards a distal end of the retention portion is greater that a size of one or more of the drainage holes, ports or perforations towards a proximal end of the retention portion.

26. The method according to claim 19, wherein a total area of the drainage holes, ports or perforations towards a distal end of the retention portion is greater than a total area of the drainage holes, ports or perforations towards a proximal end of the retention portion.

27. The method according to claim 19, wherein a sidewall of the drainage lumen is essentially free or free of openings.

28. The method according to claim 1, wherein the at least one urinary catheter comprises at least one ureteral catheter and/or a bladder catheter.

29. The method according to claim 1, further comprising a negative pressure source operatively connected to the catheter,
the negative pressure source being configured to induce negative pressure in a portion of the urinary tract of the patient which causes fluid from the urinary tract to be drawn into the catheter at least partially through the one or more protected drainage holes, ports or perforations.

30. The method according to claim 29, wherein the negative pressure source is positioned within or outside of the patient's body.

31. The method according to claim 29, wherein the negative pressure source is a pump.

32. The method according to claim 29, further comprising a controller that is operatively connected to the negative pressure source and configured to actuate the negative pressure source to control the application of the negative pressure to a proximal end of the at least one urinary catheter.

33. The method according to claim 32, wherein the controller is positioned within or outside of the patient's body.

34. The method according to claim 32, further comprising one or more physiological sensors associated with the patient,
the physiological sensors being configured to provide information representative of at least one physical parameter to the controller, and
wherein the controller is configured to actuate or cease operation of the pump based on the information representative of the at least one physical parameter.

35. The method according to claim 1, wherein negative pressure is provided with a range of about 0.5 mm Hg to about 150 mm Hg.

36. The method according to claim 1, wherein the catheter is a ureteral catheter.

37. The method according to claim 1, wherein the catheter is a bladder catheter.

38. A method for increasing urine output and/or sodium output from a patient having venous congestion, the method comprising:
(a) administering a therapeutically effective amount of a medicament comprising furosemide to the patient; and
(b) applying negative pressure to a drainage lumen of at least one ureteral catheter to extract urine from the patient, the ureteral catheter comprising a proximal portion; a distal portion, and the drainage lumen, wherein the distal portion of the catheter comprises a retention portion comprising at least one protected drainage hole(s), port(s) or perforation(s) and is configured to establish an outer periphery or protective surface area that inhibits mucosal tissue from occluding the at least one protected drainage hole(s), port(s) or perforation(s) upon application of negative pressure through the catheter;
wherein administering the loop diuretic occurs before inducing negative pressure in the urinary tract of the patient.
